(19) United States
(12) Patent Application Publication
(10) Pub. No.: US 2024/0327917 A1
(43) Pub. Date: Oct. 3, 2024

(54) BIOMARKERS FOR AMYOTROPHIC LATERAL SCLEROSIS STRATIFICATION

(71) Applicants: Barbara Smith, Tempe, AZ (US); Jarrett Eshima, Phoenix, AZ (US); Chris Plaisier, Chandler, AZ (US)

(72) Inventors: Barbara Smith, Tempe, AZ (US); Jarrett Eshima, Phoenix, AZ (US); Chris Plaisier, Chandler, AZ (US)

(73) Assignee: Arizona Board of Regents on behalf of Arizona State University, Scottsdale, AZ (US)

(21) Appl. No.: 18/621,914

(22) Filed: Mar. 29, 2024

Related U.S. Application Data
(60) Provisional application No. 63/493,429, filed on Mar. 31, 2023.

(51) Int. Cl. C12Q 1/6883

(52) U.S. Cl. CPC C12Q 1/6883; C12Q 2600/112; C12Q 2600/118; C12Q 2600/158

(57) ABSTRACT

Provided are biomarkers for diagnosis of ALS and for differential diagnosis of distinct ALS subtypes. Also provided are methods of modulating the biomarkers for the treatment of ALS.

### CROSS REFERENCE TO RELATED APPLICATIONS

This application claims priority to U.S. Provisional Application No. 63/493,429; filed Mar. 31, 2023, which is hereby incorporated by reference herein in its entirety.

### BACKGROUND OF THE INVENTION

Amyotrophic lateral sclerosis (ALS) is a heterogenous neurodegenerative disease defined by the progressive loss of motor neuron function, eventually leading to respiratory failure and death. Clinical diagnosis remains slow, hampered by an absence of disease specific biomarkers, subjective scoring metrics, and presentation of symptoms that overlap with other motor neuron disorders early in the disease course. The lack of diagnostic and prognostic biomarkers has led to the utilization of a patient classification system based on the site of symptom onset (lower, upper, and bulbar), which poorly predicts differences in patient pathology, survival, treatment responsiveness, and symptom progression. As a consequence, inadequate clinical outcomes in ALS neurodegeneration are directly tied to underlying patient heterogeneity. Recent efforts have been directed towards identifying the phenotypes and mechanisms driving clinical heterogeneity in neurodegeneration. In Alzheimer's patients, neuroimaging-derived subtypes demonstrated differences in clinical presentation, survival, age of onset, rate of progression, and age of death, providing critical new insight into disease heterogeneity. Similarly, in the context of ALS, one group has recently developed a predictive model to stratify patients and inform prognosis, using patient-derived clinical information.

Current strategies to assess the molecular foundation of ALS heterogeneity have primarily applied ‘-omic’ methodologies in combination with unsupervised clustering for disease subtype discovery. Previous studies have used frontal and motor postmortem cortex transcriptomics to stratify a cohort of 77 ALS patients into three distinct subtypes. Further, the direct interplay between TDP-43 and transposable elements using eCLIP-seq was demonstrated, providing key insight into the pathological role of transposable elements in ALS, given the near ubiquitous nature of TDP-43 cellular inclusions (˜97%). However, there is no data establishing a direct link between the ALS subtypes and clinical outcomes, such as survival and age of onset.

Thus, there remains a need in the art for biomarkers for effective diagnosis and therapeutics for ALS and other neurodegenerative diseases. The present disclosure satisfies this unmet need.

### SUMMARY OF THE INVENTION

In one aspect, the present invention relates to a method of diagnosing a subject as having ALS or a specific ALS subtype, or an increased or decreased risk of ALS or a specific ALS subtype the method comprising: a) detecting the level or activity of at least two biomarkers selected from the biomarkers listed in Table 3 in a sample from the subject; b) comparing the level or activity of the biomarker in the sample to the level or activity of the biomarker in a comparator control; and c) diagnosing the subject as having ALS or a specific ALS subtype when the level or activity of the biomarker is significantly increased or decreased as compared to the comparator control.

In some embodiments, the subtype of ALS is a subset of ALS associated with activated glial cells (ALS-Glia), a subset of ALS associated with oxidative stress (ALS-Ox) or a subset of ALS associated with transcriptional dysregulation (ALS-TD).

In some embodiments, the subtype of ALS is ALS-Glia, and the subject is diagnosed with ALS-Glia when the level or activity of one or more of APOBR and TNFRSF25 is decreased as compared to the comparator control.

In some embodiments, the subtype of ALS is ALS-Glia, and the subject is diagnosed with ALS-Glia when the level or activity of one or more of AIF1, APOC2, CD44, CHI3L2, CX3CR1, FOLH1, HLA-DRA, TLR7, TNC, TREM2, TYROBP, ALOX5AP, APOC1, CCR5, CD68, CLEC7A, CR1, MSR1, MYL9, NCF2, NINJ2, ST6GALNAC2, TAGLN, TLR8 or VRK2 is increased as compared to the comparator control. In some embodiments, the subject is diagnosed with ALS-Glia when the level or activity of one or more of MYL9, ST6GALNAC2, and TAGLN is increased as compared to the comparator control.

In some embodiments, the subtype of ALS is ALS-Ox, and the subject is diagnosed with ALS-Ox when the level or activity of one or more of COL18A1, SLC6A13, TCIRG1, CP, NDUFA4L2, NOS3, NOTCH3, and TAGLN is decreased as compared to the comparator control.

In some embodiments, the subtype of ALS is ALS-Ox, and the subject is diagnosed with ALS-Ox when the level or activity of one or more of GABRA1, GAD2, GLRA3, HTR2A, OXR1, SERPINI1, SLC17A6, UBQLN2, B4GALT6, BECN1, GABRA6, GPR22, PCSK1, and UBQLN1 is increased as compared to the comparator control. In some embodiments, the subject is diagnosed with ALS-Ox when the level or activity of one or more of GABRA1, GAD2, and SLC17A6 is increased as compared to the comparator control.

In some embodiments, the subtype of ALS is ALS-TD, and the subject is diagnosed with ALS-TD when the level or activity of one or more of COL3A1, ENSG00000273151, MIRLET7BHG, and TUB-AS1 is decreased as compared to the comparator control.

In some embodiments, the subtype of ALS is ALS-TD, and the subject is diagnosed with ALS-TD when the level or activity of one or more of AGPAT4-IT1, CHKB-CPT1B, ENSG00000205041, ENSG00000258674, HSP90AB4P, LINC01347, MIR24-2, ADAT3, EGLNIP1, ENSG00000263278, ENSG00000268670, ENSG00000279233, LINC00176, MIR219A2, RPS20P22, and SLX1B-SULT1A4 is increased as compared to the comparator control.

In some embodiments, the present invention further comprises a step of administering a therapeutic agent for the treatment of the diagnosed ALS or ALS-subtype.

In some embodiments, the treatment comprises administering a modulator of one or more biomarker of Table 3.

In some embodiments, the modulator is a nucleic acid, a peptide, a small molecule chemical compound, an siRNA, a ribozyme, an antisense nucleic acid, an aptamer, a peptidomimetic, an antibody, or an antibody fragment.

In some embodiments, the subtype of ALS is ALS-Glia, and the subject diagnosed with ALS-Glia is administered an activator of one or more of APOBR and TNFRSF25.

In some embodiments, the subtype of ALS is ALS-Glia, and the subject diagnosed with ALS-Glia is administered an inhibitor of one or more of AIF1, APOC2, CD44, CHI3L2, CX3CR1, FOLH1, HLA-DRA, TLR7, TNC, TREM2, TYROBP, ALOX5AP, APOC1, CCR5, CD68, CLEC7A, CR1, MSR1, MYL9, NCF2, NINJ2, ST6GALNAC2, TAGLN, TLR8 or VRK2. In some embodiments, the subject is diagnosed with ALS-Glia when the level or activity of one or more of MYL9, ST6GALNAC2, and TAGLN is increased as compared to the comparator control.

In some embodiments, the subtype of ALS is ALS-Ox, and the subject diagnosed with ALS-Ox is administered an activator of one or more of COL18A1, SLC6A13, TCIRG1, CP, NDUFA4L2, NOS3, NOTCH3, and TAGLN.

In some embodiments, the subtype of ALS is ALS-Ox, and the subject diagnosed with ALS-Ox is administered an inhibitor of one or more of GABRA1, GAD2, GLRA3, HTR2A, OXR1, SERPINI1, SLC17A6, UBQLN2, B4GALT6, BECN1, GABRA6, GPR22, PCSK1, and UBQLN1. In some embodiments, the subject is diagnosed with ALS-Ox when the level or activity of one or more of GABRA1, GAD2, and SLC17A6 is increased as compared to the comparator control.

In some embodiments, the subtype of ALS is ALS-TD, and the subject diagnosed with ALS-TD is administered an activator of one or more of COL3A1, ENSG00000273151, MIRLET7BHG, and TUB-AS1.

In some embodiments, the subtype of ALS is ALS-TD, and the subject diagnosed with ALS-TD is administered an inhibitor of one or more of AGPAT4-IT1, CHKB-CPT1B, ENSG00000205041, ENSG00000258674, HSP90AB4P, LINC01347, MIR24-2, ADAT3, EGLNIP1, ENSG00000263278, ENSG00000268670, ENSG00000279233, LINC00176, MIR219A2, RPS20P22, and SLX1B-SULT1A4.

In another aspect, the present invention relates to a method of differentially diagnosing a subject as having a specific subtype of ALS, or providing a prognosis to a subject diagnosed with ALS, the method comprising a) detecting the level or activity of at least two biomarkers selected from: one or more transposable element, APOBR, APOC1, or APOC2 in a sample from the subject; b) comparing the level or activity of the biomarker in the sample to the level or activity of the biomarker in a comparator control; and c) diagnosing the subject as having ALS-Glia, or a poorer prognosis when the level of APOBR, APOC1, and APOC2 is increased in the sample as compared to the comparator control or diagnosing the subject as having ALS-Ox or ALS-TD, or a better prognosis when the activation of one or more transposable element is significantly increased as compared to the comparator control.

In some embodiments, the present invention further comprises a step of administering a therapeutic agent for the treatment of the diagnosed ALS-subtype.

In another aspect, the present invention relates to a method of differentially diagnosing a subject as having ALS or frontotemporal lobar degeneration (FTLD), the method comprising a) detecting the level or activity of STH in a sample from the subject; b) comparing the level or activity of STH in the sample to the level or activity of STH in a comparator control; and c) diagnosing the subject as having ALS when the level of STH is decreased in the sample as compared to the comparator control or diagnosing the subject as having FTLD when the level of STH increased as compared to the comparator control.

In some embodiments, the present invention further comprises a step of administering a therapeutic agent for the treatment of the diagnosed ALS or FTLD.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

The following detailed description of embodiments of the invention will be better understood when read in conjunction with the appended drawings. It should be understood that the invention is not limited to the precise arrangements and instrumentalities of the embodiments shown in the drawings.

FIG. 1A and FIG. 1B depict an overview of the ALS cohort. FIG. 1A depicts the selection of the ALS cohort from the GSE153960 repository. Transcriptomes associated with the frontal and motor cortex were the only tissue sites considered in this analysis. Control samples included 93 transcriptomes from healthy control donors and 42 from frontotemporal lobar degeneration patients. FIG. 1B depicts a comparison of common ALS patient postmortem cortex samples between the foundational study GSE124439 and the repository utilized in this analysis.

FIG. 2A through FIG. 2D depict experimental results of the DESeq2 normalization metrics. FIG. 2A depicts the dispersion estimation for transcript counts in the HiSeq ALS cohort showing gene-wise estimates shrunken towards the fitted estimates. FIG. 2B depicts the dispersion estimation for transcript counts in the NovaSeq ALS cohort. FIG. 2C depicts variance stabilized, mean-standard deviation plot showing roughly the same standard deviation in transcript counts, regardless of gene rank, for the HiSeq ALS cohort. FIG. 2D depicts similar standard deviation magnitudes are observed in the variance stabilized, mean-standard deviation plot for the NovaSeq ALS cohort.

FIG. 3A through FIG. 3D depict experimental results comprising the estimation of factorization rank. Preliminary unsupervised clustering analysis indicated a factorization rank of 3 optimal for both the NovaSeq and HiSeq cohorts. FIG. 3A depicts the non-smooth non-negative matrix factorization metrics for ranks 2-6 in the HiSeq cohort. FIG. 3B depicts the clustering metrics for the NovaSeq ALS cohort, spanning ranks 2-6. FIG. 3C depicts the consensus clustering was performed for each rank considered in the HiSeq cohort. FIG. 3D depicts the consensus clustering results for ranks 2-6 in the NovaSeq cohort.

FIG. 4A through FIG. 4J depict experimental results demonstrating the unsupervised clustering analysis with ALS postmortem cortex transcriptomes. FIG. 4A depicts the heatmap of 741 genes and transposable elements selected by SAKE showing transcript overexpression in a subtype specific fashion for the NovaSeq cohort (n=255 samples). Transcript counts are z-score normalized. FIG. 4B depicts the principal component analysis showing three distinct clusters, when considering the first two principal components. FIG. 4C depicts that the sample expression of CD28 transcripts was plotted in the same PCA space, with elevated counts seen for the ALS-Glia subtype. A darker color corresponds to higher feature expression. FIG. 4D depicts the expression of the ANO3 gene showing specificity for the oxidative stress and altered synaptic signaling subtype. FIG. 4E depicts the ALS-TD subtype showing specific upregulation of transposable element chr5|760200|760576|MLTIB:ERVL-MaLR:LTR|277|+ compared to the other two subtypes. FIG. 4F depicts the heatmap of 618 genes and TEs showing subtype-specific expression in the HiSeq cohort (n=196 samples). FIG. 4G depicts PCA considering the HiSeq cohort showing three distinct clusters of ALS patient transcriptomes. FIG. 4H depicts the elevated expression of CD22 in the activated glia subtype. FIG. 4I depicts the subtype-specific expression of WNT16 in the ALS-Ox subtype. FIG. 4J depicts chr10|14102244|14102461|AluSz:Alu:SINE|138|+ is overexpressed in the ALS-TD subtype.

FIG. 5A through FIG. 5H depicts experiment results demonstrating Gene Set Enrichment Analysis identifying subtype-specific disease pathways. FIG. 5A through FIG. 5C depict the gene sets enriched in each ALS subtype along the Y-axis, with GSEA normalized enrichment score (NES) presented along the X-axis. FIG. 5D depicts the heatmap of transposable element expression, with 426 unique TEs and 544 tissue transcriptomes. Patient samples were plotted by subgroup, with the thin black lines denoting sample separation by subtype. TE count values were subject to VST, followed by z-score normalization, with the color red indicating elevated expressed. FIG. 5E through FIG. 5H depict that the pathways enriched specifically for one or more subtypes were generated using GSEA rank metric scores. Genes comprising each functional pathway are included, with subtype-specific gene enrichment scores encoded on a red-blue scale.

FIG. 6A through FIG. 6I depicts experimental results displaying enrichment heatmaps supporting subtype-dependent ALS phenotypes. FIG. 6A through FIG. 6B depict the ALS-Glia subtype shows unique enrichment for pathways related to the immune system, as compared to healthy controls. FIG. 6C through FIG. 6D depict O-glycosylation and post translational modification machinery are altered in ALS patients. FIG. 6E through FIG. 6H depict that the enrichment for 6 synaptic signalling and neuronal receptor signalling pathways in the ALS-Ox subtype show distinct expression, as compared to healthy controls. FIG. 6I depicts that a locus-specific transposable element feature set, derived from SQUIRE13 was utilized to perform TE enrichment using GSEA. Normalized enrichment scores for each of the three subtypes 10 are plotted, with healthy controls specified as the reference. Results indicate that elevated expression of TEs are characteristic of both the ALS-Ox and ALS-TD subtypes.

FIG. 7A through FIG. 7B depict experimental results displaying locus-specific transposable elements in ALS spectrum neurodegeneration. FIG. 7A depicts locus-specific transposable elements were assigned to the group which demonstrated the largest average expression on the median-of-ratios scale and reveals characteristic expression in both the TD and Ox subtypes. FIG. 7B depicts TARDBP expression, encoding TDP-43, for healthy controls, patients with frontotemporal degeneration, and each ALS subtype on the DESeq2 median-of-ratios scale. Previous works have demonstrated direct interactions between TDP-43 and transposable elements (TEs) and implicated TE subfamilies as subtype specific features, however normalized expression is relatively consistent across ALS subtypes, suggesting TARDBP expression is not a defining characteristic of a single subtype.

FIG. 8A through FIG. 81 depict experimental results demonstrating the network construction elucidating subtype-specific disease pathways and eigengenes associated with ALS patient clinical outcomes. FIG. 8A depicts the network of pathways associated with the ALS cohort, color coded by subtype, with red indicating ALS-TD, blue denoting ALS-Ox, and yellow signifying ALS-Glia. FIG. 8B depicts that the WGCNA identifies gene subsets significantly correlated with ALS patient age of disease onset, age of death, and disease duration (univariate regression, two-tailed). Eigengene labels, moving left to right in the dendrogram, are: pink, red, tan, navy (ALS-Ox), brown, green, gold (ALS-Glia), gray, maroon (ALS-TD), yellow, blue, salmon, black, and green-yellow. Eigengenes were enriched for gene ontology and Bonferroni-adjusted p-values are shown (Fisher's exact test, one-sided). Subtype-specific expression of eigengenes was determined using dummy regression (two-tailed), with the ß coefficient presented as a heatmap. A positive ß coefficient denotes subtype upregulation of transcripts comprising the particular eigengene. Bonferroni-adjusted p-values less than 0.05 are denoted with *. FIG. 8C depicts that the eigengene correlation heatmap identifies transcripts that are similarly expressed. FIG. 8D depicts that the ALS-TD (magenta) eigengene shows long non-coding RNA, pseudogene, and transposable element expression are negatively correlated with ALS age of onset and age of death (p<0.05). FIG. 8E depicts the ALS-Glia (purple) eigengene illustrating that immune-related gene expression are positively correlated with ALS age of onset and age of death, and negatively correlated with disease duration (p<0.05). FIG. 8F through FIG. 81 depict univariate plots showing gene expression levels of four features in the purple eigengene—with evidence for ALS-Glia specificity. Some transcripts are differentially expressed in a manner that would suggest simple thresholding can distinguish ALS patients from FTLD patients and healthy controls.

FIG. 9A through FIG. 9C depict experimental results displaying violin plots capturing subtype-specific gene expression. FIG. 9A depicts that the 12 selected genes reflect ALS-Glia specific disease pathways, and include: AIF1, APOC2, CD44, CHI3L2, CX3CR1, FOLH1, HLA-DRA, TLR7, TMEM125, TNC, TREM2, and TYROBP. FIG. 9B depicts ALS-Ox subtype-specific gene expression for 12 representative genes: COL18A1, GABRA1, GAD2, GLRA3, HTR2A, OXR1, SERPINI1, SLC6A13, SLC17A6, TCIRG1, UBQLN2, and UCP2. FIG. 9C depicts the 12 subtype relevant genes selected for the ALS-TD group including: AGPAT4-IT1, CHKB-CPT1B, COL3A1, ENSG00000205041, ENSG00000258674, ENSG00000273151, GATA2-AS1, HSP90AB4P, LINC01347, MIR24-2, MIRLET7BHG, NANOGP4. Subgroups are color-coded, with the label provided along the X-axis and log 2 transformed, and DESeq2 normalized transcript counts on the Y-axis. The control samples were separated into healthy control donors (light gray) and patients with frontotemporal lobal degeneration (FTLD) exclusively (dark gray). FDR-corrected p-values are included if significant differences in gene expression are observed. Sample specific expression is included as individual points within the violin.

FIG. 10A through FIG. 10C depicts experimental results demonstrating the score-based classification comprising the hybrid subtype states in the ALS cohort. FIG. 10A depicts that the subtype scoring was implemented with bootstrapping to assess the spectrum of disease phenotypes presented in ALS. Each point corresponds to a single transcriptome derived from the frontal or motor postmortem cortex, n=451 biologically independent samples. Patient samples were initially placed at the origin, moved in the direction of the subtype axis for each round of bootstrapping that passed the subtype score threshold, and could only reach the vertex if the patient sample passed the threshold in all rounds of bootstrapping. Data points are filled according to the bootstrap-based subtype assignment and borders are included to denote the patient subtype obtained from unsupervised clustering. Transcriptomes that approached the vertices shared by two subtypes are considered to express a hybrid subtype state. Patient samples are color-coded gray if they failed to pass the subtype score thresholds in ≥50% of bootstrap iterations. FIG. 10B depicts the confusion matrix showing unsupervised clustering results in each classification subtype. FIG. 10C depicts the clustering results in Glia-TD and Glia-Ox hybrids.

FIG. 11A through FIG. 11E depict four supervised machine learning classifiers in the ALS cohort. FIG. 11A depicts F1 scores from 100-fold cross validation with the NovaSeq cohort as boxplots. Four classification methods were considered (KNN, MLP, RF, and linear SVC) and predictive metrics are separated by subtype label. The MLP classifier demonstrated the highest average F1 score for the ALS-Glia subtype (0.80), while the RF classifier showed the best performance when predicting the ALS-Ox (0.93) and ALS-TD subtypes (0.90). FIG. 11B depicts the ROC plot showing false positive rate (1-specificity) versus the true positive rate (sensitivity) for the KNN classifier when applied to the holdout (HiSeq) cohort. Given the multi-class nature of this analysis, three classifiers were constructed accounting for each binary case, using a ‘one-versus-rest’ approach. FIG. 11C depicts the ROC plot showing predictive metrics for the MLP classifier. FIG. 11D depicts the sensitivity and specificity metrics for the random forest classifier when applied to the holdout cohort. FIG. 11E depicts the ROC plot for the linear SVM classifier showing similar performance to the RF and MLP models.

FIG. 12A through FIG. 12E depict experimental results demonstrating Assessment of ALS patient clinical parameters in the context of disease subtypes. FIG. 12A depicts Kaplan-Meier survival analysis for the three ALS subtypes identified in the analysis. The ALS-Glia subtype is significantly associated with a shorter survival duration (p<0.01, log-rank test). The ALS-Ox subtype had a median survival duration of 36 months, while the ALS-TD group had the longest median survival (42 months). FIG. 12B depicts age of disease onset plotted as boxplots for the three ALS subtypes. No significant differences are observed in age of onset by subtype. FIG. 12C depicts age at death plotted as boxplots for the ALS-Glia, ALS-Ox, and ALS-TD subtypes. Again, no significant differences are observed. FIG. 12D depicts ALS subtype site of symptom onset, with the ‘Other’ category comprising axial (4), axial-limb (2), bulbar-limb (4), axial-bulbar (1), generalized (1), and unknown (9) sites of onset. FIG. 12E depicts FTLD comorbidity was converted to a percentage and was plotted as a bar graph. A Chi-Square test of independence was used to assess whether ALS subtype and FTLD comorbidity were associated (c2=0.59).

FIG. 13A through FIG. 13E depict results upon including discordant patients when assessing clinical parameters. FIG. 13A depicts Kaplan-Meier survival analysis including the three ALS subtypes and ‘discordant’ patients. Pairwise comparisons showed significant differences in survival between the ALS-Glia and ALS-Ox subtypes (p=0.015) and ALS-Glia and ALS-TD subtypes (p=0.0043). FIG. 13B depicts age of ALS symptom onset are plotted as boxplots, separated by disease group. The ALS-Glia subtype shows a nonsignificant trend towards the latest symptom onset. FIG. 13C depicts age at death for the three ALS subtypes and discordant patients. FIG. 13D depicts the site of symptom onset for all ALS patients included in this analysis, and a chi-square test of independence suggests site of symptom onset and subtype are not strongly associated. The ‘other’ category is comprised of axial (4), axial-limb (2), bulbar-limb (4), axial-bulbar (2), generalized (1), and unknown (11) sites of onset. FIG. 13E depicts the frontotemporal lobar degeneration comorbidity as a percentage, for all ALS patient groups considered in this analysis. A chi-square test of independence again suggests FTLD comorbidity and ALS subtype are not strongly associated.

FIG. 14 depicts the features for the ALS-Glia subtype. Violin plots show ALS-Glia specific expression for 16 supporting genes: (top left) ALOX5AP, APOBR, APOC1, CCR5, CD68, CLEC7A, CR1, FPR3, MSR1, NCF2, NINJ2, ST6GALNAC2, TLR8, TNFRSF25, TREM1, and VRK2. Genes are generally associated with glial activation, neuroinflammation, and a pro-apoptotic phenotype.

FIG. 15 depicts the features for the ALS-Ox subtype. ALS-Ox specific gene expression is shown as violin plots, and include: (top left) B4GALT6, BECN1, COL4A6, COX412, CP, GABRA6, GPR22, MYH11, MYL9, NDUFA4L2, NOS3, NOTCH3, PCSK1, SOD1, TAGLN, and UBQLN1. Supporting genes are generally associated with synaptic signaling, blood-brain barrier integrity, oxidative stress, and proteotoxic stress.

FIG. 16 depicts the features for the ALS-TD subtype. ALS-TD specific feature expression is shown as violin plots, and include: (top left) ADAT3, COL6A3, EGLNIP1, ENSG00000263278, ENSG00000268670, ENSG00000279233, ITGBL1, KRT8P13, LINC00176, LINC00638, MIR219A2, NKX6-2, RPS20P22, SLX1B-1, SULTIA4, TP63, and TUB-AS1. Supporting genes are generally associated with transcriptional regulation.

FIG. 17 depicts the subtype-specific transposable element expression. Subtype-specific transposable element expression. Representative TE features for the ALS-Glia, ALS-Ox, and ALS-TD subtypes. ALS-Ox and ALS-TD subtypes were defined by upregulated expression of long interspersed nuclear elements (LINEs), short interspersed nuclear elements (SINEs), and long terminal repeats (LTRs). The ALS-Glia subtype was defined by downregulated expression of TEs, as compared to other ALS subtypes and controls.

FIG. 18 depicts the characteristic gene expression distinguishing ALS patients from controls. Genes strongly differentially expressed between ALS patients and controls. Violin plots indicate simple thresholding could be utilized to distinguish ALS patients from controls and some genes further show subtype-specific upregulation or downregulation. Of notable interest, elevated expression of STH in the brain is known to serve as a marker for Parkinson's and other neurodegenerative diseases, including FTLD, and is observed to be strongly downregulated in all ALS patients. These findings offer a potential marker for the stratification of FTLD patients and ALS patients with FTLD comorbidity.

FIG. 19A through FIG. 19F depict experimental results demonstrating clustering and enrichment of ALS spinal cord transcriptomes. FIG. 19A depicts a heatmap showing subtype specific gene expression in the NovaSeq cohort, comprised of 273 biologically independent tissue samples and 763 transcripts. FIG. 19B depicts gene expression in the HiSeq cohort, with 155 biologically independent tissue samples and 567 transcripts. Both heatmaps are presented after z-score normalization with features selected by SAKE (Ho, Y. J. et al. 2018. Genome Res. 28, 1353-1363). FIG. 19C and FIG. 19D depict principal component analysis showing three distinct clusters when considering the first two principal components, in both the NovaSeq and HiSeq cohorts. FIG. 19E depicts enrichment analysis using the Enrichr Fisher exact tests with FDR (Benjamini, Y. & Hochberg Y. 1995. Journal of the Royal statistical society: series B (Methodological) 57, 289-30) adjusted p-values presented on the −log 10 scale. Genes were assigned to patient clusters depending on median expression. FIG. 19F depicts the gene set enrichment analysis (GSEA) with non-neurological controls specified as the reference level for calculation of all normalized enrichment scores.

FIG. 20A through FIG. 20F depict the independent and Identically Distributed (IID) Survival Analysis. Tissue region specific survival analyses for the ALS FIG. 20A through FIG. 20F depict frontal cortex (n=193) (FIG. 20A), medial motor cortex (n=102) (FIG. 20B), lateral motor cortex (n=104) (FIG. 20C), cervical spinal cord (n=195) (FIG. 20D), thoracic spinal cord (n=55) (FIG. 20E), and lumbar spinal cord (n=178) (FIG. 20F). The “unspecified motor cortex” (n=52) was not considered. The effects due to repeat sampling from patients can be seen to contribute to the significant differences observed in survival when assigning subtype by majority agreement (Tam, O. H. et al. 2019. Cell Rep. 29, 1164-1177; Eshima, J. et al. 2023. Nature Communications, 14, 95). However, across most tissue regions, there is a general and consistent trend toward a shorter survival duration in the ALS-Glia subtype, with significant differences maintained between ALS-Glia and ALS-Ox patients in the cervical spinal cord.

FIG. 21A through FIG. 21D depict the subtype concordance between the ALS cortex and spinal cord. FIG. 21A-21D depict agreement between the subtype assigned to the cervical, thoracic, and lumbar regions (rows) (FIG. 21A) and the frontal cortex, the unspecified motor cortex (FIG. 21B), the medial motor cortex (FIG. 21C), and the lateral motor cortex—for all available samples (FIG. 21D). Pie charts are first presented as an aggregate of all paired tissue samples (light blue and pink) and in a subtype-specific manner. Concordance at the subtype level (columns) has been color coded to indicate agreement (gold, navy, and maroon) or disagreement (orange, green, and purple) between the two tissue regions compared. No patients assigned ALS-TD in the unspecified motor cortex had a corresponding thoracic spinal cord sample in this cohort.

FIG. 22A through FIG. 22C depict experimental results demonstrating differential expression identifies transcripts in the spinal cord that stratify subtypes and ALS-Ox markers shared between the cortex and spinal cord. FIG. 22A depicts a heatmap showing z-score normalized expression following transformation to the median-of-ratios scale for each subtype. For plotting, z-scores <−4 or >4 are adjusted to −4 and 4, respectively. All presented genes have mean raw counts >10 and are expressed uniquely in a single ALS subtype. A total of 519 spinal cord samples are shown along the columns, grouped by subtype. FIG. 22B depicts heatmaps showing-log 10 transformed differential expression FDR-adjusted p-values using pairwise comparisons. Gray cells indicated an adjusted p-value >0.05. FIG. 22C depicts representative ALS-Ox marker transcripts, with expression shown throughout the postmortem cortex (frontal and motor) and spinal cord (cervical, thoracic, lumbar). Expression of GABRA1, GAD2, and SLC17A6 are consistently and significantly elevated in ALS-Ox patients as compared to other subtypes and controls from comparable tissue regions. A combined total of 1,104 ALS and control samples are considered across all tissue regions presented. Using DESeq2 (Love, M. I. et al., 2014. Genome Biol. 15, 1-21), expression normalization was performed independently for the cortex and spinal cord. In both cases, effects due to sex, sequencing platform, RIN, and site of collection covariates are captured in the design equation—while the tissue source covariate was uniquely included during normalization in the spinal cord cohort.

FIG. 23A through FIG. 23C depict the supervised classification of ALS-Ox samples using expression of B4GALT6, GLRA3, and SLC17A6 marker genes. Five different classification algorithms were considered. F1 scores obtained from 100-fold cross validation in the test cohort are presented first and separated by classification level (‘Ox’ vs ‘NotOx’). A combined total of 1,104 ALS and control samples are considered, with n=377 (˜34%) assigned the ALS-Ox label. The five classifiers were constructed and applied to three different holdout/validation cohorts comprised of all postmortem spinal cord samples (n=519) (FIG. 23A), all postmortem cortex samples (n=585) (FIG. 23B), and all samples analyzed by HiSeq (n=415) (FIG. 23C). ROC plots are presented second, for each classifier, and show sensitivity and 1-specificity metrics when applied to the specified holdout cohort.

FIG. 24A and FIG. 24B depict the overview of GSE153960 spinal cord transcriptomes considered in this study. FIG. 24A depicts the sample number corresponding to each region of the spinal cord analyzed by Prudencio et al (Prudencio, M. et al. 2020. Clin. Investig. 130, e139741) on the NovaSeq and HiSeq sequencing platforms. FIG. 24B depicts the patient level analysis showing a high degree of overlap between the spinal cord ALS cohort and cortex cohort analyzed previously (Eshima, J. et al. 2023. Nature Communications, 14, 95).

FIG. 25A and FIG. 25B depict the factorization rank for nsNMF clustering. The factorization rank was determined by estimating clustering metrics from 50 iterations in the NovaSeq cohort (FIG. 25A) and the HiSeq cohort (FIG. 25B) for ranks spanning 2 to 6 (x-axis). In both cohorts, the non-smooth non-negative matrix factorization (nsNMF) method was used.

FIG. 26A and FIG. 26B depict the generalized comparison of subtypes assigned to the ALS postmortem cortex and spinal cord. FIG. 26A and FIG. 26B depicts pic charts showing patient level subtypes using the majority agreement approach described previously (Eshima, J. et al. 2023. Nature Communications, 14, 95; Tam, O. H. et al. 2019. Cell Rep. 29, 1164-1177) in the (FIG. 26A) postmortem cortex and (FIG. 26B) the postmortem spinal cord. A larger fraction of patients was found to be “Discordant” or “ALS-TD” in the spinal cord as compared to the cortex, potentially reflecting cell type composition differences in each tissue region. When accounting for sequencing platform in the assignment of patient subtype at the individual sample level, the NovaSeq cohort more closely mirrors the subtype proportion in the postmortem cortex. Yet, in both cases, the ALS-Ox subtype was not the most common, again pointing to cell composition effects given work from Humphrey's et al (Humphrey, J. et al. 2023. Nature Neuroscience, 1-13).

FIG. 27A through FIG. 27E depict the diagnostic plots showing spinal cord subtype is partially influenced by multiple covariates after dependent gene removal. FIG. 27A through FIG. 27D depict covariate diagnostic plots following assignment of sample subtype using non-smooth non-negative matrix factorization showing (FIG. 27A) sex, (FIG. 27B) site of collection, (FIG. 27C) tissue region, and (FIG. 27D) RIN. While scaled RIN does not appear to have a strong effect on the assigned subtype, the remaining covariates are seen to influence subtype despite removal of covariate-dependent genes using differential expression. Strictly considering this spinal cord cohort, the NYGC site of collection/processing and the lumbar tissue region appear more robust to covariate-dependent gene expression. FIG. 27E depicts the estimated cell type fractions in the postmortem spinal cord (n=293 unique transcriptomes, 137 cervical, 36 thoracic, 120 lumbar), considered in the context of the ALS subtypes. Estimates were previously calculated by Humphrey et al (Humphrey, J. et al. 2023. Nature Neuroscience, 1-13) using the MuSiC algorithm (Wang X, et al., 2019. Nature communications. 10 (1): 380) with reference single-nucleus RNA-seq data from Mathys et al (Mathys H, et al., 2019. Nature. 570 (7761): 332-7). Significant differences in cell type percentages were assessed using a two-sided Wilcoxon rank sum test with Bonferroni p-value adjustment. Adjusted p-values are denoted using the following scheme: *** p<1E-5; ** p<0.001; * p<0.05. Pairwise comparisons not depicted have an adjusted p-value >0.05.

FIG. 28A through FIG. 28E depict the survival and clinical parameters analysis. FIG. 28A depicts Kaplan-Meier survival analysis (Kaplan, E. L. & Meier, P. 1958. J. Am. Stat. Assoc. 53, 457-481) using patient subtypes (n=206) defined by spinal cord transcriptomes. Subtypes were assigned if the majority of available tissue regions were concordant, otherwise the patients were assigned to the ‘Discordant’ group. The ALS-Glia subtype is observed to have a significantly shorter survival duration when compared to the ALS-Ox and Discordant groups. FIG. 28B and FIG. 28C depict age of onset (n=206) (FIG. 28B) and age at death (n=206) (FIG. 28C) are presented as boxplots for each subtype. T-tests with a false discovery rate (Benjamini, Y. & Hochberg Y. 1995. Journal of the Royal statistical society: series B (Methodological) 57, 289-300) correction were applied, and the Glia subtype was seen to have a significantly later age of onset as compared to the Discordant group. FIG. 28D and FIG. 28E depicts comorbidity for (FIG. 28D) FTLD and (FIG. 28E) Alzheimer's disease are presented as bar plots. Chi-squared tests of independence were performed and neither FTLD (p=0.38) nor Alzheimer's (p=0.15) comorbidity was seen to be associated with ALS subtype.

FIG. 29A through FIG. 29F depicts the Cox Regression for Independent and Identically Distributed (IID) Survival Analysis. Tissue region specific survival analyses for the ALS (FIG. 29A) frontal cortex (n=193), (FIG. 29B) medial motor cortex (n=102), (FIG. 29C) lateral motor cortex (n=104), (FIG. 29D) cervical spinal cord (n=195), (FIG. 29E) thoracic spinal cord (n=55), and (FIG. 29F) lumbar spinal cord (n=178). The “unspecified motor cortex” (n=52) was not considered. The effects due to repeat sampling from patients can be seen to contribute to the significant differences observed in survival when assigning subtype by majority agreement. However, across most tissue regions, there is a general and consistent trend toward a lower hazard associated with the ALS-Ox subtype. Model terms are presented as hazard ratios with the 95% confidence interval shown. Terms are separated by covariate and subgroup, with reference levels indicated.

FIG. 30 depicts the Cox proportional hazard model diagnostics. Sample level observations from the postmortem cortex and spinal cord were separated by tissue region and utilized to construct Cox proportional hazard regression models. Sex, disease group, age of onset, and subtype were included as model covariates, yielding a total of 728 observations without missing data in the six Cox models shown. For each model constructed, residuals are plotted by covariate, and generally show weak or null dependency on the variable level. To assess adherence to the proportional hazard model assumption, scaled Schoenfeld residual plots are shown for each covariate level, with score test p-values determined using the ‘km’ time transformation. All covariates are seen to meet the assumption of having proportional hazards over the survival duration, excluding the disease group covariate in the lateral motor cortex model (p=0.04).

FIG. 31A through FIG. 31D depict experimental results demonstrating tissue specific concordance between the postmortem cortex and spinal cord in the NovaSeq cohort. FIG. 31A through FIG. 31D depict agreement between the subtype assigned to the cervical, thoracic, and lumbar regions (rows) and (FIG. 31A) the frontal cortex, (FIG. 31B) the unspecified motor cortex, (FIG. 31C) the medial motor cortex, and (FIG. 31D) the lateral motor cortex—for all available NovaSeq spinal cord samples. Pie charts are first presented as an aggregate of all paired tissue samples (light blue and pink) and in a subtype-specific manner. Concordance at the subtype level has been color coded to indicate agreement (gold, navy, and maroon) or disagreement (orange, green, and purple) between the two tissue regions compared. No patients assigned ALS-TD in the lateral motor cortex had a corresponding thoracic spinal cord sample in this cohort. There were no intra-patient tissue pairings between the thoracic spinal cord and unspecified motor cortex in this cohort.

FIG. 32A through FIG. 32D depict experimental results demonstrating tissue specific concordance between the postmortem cortex and spinal cord in the HiSeq cohort. FIG. 32A-FIG. 32D depict agreement between the subtype assigned to the cervical, thoracic, and lumbar regions (rows) and (FIG. 32A) the frontal cortex, (FIG. 32B) the unspecified motor cortex, (FIG. 32C) the medial motor cortex, and (FIG. 32D) the lateral motor cortex—for all available HiSeq spinal cord samples. Pie charts are first presented as an aggregate of all paired tissue samples (light blue and pink) and in a subtype-specific manner. Concordance at the subtype level has been color coded to indicate agreement (gold, navy, and maroon) or disagreement (orange, green, and purple) between the two tissue regions compared. No patients assigned ALS-TD in the unspecified motor cortex had a corresponding thoracic spinal cord sample in this cohort.

FIG. 33A through FIG. 33H depict data experimental results demonstrating the ALS patients with subtype concordance throughout the central nervous system. Multiple patients demonstrated perfect concordance across all available tissue transcriptomes and are presented using publicly available de-identified IDs. (FIG. 33A) Heatmap showing subtype assignment to each region of the cortex and spinal cord considered in this study. Gray cells correspond to unavailable or not applicable tissue transcriptomes. Patient sex is presented for each ALS subtype (FIG. 33B-FIG. 33D). Interestingly, patients concordant for the ALS-Glia subtype are primarily female, potentially indicating sex-dependent differences in the presentation of disease phenotype. Clinical parameters for concordant patients are plotted as boxplots, and show disease duration (FIG. 33E), age at onset (FIG. 33F), age at death (FIG. 33G), and site of symptom onset (FIG. 33H). Statistical tests were not performed due to limited patient number.

FIG. 34A through FIG. 34E depict experimental results demonstrating concordance between the postmortem cortex and spinal cord using the majority agreement approach. A meta level analysis comparing subtype assigned in the cortex with the spinal cord using the majority agreement approach—in which patients were assigned a subtype if a single sample was available or by majority if two or more samples were available. Patient subtype was assigned in the cortex and spinal cord independently. FIG. 34A depicts that the majority (68.2%) of patient samples did not show concordance between the postmortem cortex and spinal cord when using the majority agreement approach-indicating this method is not the optimal way to manage repeat patient sampling. FIG. 34B depicts that for patients that presented as ALS-Glia in the cortex, ˜32% of individuals were assigned the same subtype in their spinal cord by majority agreement. Discordant was the most common subtype assigned, likely reflecting limitations due to cell type composition of the spinal cord. FIG. 34C depicts patients with the oxidative stress phenotype in the cortex demonstrated similar concordance, with ˜25% of patients assigned the same subtype in their spinal cord. Encouragingly, very few patients classified as ALS-Ox in the cortex were assigned ALS-Glia in their spinal cord, suggesting cell type composition does not act as a confounding factor in the expression of ALS-Ox marker genes in the spinal cord but weakens the detectable signal. FIG. 34D depicts patients presenting as ALS-TD in their cortex showed the highest concordance with the spinal cord (˜48%), but likely reflects bias towards this subtype in the spinal cord transcriptomes. Interestingly, this bias towards ALS-TD in the spinal cord does not appear to be due to RIN, given that the mean RIN was 6.14 for all cortex samples yet 6.50 for all samples from the spinal cord. FIG. 34E depicts patients initially showing discordance for their postmortem cortex subtype are reassigned to ALS subtypes with roughly the same frequency. The concordance label in this figure indicates both the postmortem cortex and spinal cord presented as “Discordant” by the majority agreement method.

FIG. 35A through FIG. 35G depict experimental results demonstrating subtype-specific marker genes in the postmortem cortex and spinal cord. ALS-Ox and ALS-Glia marker genes show coherent expression throughout the central nervous system. FIG. 35A through FIG. 35G depict that expression is separated both by subtype and CNS region for (FIG. 35A) B4GALT6, (FIG. 35B) GABRA1, (FIG. 35C) HTR2A, (FIG. 35D) MYL9, (FIG. 35E) PCSK1, (FIG. 35F) ST6GALNAC2, and (FIG. 35G) TAGLN. All counts are presented on the log 2 median-of-ratio scale. All differential expression p-values are FDR adjusted. ALS-Ox marker genes include B4GALT6, GABRA1, GAD2, GLRA3, HTR2A, PCSK1, and SLC17A6. ALS-Glia marker genes include MYL9, ST6GALNAC2, and TAGLN although expression of these genes is less specific for ALS-Glia and are likely more susceptible to cell composition differences in the spinal cord.

FIG. 36 depicts experimental results demonstrating RPKM normalized ALS-Ox marker gene expression. Sample-level expression of ALS-Ox marker genes after grouping by ALS-Ox percentage, calculated by taking the number of intra-patient samples defined as ALS-Ox divided by the total number of samples from the patient. Transcript expression is normalized by library size to the RPKM scale. Concordant ALS-Ox patients were defined as ALS-Ox in all available tissue samples, while the ‘generally not ALS-Ox’ category is defined as less than 50% of samples classified as ALS-Ox. A total of 53 unique samples were included in the ‘Concordant ALS-Ox’ category, 393 samples in the ‘at least 50% ALS-Ox’, 410 samples in ‘generally not ALS-Ox’, and 184 control samples-corresponding to 206 ALS patients and 88 non-neurological controls.

FIG. 37A through FIG. 37C depict the expression of STMN2, truncated STMN2, and TARDBP. FIG. 37A depicts a two-sided Mann-Whitney U test used to assess statistical significance in STMN2 and truncated STMN2 expression on both TPM scale and raw count scale. After adjusting p-values for multiple hypothesis testing using the Bonferroni method, truncated STMN2 expression was elevated in the postmortem spinal cord of ALS-Ox patients when compared to the ALS-TD subtype on both count scales, further supporting phenotypic differences between ALS-Ox and ALS-TD patients. These finds are somewhat surprising, given ALS-TD pathology appears more closely linked to transcription as compared to ALS-Ox. FIG. 37B depicts truncated STMN2 counts on the TPM scale replotted for visual clarity. No statistically significant differences in the expression of STMN2 or truncated STMN2 are observed between ALS-Glia and ALS-TD subtypes. FIG. 37C depicts expression of transcript TARDBP, encoding ALS disease-associated protein TDP-43, to show transcript level differences are not observed in the postmortem spinal cord, as well as the cortex (Eshima, J. et al. 2023. Nature Communications. 14, 95). These findings further support TDP-43 pathology occurring at the protein level rather than transcript level.

FIG. 38 depicts data demonstrating that the neuroinflammatory subtype (ALS-Glia) is obscured in the ALS spinal cord. Transcripts found to stratify this ALS cohort using the postmortem cortex (Eshima, J. et al. 2023. Nature Communications. 14, 95) are reconsidered in the spinal cord. ALS-Glia cortex transcripts AIF1, CD68, HLA-DRA, TREM2, and TYROBP show weak or non-significant differences in expression compared to the other two subtypes in the spinal cord. Genes associated with proteotoxic and oxidative stress are elevated in the cortex of ALS-Ox patients but not in the spinal cord, seen in the expression of BECN1, OXR1, SERPINI1, SOD1, and UBQLN2 yet tissue composition at the cellular level may partially explain these differences. NKX6-2 but not miR24-2, both associated with the regulation of transcription, showed weak but consistent upregulation in the cortex and spinal cord of ALS-TD patients.

FIG. 39A through FIG. 39C depict the three-gene PLS-DA classifiers for ALS-Ox patients. Partial least squares discriminate analysis with expression of transcripts normalized to the RPKM scale using library size and transcript length estimates from GRCh38.p12. In each case, visualization of patients is first performed using ALS-Ox marker genes, taking the mean RPKM expression magnitude—for the three-gene combination—from each available tissue sample. Majority assigned subtype is color-coded with the postmortem cortex and spinal cord presented in the upper and lower half circles, respectively. FIG. 39A-FIG. 39C depict that the PLS-DA classifier was then trained and tested using an 80/20 split of (FIG. 39A) all postmortem cortex samples, and validated using all spinal cord samples, (FIG. 39B) all postmortem spinal cord samples, and validated on the cortex holdout, and (FIG. 39C) all NovaSeq samples, and validated on the HiSeq holdout. Following PLS-DA, the training cohort is plotted using the first two components. Test cohort F1 metrics are presented as boxplots for 100 rounds of cross validation predicting ALS-Ox against all other samples, including non-neurological controls (‘Other’). Lastly, ROC plots showing application of the PLS-DA classifier to each of the three holdout cohorts. The top gene combination is provided for each holdout cohort, and the B4GALT6, GLRA3, SLC17A6 trio was found to have the highest average AUC across all three holdout datasets.

FIG. 40A through FIG. 40D depict the PLS-DA with all seven ALS-Ox marker genes. Expression of all transcripts were normalized to the RPKM scale using library size and transcript lengths from GRCh38.p12. FIG. 40A depicts the visualization of patients using ALS-Ox marker genes, taking the mean magnitude of RPKM normalized expression from each available tissue sample. Majority assigned subtype is color-coded with the postmortem cortex and spinal cord presented in the upper and lower half circles, respectively. FIG. 40B through FIG. 40D depict that the PLS-DA classifier was then trained and tested using an 80/20 split of (FIG. 40B) all postmortem cortex samples, and validated on the spinal cord holdout, (FIG. 40C) all postmortem spinal cord samples, and validated on the cortex holdout, and (FIG. 40D) all NovaSeq samples, and validated on the HiSeq holdout. In each case, patients are visualized using the first two components, followed by presentation of F1 scores in the ALS-Ox and ‘NotOx’ (Other) groups. ROC plots then show sensitivity and 1-specificity of the PLS-DA classifier when applied to each of the three validation cohorts.

FIG. 41A through FIG. 41C depict the supervised machine learning classifiers using all seven ALS-Ox marker genes. Five different classifiers were constructed, using RPKM normalized expression of all seven ALS-Ox marker genes. F1 scores from 100 rounds of cross validation are presented as boxplots, for each classifier considered. F1 scores are separated by class level (ALS-Ox and ‘Not ALS-Ox’). A combined total of 1,104 ALS and control samples are considered, with n=377 (˜34%) assigned the ALS-Ox label. FIG. 41A-FIG. 41C depict the classifiers constructed and applied to three different holdout cohorts comprised of (FIG. 41A) all postmortem spinal cord samples (n=519), (FIG. 41B) all postmortem cortex samples (n=585), and (FIG. 41C) all samples analyzed by HiSeq (n=415). ROC plots are presented second, for each classifier, and show sensitivity vs 1-specificity metrics when applied to the specified holdout cohort.

FIG. 42A and FIG. 42B depict bulk tissue cell deconvolution in ALS subtypes. FIG. 42A depicts cell type percentages in the prefrontal and motor cortices for all patient samples considered in this study. FIG. 42B depicts fractions of cell types in the frontal and motor postmortem cortex, that were considered in the context of the ALS subtypes. Significant differences in cell type percentages were assessed using a two-sided Wilcoxon rank sum test with Bonferroni p-value adjustment. Adjusted p-values are denoted using the following scheme: *** p<0.001; ** p<0.01; * p<0.05. The median is indicated by the solid black line, and first and third quartiles are captured by the bounds of the box. Boxplot whiskers are defined as the first and third quartiles±interquartile range times 1.5, respectively, and outliers are denoted as solid black points. Minimum and maximum values are captured by the lowermost and uppermost points, respectively, or whisker bound if no outliers are shown.

FIG. 43A through FIG. 43B depict the representative intra-patient concordance p-values, estimated from 10,000 rounds of bootstrapping after independently adjusting for the proportion of patient subtypes observed in the cortex and spinal cord cohorts. The red line indicates the true number of concordant samples observed for a given cortex-spinal tissue pairing. P-value estimates are provided for (FIG. 43A) all patient samples and (FIG. 43B) the NovaSeq subset exclusively. The NovaSeq platform generally outperforms the HiSeq, with no tissue pairings having statistical significance in the HiSeq subset. No significant agreement was observed in the thoracic spinal cord in any cohort.

FIG. 44A through FIG. 44D depict representative retrotransposon expression in the ALS spinal cord. TEs were only considered if they passed filtering by median absolute deviation and were shared between sequencing platform subgroups, totaling 86 non-redundant transcripts. FIG. 44A depicts a representative heatmap showing spinal cord TE expression in 519 samples (n=428 ALS and 91 controls) with count values normalized by DESeq2 and z-score transformed before plotting. Three differentially expressed TEs are shown for ALS-Glia samples (FIG. 44B), ALS-Ox samples (FIG. 44C), and ALS-TD samples (FIG. 44D). Expression values are normalized to the DESeq2 median-of-ratios scale and log 2 transformed prior to plotting. P, DESeq2 differential expression using the negative binomial distribution, two-tailed, FDR method for multiple hypothesis test correction.

### DETAILED DESCRIPTION

In one aspect, the invention is partly based on the discovery of amyotrophic lateral sclerosis (ALS) and ALS subtype specific upregulation and downregulation of genes in the frontal and motor cortex which provides a novel set of transcripts associated with patient prognosis. Thus, the invention provides biomarkers for ALS and ALS subtype stratification.

The disclosure presented herein identifies biomarkers for an ALS-Glia subtype, including enrichment for immunological signaling and activation, downregulated expression of APOBR and TNFRSF25, and upregulated expression of AIF1, APOC2, CD44, CHI3L2, CX3CR1, FOLH1, HLA-DRA, TLR7, TNC, TREM2, TYROBP, ALOX5AP, APOC1, CCR5, CD68, CLEC7A, CR1, MSR1, MYL9, NCF2, NINJ2, ST6GALNAC2, TAGLN, TLR8 or VRK2 transcripts. In some embodiments, the biomarkers for the ALS-Glia subtype include downregulated expression of APOBR and TNFRSF25, and upregulated expression of AIF1, APOC2, CD44, CHI3L2, CX3CR1, FOLH1, HLA-DRA, TLR7, TNC, TREM2, TYROBP, ALOX5AP, APOC1, CCR5, CD68, CLEC7A, CR1, MSR1, MYL9, NCF2, NINJ2, ST6GALNAC2, TAGLN, TLR8 or VRK2 transcripts. In some embodiments, the biomarkers for the ALS-Glia subtype include upregulated expression of MYL9, ST6GALNAC2, or TAGLN transcripts as compared to the comparator control.

The disclosure presented herein identifies biomarkers for the ALS-Ox subtype, including oxidative stress, proteotoxic stress, strongly overexpressed transposable elements (TEs) chr2|130338399|130338546|LIME4b:L1:LINE|212|+, chr6|49430916|49431136|LTR86A1:ERVL:LTR|291|−, chr6|116277660|116277934|AluSg:Alu:SINE|44|+, chr8|56958199|56958343|L2b:L2:LINE|303|−, chr14|62107151|62107446|AluJb:Alu:SINE|169|+, chr15|65891440|65891604|MIR3:MIR:SINE|247|+, chr19|46427065|46427223|L2c:L2:LINE|284|+, and chr20|36652130|36652423|AluSx1:Alu:SINE|106|+, as well as downregulated expression of COL18A1, SLC6A13, TCIRG1, COL4A6, COX412, CP, MYH11, MYL9, NDUFA4L2, NOS3, NOTCH3, and TAGLN transcripts and upregulated expression of GABRA1, GAD2, GLRA3, HTR2A, OXR1, SERPINI1, SLC17A6, UBQLN2, UCP2, B4GALT6, BECN1, GABRA6, GPR22, PCSK1, SOD1, and UBQLN1 transcripts. In some embodiments, the biomarkers for the ALS-Ox subtype include downregulated expression of COL18A1, SLC6A13, TCIRG1, CP, NDUFA4L2, NOS3, NOTCH3, and TAGLN transcripts and upregulated expression of GABRA1, GAD2, GLRA3, HTR2A, OXR1, SERPINI1, SLC17A6, UBQLN2, B4GALT6, BECN1, GABRA6, GPR22, PCSK1, and UBQLN1 transcripts. In some embodiments, the biomarkers for the ALS-Ox subtype comprises an increase in the level or activity of at least one of GABRA1, GAD2, and SLC17A6 as compared to the comparator control.

The disclosure presented herein identifies biomarkers for the ALS-TD subtype, including unique expression of transcription and translation associated genes, including transcription factors, regulatory microRNAs, mRNA traditionally marked for nonsense mediated decay, pseudogenes, antisense, intronic, and long non-coding RNAs and strongly overexpressed TEs Chr17|9935956|9936183|LIM4:L1:LINE|302|+, and ChrX|54815877|54816014|MER117:hAT-Charlie:DNA|248|−, as well as downregulated expression of COL3A1, ENSG00000273151, MIRLET7BHG, COL6A3, ITGBL1, LINC00638, TP63 and TUB-AS1 and upregulated expression of AGPAT4-IT1, CHKB-CPT1B, ENSG00000205041, ENSG00000258674, GATA2-AS1, HSP90AB4P, LINC01347, MIR24-2, NANOGP4, ADAT3, EGLNIP1, ENSG00000263278, ENSG00000268670, ENSG00000279233, KRT8P13, LINC00176, MIR219A2, NKX6-2, RPS20P22, and SLX1B-SULTIA4 transcripts. In some embodiments, the biomarkers for the ALS-TD subtype include downregulated expression of COL3A1, ENSG00000273151, MIRLET7BHG, and TUB-AS1 and upregulated expression of AGPAT4-IT1, CHKB-CPT1B, ENSG00000205041, ENSG00000258674, HSP90AB4P, LINC01347, MIR24-2, ADAT3, EGLNIP1, ENSG00000263278, ENSG00000268670, ENSG00000279233, LINC00176, MIR219A2, RPS20P22, and SLX1B-SULTIA4 transcripts.

The invention provides biomarkers for ALS subtype stratification including APOBR, APOC1, and APOC2 overexpression in ALS-Glia compared to ALS-Ox and ALS-TD subtypes, and an increased activation of transposable elements in ALS-Ox and ALS-TD as compared to ALS-Glia.

In some embodiments, the invention provides a diagnostic or prognostic test for ALS-Glia comprising a combinatorial measurement of APOBR, and TNFRSF25, to validate downregulated expression, and of AIF1, APOC2, CD44, CHI3L2, CX3CR1, FOLH1, HLA-DRA, TLR7, TNC, TREM2, TYROBP, ALOX5AP, APOC1, CCR5, CD68, CLEC7A, CR1, MSR1, MYL9, NCF2, NINJ2, ST6GALNAC2, TAGLN, TLR8 or VRK2 to validate upregulated expression.

In some embodiments, the invention provides a biomarker for differentially diagnosing subjects as having or being at risk of frontotemporal lobar degeneration (FTLD) verses ALS with FTLD comorbidity. In some embodiments, downregulation of STH in the brain is associated with ALS whereas elevated expression of STH in the brain is associated with FTLD and other neurodegenerative disorders (e.g., Parkinson's Disease.)

Detection of one or more biomarkers of the invention is a useful diagnostic for diagnosing ALS or differentially diagnosing the subtype of ALS. In one embodiment, the biomarker for an ALS subtype can be a biomarker for diagnostics and a target for therapy for ALS or a comorbidity thereof (e.g., frontotemporal dementia, cardiovascular diseases).

In one embodiment, the invention includes detection of one or more biomarkers of the invention as a diagnostic tool for the detection of ALS or a comorbidity thereof. In one embodiment, the invention provides a differential diagnostic test and target for therapy for a specific ALS subset.

In one embodiment, the invention relates to modulating one or more mRNA and/or protein of an ALS biomarker of the invention to modulate disease state or disease progression.

In one embodiment, inhibition of a biomarker that shows upregulation or increased expression in one or more ALS subsets can be used to modulate disease state or disease progression. In one embodiment, an inhibitor of a biomarker that shows upregulation or increased expression in one or more ALS subsets includes but is not limited to a nucleic acid, a peptide, a small molecule chemical compound, an siRNA, a ribozyme, an antisense nucleic acid, an aptamer, a peptidomimetic, an antibody, an antibody fragment, induced protein degradation, or any combination thereof.

In one embodiment, activation of a biomarker that shows downregulation or decreased expression in one or more ALS subsets can be used to modulate disease state or disease progression. In one embodiment, an activator of a biomarker that shows downregulation or decreased expression in one or more ALS subsets includes but is not limited to a nucleic acid, a peptide, a small molecule chemical compound, a peptidomimetic, a transcriptional activator or any combination thereof.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “control or reference standard” describes a material comprising none, or a normal, low, or high level of one of more of the marker (or biomarker) expression products of one or more the markers (or biomarkers) of the invention, such that the control or reference standard may serve as a comparator against which a sample can be compared.

By the phrase “determining the level of marker (or biomarker) expression” is meant an assessment of the degree of expression of a marker in a sample at the nucleic acid or protein level, using technology available to the skilled artisan to detect a sufficient portion of any marker expression product.

“Differentially increased expression” or “up regulation” refers to biomarker product levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween than a control.

“Differentially decreased expression” or “down regulation” refers to biomarker product levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments therebetween than a control.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

The term “microarray” refers broadly to both “DNA microarrays” and “DNA chip(s),” and encompasses all art-recognized solid supports, and all art-recognized methods for affixing nucleic acid molecules thereto or for synthesis of nucleic acids thereon.

The “level” of one or more biomarkers means the absolute or relative amount or concentration of the biomarker in the sample.

The term “marker (or biomarker) expression” as used herein, encompasses the transcription, translation, post-translation modification, and phenotypic manifestation of a gene, including all aspects of the transformation of information encoded in a gene into RNA or protein. By way of non-limiting example, marker expression includes transcription into messenger RNA (mRNA) and translation into protein, as well as transcription into types of RNA such as transfer RNA (tRNA) and ribosomal RNA (rRNA) that are not translated into protein.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “providing a prognosis” refers to providing a prediction of the probable course and outcome of ALS and/or ALS subtype, including prediction of severity, duration, chances of recovery, etc. The methods can also be used to devise a suitable therapeutic plan, e.g., by indicating whether or not the condition is still at an early stage or if the condition has advanced to a stage where aggressive therapy would be ineffective.

A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype.

“Sample” or “biological sample” as used herein means a biological material isolated from an individual. The biological sample may contain any biological material suitable for detecting the desired biomarkers, and may comprise cellular and/or non-cellular material obtained from the individual.

“Standard control value” as used herein refers to a predetermined amount of a particular protein or nucleic acid that is detectable in a sample, such as a saliva sample, either in whole saliva or in saliva supernatant. The standard control value is suitable for the use of a method of the present invention, in order for comparing the amount of a protein or nucleic acid of interest that is present in a saliva sample. An established sample serving as a standard control provides an average amount of the protein or nucleic acid of interest in the saliva that is typical for an average, healthy person of reasonably matched background, e.g., gender, age, ethnicity, and medical history. A standard control value may vary depending on the biomarker of interest and the nature of the sample.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient.

The phrase “therapeutically effective amount,” as used herein, refers to an amount that is sufficient or effective to prevent or treat (delay or prevent the onset of, prevent the progression of, inhibit, decrease or reverse) a disease or disorder including alleviating symptoms of such diseases.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Modulator Compositions

In some embodiments, the invention provides compositions for modulating the level or activity of one or more biomarker of ALS and/or an ALS subtype. In one embodiment, the invention provides a modulator (e.g., an inhibitor or activator) of one or more gene, pseudogene, mRNA, protein, or transposable element identified as being a biomarker of ALS and/or an ALS subtype. Exemplary biomarkers of ALS and/or an ALS subtype include, but are not limited to, the markers presented in Table 3 as being associated with one or more ALS subtype.

In various embodiments, the present invention includes compositions for modulating the level or activity of a gene, a pseudogene, a transposable element, or a gene product in a subject, a cell, a tissue, or an organ in need thereof. In various embodiments, the compositions of the invention modulate the amount of a polypeptide, the amount of mRNA, or the activity of a biomarker of ALS and/or an ALS subset, or a combination thereof.

The compositions of the invention include compositions for diagnosing, treating or preventing ALS, a subtype of ALS, or an ALS comorbidity in a subject in need thereof.

In one embodiment, the invention includes methods and compositions for diagnosing, treating or preventing the ALS-Glia subtype of ALS which is associated with significantly elevated expression of microglia, astrocyte, and oligodendrocyte marker genes. In some embodiments, the biomarker of the ALS-Glia subtype of ALS is a biomarker as set forth in Table 3. In some embodiments, the biomarker of ALS-Glia is downregulated expression of APOBR or TNFRSF25, or any combination thereof. Therefore, in some embodiments, the invention provides compositions and methods for activating expression of or increasing the activity of one or more of APOBR or TNFRSF25. In some embodiments, the biomarker of ALS-Glia is upregulated expression of AIF1, APOC2, CD44, CHI3L2, CX3CR1, FOLH1, HLA-DRA, TLR7, TNC, TREM2, TYROBP, ALOX5AP, APOC1, CCR5, CD68, CLEC7A, CR1, MSR1, MYL9, NCF2, NINJ2, ST6GALNAC2, TAGLN, TLR8 or VRK2, or any combination thereof. In some embodiments, the biomarkers for the ALS-Glia subtype include upregulated expression of MYL9, ST6GALNAC2, or TAGLN transcripts as compared to the comparator control. Therefore, in some embodiments, the invention provides compositions and methods for inhibiting the expression or activity of one or more of AIF1, APOC2, CD44, CHI3L2, CX3CR1, FOLH1, HLA-DRA, TLR7, TNC, TREM2, TYROBP, ALOX5AP, APOC1, CCR5, CD68, CLEC7A, CR1, MSR1, MYL9, NCF2, NINJ2, ST6GALNAC2, TAGLN, TLR8 or VRK2. In some embodiments, the biomarkers for the ALS-Glia subtype include downregulated expression of APOBR and TNFRSF25, and upregulated expression of AIF1, APOC2, CD44, CHI3L2, CX3CR1, FOLH1, HLA-DRA, TLR7, TNC, TREM2, TYROBP, ALOX5AP, APOC1, CCR5, CD68, CLEC7A, CR1, MSR1, MYL9, NCF2, NINJ2, ST6GALNAC2, TAGLN, TLR8 or VRK2 transcripts, or any combination thereof. In some embodiments, the biomarkers for the ALS-Glia subtype include upregulated expression of MYL9, ST6GALNAC2, or TAGLN transcripts as compared to the comparator control.

In one embodiment, the invention includes methods and compositions for diagnosing, treating or preventing the ALS-Ox subtype of ALS which is associated with oxidative stress, proteotoxic stress, impaired blood-brain barrier function, and alterations to synaptic signaling. In some embodiments, the biomarker of the ALS-Ox subtype of ALS is a biomarker as set forth in Table 3. In some embodiments, the biomarker of ALS-Ox is overexpression of transposable elements (TEs) chr2|130338399|130338546|LIME4b:L1:LINE|212|+, chr6|49430916|49431136|LTR86A1:ERVL:LTR|291|−, chr6|116277660|116277934|AluSg:Alu:SINE|44|+, chr8|56958199|56958343|L2b:L2:LINE|303|−, chr14|62107151|62107446|AluJb:Alu:SINE|169|+, chr15|65891440|65891604|MIR3:MIR:SINE|247|+, chr19|46427065|46427223|L2c:L2:LINE|284|+, and chr20|36652130|36652423|AluSx1:Alu:SINE|106|+. In some embodiments, the biomarker of ALS-Ox is downregulated expression of COL18A1, SLC6A13, TCIRG1, COL4A6, COX412, CP, MYH11, MYL9, NDUFA4L2, NOS3, NOTCH3, or TAGLN, or any combination thereof. Therefore, in some embodiments, the invention provides compositions and methods for activating expression of or increasing the activity of one or more of COL18A1, SLC6A13, TCIRG1, COL4A6, COX412, CP, MYH11, MYL9, NDUFA4L2, NOS3, NOTCH3, or TAGLN. In some embodiments, the biomarker of ALS-Ox is upregulated expression of GABRA1, GAD2, GLRA3, HTR2A, OXR1, SERPINI1, SLC17A6, UBQLN2, UCP2, B4GALT6, BECN1, GABRA6, GPR22, PCSK1, SOD1, or UBQLN1, or any combination thereof. Therefore, in some embodiments, the invention provides compositions and methods for inhibiting the expression or activity of one or more of GABRA1, GAD2, GLRA3, HTR2A, OXR1, SERPINI1, SLC17A6, UBQLN2, UCP2, B4GALT6, BECN1, GABRA6, GPR22, PCSK1, SOD1, or UBQLN1. In some embodiments, the biomarkers for the ALS-Ox subtype include downregulated expression of COL18A1, SLC6A13, TCIRG1, CP, NDUFA4L2, NOS3, NOTCH3, and TAGLN transcripts and upregulated expression of GABRA1, GAD2, GLRA3, HTR2A, OXR1, SERPINI1, SLC17A6, UBQLN2, B4GALT6, BECN1, GABRA6, GPR22, PCSK1, and UBQLN1 transcripts. In some embodiments, the biomarkers for the ALS-Ox subtype comprises an increase in the level or activity of at least one of GABRA1, GAD2, and SLC17A6 as compared to the comparator control.

In one embodiment, the invention includes methods and compositions for diagnosing, treating or preventing the ALS-TD subtype of ALS which is associated with dysregulation of transcription, and overexpression of pseudogenes, intronic and antisense transcripts, long non-coding RNA, and nonsense-mediated decay mRNA. In some embodiments, the biomarker of the ALS-TD subtype of ALS is a biomarker as set forth in Table 3. In some embodiments, the biomarker of ALS-TD is overexpression of TEs Chr17|9935956|9936183|LIM4:L1:LINE|302|+, and ChrX|54815877|54816014|MER117:hAT-Charlie:DNA|248|−. In some embodiments, the biomarker of ALS-TD is downregulated expression of COL3A1, ENSG00000273151, MIRLET7BHG, COL6A3, ITGBL1, LINC00638, TP63 or TUB-AS1, or any combination thereof. Therefore, in some embodiments, the invention provides compositions and methods for activating expression of or increasing the activity of one or more of COL3A1, ENSG00000273151, MIRLET7BHG, COL6A3, ITGBL1, LINC00638, TP63 and TUB-AS1. In some embodiments, the biomarker of ALS-TD is upregulated expression of AGPAT4-IT1, CHKB-CPT1B, ENSG00000205041, ENSG00000258674, GATA2-AS1, HSP90AB4P, LINC01347, MIR24-2, NANOGP4, ADAT3, EGLNIP1, ENSG00000263278, ENSG00000268670, ENSG00000279233, KRT8P13, LINC00176, MIR219A2, NKX6-2, RPS20P22, or SLX1B-SULTIA4, or any combination thereof. Therefore, in some embodiments, the invention provides compositions and methods for inhibiting the expression or activity of one or more of AGPAT4-IT1, CHKB-CPT1B, ENSG00000205041, ENSG00000258674, GATA2-AS1, HSP90AB4P, LINC01347, MIR24-2, NANOGP4, ADAT3, EGLNIP1, ENSG00000263278, ENSG00000268670, ENSG00000279233, KRT8P13, LINC00176, MIR219A2, NKX6-2, RPS20P22, or SLX1B-SULTIA4. In some embodiments, the biomarkers for the ALS-TD subtype include downregulated expression of COL3A1, ENSG00000273151, MIRLET7BHG, and TUB-AS1 and upregulated expression of AGPAT4-IT1, CHKB-CPT1B, ENSG00000205041, ENSG00000258674, HSP90AB4P, LINC01347, MIR24-2, ADAT3, EGLNIP1, ENSG00000263278, ENSG00000268670, ENSG00000279233, LINC00176, MIR219A2, RPS20P22, and SLX1B-SULT1A4 transcripts.

In one embodiment, the invention includes methods and compositions for differentially diagnosing ALS-Glia vs the ALS-Ox/ALS-TD subtypes of ALS. In some embodiments, the biomarker for differential diagnosis of ALS-Glia vs the ALS-Ox/ALS-TD subtypes is activation of transposable elements which is found in ALS-Ox/ALS-TD subtypes but not ALS-Glia or overexpression of APOBR, APOC1, and APOC2, which is found in ALS-Glia, but not ALS-Ox/ALS-TD subtypes. ALS-Glia is associated with poorer prognosis, therefore, in some embodiments, the biomarkers for differential diagnosis of ALS-subtype can be used for providing a prognosis of a subject having ALS. In some embodiments, the invention provides methods of treating a subject identified as having an ALS subtype, for example, in some embodiments, the invention provides a method of administering a treatment regimen to the subject based on the differential diagnosis of ALS subtype or prognosis.

In one embodiment, the invention includes methods and compositions for differentially diagnosing ALS vs FTLD. In some embodiments, the biomarker for differential diagnosis of ALS vs FTLD is the level of STH in the brain. In some embodiments, downregulation of STH in the brain is associated with ALS whereas elevated expression of STH in the brain is associated with FTLD. Therefore, in some embodiments, the methods of the invention include measuring the level of STH in the brain and diagnosing the subject as having ALS when the level of STH is decreased or diagnosing the subject as having FTLD when the level of STH is increased. In some embodiments, the method of the invention further comprises administering a therapeutic agent for the diagnosed ALS or FTLD. In some embodiments, the subject diagnosed as having ALS is administered an activator of STH, whereas the subject diagnosed as having FTLD is administered an inhibitor of STH.

### Activators

In various embodiments, the composition comprises an activator of one or more gene or protein identified in Table 3 as being associated with ALS and/or a subtype of ALS. In various embodiments, the composition comprises an activator of one or more biomarker (e.g., gene, protein, mRNA, lncRNA, pseudogene, SINE, LINE) identified as being downregulated or as having decreased expression in ALS and/or an ALS subtype. In one embodiment, the activator of the invention increases the amount of polypeptide, the amount of mRNA, the amount of activity, or a combination thereof of the biomarker identified as being downregulated or as having decreased expression in ALS and/or an ALS subtype.

It will be understood by one skilled in the art, based upon the disclosure provided herein, that an increase in the level of a biomarker encompasses the increase in biomarker expression, including transcription, translation, or both. The skilled artisan will also appreciate, once armed with the teachings of the present invention, that an increase in the level of a biomarker includes an increase in biomarker activity. Thus, increasing the level or activity of a biomarker includes, but is not limited to, increasing the amount of polypeptide, increasing transcription, translation, or both, of a nucleic acid encoding the biomarker; and it also includes increasing any activity of a biomarker as well.

In some embodiments, the present invention relates to the prevention and treatment of a disease or disorder by administration of a polypeptide, a recombinant polypeptide, an active polypeptide fragment, or an activator of expression or activity of one or more biomarker of the invention.

Activation of one or more biomarker of the invention identified as being downregulated or as having decreased expression in ALS and/or an ALS subtype can be assessed using a wide variety of methods, including those disclosed herein, as well as methods well-known in the art or to be developed in the future. That is, the routineer would appreciate, based upon the disclosure provided herein, that increasing the level or activity of a biomarker can be readily assessed using methods that assess the level of a nucleic acid encoding the biomarker (e.g., mRNA) and/or the biomarker polypeptide in a biological sample obtained from a subject.

An activator can include, but should not be construed as being limited to, a chemical compound, a protein, a peptidomemetic, an antibody, a nucleic acid molecule. One of skill in the art would readily appreciate, based on the disclosure provided herein, that an activator encompasses a chemical compound that increases the level, enzymatic activity, or the like of one or more biomarker. Additionally, an activator encompasses a chemically modified compound, and derivatives, as is well known to one of skill in the chemical arts.

Further, one of skill in the art would, when equipped with this disclosure and the methods exemplified herein, appreciate that an activator of the invention includes such activators as discovered in the future, as can be identified by well-known criteria in the art of pharmacology, such as the physiological results of activation of one or more biomarker as described in detail herein and/or as known in the art. Therefore, the present invention is not limited in any way to any particular activator; rather, the invention encompasses those activators that would be understood to be useful as are known in the art and as are discovered in the future.

Further methods of identifying and producing an activator are well known to those of ordinary skill in the art, including, but not limited, obtaining an activator from a naturally occurring source. Alternatively, an activator can be synthesized chemically. Further, an activator can be obtained from a recombinant organism. Compositions and methods for chemically synthesizing activators and for obtaining them from natural sources are well known in the art and are described in the art.

One of skill in the art will appreciate that an activator can be administered as a small molecule chemical, a protein, a nucleic acid construct encoding a protein, or combinations thereof. Numerous vectors and other compositions and methods are well known for administering a protein or a nucleic acid construct encoding a protein to cells or tissues. Therefore, the invention includes a method of administering a protein or a nucleic acid encoding a protein that is an activator of a biomarker identified as being downregulated or as having decreased expression in ALS and/or an ALS subtype.

One of skill in the art will realize that diminishing the amount or activity of a molecule that itself diminishes the amount or activity of one or more biomarker identified as being downregulated or as having decreased expression in ALS and/or an ALS subtype can serve to increase the amount or activity of the one or more biomarker. Any inhibitor of a regulator of one or more biomarker identified as being downregulated or as having decreased expression in ALS and/or an ALS subtype is encompassed in the invention. As a non-limiting example, antisense nucleic acid molecules are described as one means of inhibiting a regulator of one or more biomarker of the invention in order to increase the amount or activity of one or more biomarker identified as being downregulated or as having decreased expression in ALS and/or an ALS subtype. Antisense oligonucleotides are DNA or RNA molecules that are complementary to some portion of a mRNA molecule. When present in a cell, antisense oligonucleotides hybridize to an existing mRNA molecule and inhibit translation into a gene product. Inhibiting the expression of a gene using an antisense oligonucleotide is well known in the art, as are methods of expressing an antisense oligonucleotide in a cell. The methods of the invention include the use of antisense oligonucleotide to diminish the amount of a molecule that causes a decrease in the amount or activity of one or more biomarker identified as being downregulated or as having decreased expression in ALS and/or an ALS subtype, thereby increasing the amount or activity of the one or more biomarker. Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides.

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system.

Alternatively, inhibition of a gene expressing a protein that diminishes the level or activity of one or more biomarker identified as being downregulated or as having decreased expression in ALS and/or an ALS subtype can be accomplished through the use of a ribozyme. Using ribozymes for inhibiting gene expression is well known to those of skill in the art. Ribozymes are catalytic RNA molecules with the ability to cleave other single-stranded RNA molecules. Ribozymes are known to be sequence specific, and can therefore be modified to recognize a specific nucleotide sequence, allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

One of skill in the art will appreciate that an activator of one or more biomarker of the invention can be administered singly or in combination with one or more additional agent. In some embodiments, the one or more additional agent is one or more additional modulator of a biomarker that is dysregulated in ALS and/or an ALS subtype. Further, an activator of one or more biomarker of the invention can be administered singly or in combination with one or more additional agent in a temporal sense, in that they may be administered simultaneously, before, and/or after each other. One of ordinary skill in the art will appreciate, based on the disclosure provided herein, that an activator of one or more biomarker of the invention can be used to prevent or treat ALS and/or an ALS subtype, and that an activator can be used alone or in any combination with another agent for the treatment of ALS and/or an ALS subtype to effect a therapeutic result.

One of skill in the art, when armed with the disclosure herein, would appreciate that the treating ALS and/or an ALS subtype encompasses administering to a subject an activator of one or more biomarker identified as being downregulated or as having decreased expression in ALS and/or an ALS subtype as a preventative measure against a development or progression of ALS and/or an ALS subtype. Thus, the invention encompasses administration of a polypeptide, a recombinant polypeptide, an active polypeptide fragment, a transcriptional activator, an inhibitor of a transcriptional repressor, or any other activator of one or more biomarker identified as being downregulated or as having decreased expression in ALS and/or an ALS subtype to practice the methods of the invention. The skilled artisan would understand, based on the disclosure provided herein, and general knowledge in the art, how to formulate and administer the appropriate activator to a subject. However, the present invention is not limited to any particular method of administration or treatment regimen. This is especially true where it would be appreciated by one skilled in the art, equipped with the disclosure provided herein, that methods of administering an activator can be determined by one of skill in the pharmacological arts.

### Inhibitors

In various embodiments, the composition comprises an inhibitor of one or more gene or protein identified in Table 3 as being associated with ALS and/or a subtype of ALS. In various embodiments, the composition comprises an inhibitor of one or more biomarker (e.g., gene, protein, mRNA, lncRNA, pseudogene, SINE, LINE) identified as being upregulated or as having increased expression in ALS and/or an ALS subtype. In various embodiments, the present invention includes compositions and methods of decreasing the level or activity of a biomarker identified as being upregulated or as having increased expression in ALS and/or an ALS subtype.

It will be understood by one skilled in the art, based upon the disclosure provided herein, that a decrease in the level or activity of a biomarker identified as being upregulated or as having increased expression in ALS and/or an ALS subtype encompasses the decrease in the expression of the biomarker, including transcription, translation, or both. The skilled artisan will also appreciate, once armed with the teachings of the present invention, that a decrease in the level or activity of a biomarker identified as being upregulated or as having increased expression in ALS and/or an ALS subtype includes a decrease in the activation with respect to transposable elements. Thus, decrease in the level or activity of a biomarker identified as being upregulated or as having increased expression in ALS and/or an ALS subtype includes, but is not limited to, decreasing the amount of polypeptide, decreasing the amount of mRNA, decreasing the amount of lncRNA, decreasing activation of TEs, and decreasing transcription, translation, or both, of a nucleic acid encoding a biomarker; and it also includes decreasing any activity of a biomarker as well.

In one embodiment, the invention provides a generic concept for inhibiting the level or activity of a biomarker identified as being upregulated or as having increased expression in ALS and/or an ALS subtype as an ALS therapy.

In one embodiment, the composition of the invention comprises an inhibitor of the level or activity of a biomarker identified as being upregulated or as having increased expression in ALS and/or an ALS subtype. In one embodiment, the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule.

One skilled in the art will appreciate, based on the disclosure provided herein, that one way to decrease the mRNA and/or protein levels of a biomarker in a cell is by reducing or inhibiting expression of the nucleic acid encoding the biomarker. Thus, the protein level of a biomarker in a cell can be decreased using a molecule or compound that inhibits or reduces gene expression such as, for example, siRNA, an antisense molecule or a ribozyme. However, the invention should not be limited to these examples.

In one embodiment, RNAi is used to decrease the level or activity of a biomarker identified as being upregulated or as having increased expression in ALS and/or an ALS subtype. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. Chemical modification to siRNAs can aid in intravenous systemic delivery. Optimizing siRNAs involves consideration of overall G/C content, C/T content at the termini, Tm and the nucleotide content of the 3′ overhang. Therefore, the present invention also includes methods of decreasing levels of one or more biomarker identified as being upregulated or as having increased expression in ALS and/or an ALS subtype using RNAi technology.

In some embodiments, the invention includes an isolated nucleic acid encoding an inhibitor, wherein an inhibitor such as an siRNA or antisense molecule, inhibits one or more biomarker identified as being upregulated or as having increased expression in ALS and/or an ALS subtype, operably linked to a nucleic acid comprising a promoter/regulatory sequence such that the nucleic acid is preferably capable of directing expression of the inhibitor encoded by the nucleic acid. Thus, the invention encompasses expression vectors and methods for the introduction of exogenous DNA into cells with concomitant expression of the exogenous DNA in the cells.

The siRNA or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA or antisense polynucleotide, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

In one embodiment of the invention, an antisense nucleic acid sequence which is expressed by a plasmid vector is used to inhibit one or more biomarker identified as being upregulated or as having increased expression in ALS and/or an ALS subtype. The antisense expressing vector is used to transfect a mammalian cell or the mammal itself, thereby causing reduced endogenous expression of one or more biomarker identified as being upregulated or as having increased expression in ALS and/or an ALS subtype.

Antisense molecules and their use for inhibiting gene expression are well known in the art. Antisense nucleic acids are DNA or RNA molecules that are complementary, as that term is defined elsewhere herein, to at least a portion of a specific mRNA molecule. In the cell, antisense nucleic acids hybridize to the corresponding mRNA, forming a double-stranded molecule thereby inhibiting the translation of genes.

The use of antisense methods to inhibit the translation of genes is known in the art. Such antisense molecules may be provided to the cell via genetic expression using DNA encoding the antisense molecule.

Alternatively, antisense molecules of the invention may be made synthetically and then provided to the cell. Antisense oligomers are generally between about 10 to about 30 nucleotides since they are easily synthesized and introduced into a target cell. Synthetic antisense molecules contemplated by the invention include oligonucleotide derivatives known in the art which have improved biological activity compared to unmodified oligonucleotides.

Compositions and methods for the synthesis and expression of antisense nucleic acids are as described elsewhere herein.

Ribozymes and their use for inhibiting gene expression are also well known in the art. Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it. A major advantage of this approach is the fact that ribozymes are sequence-specific.

There are two basic types of ribozymes, namely, tetrahymena-type and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit one or more biomarker identified as being upregulated or as having increased expression in ALS and/or an ALS subtype. Ribozymes useful for inhibiting the expression of a target molecule may be designed by incorporating target sequences into the basic ribozyme structure which are complementary, for example, to the mRNA sequence of one or more biomarker identified as being upregulated or as having increased expression in ALS and/or an ALS subtype of the present invention. Ribozymes targeting one or more biomarker identified as being upregulated or as having increased expression in ALS and/or an ALS subtype may be synthesized using commercially available reagents or they may be genetically expressed from DNA encoding them.

When the inhibitor of the invention is a small molecule, a small molecule antagonist may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

In another aspect of the invention, one or more biomarker identified as being upregulated or as having increased expression in ALS and/or an ALS subtype can be inhibited by way of inactivating and/or sequestering the biomarker(s). As such, inhibiting the effects of one or more biomarker identified as being upregulated or as having increased expression in ALS and/or an ALS subtype can be accomplished by using a transdominant negative mutant.

In one embodiment, an antibody specific for one or more biomarker identified as being upregulated or as having increased expression in ALS and/or an ALS subtype may be used. As will be understood by one skilled in the art, any antibody that can recognize and bind to an antigen of interest is useful in the present invention. Methods of making and using antibodies are well known in the art. For example, polyclonal antibodies useful in the present invention are generated by immunizing rabbits according to standard immunological techniques well-known in the art. Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magnetic affinity cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods.

Monoclonal antibodies directed against full length or peptide fragments of a protein or peptide may be prepared using any well-known monoclonal antibody preparation procedures. Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art. Further, the antibody of the invention may be “humanized” using methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired.

The invention also includes functional equivalents of the antibodies described herein. Functional equivalents have binding characteristics comparable to those of the antibodies, and include, for example, hybridized and single chain antibodies, as well as fragments thereof.

Functional equivalents include polypeptides with amino acid sequences substantially the same as the amino acid sequence of the variable or hypervariable regions of the antibodies. “Substantially the same” amino acid sequence is defined herein as a sequence with at least 70%, 80%, 90%, 95%, or 99% identity to another amino acid sequence (or any integer in between 70 and 99), as determined by a sequence similarity search algorithm. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′) 2 fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H2L2) formed of two dimers associated through at least one disulfide bridge.

### Methods

In one embodiment, the present invention provides methods for diagnosis of ALS and/or an ALS subtype by detecting a biomarker of the invention in a sample from a subject having or at risk of ALS. In one embodiment, the present invention provides methods for treatment, inhibition, prevention, or reduction of ALS and/or an ALS subtype using a modulator of one or more biomarker of the invention. In certain embodiments, the method of the invention comprises administering to a subject an effective amount of a composition that modulates the expression, activity, or both, of a biomarker of the invention in a cell of the subject.

In one embodiment, the invention provides a method to treat ALS and/or an ALS subtype in a subject in need thereof, comprising detecting the level or activity of one or more biomarker of the invention, diagnosing the subject as having ALS and/or an ALS subtype and treating the subject with a therapy for diagnosed ALS or ALS subtype.

In one embodiment, the invention provides a method of providing a prognosis for a subject diagnosed as having ALS comprising detecting the level or activity of one or more ALS subtype specific biomarker of the invention, diagnosing the subject as having a poor prognosis based on detection of one or more ALS-Glia biomarker or diagnosing the subject as having a good prognosis based on detection of one or more ALS-Ox or ALS-TD biomarker. In some embodiments, the method further comprises and treating the subject with a therapy for diagnosed ALS subtype.

In one embodiment, the method comprises detecting one or more markers in a biological sample of the subject. In various embodiments, the level of one or more of markers of the invention in the biological sample of the subject is compared with the level of a corresponding biomarker in a comparator. Non-limiting examples of comparators include, but are not limited to, a negative control, a positive control, an expected normal background value of the subject, a historical normal background value of the subject, an expected normal background value of a population that the subject is a member of, or a historical normal background value of a population that the subject is a member of.

The invention provides improved diagnosis and prognosis of ALS. The risk of developing ALS and/or the prognosis of ALS can be assessed by measuring one or more of the biomarkers described herein, and comparing the measured values to reference or index values. Such a comparison can be undertaken with mathematical algorithms or formula in order to combine information from results of multiple individual biomarkers and other parameters into a single measurement or index. Subjects identified as having an increased risk of ALS or poor prognosis can optionally be selected to receive treatment regimens, such as administration of prophylactic or therapeutic compounds for ALS. In certain instances, monitoring the levels of at least one biomarker also allows for the course of treatment of ALS to be monitored. For example, a sample can be provided from a subject undergoing treatment regimens or therapeutic interventions, e.g., drug treatments, etc. for ALS. Samples can be obtained from the subject at various time points before, during, or after treatment.

The biomarkers of the present invention can thus be used to generate a biomarker profile or signature of subjects: (i) who have or are expected to develop ALS-Glia and/or (ii) who have or are expected to develop ALS-Ox and/or (iii) who have or are expected to develop ALS-TD. The biomarker profile of a subject can be compared to a predetermined or reference biomarker profile to diagnose or identify subjects at risk for developing a specific ALS subtype, to monitor the progression of disease, as well as the rate of progression of disease, and to monitor the effectiveness of ALS treatments. Data concerning the biomarkers of the present invention can also be combined or correlated with other data or test results, including but not limited to imaging data, medical history and any relevant family history.

The present invention also provides methods for identifying agents for treating ALS that are appropriate or otherwise customized for a specific subject. In this regard, a test sample from a subject, exposed to a therapeutic agent, drug, or other treatment regimen, can be taken and the level of one or more biomarkers can be determined. The level of one or more biomarkers can be compared to a sample derived from the subject before and after treatment, or can be compared to samples derived from one or more subjects who have shown improvements in risk factors as a result of such treatment or exposure.

In another embodiment, the invention provides a method of monitoring the progression of ALS in a subject by assessing the level of one or more of the markers of the invention in a biological sample of the subject.

In various embodiments, the subject is a human subject, and may be of any race, sex and age. Information obtained from the methods of the invention described herein can be used alone, or in combination with other information (e.g., disease status, disease history, vital signs, blood chemistry, etc.) from the subject or from the biological sample obtained from the subject.

In some embodiments, a biological sample from a subject is assessed for the level of one or more of the markers of the invention in the biological sample obtained from the patient. In some embodiments, these methods may utilize a biological sample (such as Cerebrospinal fluid (CSF), spinal cord biopsy samples, motor or frontal cortex tissue samples, urine, saliva, blood, serum, plasma, amniotic fluid, or tears), for the detection of one or more markers of the invention in the sample. In some embodiments the sample will be a “clinical sample” which is a sample derived from a patient. In one embodiment, the biological sample is a motor or frontal cortex tissue sample. In certain embodiments, the biological sample is a CSF fluid sample.

The level of one or more of the markers of the invention in the biological sample can be determined by assessing the amount of polypeptide of one or more of the biomarkers of the invention in the biological sample, the amount of mRNA of one or more of the biomarkers of the invention in the biological sample, the amount of DNA of one or biomarkers of the invention in the biological sample, the amount of enzymatic activity of one or more of the biomarkers of the invention in the biological sample, or a combination thereof.

In some embodiments, the level of one or more markers of the invention is determined to be increased when the level of one or more of the markers of the invention is increased by at least 2%, at least 5%, at least 10%, by at least 20%, by at least 30%, by at least 40%, by at least 50%, by at least 60%, by at least 70%, by at least 80%, by at least 90%, or by at least 100%, when compared to with a comparator control.

In some embodiments, the level of one or more markers of the invention is determined to be decreased when the level of one or more of the markers of the invention is decreased by at least 2%, at least 5%, at least 10%, by at least 20%, by at least 30%, by at least 40%, by at least 50%, by at least 60%, by at least 70%, by at least 80%, by at least 90%, or by at least 100%, when compared to with a comparator control.

### Pharmaceutical Compositions

The present invention includes pharmaceutical compositions comprising one or more modulators of the invention. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intratumoral, epidural, intracerebral, intracerebroventricular, or another route of administration. Other contemplated formulations include projected nanoparticles, liposomal preparations, rescaled erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Kits

The present invention further provides kits for practicing the present methods. Accordingly, in certain embodiments, the invention provides a kit for detecting one or more biomarker in a sample. For example, in some embodiments, the kit comprises one or more nucleic acid binding molecule specific for binding to a biomarker of the invention. In certain embodiments, the invention provides a kit comprising two or more nucleic acid binding molecules specific for binding to two or more biomarkers of the invention. In some embodiments, the two or more biomarkers are associated with two or more different ALS-subtypes to allow for differential diagnosis of the ALS subtype. In certain embodiments, the invention provide a kit for modulating the level or activity of one or more biomarker of the invention.

In some embodiments, the kit may optionally contain one or more of: a positive and/or negative control, materials for isolation and preparation of a nucleic acid sample (e.g., RNase-free water, and one or more buffers), and RNase-free laboratory plasticware (e.g., a plate(s), such a multi-well plate(s), such as a 96 well plate(s), a petri dish(es), a test tube(s), a cuvette(s), etc.).

Any kit of the invention may also include suitable storage containers, e.g., ampules, vials, tubes, etc., for each reagent disclosed herein. The reagents may be present in the kits in any convenient form, such as, e.g., in a solution or in a powder form. The kits may further include a packaging container, optionally having one or more partitions for housing the various reagents or components.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Molecular Subtypes of ALS are Associated with Differences in Patient Prognosis

This study demonstrates that a large cohort of ALS patient transcriptomes can be stratified into three subtypes defined by distinct molecular phenotypes, termed ALS-Glia, ALS-Ox, and ALS-TD. Gene expression associated with activated glial cells are observed in the ALS-Glia subtype, while the ALS-Ox subtype is characterized by oxidative stress, proteotoxic stress, and increased inhibition in the frontal and motor cortices. Consideration of locus-specific transposable elements revealed that both the ALS-TD and ALS-Ox subtypes strongly overexpressed TEs compared to healthy control donors and ALS-Glia patients. Guided by enrichment, the unique expression of transcription and translation associated genes were observed, including transcription factors, regulatory microRNAs, mRNA traditionally marked for nonsense mediated decay, pseudogenes, antisense, intronic, and long non-coding RNAs. These findings led us to define the final subtype by transcriptional dysregulation (ALS-TD). These subtypes had significant differences in survival, and eigengene analysis provides new insight into the variability observed in ALS patient age at symptom onset and age at death. Given these results, ALS-Glia specific upregulation and downregulation of genes in the frontal and motor cortex provides a novel set of transcripts associated with patient prognosis.

The exceptionally large patient cohort NCBI Gene Expression Omnibus (GEO) accession GSE153960 was used to analyze the subtype-driven heterogeneity in ALS. Patient stratification analysis was performed using RNA-sequencing (RNA-seq) expression data from the frontal and motor cortex of 208 ALS patients, corresponding to 451 unique tissue samples. Transposable elements (TE) were quantified at the locus-specific level, which resulted in the redefinition of one ALS subtype. Three distinct molecular subtypes were identified, with significant differences in survival, defined by i) glial activation (ALS-Glia), ii) oxidative stress and altered synaptic signaling (ALS-Ox), and iii) transcriptional dysregulation (ALS-TD). Importantly, these subtypes capture most of the existing disease mechanisms previously associated with ALS neurodegeneration. In addition, some of the subtype-specific genes and transcripts identified in this study have not been previously associated with ALS, offering new insight into disease pathologies and potential targets for diagnostic or personalized therapeutic development.

### ALS Patients

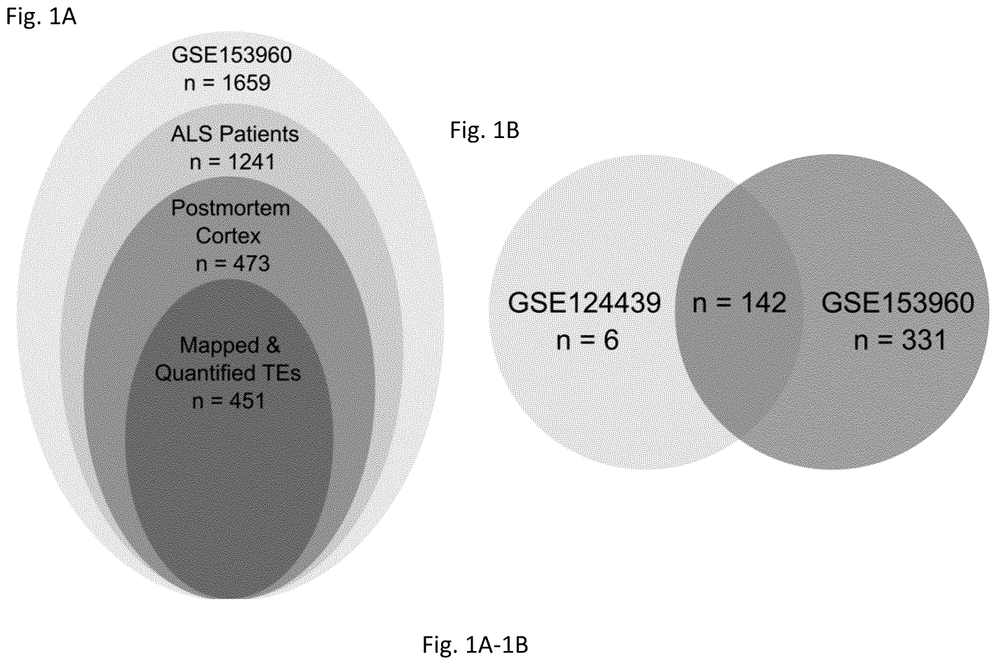

Within the GEO data repository, GSE153960 was identified as the ideal study to further probe the existence of ALS subtypes. GSE153960 contains RNA-seq data from 1659 tissue samples, spanning 11 regions of the CNS, from 439 patients with ALS, frontotemporal lobar degeneration (FTLD), or comorbidities for ALS-Alzheimer's (ALS/AD) or ALS-FTLD. These 1659 tissue samples were filtered such that only the individuals belonging to the groups “ALS-TDP”, “ALS/FTLD”, “ALS/AD”, and “ALS-SOD1” were considered. Furthermore, RNA-seq samples derived from regions of the CNS other than the frontal or motor cortex, such as cerebellum and spinal cord, were not included in the analysis-yielding 473 cortex transcriptomes FIG. 1A.

Raw FASTQ files for the 473 ALS patient samples were downloaded from the European Bioinformatics Institute data repository (NCBI mirror) using NIH's Globus software. Of the 473 selected RNA-seq samples, five had incomplete or missing paired end FASTQ files, and were subsequently excluded from the analysis. An additional 13 samples were mapped to the human reference genome build hg38 via STAR 2.5.3a but TEs were not successfully quantified using the SQUIRE pipeline and were therefore excluded from the analysis. A final 4 samples were poorly mapped to the RepeatMasker transposable element reference genome retaining these four subjects would have resulted in a reduction of ‘shared’ TEs by >60% (557/1474). The final ALS cohort contained 451 frontal and motor cortex transcriptomes, corresponding to 208 unique patients. Subject demographics for this analysis are included in Table 1.

TABLE 1
Subject Demographics

| A-L: Axial and Limb Onset | Cohort Demographics | | | | |
|---|---|---|---|---|---|
| B-L: Bulbar and Limb Onset | ALS Spectrum | | FTLD | | Healthy Control Donors |
| A-B: Axial and Bulbar Onset | (n = 208) | | (n = 42) | | (n = 58) |
| Sex | | | | | |
| Female | 95 | (45.7%) | 18 | (42.9%) | 28 (48.3%) |
| Male | 113 | (54.3%) | 24 | (57.1%) | 30 (51.7%) |
| Tissue site | n = 451 | | n = 42 | | n = 93 |
| Frontal Cortex | 193 | (42.8%) | 42 | (100%) | 56 (60.2%) |
| Lateral Motor Cortex | 104 | (23.1%) | 0 | | 18 (19.4%) |
| Medial Motor Cortex | 102 | (22.6%) | 0 | | 19 (20.4%) |
| Motor Cortex Unspecified | 52 | (11.5%) | 0 | | 0 |
| ALS subtype | | | NA | | NA |
| ALS-Glia | 33 | (15.9%) | — | | — |
| ALS-TD | 56 | (26.9%) | — | | — |
| ALS-OX | 89 | (42.8%) | — | | — |
| ALS-Discordant | 30 | (14.4%) | — | | — |
| Disease duration (months) | | | Not Available | | NA |
| ALS-Glia | 29.1 ± 3.81 | | — | | — |
| ALS-TD | 38.1 ± 3.40 | | — | | — |
| ALS-OX | 41.8 ± 3.18 | | — | | — |
| ALS-Discordant | 42.4 ± 6.35 | | — | | — |
| Age of onset (years) | | | Not Available | | NA |
| ALS-Glia | 63.2 ± 1.83 | | — | | — |
| ALS-TD | 62.7 ± 1.68 | | — | | — |
| ALS-OX | 60.4 ± 1.16 | | — | | — |
| ALS-Discordant | 60.9 ± 1.87 | | — | | — |
| Site of onset | | | NA | | NA |
| ALS-Glia | Bulbar: 11; Limb: 20; | | — | | — |
| | Unknown: 2 | | | | |
| ALS-TD | Bulbar: 17; Limb: 35; | | — | | — |
| | Axial: 2; A-L: 1 | | | | |
| | Unknown: 1 | | | | |
| ALS-OX | Bulbar: 23; Limb: 51; | | — | | — |
| | Axial: 2; A-B: 1; | | | | |
| | A-L: 1; B-L: 4; | | | | |
| | Generalized: 1; | | | | |
| | Unknown: 6 | | | | |
| ALS-Discordant | Bulbar: 8; Limb: 19; | | — | | — |
| | Unknown: 2; A-B: 1 | | | | |
| Age of death (years) | | | 66.5 ± 9.5 | | 64.8 ± 15.7 * |
| ALS-Glia | 66.1 ± 1.60 | | — | | — |
| ALS-TD | 66.7 ± 1.33 | | — | | — |
| ALS-OX | 64.0 ± 1.05 | | — | | — |
| ALS-Discordant | 64.0 ± 1.65 | | — | | — |
| FTLD comorbidity | 27/208 | (13.0%) | 42/42 | (100%) | NA |

| | | | | |
|---|---|---|---|---|
| ALS-Glia | 6/33 | (18.2%) | — | — |
| ALS-TD | 8/56 | (14.3%) | — | — |
| ALS-OX | 10/89 | (11.2%) | — | — |
| ALS-Discordant | 4/30 | (13.3%) | — | — |

* Three healthy control samples had an age of death listed as “90 or Older”. A conservative estimate of 90 years was used for all samples listed as such.
Disease Duration, Age of Onset, and Age of Death metrics presented as mean ± standard error

### Control Subjects

Control sample transcriptomes were comprised of healthy control donors (HC; n=93) and patients diagnosed with FTLD exclusively (n=42), corresponding to 58 HC and 42 FTLD subjects. Equivalent to the ALS subject processing pipeline, raw FASTQ files were downloaded from the European Bioinformatics Institute data repository. One RNA-seq sample had missing paired-end FASTQ files and was excluded from the analysis. The remaining 135 control samples were mapped to the human reference genome build hg38 using STAR 2.5.3a and TEs were quantified using SQUIRE's Count function. TEs missing from the control sample cohort were replaced with a count value of 0. Controls were considered during development of the supervised classifiers to assess predictive accuracy in the likely event of clinical misclassification. Transcriptomes from the control cohort were implemented during GSEA for the identification of enriched pathways associated with each of the three subtypes. Control samples were further utilized to assess differentially expressed genes and TEs in each ALS subtype.

### Quantification

Quantification of gene expression was performed using RSEM. The processed gene count matrix was accessed directly from the GEO Accession (GSE153960) and counts were rounded to integers as recommended by the authors of RSEM and required by DESeq2 differential expression.

SQUIRE was selected for transposable element quantification, as this alignment pipeline provides locus-specific TE counts, allowing for a deeper analysis beyond TE subfamilies. Similar to RSEM, SQUIRE applies the Expectation Maximization (EM) algorithm to optimize the allocation of multi-mapped reads. SQUIRE's Fetch, Clean, Map, and Count functions were utilized to align and quantify locus-specific transposable elements. The EM ‘tot_counts’ values were selected as the estimate for sequencing reads attributed to the transposable elements. The hg38 build was used during mapping, with default trim and EM parameters, and a read length of 100 or 125 base pairs depending on the sequencing platform specified. A scoring threshold of >99 was used to restrict the number of false positive TEs (1%), with few uniquely mapping reads. Only the locus specific TEs with at least one count for all ALS samples (n=451) were included in downstream analysis, resulting in 1474 unique TE features. The naming scheme for the locus-specific transposable elements is presented in SQUIRE. In brief, TE feature names included the mapping chromosome, start and stop base pairs, transposable element subfamily, family and superfamily identifiers, base mismatches in parts per thousand, and sense or antisense stand annotation.

### Differential Expression

The large ALS cohort size required the utilization of two different sequencing platforms (HiSeq 2500 and NovaSeq 6000, Illumina, San Diego, CA) to complete the analysis. Exploratory differential expression considering sequencing platforms as the design equation factor revealed strong batch effects in gene expression, evident by nearly one-third of all genes falling below the Benjamini-Hochberg corrected p-value threshold. To correct for these batch effects, the ALS cohort was split based on sequencing platform. The NovaSeq cohort contained 255 patient transcriptomes, while the HiSeq cohort contained 196. The control cohort was processed in an analogous manner.

DESeq2 was initially applied to perform a preliminary differential expression on gene and TE counts. Differential expression was utilized to guide the removal of sex dependent genes prior to clustering. As described by the authors of GSE153960, sex was determined using XIST and UTY expression. Default parameters were used for DESeq2 differential expression, with “male” specified as the reference level and the “betaPrior” argument in the DESeq( ) function set to true. A Benjamini-Hochberg corrected p-value ≤0.05 was selected as the threshold for removal of sex-dependent genes.

### Clustering

Following the removal of sex-dependent genes using differential expression, the raw count matrix was subject to a variance stabilizing transformation (VST) to address heteroskedasticity in gene counts. The VST counts were then subject to rank ordering by median absolute deviation (MAD) and the top 10,000 features were retained for unsupervised clustering analysis by non-negative matrix factorization (NMF). This process was completed for both sequencing platform cohorts.

### Rank Estimation

Factorization rank was estimated in R, Version 4.0.3 (The R Foundation for Statistical Computing, Vienna, Austria) using the ‘NMF’ package. A rank of 3 was selected for clustering analysis, based on the plots of the cophenetic correlation coefficient for ranks spanning 2 to 6. Quality measures were estimated using iterations at each rank and the default seeding method. The ‘nsNMF’ (non-smooth non-negative matrix factorization) method was utilized for all NMF clustering.

### Non-Negative Matrix Factorization

Non-negative matrix factorization was performed in SAKE, a convenient tool for RNA-seq sample pre-processing, filtering, clustering and visualization (Version 0.4.0). The top 10,000 MAD genes, after a variance stabilizing transformation, were utilized as the input into SAKE. No samples were removed during the quality control step, and further transformations in the filtering step were not necessary. During non-negative matrix factorization, selected parameters include factorization rank=3, iterations=200, and NMF method set to ‘nsNMF’.

To robustly assign ALS sample subtypes, rounds of NMF clustering were performed in SAKE. For each patient sample, the ALS subtype with a simple majority was selected. For a very small number of ‘edge’ cases (5/451), an eleventh round of NMF clustering was used as a tiebreaker to reach the simple majority threshold. This process was completed for both sequencing platform groups.

### Feature Selection

After each replicate of NMF clustering, gene and TE feature scores were calculated for all 10,000 MAD transcripts. Feature scores were averaged across the clustering replicates and reordered. The top 1000 features from both sequencing platform cohorts were combined, and after the removal of duplicates, 1681 genes and TEs remained for enrichment, networking, and univariate analysis.

### Gene Set Enrichment Analysis

Following supervised classification, the gene and TE feature sets were then enriched using GSEA. Healthy control donors were selected as the reference phenotype during enrichment. Transcripts without a corresponding gene symbol (HGNC) were excluded from the enrichment analysis, including TEs, leaving 891 total genes. The minimum gene set size was adjusted to, and all other parameters were maintained as the default. For the enrichment, the canonical pathways contained in the Reactome database was leveraged, including a custom gene set containing markers of disease-associated microglia, and curated gene sets for Alzheimer's, Parkinson's, and ALS. Pathway heatmaps reflecting gene enrichment by phenotype were built using the “Rank Metric Score” tabulated during GSEA.

A custom gene set for the enrichment of locus-specific transposable elements was also considered, however GSEA rank-based scoring may be biased by the size of the TE set (>400 features). The collapse of locus-specific TEs to the subfamily level was also considered, to allow enrichment using Repbase, however subfamily co-expression was not observed following a hierarchical clustering analysis considering TE features exclusively.

### Networks

Network development was carried out in two different, yet complementary, approaches. For the visualization of gene enrichment pathways by ALS phenotype, Cytoscape (Version 3.8.2, Institute for Systems Biology, Seattle, WA) was leveraged for analysis. Result files from GSEA were utilized as the input into Cytoscape. Additional pathway enrichment was performed using the custom and curated gene sets from the previous step. Nodes were color-coded according to ALS subtype specificity, guided by GSEA enrichment score magnitude and univariate analysis. A small number of unrelated or synonymous pathways were manually trimmed.

Co-expressed gene subsets (“eigengenes”) associated with disease duration, age of symptom onset, and age at death were assessed using the Weighted Gene Co Expression Network Analysis (WGCNA) package in R. The minimum module size was set to 25 and a ‘soft power’ of 13 was selected given the assessment of scale-free topology. All 1681 features were considered during construction of the eigengene heatmap. Eigengenes of interest were subject to network visualization in VisANT using edge weights obtained from WGCNA. A weight threshold of 0.05 was set to filter genes weakly co-expressed. Unconnected nodes were manually trimmed from the networks.

### Classification

Subtype predictor gene sets were derived from the purple, turquoise, and magenta eigengenes, given their specific associations with the ALS-Glia, ALS-Ox, and ALS-TD subtypes, respectively. Subtype scores, defined as the average expression of subtype specific predictor genes minus the average expression of the 1681 features selected by NMF, were calculated for 100 different sets of predictors (per subtype) and used to define a 5% cutoff for the expected subtype score. Each sampled predictor gene set contained the same number of features as the original set of subtype predictors and were generated by randomly sampling the eigengenes with replacement. Expected subtype scores were rank ordered and used to define a classification threshold for each subtype, weighted according to the observed proportion of patient samples obtained from the clustering analysis. Bootstrapping was then applied, involving the sampling of predictor gene sets (with replacement) and calculation of subtype score across 1,000 iterations. Patient samples were initially placed at the origin and moved in the direction of the subtype vertex after passing the corresponding subtype threshold. Therefore, the x, y, and z axis vertices reflect the expression of a single subtype, while the other three vertices capture a combination of two subtypes. Individual points that passed a given subtype threshold >50% of the time were filled with their respective subtype colors. Samples were considered to express a hybrid subtype state if one subtype threshold was passed >50% of the time and simultaneously passed a second subtype threshold >40% of the time. All machine learning classifiers were developed in Python using the Scikit-learn framework. Four different models were considered, k-nearest neighbors (KNN), linear support vector classification (Linear SVC), multilayer perceptron (MLP), and random forest (RF). To limit the inclusion of platform-dependent genes, the top 1000 features were further filtered so that only genes and TEs shared between the two sequencing platform cohorts were retained, totaling 299. The k-nearest neighbor classifier was built with k neighbors=5, distance calculated using the Manhattan metric, weights=‘distance’, and all other parameters as default. The linear SVC classifier was constructed using class weights defined by the proportion of subtypes in the NovaSeq cohort, max iterations=100000 and default for all other parameters. The multilayer perceptron neural network was built using three hidden layers (five total), with 100 ‘neurons’ comprising each hidden layer, learning rate=0.0001, hyperbolic tangent activation function, random state=1, max iterations=10000 and default settings for all remaining parameters. Finally, the random forest was developed using n estimators=1000, oob score=‘True’, class weights defined by the proportion of subtypes in the NovaSeq cohort, and default for all other parameters. All models were constructed using the ‘one-vs-rest’ multi-class strategy.

Supervised classifiers were constructed using training and testing datasets generated from a 70%/30% split of the ALS NovaSeq cohort. 100-fold cross validation was applied to assess performance in the testing cohort. The ALS HiSeq cohort was designated as the holdout dataset to assess performance metrics when classifying new patient samples. Transcript counts on the VST scale were utilized during classifier development. Classifier recall, precision, and F1 scores were calculated for all ALS subtypes after each round of cross validation.

### Clinical Parameters

For many patients in the cohort, multiple tissue samples from the frontal and motor cortex were characterized by RNA-seq. As a result, patients were assigned a label only if there was a majority consensus among their frontal and motor cortex samples, or if there was a single sample characterized. ALS patients which displayed multiple subtypes among their frontal and motor cortex samples were labeled ‘Discordant’. Among the 208 unique patients in this cohort, 30 were found to be discordant.

Differences in ALS survival by subtype was assessed using the Kaplan-Meier analysis with application of the log-rank statistical test. Subtype-specific differences in age of symptom onset and age at death were analyzed using ANOVA tests. A Chi-Squared Test of Independence was applied to assess subtype specificity for FTLD comorbidity. All analysis was performed with and without discordant ALS patients.

### Subtype Concordance

Two studies are associated with the New York Genome Center (NYGC) ALS Consortium (GEO Superseries GSE137810), so a large majority (˜95%; n=140) of postmortem tissue samples originally analyzed by the original study were also reanalyzed. The previous analysis proved useful as this repeat analysis utilized the work from the original study as a reference to assess patient subtype concordance. A subtype concordance matrix highlights the strong agreement of subtype labels (85%) between this analysis and the foundational work for the 140 samples in common (Table 2).

TABLE 2
Concordance Matrix

Eshima et al.

| | | | |
|---|---|---|---|
| Concordance Matrix | ALS-TD | ALS-Ox | ALS-Glia |

| | | | | |
|---|---|---|---|---|
| Tam et al. | ALS-TE | 21 | 6 | 0 |
| | ALS-Ox | 9 | 79 | 1 |
| | ALS-Glia | 1 | 4 | 19 |

### Univariate Analysis

Transcript counts were normalized using DESeq2 size factor estimation (median-of ratios) to better allow comparison between patient samples. Subtype-specific differential expression of transcripts was determined using a multifactor design equation, accounting for sequencing platform count-dependencies and patient subtype. Pairwise analysis was performed using the constrast( ) argument, for all combinations. Genes and TEs with an FDR adjusted p-value≤0.05 were considered to be significant. All patient samples (n=586) were considered during normalization. Counts on the median-of-ratios scale were log 2 transformed before plotting.

A few additional genes not included in the 1681 features used for classification, enrichment, and networking, were also considered during the univariate analysis out of disease relevance and include: TARDBP, OXR1, BECN1, BECN2, SOD1, UBQLN1, UBQLN2, UCP2 and TXN. Many of these added genes were used during unsupervised clustering as some of the top 10,000 most variable features calculated by median absolute deviation.

### Data Availability

All raw data files and the RSEM processed gene count matrix utilized in this study are accessible through Gene Expression Omnibus accession: GSE153960.

### The Experimental Results are Now Described

### Unsupervised Clustering Identifies Three Molecular Subtypes in the Frontal and Motor Cortices of ALS Patients

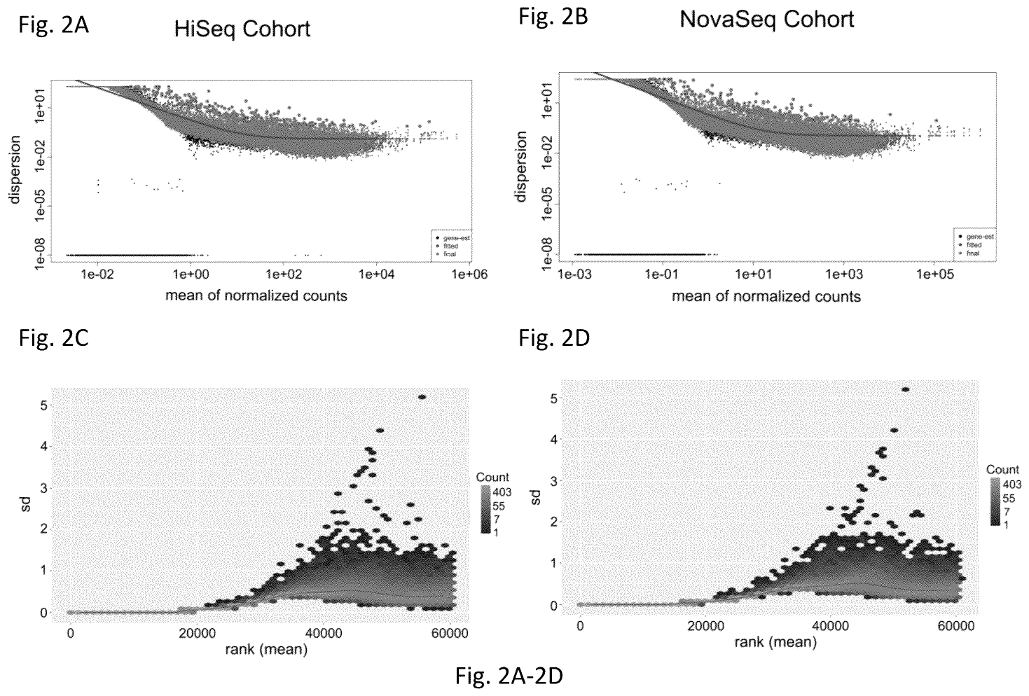

An unsupervised clustering analysis was performed using 451 ALS postmortem cortex transcriptomes (FIG. 1, Table 1). SQUIRE was implemented to quantify transposable element expression with chromosomal locus specificity. TE features were filtered to ensure the retained transcripts had unique mapping reads and quantifiable expression in all ALS patient samples. Prior to clustering, a variance stabilizing transformation was applied (FIG. 2) and the removal of sex-dependent genes was performed using DESeq2 differential expression.

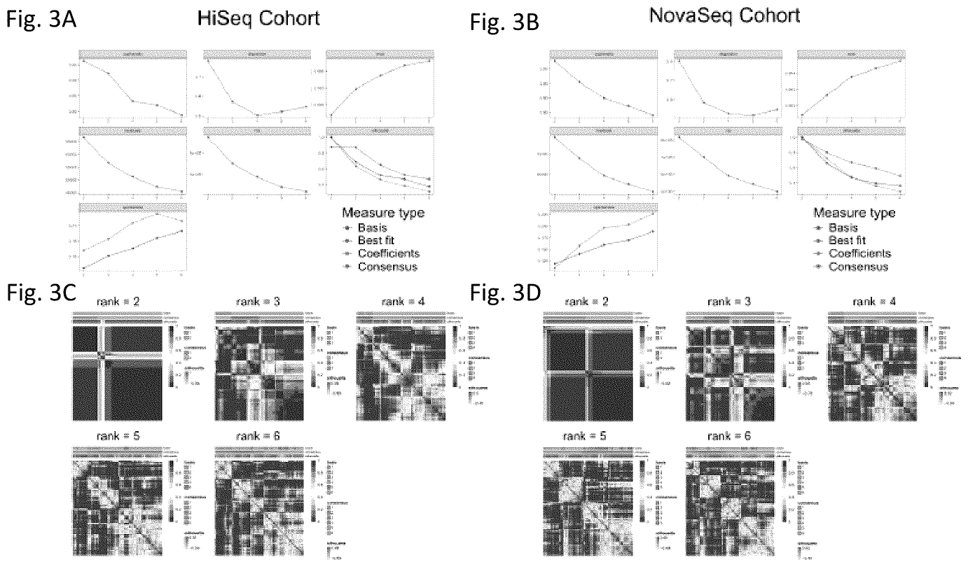

Estimation of factorization rank was then performed in R, and a rank of 3 was chosen considering the quality metrics (FIG. 3). The cohort was split by sequencing platform (HiSeq 2500 and NovaSeq 6000, Illumina, San Diego, CA), to account for substantial batch effects in gene expression due to the use of different sequencing instruments.

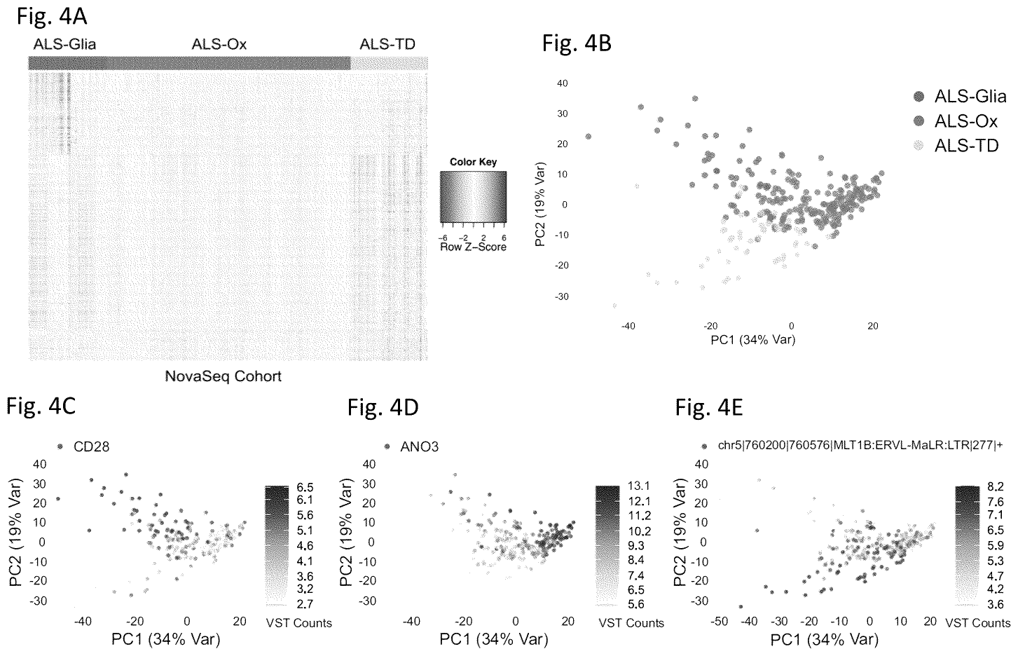

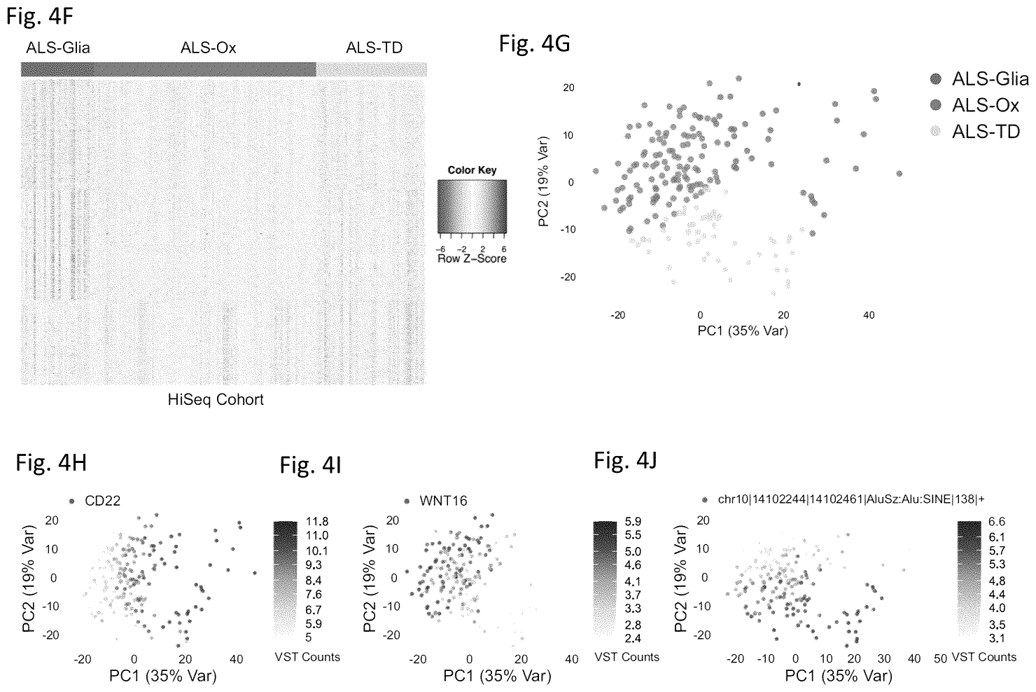

After filtering for the top 10,000 most variably expressed genes, non-smooth non-negative matrix factorization (nsNMF) was applied to identify subgroups of ALS patients based on gene expression in the postmortem cortex. Three distinct patterns of gene expression were identified in both the NovaSeq and HiSeq ALS cohorts (FIGS. 4A and 4F). In the NovaSeq cohort there was roughly a ratio of 3:1.4:1 observed for the ALS-Ox, ALS TD, and ALS-Glia subtypes, respectively. The HiSeq cohort showed a similar proportion of ALS subtypes, with an approximate 3:1.9:1 ratio observed for the ALS-Ox, ALS-TD, and ALS-Glia subtypes, respectively. Principal component analysis (PCA) demonstrated the ability to separate the putative ALS subtypes into three distinct clusters when considering the first and second principal components (FIGS. 4B and 4G). Six transcripts associated with ALS-Glia, ALS-Ox, and ALS-TD were considered in the principal component space, and subtype-specific expression can be identified in both sequencing platform cohorts (FIGS. 4C-4E and 4H-4J). Taken together, these results support the existence of three distinct patterns of gene and TE expression within the ALS postmortem cortex transcriptome.

### Gene Set Enrichment Analysis Reveals Subtype-Specific Phenotypes

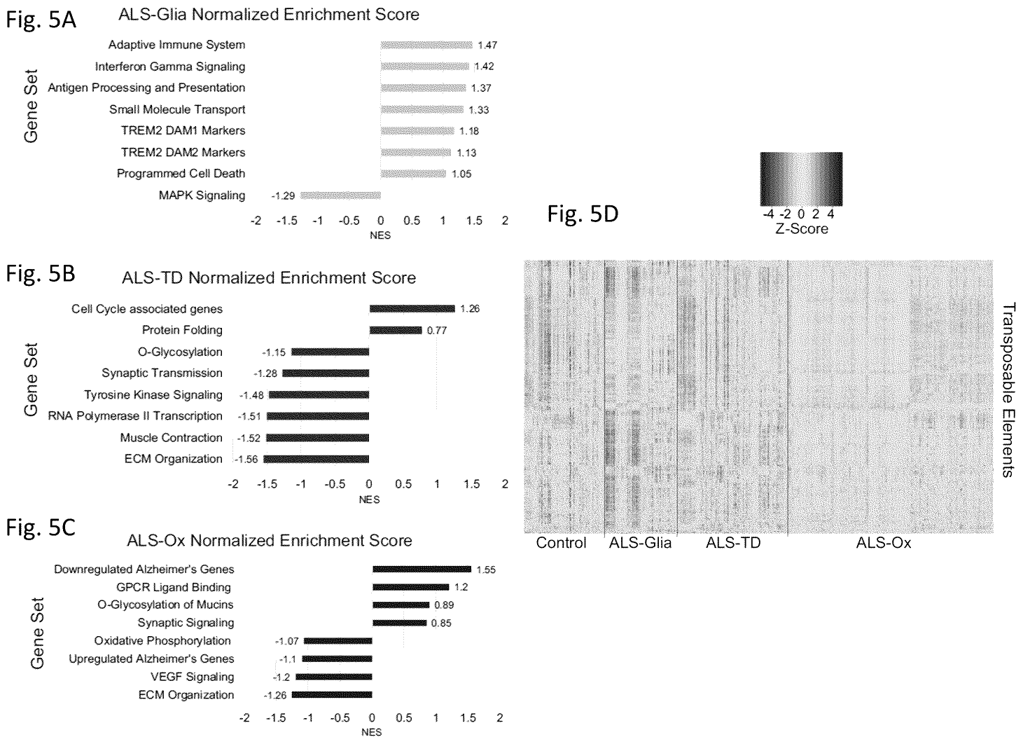

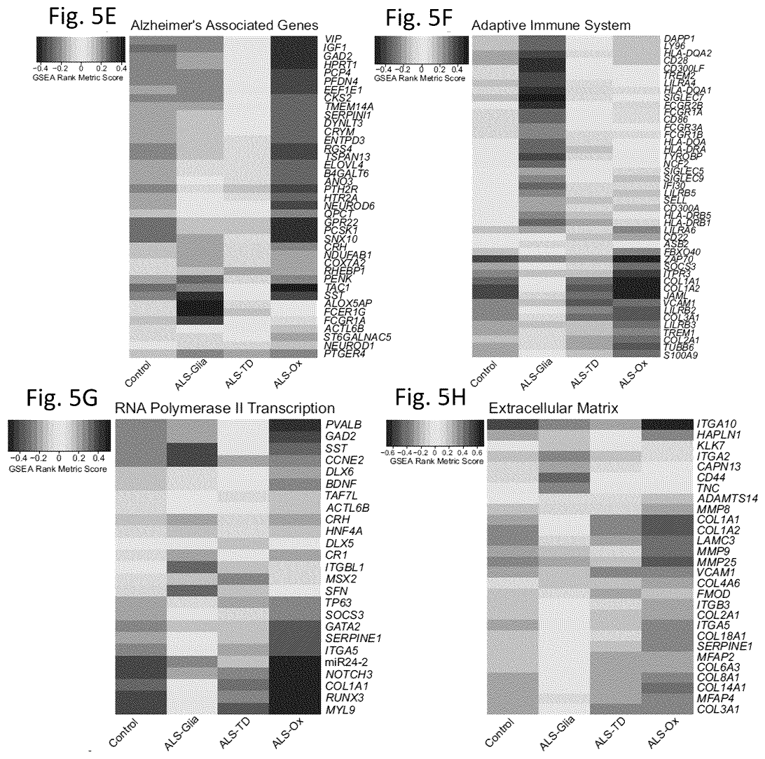

To elucidate subtype-specific molecular phenotypes, Gene Set Enrichment Analysis (GSEA) was performed using the top 1000 features from each sequencing platform cohort, leaving 1681 unique genes and TEs. Subtype-specific pathway enrichment was observed for each ALS subtype (FIG. 5).

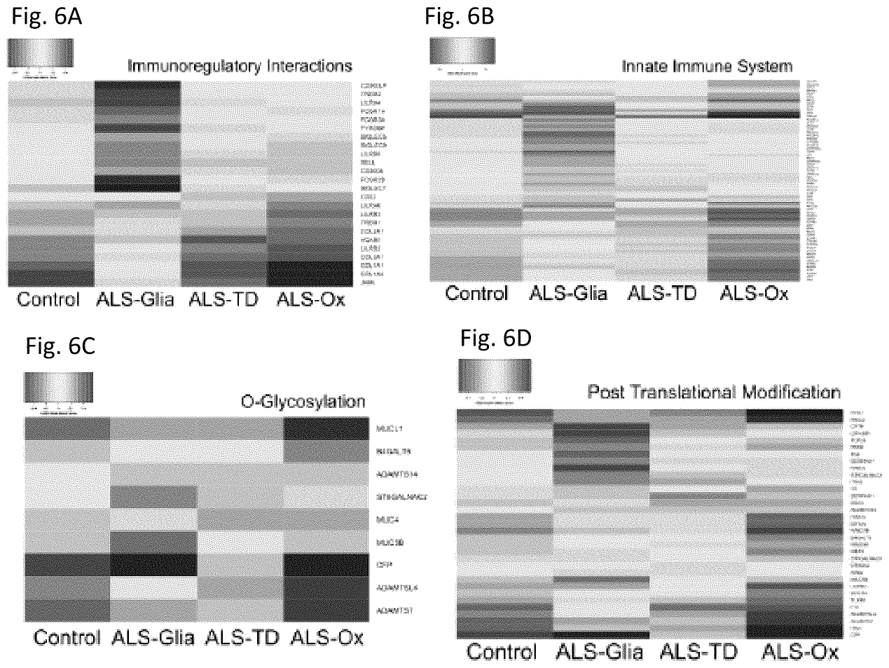

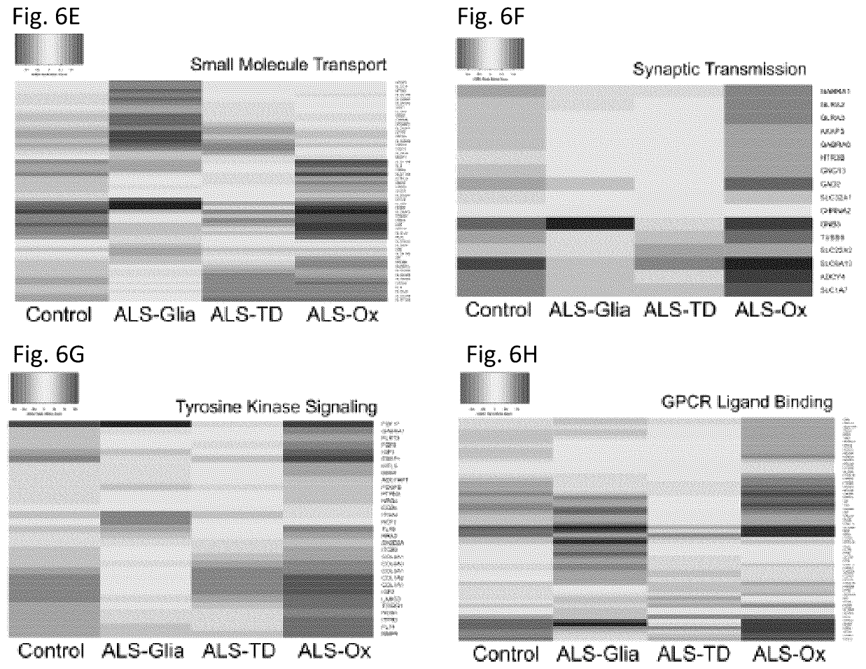

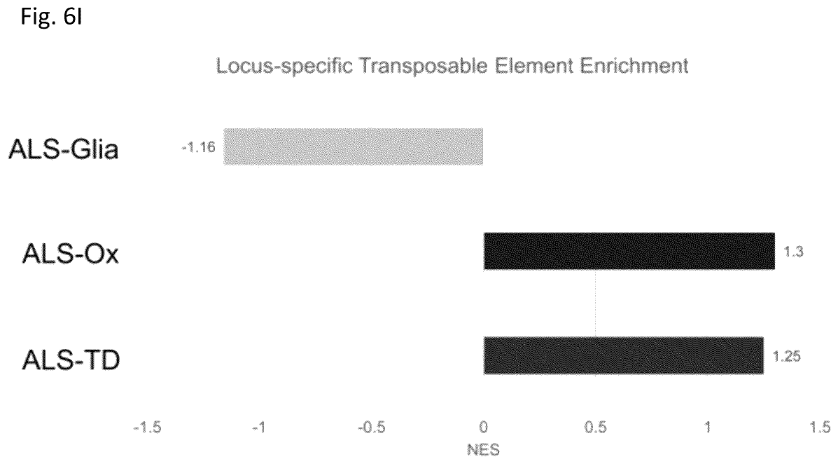

In ALS-Glia samples, enrichment for immunological signaling and activation, genes implicated in a pro-neuroinflammatory microglia state in Alzheimer's (Disease-Associated Microglia, DAM), and markers of neural cell death were observed (FIGS. 5A and 5F; FIG. 6A-6B). Transposable element expression was greatly reduced in ALS-Glia samples compared to the other two subtypes (FIG. 5D).

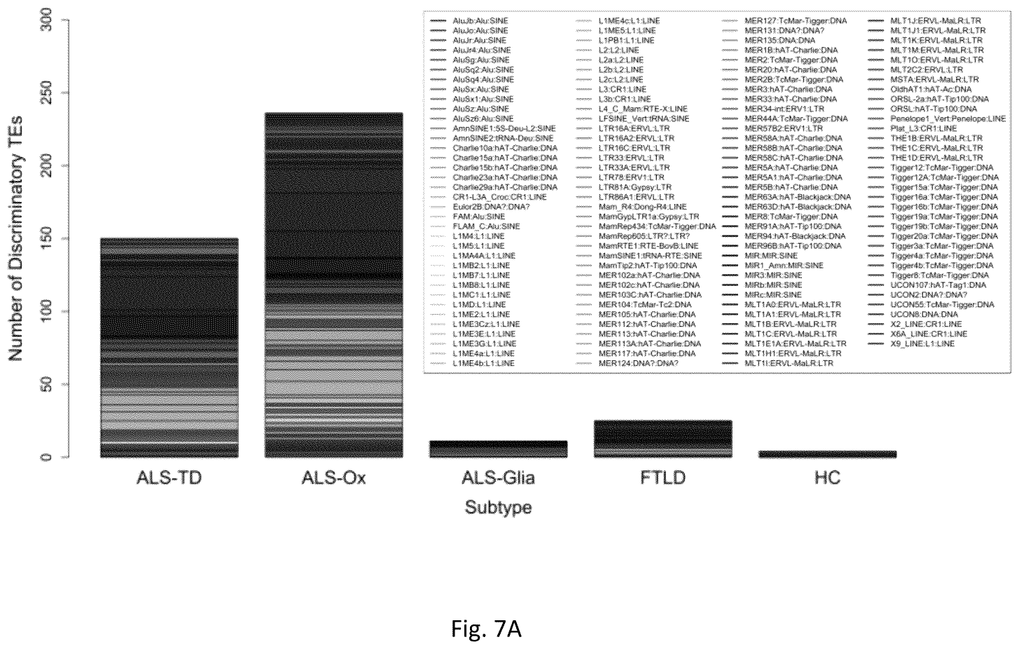

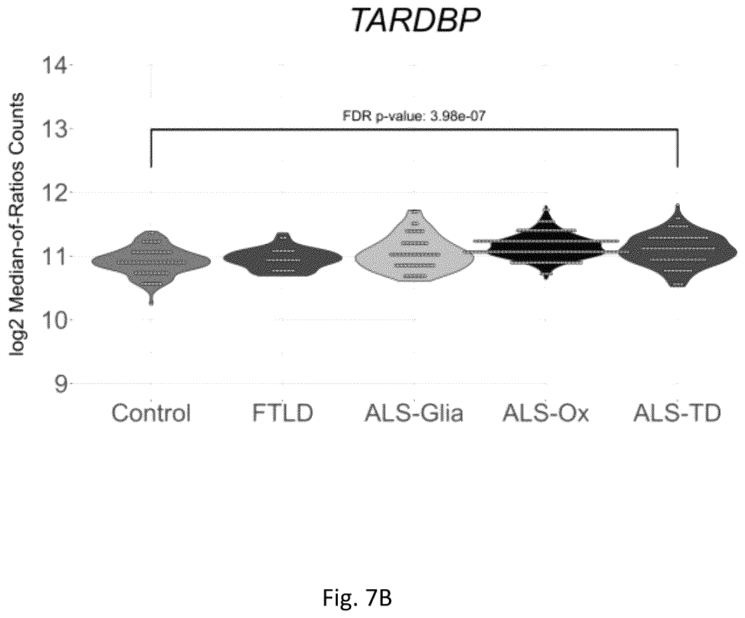

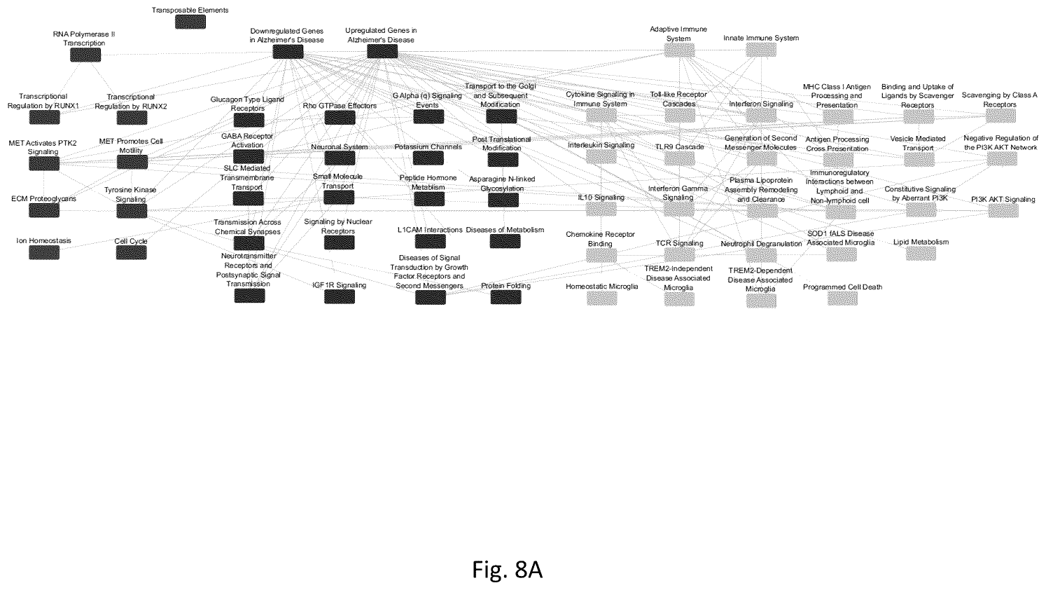

Enrichment of the ALS-TD and ALS-Ox subtypes suggested some overlapping disease mechanisms, such as altered ECM maintenance and the influence of post translational modification machinery (FIG. 5B, 5C, 5H, and FIG. 6C-6D). Furthermore, while the ALS-Ox subtype had the strongest expression of the locus-specific TEs (FIG. 5D), the ALS-TD subtype showed elevated TE expression more often than the control groups and ALS-Glia subtype (FIG. 7A). The unique downregulation of RNA Polymerase II transcriptional genes was observed to distinguish the ALS-TD subtype from ALS-Ox (FIG. 5G). This evidence was utilized along with univariate features considered later, to determine that this ALS subgroup is defined by transcriptional dysregulation (TD), rather than TE expression.

In the ALS-Ox subtype distinct enrichment of Alzheimer's associated genes, but not genes previously associated with ALS or Parkinson's disease, were noted. This may reflect on the NMF score-based feature selection strategy. There was negative enrichment for genes involved in oxidative phosphorylation (FIG. 5C), and positive enrichment for synaptic signaling and neuronal membrane receptors (FIG. 6E-6H), when compared to the control cohort. It is worth noting that the subtype enrichment generally agrees with previous studies, despite the increased size of the patient cohort, despite not including the custom TE enrichment (FIG. 6I) and some differences are observed for the ALS-Ox group. The ALS subtype naming conventions were maintained where appropriate.

Network Development Reveals Gene Subsets Correlated with ALS Disease Duration, Age of Symptom Onset, and Age at Death

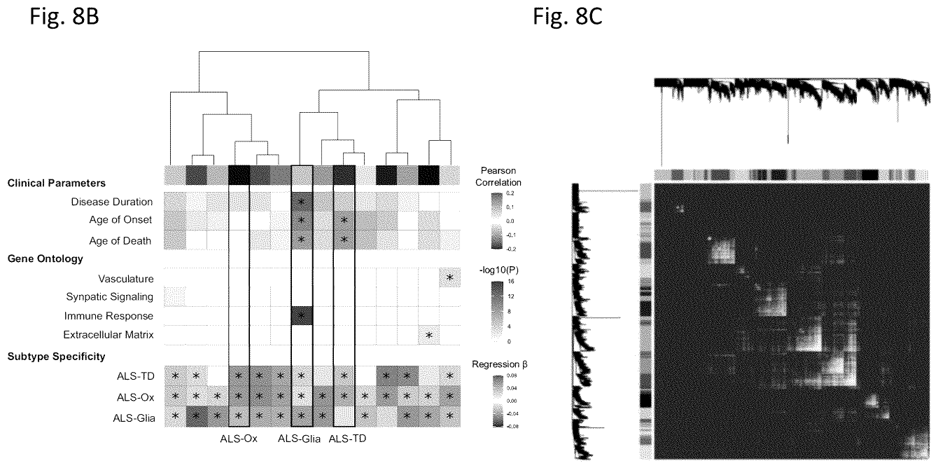

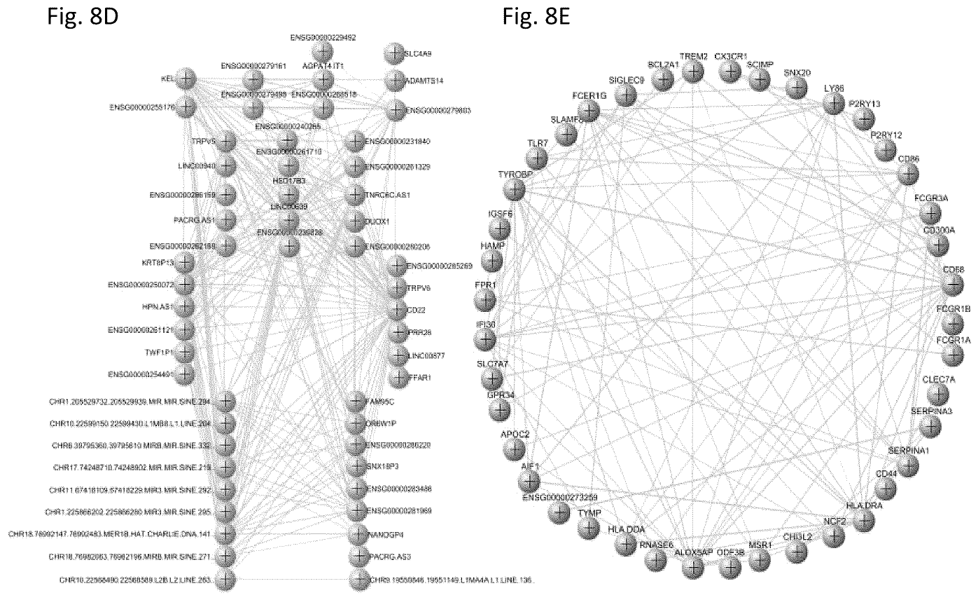

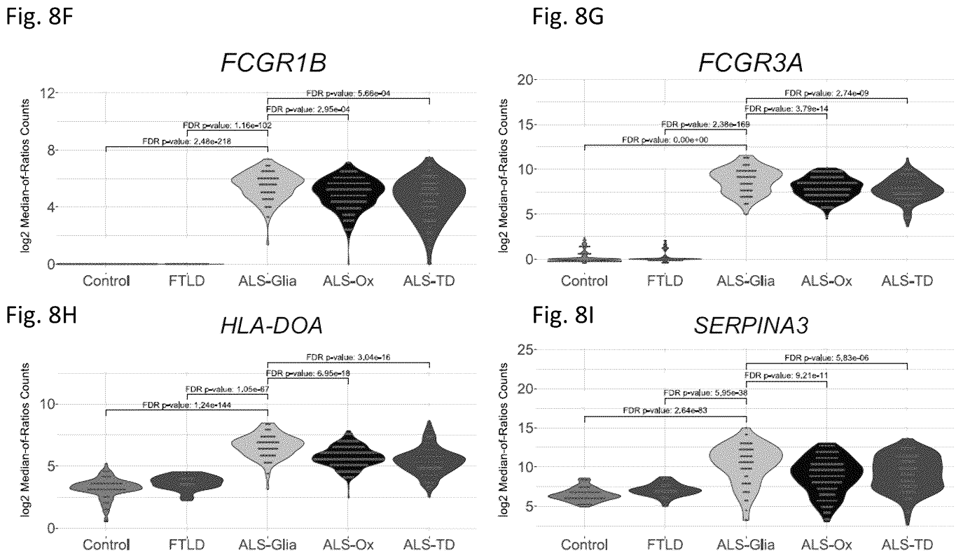

A network in Cytoscape was constructed to facilitate the interpretation of subtype-specific pathway enrichment, utilizing the results from GSEA (FIG. 8A). Pathway nodes were manually color coded by subtype and edges denote overlapping genes between pathways. The ‘Transposable Elements’ node is color coded purple to signify specificity for both the ALS-Ox and ALS-TD subtypes (FIG. 7A). Although informative, this analysis was complemented by identifying co-expressed genes associated with patient clinical parameters, such as age of symptom onset, age of death, and disease duration using a weighted gene co-expression network analysis (WGCNA).

The analysis indicated that the magenta and purple eigengenes are significantly correlated with ALS clinical parameters (FIG. 8B). Identification of eigengene clusters based on the correlation of gene expression is shown as the dendrogram and heatmap plot (FIG. 8C) and calculation of module membership is presented. Genes and TEs comprising the magenta eigengene are shown as a network (FIG. 8D) and expression of this eigengene is seen to be negatively correlated with age of symptom onset and age at death. Conversely, the purple eigengene (FIG. 8E) is seen to be positively correlated with age of onset and death, yet negatively correlated with disease duration. The observed relationship between the purple eigengene and patient clinical parameters indicates that elevated expression drives a later disease onset but a shorter survival duration.

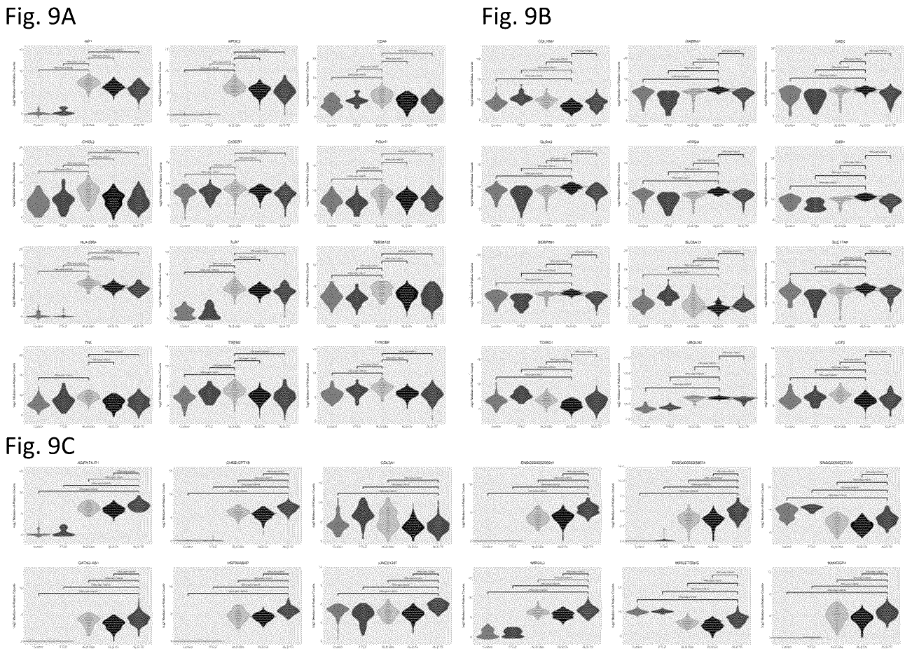

Eigengenes were enriched for gene ontology, and the purple eigengene was seen to be strongly linked to the immune system (p<5×10−16, Fisher exact test, Bonferroni-corrected). Importantly, ALS-Glia specific overexpression was observed for the majority of features included in the purple eigengene (FIG. 8F-81, FIG. 9). The magenta eigengene—primarily composed of transposable elements, long non-coding RNA, pseudogenes, and poorly characterized transcripts (Ensembl IDs)—was not significantly linked to any gene ontologies, although a general association with transcription is perhaps a reasonable interpretation. ALS-TD specific expression was observed for many of the features comprising the magenta eigengene.

### Patient Classification Highlights Hybrid Subtype States

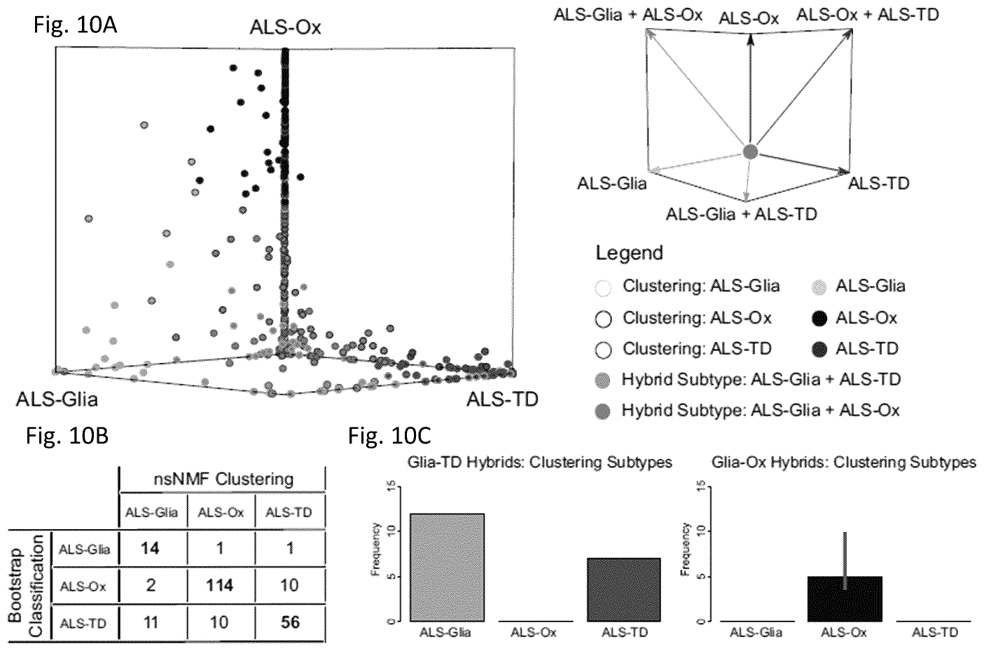

There was evidence for the co-expression of subtype phenotypes within this ALS cohort, guided by the clustering, enrichment, and network results (FIGS. 4A and 4F; FIGS. 5B-5C and 5D-5H; and FIG. 8A). To understand the transcriptional landscape of these molecular subtypes of ALS, the classification approach was leveraged for subtype scores. Subtype scores were calculated using predictor gene sets derived from the ALS-Glia (purple), ALS-Ox (turquoise), and ALS-TD (magenta) eigengenes. The majority of classified patient samples were observed to demonstrate gene expression characteristic of a single subtype (220/244; FIG. 10). However, for a subset of patients, hybrid gene expression characteristic of both the ALS-Glia and ALS-TD subtypes (n=19), as well as the ALS-Glia and ALS-Ox subtypes (n=5) was observed. Interestingly, despite shared disease themes between the ALS-Ox and ALS-TD groups (FIGS. 5B-5D and 5H), these two subtypes are generally expressed independently. Furthermore, no patient samples were seen to express all three subtypes simultaneously, evident by the fact that all samples fall along one of the three faces of the hexagonal plot (FIG. 10). Sample subtypes obtained from the unsupervised clustering analysis are encoded as border colors, and generally show agreement between the two approaches. All patient samples shown to express a hybrid ALS phenotype were initially clustered into one of the two subtypes comprising the hybrid state (FIG. 10), further supporting the interpretation of this analysis. Taken together, the results capture the heterogeneous spectrum of ALS disease phenotypes in this cohort and reveal that a subset of ALS postmortem cortex transcriptomes show evidence for hybrid subtype states.

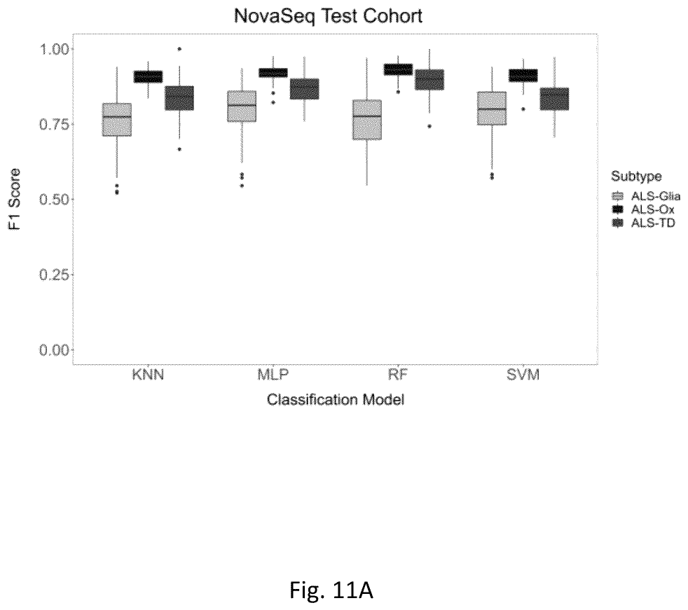

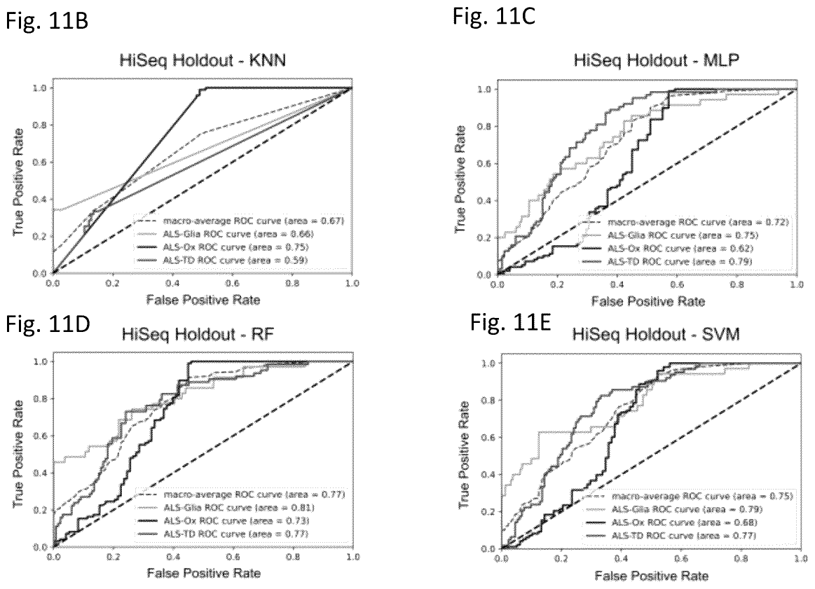

Four different supervised classifiers developed to assess the ability to stratify new patients, given the postmortem frontal or motor cortex transcriptome. As may be expected given the bootstrap-based classification results (FIG. 10), sensitivity and specificity metrics were relatively poor for all classifiers constructed (FIG. 11).

The ALS-Glia Subtype is Associated with a Worse Prognosis

The patient clinical parameters were considered in the context of the three subtypes. A survival analysis was performed to determine whether the three molecular subtypes of ALS capture the clinical heterogeneity seen in patient disease duration. ALS patients (n=208) were only assigned a subtype if there was a majority consensus among frontal and motor cortex samples or a single tissue sample was characterized for a given patient.

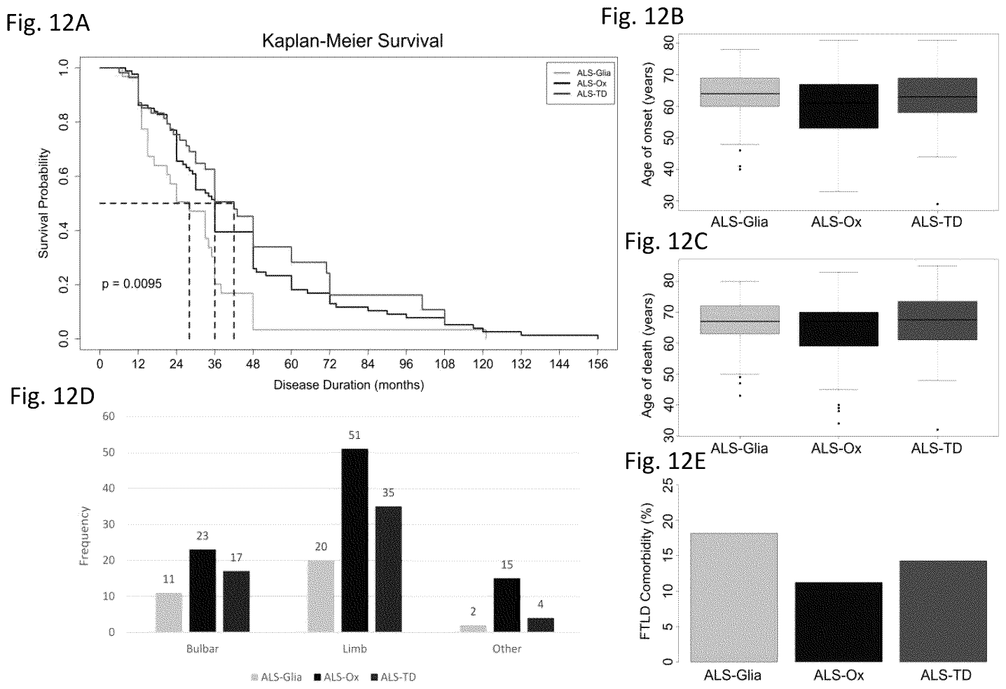

The results show significant differences in patient survival, with the ALS-Glia subtype associated with the shortest disease duration and a median survival of 28 months (FIG. 12A). Pairwise comparisons using the log-rank test showed significant differences in survival between ALS-Glia and ALS-Ox subtypes (p<0.02) and ALS-Glia and ALS-TD subtypes (p<0.005) but not between the ALS-Ox and ALS-TD subtypes (p<0.30). Consideration of patient age of symptom onset showed a nonsignificant trend toward the latest disease onset for the ALS-Glia subtype (63.2±1.83 years; presented as mean±standard error) and earliest disease onset for the ALS-Ox subtype (60.4±1.16 years; FIG. 12B, Table 1). The oldest median age at death for the ALS-TD subtype (66.7±1.33 years) and youngest median age at death for the ALS-Ox subtype (64.0±1.05 years), reflected some dependency on the age of symptom onset (FIG. 12C, Table 1). The site of symptom onset shows roughly the same proportion of patients with bulbar and limb onset across the three subtypes (FIG. 12D). Subtype comorbidity for FTLD was analyzed using a Chi-Square Test of Independence, although subtype dependency in the co-presentation of ALS and FTLD was not observed (c2=0.59). The clinical parameter analysis is further supported by WGCNA results (FIG. 8B), given the ALS-Glia subtype shows the oldest median age of onset and a significantly shorter disease duration—as captured by the purple eigengene (FIG. 12A, 14B). Taken together, these results lend support to the existence of subtype-driven clinical heterogeneity in ALS neurodegeneration.

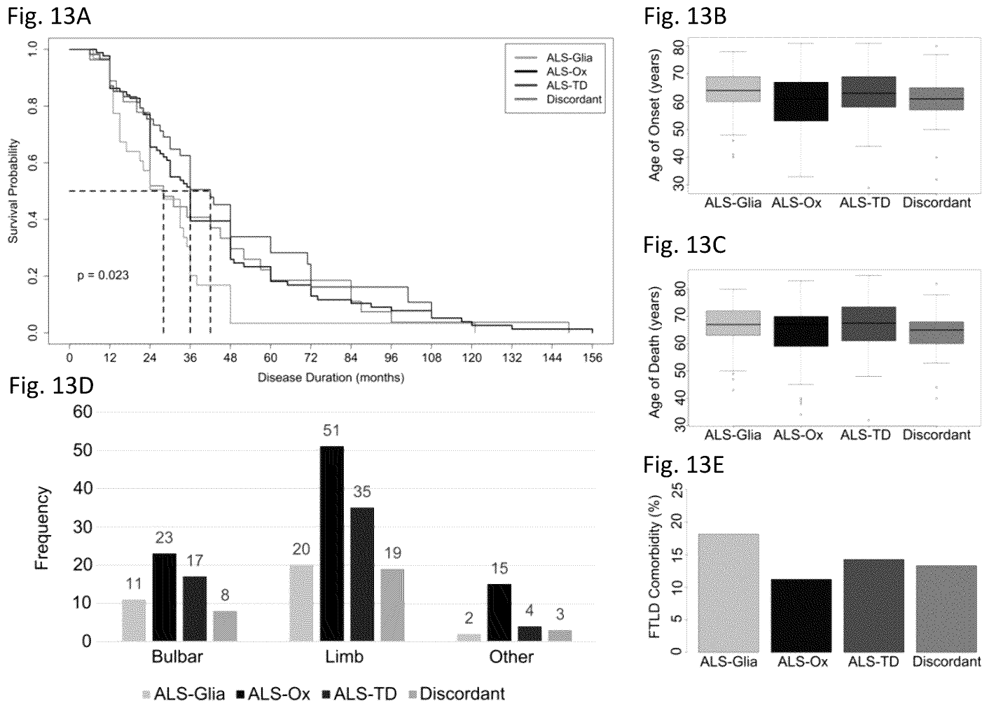

This analysis was also performed with ALS patients that were classified as having a different subtype in each tissue sample transcriptome, termed ALS-Discordant (FIG. 13). Similar results were observed, with significant differences in patient survival (p<0.05) and the latest age of onset maintained for the ALS-Glia subtype (nonsignificant). The agreement of subtype labels was assessed for the 140 samples in common (FIG. 1). An 85% agreement was observed (119/140) in sample classification, despite differences in the features used for patient stratification.

### Subtype-Specific Gene Expression

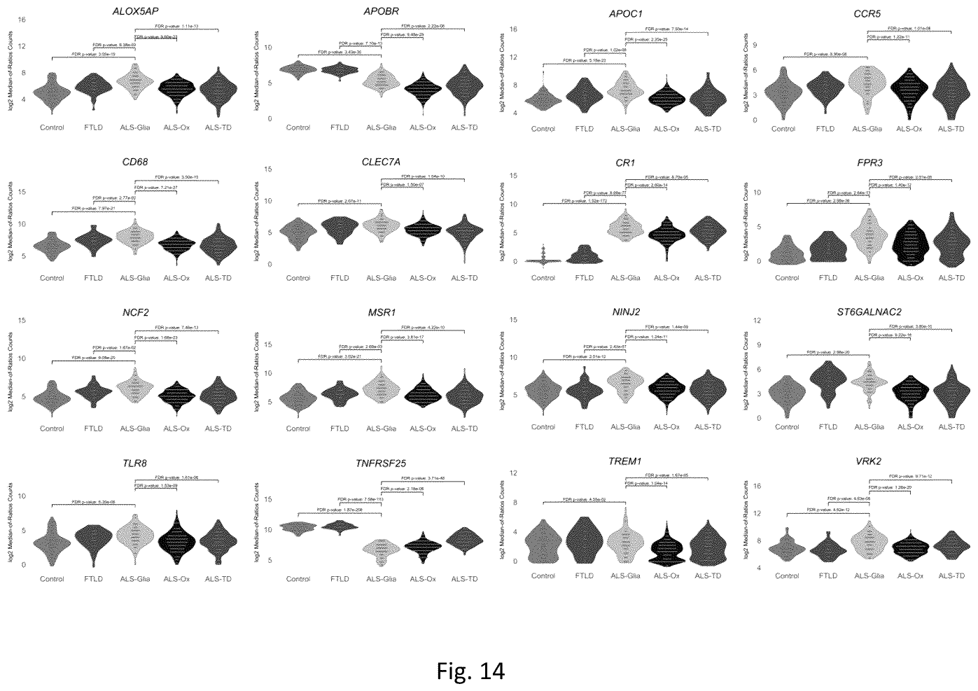

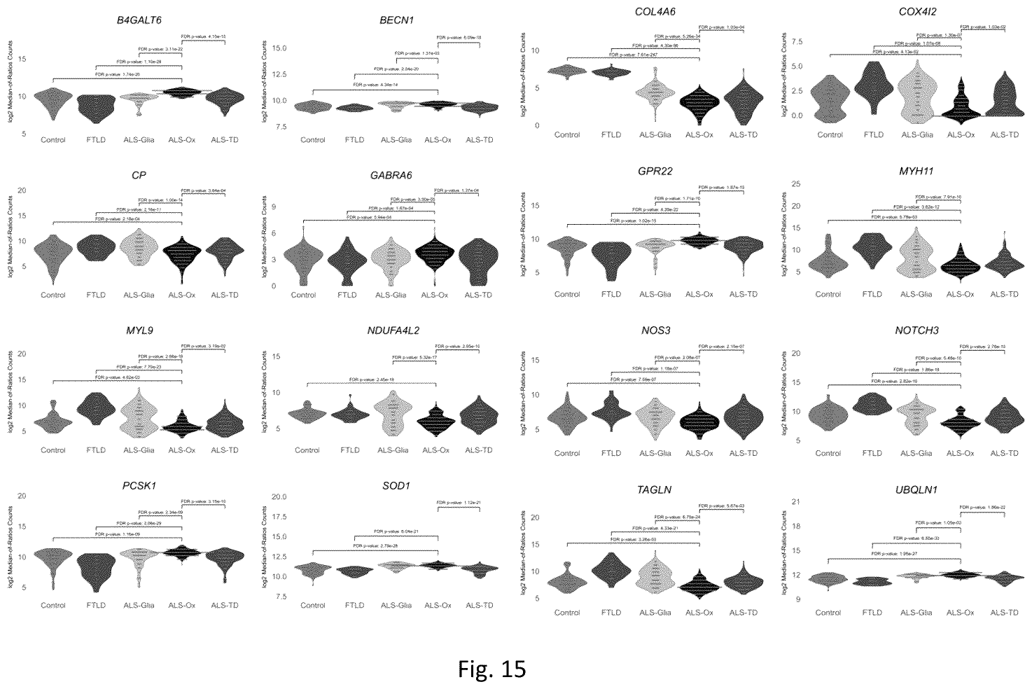

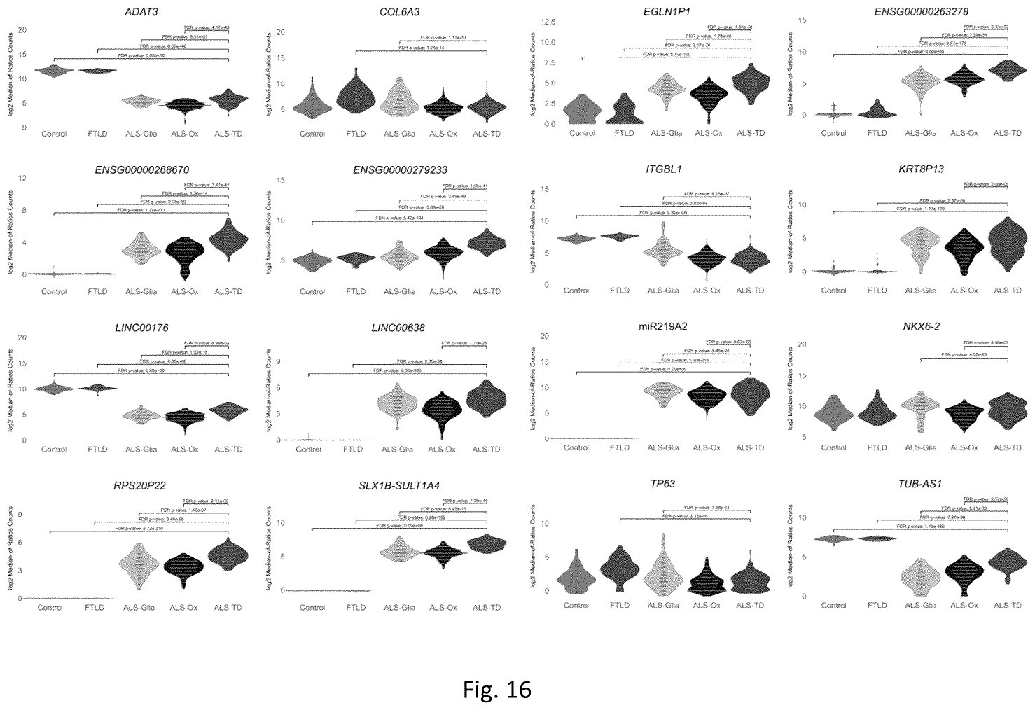

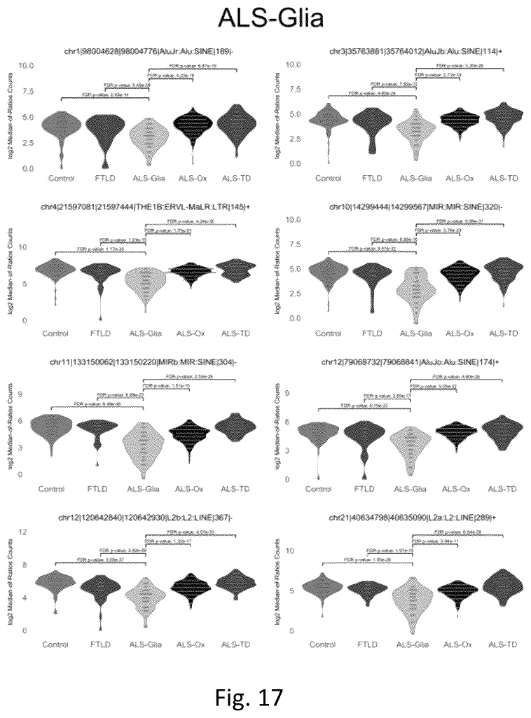

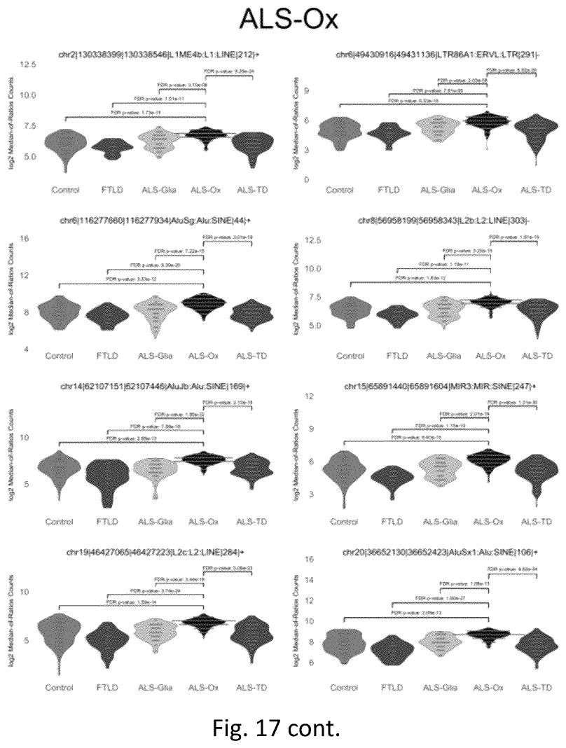

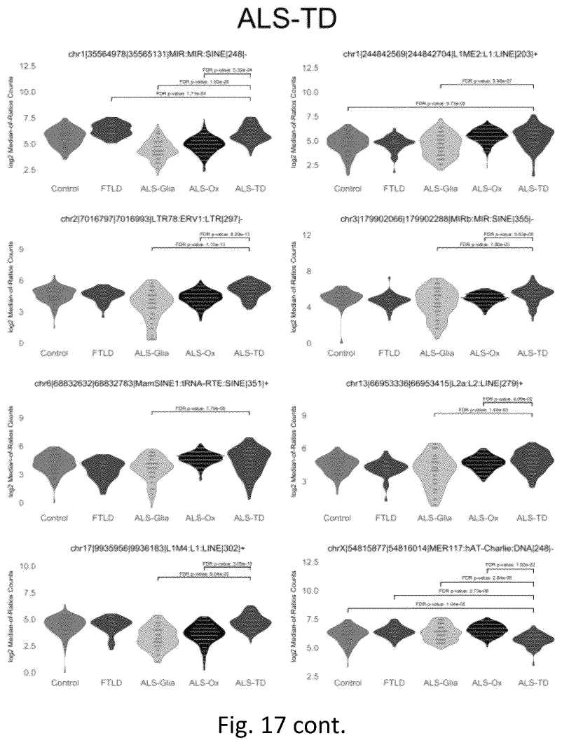

To provide additional insight into subtype-specific gene expression, a univariate analysis was performed, considering the 1681 genes and TEs used in classification, enrichment, and network construction. Transcript counts were normalized using DESeq2 size factor estimation and log 2 transformed. Violin plots reflect ALS-Glia (FIG. 9A, FIG. 14), ALS-Ox (FIG. 9B, FIG. 15), and ALS-TD (FIG. 9C, FIG. 16) specific gene and TE expression (FIG. 17, Table 3).

TABLE 3
Pairwise differential expression analysis for ALS subtypes and controls. A multi-factor
design equation was utilized for this analysis, accounting for sequencing platform and
disease subgroup. A full list of subtype-associated features is provided as well as the
ALS subtype assigned to each feature during clustering with the NovaSeq and HiSeq cohorts.

| | Novaseq NMF | Hiseq NMF |
|---|---|---|
| Symbol | Discrimitory for: | Discrimitory for: |
| A4GALT | ALS-Glia | ALS-Glia |
| ABCC12 | ALS-Ox | ALS-Ox |
| ABRACL | NA | ALS-Ox |
| ACAP2-IT1 | ALS-Ox | ALS-Ox |
| ACP5 | ALS-Glia | ALS-Glia |
| ACP7 | ALS-TD | ALS-Glia |
| ACTA2 | ALS-Glia | ALS-Glia |
| ACTBP7 | ALS-TD | ALS-TD |
| ACTG1P14 | ALS-TD | ALS-TD |
| ACTG1P4 | ALS-TD | ALS-TD |
| ACTG2 | ALS-Glia | ALS-Ox |
| ACTL6B | NA | ALS-Ox |
| ADAM1A | ALS-TD | ALS-TD |
| ADAM33 | ALS-Glia | ALS-TD |
| ADAMTS14 | ALS-TD | ALS-Glia |
| ADAMTS7 | ALS-TD | ALS-Glia |
| ADAMTS7P1 | ALS-TD | ALS-TD |
| ADAMTS9-AS1 | ALS-Ox | ALS-Ox |
| ADAMTSL4 | ALS-Glia | ALS-Glia |
| ADAT3 | ALS-TD | ALS-Glia |
| ADCY4 | ALS-Glia | ALS-TD |
| ADCYAP1 | ALS-Ox | ALS-Ox |
| ADH1B | ALS-Glia | ALS-Glia |
| ADRA1D | ALS-Ox | ALS-Ox |
| ADRA2B | ALS-TD | ALS-Glia |
| AGAP12P | ALS-TD | ALS-TD |
| AGER | ALS-TD | ALS-Glia |
| AGPAT4-IT1 | ALS-TD | ALS-Glia |
| AIF1 | ALS-Glia | ALS-Ox |
| AKAIN1 | ALS-Ox | ALS-Ox |
| AKAP5 | ALS-Ox | ALS-Ox |
| ALALS-Ox12B | ALS-Ox | ALS-Ox |
| ALALS-Ox5AP | ALS-Glia | ALS-Glia |
| ALS2CL | ALS-TD | ALS-TD |
| ALX4 | ALS-Glia | ALS-Ox |
| AMDHD1 | NA | ALS-Ox |
| AMH | ALS-TD | ALS-TD |
| ANKRD20A11P | ALS-TD | ALS-TD |
| ANKRD22 | ALS-Glia | ALS-Glia |
| ANKRD34C | ALS-Ox | ALS-Ox |
| ANO3 | ALS-Ox | ALS-Ox |
| ANPEP | ALS-Glia | ALS-TD |
| ANXA13 | ALS-TD | ALS-Glia |
| AOC3 | ALS-Glia | ALS-TD |
| APLNR | ALS-Glia | ALS-Glia |
| APOBR | ALS-Glia | ALS-Glia |
| APOC1 | ALS-Glia | ALS-Glia |
| APOC2 | ALS-Glia | ALS-Glia |
| AQP1 | ALS-TD | ALS-Glia |
| ARHGAP19-SLIT1 | ALS-TD | ALS-TD |
| ARHGEF34P | ALS-Glia | ALS-Glia |
| ARHGEF5 | ALS-Glia | ALS-Glia |
| ARL6 | ALS-Ox | ALS-Ox |
| ASAH2 | ALS-Ox | ALS-Ox |
| ASAH2B | ALS-Ox | NA |
| ASB2 | ALS-Ox | ALS-Ox |
| ATP12A | ALS-Ox | ALS-Ox |
| B4GALT6 | ALS-Ox | ALS-Ox |
| BCL2A1 | ALS-Glia | ALS-Glia |
| BDNF | ALS-Ox | ALS-Ox |
| BECN1P2 | ALS-TD | ALS-Glia |
| BEST3 | ALS-TD | ALS-Glia |
| BEX5 | ALS-Ox | ALS-Ox |
| BMP3 | ALS-Ox | ALS-Ox |
| BMP5 | ALS-Glia | ALS-Ox |
| BNC2 | ALS-Glia | ALS-Glia |
| BRCC3P1 | ALS-TD | ALS-TD |
| BRS3 | ALS-Glia | ALS-Ox |
| BTBD16 | ALS-Glia | ALS-Glia |
| BUB1 | NA | ALS-Ox |
| C10orf105 | ALS-TD | ALS-Glia |
| C10orf107 | ALS-Ox | ALS-Ox |
| C10orf128 | ALS-TD | ALS-Glia |
| C10orf62 | ALS-TD | ALS-Glia |
| C11orf96 | ALS-Glia | ALS-Glia |
| C17orf102 | ALS-Ox | ALS-Ox |
| C19orf33 | ALS-Glia | ALS-Ox |
| C1D | ALS-Ox | ALS-Ox |
| C1orf145 | ALS-Glia | ALS-Ox |
| C1QTNF3-AMACR | ALS-TD | ALS-Glia |
| C21orf62 | ALS-Glia | ALS-Glia |
| C22orf34 | ALS-TD | ALS-TD |
| C2CD4B | ALS-Glia | ALS-TD |
| C2orf80 | ALS-Ox | ALS-Ox |
| C3orf80 | ALS-Ox | ALS-Ox |
| C7orf55-LUC7L2 | ALS-TD | ALS-Glia |
| C7orf61 | ALS-TD | ALS-Glia |
| C9orf139 | ALS-TD | ALS-Glia |
| CA14 | ALS-Glia | ALS-Glia |
| CA4 | ALS-Glia | ALS-Ox |
| CALB1 | ALS-Ox | ALS-Ox |
| CALB2 | ALS-Glia | ALS-Ox |
| CALCR | ALS-Glia | ALS-Ox |
| CALHM1 | ALS-Ox | ALS-Ox |
| CAPN13 | ALS-TD | ALS-Glia |
| CARD10 | ALS-Glia | ALS-TD |
| CARTPT | ALS-Ox | ALS-Ox |
| CASQ1 | ALS-Ox | ALS-Ox |
| CASQ2 | ALS-Glia | ALS-Glia |
| CBLN4 | ALS-Ox | ALS-Ox |
| CCDC103 | ALS-Ox | ALS-Ox |
| CCDC13-AS1 | ALS-TD | ALS-Glia |
| CCDC154 | ALS-TD | ALS-TD |
| CCDC17 | ALS-TD | ALS-Glia |
| CCDC68 | ALS-Ox | ALS-Ox |
| CCKBR | ALS-Ox | ALS-Ox |
| CCL19 | ALS-Glia | NA |
| CCL2 | ALS-Glia | ALS-Glia |
| CCL5 | ALS-Glia | ALS-Glia |
| CCNE2 | ALS-TD | ALS-Glia |
| CCNO | ALS-Glia | ALS-Ox |
| CCR5 | ALS-Glia | ALS-Glia |
| CD177 | ALS-Glia | ALS-Glia |
| CD22 | ALS-TD | ALS-Glia |
| CD248 | ALS-Glia | ALS-TD |
| CD28 | ALS-Glia | ALS-Ox |
| CD300A | ALS-Glia | ALS-Glia |
| CD300LF | ALS-Glia | ALS-Glia |
| CD44 | ALS-Glia | ALS-Glia |
| CD68 | ALS-Glia | ALS-Glia |
| CD69 | ALS-Glia | ALS-Ox |
| CD86 | ALS-Glia | ALS-Glia |
| CDC42EP5 | ALS-Glia | ALS-Glia |
| CDH19 | ALS-TD | ALS-Glia |
| CDKN3 | NA | ALS-Ox |
| CEL | ALS-TD | ALS-TD |
| CEP295NL | ALS-TD | ALS-TD |
| CEP55 | ALS-Glia | ALS-Ox |
| CERKL | ALS-Ox | ALS-Ox |
| CES1 | ALS-Glia | ALS-Glia |
| CFB | ALS-Glia | ALS-Glia |
| CFP | ALS-TD | ALS-TD |
| CFTR | ALS-TD | ALS-Glia |
| CHI3L2 | ALS-Glia | ALS-Glia |
| CHKB-CPT1B | ALS-TD | ALS-TD |
| CHMP4BP1 | ALS-TD | ALS-TD |
| chr1\|110598071\|110598366\|MamSINE1:tR-RALS-TE:SINE\|338\|+ | ALS-Ox | NA |
| chr1\|117526213\|117526301\|MIRb:MIR:SINE\|313\|− | ALS-Ox | ALS-Ox |
| chr1\|14687236\|14687564\|L2b:L2:LINE\|317\|− | ALS-TD | ALS-TD |
| chr1\|171700737\|171700843\|L2b:L2:LINE\|286\|− | ALS-Ox | NA |
| chr1\|183250536\|183250702\|MIR3:MIR:SINE\|317\|+ | ALS-Ox | NA |
| chr1\|184623937\|184624061\|MIRb:MIR:SINE\|281\|− | ALS-Ox | NA |
| chr1\|197157988\|197158152\|MamRep434:TcMar-Tigger:D\|267\|+ | ALS-Ox | ALS-Ox |
| chr1\|204946255\|204946364\|MIRb:MIR:SINE\|327\|− | ALS-TD | ALS-TD |
| chr1\|204949621\|204949775\|MER5A1:hAT-Charlie:D\|195\|+ | ALS-TD | ALS-TD |
| chr1\|204965807\|204966058\|MIRb:MIR:SINE\|282\|+ | ALS-TD | ALS-Glia |
| chr1\|204982230\|204982386\|MIR3:MIR:SINE\|289\|− | ALS-TD | ALS-TD |
| chr1\|205001762\|205001923\|MamSINE1:tR-RALS-TE:SINE\|317\|− | ALS-TD | ALS-TD |
| chr1\|205006226\|205006443\|L2c:L2:LINE\|350\|− | ALS-TD | ALS-TD |
| chr1\|205529732\|205529939\|MIR:MIR:SINE\|294\|+ | ALS-TD | ALS-Glia |
| chr1\|205716309\|205716381\|MER131:D?:D?\|296\|− | ALS-Ox | ALS-Ox |
| chr1\|218335472\|218335648\|MER3:hAT-Charlie:D\|188\|+ | ALS-Ox | NA |
| chr1\|220150225\|220150314\|MIR:MIR:SINE\|244\|− | ALS-Ox | NA |
| chr1\|225866202\|225866280\|MIR3:MIR:SINE\|295\|− | ALS-TD | ALS-Glia |
| chr1\|229321782\|229322128\|MER2:TcMar-Tigger:D\|134\|+ | ALS-Ox | NA |
| chr1\|235110540\|235110668\|MIR3:MIR:SINE\|352\|− | ALS-Ox | ALS-Ox |
| chr1\|241354542\|241354707\|OldhAT1:hAT-Ac:D\|293\|− | ALS-Ox | NA |
| chr1\|244842569\|244842704\|L1ME2:L1:LINE\|203\|+ | ALS-Ox | ALS-Ox |
| chr1\|33821639\|33822019\|L2b:L2:LINE\|301\|− | ALS-TD | ALS-TD |
| chr1\|35564978\|35565131\|MIR:MIR:SINE\|248\|− | ALS-TD | ALS-TD |
| chr1\|35714013\|35714123\|MIR3:MIR:SINE\|198\|− | ALS-Ox | ALS-Ox |
| chr1\|39679048\|39679132\|L2b:L2:LINE\|337\|+ | ALS-Ox | ALS-Ox |
| chr1\|39681933\|39682041\|L2c:L2:LINE\|370\|+ | ALS-Ox | ALS-Ox |
| chr1\|4783372\|4783444\|MamSINE1:tR-RALS-TE:SINE\|377\|− | ALS-Ox | NA |
| chr1\|4787110\|4787329\|L2b:L2:LINE\|333\|− | ALS-Ox | ALS-Ox |
| chr1\|62818654\|62818897\|L1MB8:L1:LINE\|188\|− | ALS-Ox | ALS-Glia |
| chr1\|69981810\|69982018\|MIR:MIR:SINE\|269\|− | ALS-Ox | NA |
| chr1\|71539018\|71539237\|MIR:MIR:SINE\|307\|+ | ALS-Ox | ALS-Ox |
| chr1\|84645944\|84646020\|MamRALS-TE1:RALS-TE- | ALS-Ox | ALS-Ox |
| BovB:LINE\|373\|+ | | |
| chr1\|98004628\|98004776\|AluJr:Alu:SINE\|189\|− | ALS-Ox | ALS-TD |
| chr1\|98023019\|98023175\|AluJo:Alu:SINE\|205\|+ | ALS-Ox | ALS-TD |
| chr1\|98036577\|98036920\|Tigger3a:TcMar-Tigger:D\|188\|+ | ALS-Ox | ALS-Ox |
| chr1\|99922950\|99923151\|Tigger15a:TcMar-Tigger:D\|286\|+ | ALS-Ox | ALS-Ox |
| chr10\|11025282\|11025655\|THE1D:ERVL-MaLR:LTR\|168\|+ | ALS-Ox | ALS-TD |
| chr10\|11030204\|11030346\|MIRb:MIR:SINE\|371\|− | ALS-Ox | ALS-TD |
| chr10\|11037047\|11037403\|THE1B:ERVL-MaLR:LTR\|122\|− | ALS-Ox | ALS-TD |
| chr10\|11083685\|11083899\|MIR:MIR:SINE\|330\|+ | ALS-Ox | ALS-TD |
| chr10\|11125686\|11125945\|X2_LINE:CR1:LINE\|389\|− | ALS-Ox | ALS-TD |
| chr10\|11168978\|11169168\|MIR:MIR:SINE\|300\|+ | ALS-Ox | ALS-TD |
| chr10\|115747698\|115748065\|MLT1A0:ERVL-MaLR:LTR\|224\|− | ALS-Ox | ALS-Ox |
| chr10\|121742963\|121743085\|MER94:hAT-Blackjack:D\|223\|− | ALS-Ox | NA |
| chr10\|124605271\|124605664\|L2b:L2:LINE\|350\|− | ALS-TD | ALS-Glia |
| chr10\|14102244\|14102461\|AluSz:Alu:SINE\|138\|+ | ALS-Ox | ALS-TD |
| chr10\|14299444\|14299567\|MIR:MIR:SINE\|320\|− | ALS-Ox | ALS-TD |
| chr10\|22568490\|22568589\|L2b:L2:LINE\|263\|− | ALS-TD | ALS-Glia |
| chr10\|22599150\|22599430\|L1MB8:L1:LINE\|204\|− | ALS-TD | ALS-Glia |
| chr10\|63199839\|63200089\|MIRb:MIR:SINE\|315\|− | ALS-Ox | ALS-Ox |
| chr10\|66932891\|66933081\|MIRc:MIR:SINE\|363\|+ | ALS-Ox | NA |
| chr10\|69633231\|69633457\|MER113:hAT-Charlie:D\|292\|+ | ALS-Ox | ALS-Ox |
| chr10\|76987405\|76987627\|MER58A:hAT-Charlie:D\|225\|− | ALS-Ox | ALS-TD |
| chr10\|81919299\|81919431\|MIRb:MIR:SINE\|302\|+ | ALS-Ox | ALS-TD |
| chr10\|82549010\|82549231\|MIRb:MIR:SINE\|341\|+ | ALS-Ox | ALS-TD |
| chr10\|82566891\|82567059\|MLT11:ERVL-MaLR:LTR\|267\|− | ALS-Ox | ALS-TD |
| chr10\|87970504\|87970627\|UCON107:hAT-Tag1:D\|238\|− | ALS-Ox | ALS-Ox |
| chr10\|92353298\|92353476\|MIRb:MIR:SINE\|318\|+ | ALS-Ox | NA |
| chr11\|111909864\|111910071\|MER5B:hAT-Charlie:D\|138\|− | ALS-TD | ALS-Glia |
| chr11\|113245172\|113245450\|AluJb:Alu:SINE\|166\|+ | ALS-TD | ALS-Glia |
| chr11\|114161357\|114161537\|L2b:L2:LINE\|260\|− | ALS-TD | ALS-TD |
| chr11\|118165463\|118165762\|AluSx:Alu:SINE\|139\|− | ALS-Ox | NA |
| chr11\|123632507\|123632815\|AluSz:Alu:SINE\|123\|+ | ALS-Ox | ALS-Ox |
| chr11\|133150062\|133150220\|MIRb:MIR:SINE\|304\|− | ALS-Ox | ALS-TD |
| chr11\|134409107\|134409239\|MIRb:MIR:SINE\|312\|− | ALS-TD | ALS-TD |
| chr11\|30008564\|30008741\|MIRc:MIR:SINE\|314\|− | ALS-Ox | NA |
| chr11\|33672154\|33672567\|MLT1M:ERVL-MaLR:LTR\|278\|− | ALS-Ox | NA |
| chr11\|33673504\|33673627\|MIRc:MIR:SINE\|193\|+ | ALS-Ox | ALS-Ox |
| chr11\|34098934\|34099061\|L2a:L2:LINE\|98\|+ | ALS-Ox | ALS-Ox |
| chr11\|45243462\|45243579\|MER5A:hAT-Charlie:D\|316\|+ | ALS-Ox | ALS-Ox |
| chr11\|64321600\|64321732\|MIR:MIR:SINE\|288\|+ | ALS-Ox | ALS-Ox |
| chr11\|66345711\|66345954\|MIRc:MIR:SINE\|220\|− | ALS-Ox | ALS-Ox |
| chr11\|67418109\|67418229\|MIR3:MIR:SINE\|292\|− | ALS-TD | ALS-Glia |
| chr11\|67425186\|67425325\|MIRc:MIR:SINE\|364\|− | ALS-TD | ALS-Glia |
| chr11\|72793261\|72793497\|MIRb:MIR:SINE\|301\|− | ALS-Ox | NA |
| chr11\|8105552\|8105631\|MIRc:MIR:SINE\|291\|+ | ALS-Ox | NA |
| chr11\|8982894\|8983038\|MIR3:MIR:SINE\|276\|+ | ALS-Ox | ALS-Ox |
| chr12\|101394834\|101395068\|MIRc:MIR:SINE\|289\|+ | ALS-Ox | ALS-Ox |
| chr12\|120642840\|120642930\|L2b:L2:LINE\|367\|− | ALS-Ox | ALS-TD |
| chr12\|123964435\|123964597\|L2a:L2:LINE\|283\|− | ALS-Ox | ALS-TD |
| chr12\|124780216\|124780341\|MER91A:hAT-Tip100:D\|320\|+ | ALS-TD | ALS-TD |
| chr12\|125656270\|125656450\|Plat_L3:CR1:LINE\|354\|− | ALS-Ox | NA |
| chr12\|13550001\|13550129\|MIRc:MIR:SINE\|324\|+ | ALS-Ox | ALS-Ox |
| chr12\|27799571\|27799676\|L2b:L2:LINE\|245\|− | ALS-Ox | NA |
| chr12\|27801558\|27801735\|MER117:hAT-Charlie:D\|318\|− | ALS-Ox | NA |
| chr12\|46185953\|46186190\|Tigger16b:TcMar-Tigger:D\|284\|+ | ALS-Ox | NA |
| chr12\|48996101\|48996278\|MIR3:MIR:SINE\|269\|− | ALS-Ox | ALS-Ox |
| chr12\|49896418\|49896641\|MIRb:MIR:SINE\|282\|+ | ALS-TD | ALS-TD |
| chr12\|54950114\|54950247\|MIR1_Amn:MIR:SINE\|372\|− | ALS-Ox | ALS-Ox |
| chr12\|77578177\|77578321\|MER5A1:hAT-Charlie:D\|215\|+ | ALS-Ox | ALS-TD |
| chr12\|77578313\|77578540\|LTR16C:ERVL:LTR\|299\|+ | ALS-Ox | NA |
| chr12\|79068732\|79068841\|AluJo:Alu:SINE\|174\|+ | ALS-Ox | ALS-TD |
| chr12\|88199201\|88199451\|MIR:MIR:SINE\|292\|+ | ALS-Ox | ALS-Ox |
| chr12\|89618275\|89618470\|MIRb:MIR:SINE\|376\|− | ALS-Ox | NA |
| chr12\|98534892\|98535122\|Charlie10a:hAT-Charlie:D\|272\|− | ALS-Ox | ALS-Ox |
| chr13\|101847107\|101847303\|L3b:CR1:LINE\|296\|+ | ALS-Ox | ALS-TD |
| chr13\|102028182\|102028551\|MLT1A0:ERVL-MaLR:LTR\|231\|+ | ALS-Ox | NA |
| chr13\|102357507\|102357734\|MIR:MIR:SINE\|293\|+ | ALS-Ox | ALS-TD |
| chr13\|102376080\|102376433\|THE1B:ERVL-MaLR:LTR\|122\|+ | ALS-Ox | ALS-TD |
| chr13\|102384423\|102384596\|L2b:L2:LINE\|429\|+ | ALS-Ox | ALS-TD |
| chr13\|107426068\|107426261\|L2b:L2:LINE\|322\|+ | ALS-Ox | NA |
| chr13\|24298816\|24299096\|L3:CR1:LINE\|314\|− | ALS-TD | ALS-Glia |
| chr13\|35773606\|35773823\|UCON8:D:D\|299\|− | ALS-Ox | ALS-Ox |
| chr13\|66491949\|66492118\|MLT1K:ERVL-MaLR:LTR\|225\|+ | ALS-Ox | ALS-Glia |
| chr13\|66537443\|66537692\|L2a:L2:LINE\|342\|− | ALS-Ox | ALS-Glia |
| chr13\|66628526\|66628665\|AluJo:Alu:SINE\|187\|− | ALS-Ox | NA |
| chr13\|66811921\|66812122\|L1PB1:L1:LINE\|196\|− | ALS-Ox | ALS-TD |
| chr13\|66908053\|66908339\|L2:L2:LINE\|325\|+ | ALS-Ox | ALS-Glia |
| chr13\|66915981\|66916068\|MIR3:MIR:SINE\|264\|− | ALS-Ox | ALS-Glia |
| chr13\|66953336\|66953415\|L2a:L2:LINE\|279\|+ | ALS-Ox | ALS-Glia |
| chr13\|66985990\|66986360\|THE 1C:ERVL-MaLR:LTR\|101\|− | ALS-Ox | NA |
| chr13\|67010075\|67010306\|MIR:MIR:SINE\|273\|− | ALS-Ox | ALS-Glia |
| chr13\|67014384\|67014507\|L2:L2:LINE\|425\|+ | ALS-Ox | ALS-Glia |
| chr13\|67093175\|67093457\|AluSq2:Alu:SINE\|128\|+ | ALS-Ox | NA |
| chr13\|67136021\|67136385\|Tigger3a:TcMar-Tigger:D\|161\|+ | ALS-Ox | ALS-Glia |
| chr13\|67200343\|67200500\|MamRep605:LTR?:LTR?\|338\|− | ALS-Ox | ALS-Ox |
| chr14\|100849279\|100849392\|L1ME5:L1:LINE\|274\|+ | ALS-Ox | ALS-TD |
| chr14\|21209128\|21209276\|MER2:TcMar-Tigger:D\|169\|+ | ALS-Ox | NA |
| chr14\|32831511\|32831619\|L2a:L2:LINE\|287\|− | ALS-Ox | NA |
| chr14\|57204956\|57205187\|MIRb:MIR:SINE\|272\|+ | ALS-Ox | ALS-Ox |
| chr14\|57999209\|57999546\|L3:CR1:LINE\|287\|− | ALS-Ox | NA |
| chr14\|59370309\|59370441\|L2a:L2:LINE\|190\|− | ALS-Ox | ALS-Ox |
| chr14\|62101015\|62101231\|Charlie15b:hAT-Charlie:D\|211\|+ | ALS-Ox | ALS-Ox |
| chr14\|62104691\|62104908\|MIR:MIR:SINE\|277\|+ | ALS-Ox | ALS-Ox |
| chr14\|62107151\|62107446\|AluJb:Alu:SINE\|169\|+ | ALS-Ox | ALS-Ox |
| chr14\|62110149\|62110334\|MIRc:MIR:SINE\|371\|+ | ALS-Ox | ALS-Ox |
| chr14\|79010748\|79010913\|MIRb:MIR:SINE\|342\|− | ALS-Ox | NA |
| chr14\|85541511\|85541646\|MIR:MIR:SINE\|298\|+ | ALS-Ox | ALS-TD |
| chr14\|85550824\|85550925\|L3b:CR1:LINE\|333\|+ | ALS-Ox | ALS-TD |
| chr14\|85568775\|85568895\|MIRb:MIR:SINE\|350\|+ | ALS-Ox | ALS-TD |
| chr14\|85582440\|85582674\|MIR:MIR:SINE\|302\|+ | ALS-Ox | ALS-TD |
| chr14\|85626237\|85626418\|MamSINE1:tR-RALS-TE:SINE\|384\|− | ALS-Ox | NA |
| chr14\|85638247\|85638594\|LTR 16A:ERVL:LTR\|299\|+ | ALS-Ox | ALS-TD |
| chr15\|24987476\|24987621\|MER5A:hAT-Charlie:D\|252\|− | ALS-Ox | ALS-Ox |
| chr15\|24994372\|24994570\|MER8:TcMar-Tigger:D\|293\|+ | ALS-Ox | NA |
| chr15\|25021991\|25022161\|MER58A:hAT-Charlie:D\|251\|− | ALS-Ox | ALS-Ox |
| chr15\|25027967\|25028264\|AluSq2:Alu:SINE\|91\|+ | ALS-Ox | ALS-Ox |
| chr15\|25035823\|25035937\|L1ME3G:L1:LINE\|241\|+ | ALS-Ox | ALS-Ox |
| chr15\|25162223\|25162324\|MER34-int:ERV1:LTR\|317\|+ | ALS-Ox | ALS-Ox |
| chr15\|25329414\|25329514\|MIR3:MIR:SINE\|330\|+ | ALS-Ox | ALS-TD |
| chr15\|34978746\|34979041\|MER2B:TcMar-Tigger:D\|184\|− | ALS-Ox | NA |
| chr15\|42743728\|42743868\|Penelope1_Vert:Penelope:LINE\|290\|+ | ALS-Ox | ALS-Ox |
| chr15\|48879668\|48879879\|Tigger16a:TcMar-Tigger:D\|222\|+ | ALS-Ox | ALS-Ox |
| chr15\|51808935\|51809047\|MamSINE1:tR-RALS-TE:SINE\|357\|+ | ALS-Ox | ALS-Ox |
| chr15\|52312366\|52312657\|AluSz6:Alu:SINE\|96\|− | ALS-Ox | NA |
| chr15\|60974800\|60975029\|L2c:L2:LINE\|342\|− | ALS-TD | ALS-TD |
| chr15\|65891440\|65891604\|MIR3:MIR:SINE\|247\|+ | ALS-Ox | ALS-Ox |
| chr16\|14211719\|14211936\|MIRb:MIR:SINE\|300\|+ | ALS-Ox | ALS-TD |
| chr16\|24216784\|24216906\|MIR3:MIR:SINE\|265\|− | ALS-Ox | ALS-Ox |
| chr16\|24218019\|24218117\|L2a:L2:LINE\|245\|− | ALS-Ox | ALS-Ox |
| chr16\|4801042\|4801226\|MIRb:MIR:SINE\|303\|− | ALS-TD | ALS-TD |
| chr16\|56288823\|56288917\|MIR1_Amn:MIR:SINE\|362\|+ | ALS-TD | ALS-TD |
| chr16\|6368792\|6369033\|L1ME3E:L1:LINE\|234\|+ | ALS-Ox | ALS-TD |
| chr16\|69462961\|69463087\|AluJr:Alu:SINE\|127\|+ | ALS-Ox | NA |
| chr16\|7293102\|7293406\|MER2:TcMar-Tigger:D\|166\|+ | ALS-Ox | ALS-TD |
| chr16\|7335378\|7335500\|L3:CR1:LINE\|246\|+ | ALS-Ox | ALS-TD |
| chr16\|83811570\|83811643\|AmnSINE1:5S-Deu-L2:SINE\|233\|+ | ALS-Ox | NA |
| chr16\|9111744\|9111980\|L1ME4a:L1:LINE\|244\|− | ALS-Ox | ALS-Ox |
| chr16\|9756601\|9756675\|MIRb:MIR:SINE\|203\|+ | ALS-Ox | ALS-Ox |
| chr17\|16028246\|16028372\|MER5B:hAT-Charlie:D\|349\|+ | ALS-Ox | NA |
| chr17\|45899366\|45899549\|MIR:MIR:SINE\|200\|− | ALS-TD | ALS-TD |
| chr17\|58345196\|58345319\|MIRc:MIR:SINE\|325\|− | ALS-Ox | NA |
| chr17\|59948860\|59948978\|OldhAT1:hAT-Ac:D\|330\|− | ALS-Ox | NA |
| chr17\|60446109\|60446219\|MER131:D?:D?\|224\|− | ALS-Ox | NA |
| chr17\|74248710\|74248902\|MIR:MIR:SINE\|219\|− | ALS-TD | ALS-Glia |
| chr17\|82658820\|82659186\|MLT1C:ERVL-MaLR:LTR\|238\|+ | ALS-TD | ALS-Glia |
| chr17\|9935956\|9936183\|L1M4:L1:LINE\|302\|+ | ALS-TD | ALS-TD |
| chr18\|21629396\|21629777\|MER57B2:ERV1:LTR\|152\|− | ALS-Ox | NA |
| chr18\|21868118\|21868293\|L1MB2:L1:LINE\|167\|− | ALS-Ox | NA |
| chr18\|46264565\|46264660\|L2b:L2:LINE\|279\|− | ALS-Ox | NA |
| chr18\|57363754\|57363888\|MIR3:MIR:SINE\|358\|− | ALS-Ox | ALS-Ox |
| chr18\|76982063\|76982196\|MIRb:MIR:SINE\|271\|− | ALS-TD | ALS-Glia |
| chr18\|76992147\|76992483\|MER1B:hAT-Charlie:D\|141\|− | ALS-TD | ALS-Glia |
| chr18\|9956101\|9956262\|MIR3:MIR:SINE\|308\|− | ALS-Ox | NA |
| chr19\|2715361\|2715715\|MLT1E1A:ERVL-MaLR:LTR\|252\|+ | ALS-Ox | ALS-Ox |
| chr19\|29615400\|29615473\|L1ME4c:L1:LINE\|275\|+ | ALS-Ox | NA |
| chr19\|34382377\|34382566\|MER105:hAT-Charlie:D\|176\|+ | ALS-TD | ALS-TD |
| chr19\|36015624\|36015709\|L2:L2:LINE\|345\|− | ALS-Ox | NA |
| chr19\|46427065\|46427223\|L2c:L2:LINE\|284\|+ | ALS-Ox | ALS-Ox |
| chr19\|50313416\|50313512\|MER113A:hAT-Charlie:D\|281\|− | ALS-Ox | ALS-Ox |
| chr19\|54459141\|54459248\|MER20:hAT-Charlie:D\|215\|+ | ALS-TD | ALS-TD |
| chr19\|56811359\|56811474\|L1ME4c:L1:LINE\|239\|− | ALS-Ox | ALS-Ox |
| chr2\|114527646\|114527989\|Tigger3a:TcMar-Tigger:D\|152\|− | ALS-Ox | ALS-TD |
| chr2\|130338399\|130338546\|L1ME4b:L1:LINE\|212\|+ | ALS-Ox | NA |
| chr2\|141592215\|141592569\|MLT1A0:ERVL-MaLR:LTR\|185\|− | ALS-Ox | ALS-TD |
| chr2\|141711189\|141711486\|MLT10:ERVL-MaLR:LTR\|276\|+ | ALS-Ox | ALS-TD |
| chr2\|143942935\|143943213\|L1ME3E:L1:LINE\|333\|− | ALS-Ox | NA |
| chr2\|148981816\|148982036\|MIRb:MIR:SINE\|300\|+ | ALS-Ox | ALS-TD |
| chr2\|157319289\|157319489\|MamSINE1:tR-RALS-TE:SINE\|379\|+ | ALS-Ox | ALS-Glia |
| chr2\|16551719\|16551867\|MIRb:MIR:SINE\|350\|− | ALS-Ox | ALS-Ox |
| chr2\|166038047\|166038118\|MamRALS-TE1:RALS-TE- | ALS-Ox | ALS-Ox |
| BovB:LINE\|254\|− | | |
| chr2\|182777287\|182777505\|MER58C:hAT-Charlie:D\|234\|− | ALS-Ox | NA |
| chr2\|190913797\|190914080\|AluJr:Alu:SINE\|184\|− | ALS-Ox | ALS-Ox |
| chr2\|199926495\|199926665\|FAM:Alu:SINE\|206\|+ | ALS-Ox | NA |
| chr2\|206123472\|206123715\|MIRb:MIR:SINE\|245\|+ | ALS-Ox | ALS-Ox |
| chr2\|209684991\|209685079\|MIRb:MIR:SINE\|268\|− | ALS-Ox | ALS-TD |
| chr2\|209974570\|209974803\|MIR:MIR:SINE\|234\|− | ALS-Ox | ALS-TD |
| chr2\|230037266\|230037348\|MIRb:MIR:SINE\|243\|− | ALS-Ox | NA |
| chr2\|231072200\|231072280\|L3:CR1:LINE\|266\|− | ALS-Ox | NA |
| chr2\|28801877\|28801998\|Tigger12A:TcMar-Tigger:D\|265\|+ | ALS-Ox | NA |
| chr2\|38563158\|38563317\|L1M5:L1:LINE\|248\|+ | ALS-Ox | ALS-Ox |
| chr2\|40101766\|40101958\|MER5A:hAT-Charlie:D\|207\|− | ALS-Ox | ALS-Ox |
| chr2\|44319280\|44319536\|L1ME4c:L1:LINE\|256\|+ | ALS-Ox | ALS-Ox |
| chr2\|47171391\|47171495\|MIRb:MIR:SINE\|206\|− | ALS-Ox | ALS-Ox |
| chr2\|50793908\|50794126\|MER20:hAT-Charlie:D\|217\|+ | ALS-Ox | ALS-TD |
| chr2\|50874327\|50874481\|MIRb:MIR:SINE\|281\|− | ALS-Ox | ALS-TD |
| chr2\|50878959\|50879325\|MLT2C2:ERVL:LTR\|158\|+ | ALS-Ox | ALS-TD |
| chr2\|50899087\|50899251\|MER127:TcMar-Tigger:D\|288\|+ | ALS-Ox | ALS-TD |
| chr2\|69469481\|69469579\|MamSINE1:tR-RALS-TE:SINE\|385\|− | ALS-Ox | NA |
| chr2\|7016797\|7016993\|LTR78:ERV1:LTR\|297\|− | ALS-TD | ALS-TD |
| chr2\|70661824\|70662049\|MIRb:MIR:SINE\|276\|+ | ALS-Ox | NA |
| chr2\|76971183\|76971359\|MER5A:hAT-Charlie:D\|281\|+ | ALS-Ox | ALS-TD |
| chr2\|76984567\|76984821\|MLT1J:ERVL-MaLR:LTR\|328\|+ | ALS-Ox | NA |
| chr2\|77102784\|77103005\|MER58A:hAT-Charlie:D\|304\|+ | ALS-Ox | ALS-TD |
| chr2\|77460300\|77460435\|MIRb:MIR:SINE\|353\|+ | ALS-Ox | ALS-TD |
| chr2\|80044972\|80045321\|MLT1H1:ERVL-MaLR:LTR\|267\|− | ALS-Ox | ALS-TD |
| chr2\|86775394\|86775466\|Tigger19b:TcMar-Tigger:D\|304\|+ | ALS-Ox | NA |
| chr2\|97161188\|97161419\|L2:L2:LINE\|305\|+ | ALS-Ox | ALS-Ox |
| chr2\|97579795\|97579997\|MIRb:MIR:SINE\|286\|− | ALS-Ox | ALS-Ox |
| chr20\|11925425\|11925501\|Tigger19a:TcMar-Tigger:D\|333\|+ | ALS-Ox | NA |
| chr20\|1926587\|1926776\|MIR:MIR:SINE\|261\|− | ALS-TD | ALS-TD |
| chr20\|36652130\|36652423\|AluSx1:Alu:SINE\|106\|+ | ALS-Ox | ALS-Ox |
| chr20\|37523447\|37523613\|L2c:L2:LINE\|256\|+ | ALS-Ox | ALS-Ox |
| chr20\|44907296\|44907477\|FAM:Alu:SINE\|155\|+ | ALS-Ox | NA |
| chr20\|45092738\|45093020\|L2b:L2:LINE\|360\|+ | ALS-Ox | ALS-Ox |
| chr20\|45094391\|45094533\|MIRb:MIR:SINE\|242\|+ | ALS-Ox | ALS-Ox |
| chr20\|5194848\|5194988\|MIRb:MIR:SINE\|183\|− | ALS-Ox | NA |
| chr20\|58449601\|58449837\|Eulor2B:D?:D?\|225\|+ | ALS-Ox | NA |
| chr20\|59725550\|59725839\|LTR16A2:ERVL:LTR\|269\|− | ALS-TD | ALS-TD |
| chr20\|8306441\|8306661\|MIRb:MIR:SINE\|353\|+ | ALS-Ox | NA |
| chr20\|8459911\|8460052\|MamRALS-TE1:RALS-TE- | ALS-Ox | ALS-Ox |
| BovB:LINE\|365\|+ | | |
| chr21\|21124867\|21125188\|MER63D:hAT-Blackjack:D\|232\|+ | ALS-Ox | ALS-Glia |
| chr21\|21538195\|21538272\|L3:CR1:LINE\|278\|− | ALS-Ox | ALS-Ox |
| chr21\|34099753\|34100051\|MER124:D?:D?\|239\|+ | ALS-Ox | ALS-Ox |
| chr21\|40634798\|40635090\|L2a:L2:LINE\|289\|+ | ALS-Ox | ALS-TD |
| chr22\|17885243\|17885496\|L1ME4a:L1:LINE\|352\|+ | ALS-Ox | ALS-TD |
| chr22\|18993626\|18993739\|MIRb:MIR:SINE\|240\|− | ALS-Ox | NA |
| chr22\|32512553\|32512684\|MER103C:hAT-Charlie:D\|254\|+ | ALS-Ox | ALS-Ox |
| chr22\|41995604\|41995899\|L4_C_Mam:RALS-TE-X:LINE\|296\|− | ALS-Ox | NA |
| chr22\|43506825\|43506998\|MER96B:hAT-Tip 100:D\|195\|+ | ALS-Ox | NA |
| chr22\|46084397\|46084579\|MIRb:MIR:SINE\|282\|− | ALS-TD | ALS-TD |
| chr3\|112361783\|112361898\|MIRc:MIR:SINE\|297\|+ | ALS-Ox | ALS-Ox |
| chr3\|115804301\|115804475\|MIR3:MIR:SINE\|263\|− | ALS-Ox | NA |
| chr3\|115815061\|115815380\|MER44A:TcMar-Tigger:D\|142\|+ | ALS-Ox | ALS-TD |
| chr3\|116304116\|116304230\|L2c:L2:LINE\|286\|− | ALS-Ox | ALS-Ox |
| chr3\|116652799\|116653163\|THE1D:ERVL-MaLR:LTR\|135\|− | ALS-Ox | ALS-TD |
| chr3\|116712449\|116712668\|CR1-L3A_Croc:CR1:LINE\|325\|+ | ALS-Ox | ALS-TD |
| chr3\|117249084\|117249202\|MIR3:MIR:SINE\|314\|+ | ALS-Ox | ALS-TD |
| chr3\|117455995\|117456224\|MIR:MIR:SINE\|288\|− | ALS-Ox | ALS-TD |
| chr3\|117553131\|117553299\|MIR3:MIR:SINE\|284\|+ | ALS-Ox | ALS-TD |
| chr3\|117695141\|117695281\|L2:L2:LINE\|316\|+ | ALS-Ox | ALS-TD |
| chr3\|124452318\|124452596\|L2c:L2:LINE\|310\|− | ALS-Ox | ALS-TD |
| chr3\|124463468\|124463676\|MER3:hAT-Charlie:D\|140\|+ | ALS-Ox | ALS-TD |
| chr3\|124721676\|124721972\|AluSq4:Alu:SINE\|98\|+ | ALS-Ox | NA |
| chr3\|131001755\|131001818\|MamTip2:hAT-Tip100:D\|286\|+ | ALS-Ox | ALS-Ox |
| chr3\|134485316\|134485487\|L2b:L2:LINE\|380\|+ | ALS-Ox | NA |
| chr3\|143991288\|143991374\|L2a:L2:LINE\|212\|− | ALS-Ox | ALS-Ox |
| chr3\|150460922\|150461027\|L1ME3G:L1:LINE\|171\|+ | ALS-Ox | NA |
| chr3\|150628880\|150629057\|Tigger4a:TcMar-Tigger:D\|109\|− | ALS-Ox | ALS-Ox |
| chr3\|160871235\|160871527\|L3b:CR1:LINE\|326\|+ | ALS-Ox | NA |
| chr3\|172631518\|172631624\|MIR3:MIR:SINE\|231\|− | ALS-Ox | NA |
| chr3\|173835878\|173836091\|MIRc:MIR:SINE\|315\|− | ALS-Ox | NA |
| chr3\|179902066\|179902288\|MIRb:MIR:SINE\|355\|− | ALS-Ox | ALS-Glia |
| chr3\|24120403\|24120790\|Tigger19b:TcMar-Tigger:D\|264\|− | ALS-Ox | NA |
| chr3\|33867725\|33867801\|X6A_LINE:CR1:LINE\|316\|− | ALS-Ox | NA |
| chr3\|35695768\|35696006\|MIR:MIR:SINE\|338\|− | ALS-Ox | ALS-TD |
| chr3\|35718485\|35718675\|MamSINE1:tR-RALS-TE:SINE\|372\|+ | ALS-Ox | NA |
| chr3\|35730312\|35730531\|MER20:hAT-Charlie:D\|232\|+ | ALS-Ox | NA |
| chr3\|35752606\|35752728\|MER5A1:hAT-Charlie:D\|172\|− | ALS-Ox | ALS-TD |
| chr3\|35763881\|35764012\|AluJb:Alu:SINE\|114\|+ | ALS-Ox | ALS-TD |
| chr3\|35777269\|35777438\|MIRb:MIR:SINE\|309\|− | ALS-Ox | NA |
| chr3\|39515660\|39516008\|L2a:L2:LINE\|307\|+ | ALS-TD | ALS-Glia |
| chr3\|408347\|408547\|MIRc:MIR:SINE\|342\|+ | ALS-Ox | ALS-Ox |
| chr3\|53668661\|53668998\|MER58B:hAT-Charlie:D\|264\|− | ALS-Ox | ALS-TD |
| chr3\|68196775\|68197037\|MLT1K:ERVL-MaLR:LTR\|323\|− | ALS-Ox | ALS-TD |
| chr3\|75958297\|75958644\|L2a:L2:LINE\|313\|− | ALS-Ox | ALS-TD |
| chr3\|85007279\|85007481\|MER58A:hAT-Charlie:D\|237\|− | ALS-Ox | ALS-TD |
| chr3\|85068838\|85068983\|LFSINE_Vert:tR:SINE\|338\|− | ALS-Ox | ALS-TD |
| chr3\|85122831\|85122962\|L2c:L2:LINE\|372\|+ | ALS-Ox | ALS-TD |
| chr3\|85277169\|85277309\|MIRb:MIR:SINE\|314\|− | ALS-Ox | ALS-Ox |
| chr3\|85386562\|85386732\|MIRb:MIR:SINE\|364\|+ | ALS-Ox | ALS-TD |
| chr3\|85633458\|85633629\|MER5B:hAT-Charlie:D\|278\|+ | ALS-Ox | ALS-Ox |
| chr4\|101342495\|101342676\|MamRep434:TcMar-Tigger:D\|284\|− | ALS-Ox | ALS-Ox |
| chr4\|143471397\|143471734\|Charlie15b:hAT-Charlie:D\|252\|− | ALS-Ox | ALS-Ox |
| chr4\|144739655\|144739957\|LFSINE_Vert:tR:SINE\|315\|+ | ALS-Ox | ALS-Glia |
| chr4\|153324878\|153325035\|MER113:hAT-Charlie:D\|229\|+ | ALS-Ox | ALS-Glia |
| chr4\|157334489\|157334624\|L2c:L2:LINE\|362\|+ | ALS-Ox | ALS |
| | | ALS-Ox |
| chr4\|175740967\|175741134\|L2:L2:LINE\|374\|+ | ALS-Ox | NA |
| chr4\|175787512\|175787708\|L2:L2:LINE\|335\|+ | ALS-Ox | ALS-TD |
| chr4\|175797930\|175798041\|MER5A:hAT-Charlie:D\|243\|+ | ALS-Ox | ALS-TD |
| chr4\|176329213\|176329427\|MIRc:MIR:SINE\|322\|− | ALS-Ox | ALS-Ox |
| chr4\|185586261\|185586440\|MER135:D:D\|301\|+ | ALS-Ox | NA |
| chr4\|21317675\|21318020\|THE1B:ERVL-MaLR:LTR\|109\|+ | ALS-Ox | ALS-TD |
| chr4\|21342380\|21342562\|MIRb:MIR:SINE\|311\|− | ALS-Ox | ALS-TD |
| chr4\|21343628\|21343765\|MER5A1:hAT-Charlie:D\|256\|− | ALS-Ox | ALS-TD |
| chr4\|21436568\|21436794\|MIR:MIR:SINE\|269\|+ | ALS-Ox | ALS-TD |
| chr4\|21500323\|21500507\|MIRb:MIR:SINE\|326\|+ | ALS-Ox | ALS-Ox |
| chr4\|21569657\|21569808\|LTR33A:ERVL:LTR\|267\|− | ALS-Ox | ALS-TD |
| chr4\|21570770\|21571010\|MIR:MIR:SINE\|312\|− | ALS-Ox | ALS-TD |
| chr4\|21580197\|21580406\|MIRb:MIR:SINE\|383\|− | ALS-Ox | ALS-Ox |
| chr4\|21597081\|21597444\|THE1B:ERVL-MaLR:LTR\|145\|+ | ALS-Ox | ALS-TD |
| chr4\|21708301\|21708416\|MER5A:hAT-Charlie:D\|319\|+ | ALS-Ox | ALS-TD |
| chr4\|21812601\|21812915\|MamGypLTR1a:Gypsy:LTR\|309\|− | ALS-Ox | ALS-Ox |
| chr4\|24081793\|24082025\|MIRc:MIR:SINE\|320\|+ | ALS-Ox | ALS-TD |
| chr4\|44692030\|44692202\|Charlie23a:hAT-Charlie:D\|235\|− | ALS-Ox | ALS-Ox |
| chr4\|47433154\|47433223\|MIRc:MIR:SINE\|273\|+ | ALS-Ox | ALS-Ox |
| chr4\|56328622\|56328732\|MER5A:hAT-Charlie:D\|283\|+ | ALS-Ox | NA |
| chr4\|61384742\|61385088\|L1MB7:L1:LINE\|195\|+ | ALS-Ox | ALS-TD |
| chr4\|75047494\|75047704\|MER20:hAT-Charlie:D\|176\|+ | ALS-Ox | NA |
| chr5\|107593523\|107593683\|MER5B:hAT-Charlie:D\|277\|+ | ALS-Ox | ALS-TD |
| chr5\|112846029\|112846107\|MIRb:MIR:SINE\|320\|− | ALS-Ox | NA |
| chr5\|11389727\|11390087\|Tigger4b:TcMar-Tigger:D\|170\|+ | ALS-Ox | NA |
| chr5\|11554392\|11554635\|MIRb:MIR:SINE\|322\|+ | ALS-Ox | NA |
| chr5\|11899659\|11899883\|MIRc:MIR:SINE\|311\|− | ALS-Ox | ALS-Ox |
| chr5\|131953331\|131953625\|AluJb:Alu:SINE\|157\|+ | ALS-Ox | ALS-Ox |
| chr5\|137489774\|137490129\|L2c:L2:LINE\|341\|+ | ALS-TD | ALS-Glia |
| chr5\|142152915\|142153329\|Tigger8:TcMar-Tigger:D\|284\|− | ALS-Ox | ALS-Ox |
| chr5\|146798658\|146798908\|MIR:MIR:SINE\|325\|− | ALS-Ox | NA |
| chr5\|151100786\|151100954\|MIR3:MIR:SINE\|295\|− | ALS-Ox | NA |
| chr5\|157394544\|157394671\|MER5A:hAT-Charlie:D\|270\|+ | ALS-Ox | ALS-Ox |
| chr5\|177306847\|177306946\|L2a:L2:LINE\|162\|+ | ALS-Ox | NA |
| chr5\|44817550\|44817906\|MLT1A0:ERVL-MaLR:LTR\|206\|+ | ALS-Ox | ALS-Ox |
| chr5\|59533641\|59533806\|MER5A:hAT-Charlie:D\|261\|+ | ALS-Ox | ALS-TD |
| chr5\|59579145\|59579312\|L2c:L2:LINE\|325\|+ | ALS-Ox | ALS-TD |
| chr5\|71566571\|71566678\|MER58B:hAT-Charlie:D\|215\|− | ALS-Ox | NA |
| chr5\|760200\|760576\|MLT1B:ERVL-MaLR:LTR\|277\|+ | ALS-TD | ALS-TD |
| chr5\|81418515\|81418848\|Tigger3a:TcMar-Tigger:D\|153\|− | ALS-Ox | NA |
| chr5\|95814114\|95814267\|Charlie29a:hAT-Charlie:D\|268\|+ | ALS-Ox | ALS-Ox |
| chr6\|101529591\|101529870\|L2c:L2:LINE\|355\|− | ALS-Ox | ALS-Ox |
| chr6\|105098875\|105099158\|AluJb:Alu:SINE\|159\|− | ALS-TD | ALS-Glia |
| chr6\|116277660\|116277934\|AluSg:Alu:SINE\|44\|+ | ALS-Ox | ALS-Ox |
| chr6\|117561326\|117561397\|OldhAT1:hAT-Ac:D\|282\|+ | ALS-Ox | NA |
| chr6\|12766476\|12766667\|MER63A:hAT-Blackjack:D\|234\|+ | ALS-Ox | ALS-TD |
| chr6\|142928062\|142928263\|L1MC1:L1:LINE\|155\|+ | ALS-Ox | NA |
| chr6\|142932704\|142932875\|L3:CR1:LINE\|343\|+ | ALS-Ox | NA |
| chr6\|145885061\|145885269\|MIRc:MIR:SINE\|327\|+ | ALS-Ox | NA |
| chr6\|147312427\|147312669\|MER20:hAT-Charlie:D\|203\|+ | ALS-Ox | NA |
| chr6\|147321348\|147321596\|AluJr4:Alu:SINE\|189\|+ | ALS-Ox | ALS-TD |
| chr6\|24129404\|24129646\|L2a:L2:LINE\|285\|− | ALS-Ox | NA |
| chr6\|27357958\|27358126\|MIR3:MIR:SINE\|303\|− | ALS-Ox | ALS-Ox |
| chr6\|30724489\|30724580\|L2a:L2:LINE\|198\|+ | ALS-Ox | ALS-Ox |
| chr6\|31544893\|31545138\|MIRb:MIR:SINE\|290\|− | ALS-Ox | ALS-Ox |
| chr6\|39795360\|39795610\|MIRb:MIR:SINE\|332\|+ | ALS-TD | ALS-Glia |
| chr6\|49430916\|49431136\|LTR86A1:ERVL:LTR\|291\|− | ALS-Ox | NA |
| chr6\|68649850\|68650067\|LTR33:ERVL:LTR\|283\|+ | ALS-Ox | ALS-TD |
| chr6\|68651672\|68651794\|Charlie15a:hAT-Charlie:D\|277\|− | ALS-Ox | ALS-TD |
| chr6\|68690583\|68690783\|MIRb:MIR:SINE\|314\|+ | ALS-Ox | ALS-Ox |
| chr6\|68696121\|68696309\|LTR81A:Gypsy:LTR\|308\|− | ALS-Ox | ALS-Ox |
| chr6\|68708138\|68708428\|ORSL:hAT-Tip 100:D\|242\|− | ALS-Ox | ALS-Ox |
| chr6\|68728646\|68728841\|MIR:MIR:SINE\|297\|− | ALS-Ox | ALS-Ox |
| chr6\|68832632\|68832783\|MamSINE1:tR-RALS-TE:SINE\|351\|+ | ALS-Ox | ALS-Ox |
| chr6\|68994190\|68994255\|AmnSINE1:5S-Deu-L2:SINE\|274\|+ | ALS-Ox | NA |
| chr6\|69023699\|69023909\|L2:L2:LINE\|350\|− | ALS-Ox | ALS-TD |
| chr6\|7288091\|7288229\|Tigger12:TcMar-Tigger:D\|276\|+ | ALS-Ox | NA |
| chr6\|89328333\|89328482\|Tigger16b:TcMar-Tigger:D\|232\|+ | ALS-Ox | NA |
| chr6\|96212898\|96212972\|MIR3:MIR:SINE\|270\|+ | ALS-Ox | ALS-Ox |
| chr6\|96795093\|96795198\|MER5A1:hAT-Charlie:D\|255\|+ | ALS-Ox | NA |
| chr6\|96889945\|96890143\|MIRb:MIR:SINE\|313\|− | ALS-Ox | ALS-Ox |
| chr7\|103101598\|103101760\|MIR:MIR:SINE\|248\|− | ALS-Ox | NA |
| chr7\|111123071\|111123147\|X9_LINE:L1:LINE\|297\|− | ALS-Ox | NA |
| chr7\|12234751\|12234992\|MIRc:MIR:SINE\|386\|− | ALS-Ox | NA |
| chr7\|130515142\|130515533\|MSTA:ERVL-MaLR:LTR\|137\|− | ALS-Ox | ALS-Ox |
| chr7\|130518046\|130518269\|L1ME3Cz:L1:LINE\|236\|+ | ALS-Ox | ALS-Ox |
| chr7\|130886885\|130887046\|MIRb:MIR:SINE\|333\|+ | ALS-Ox | ALS-TD |
| chr7\|130917112\|130917401\|AluJo:Alu:SINE\|166\|− | ALS-Ox | ALS-TD |
| chr7\|130942710\|130942786\|L1MD:L1:LINE\|240\|− | ALS-Ox | ALS-TD |
| chr7\|134290416\|134290501\|L2c:L2:LINE\|171\|− | ALS-Ox | NA |
| chr7\|141280292\|141280651\|THE1B:ERVL-MaLR:LTR\|143\|+ | ALS-Ox | ALS-TD |
| chr7\|141478488\|141478813\|MER33:hAT-Charlie:D\|199\|− | ALS-Ox | ALS-Ox |
| chr7\|146414287\|146414621\|MER102c:hAT-Charlie:D\|293\|+ | ALS-Ox | ALS-TD |
| chr7\|22318698\|22318981\|L2c:L2:LINE\|340\|+ | ALS-Ox | ALS-TD |
| chr7\|30923921\|30924027\|Mam Tip2:hAT-Tip100:D\|324\|+ | ALS-TD | ALS-Glia |
| chr7\|80217578\|80217733\|UCON55:TcMar-Tigger:D\|312\|+ | ALS-Ox | ALS-Ox |
| chr7\|90236977\|90237152\|MLT1J1:ERVL-MaLR:LTR\|214\|+ | ALS-Ox | NA |
| chr8\|100918736\|100918836\|L2c:L2:LINE\|320\|− | ALS-Ox | ALS-Ox |
| chr8\|106326575\|106326876\|L2a:L2:LINE\|244\|− | ALS-Ox | ALS-TD |
| chr8\|10881700\|10881887\|MIRb:MIR:SINE\|274\|− | ALS-Ox | NA |
| chr8\|118192750\|118192931\|ORSL-2a:hAT-Tip 100:D\|328\|+ | ALS-Ox | NA |
| chr8\|135528624\|135528968\|MLT1A1:ERVL-MaLR:LTR\|166\|− | ALS-Ox | ALS-TD |
| chr8\|17232055\|17232221\|MIR3:MIR:SINE\|303\|+ | ALS-Ox | NA |
| chr8\|26587832\|26587969\|MIR1_Amn:MIR:SINE\|336\|− | ALS-TD | ALS-TD |
| chr8\|27733718\|27733865\|Tigger19b:TcMar-Tigger:D\|331\|− | ALS-Ox | NA |
| chr8\|28564771\|28564985\|MIRc:MIR:SINE\|211\|+ | ALS-Ox | ALS-Ox |
| chr8\|28569310\|28569496\|MIR3:MIR:SINE\|315\|+ | ALS-Ox | ALS-Ox |
| chr8\|4233375\|4233725\|MLT1A0:ERVL-MaLR:LTR\|171\|+ | ALS-Ox | ALS-TD |
| chr8\|4353125\|4353476\|THE1B:ERVL-MaLR:LTR\|128\|+ | ALS-Ox | ALS-TD |
| chr8\|4407018\|4407190\|MER58A:hAT-Charlie:D\|215\|+ | ALS-Ox | ALS-TD |
| chr8\|4812350\|4812580\|L1MA4A:L1:LINE\|159\|+ | ALS-Ox | ALS-TD |
| chr8\|4821881\|4821985\|MIRc:MIR:SINE\|327\|+ | ALS-Ox | ALS-TD |
| chr8\|56958199\|56958343\|L2b:L2:LINE\|303\|− | ALS-Ox | NA |
| chr8\|63211720\|63211856\|UCON2:D?:D?\|273\|− | ALS-Ox | NA |
| chr8\|74442122\|74442232\|MamTip2:hAT-Tip 100:D\|283\|− | ALS-Ox | ALS-Ox |
| chr8\|78604183\|78604412\|MIRb:MIR:SINE\|297\|− | ALS-Ox | ALS-Ox |
| chr8\|78604415\|78604567\|MIR3:MIR:SINE\|318\|− | ALS-Ox | ALS-Ox |
| chr8\|81702186\|81702445\|MLT11:ERVL-MaLR:LTR\|378\|+ | ALS-Ox | ALS-Ox |
| chr8\|89925920\|89926069\|MER5A1:hAT-Charlie:D\|150\|+ | ALS-Ox | NA |
| chr9\|105551331\|105551532\|MIRc:MIR:SINE\|293\|+ | ALS-Ox | NA |
| chr9\|123119717\|123119799\|MIR:MIR:SINE\|268\|− | ALS-Ox | NA |
| chr9\|127682748\|127682914\|MIRb:MIR:SINE\|364\|+ | ALS-TD | ALS-TD |
| chr9\|19550846\|19551149\|L1MA4A:L1:LINE\|136\|− | ALS-TD | ALS-Glia |
| chr9\|19782704\|19782952\|MIRb:MIR:SINE\|314\|− | ALS-Ox | ALS-Glia |
| chr9\|19784296\|19784384\|MamRep434:TcMar-Tigger:D\|341\|− | ALS-Ox | ALS-Glia |
| chr9\|74690316\|74690454\|MER102a:hAT-Charlie:D\|267\|− | ALS-Ox | ALS-Ox |
| chr9\|76386869\|76387001\|FLAM_C:Alu:SINE\|114\|− | ALS-Ox | ALS-Ox |
| chr9\|77419191\|77419438\|MER112:hAT-Charlie:D\|244\|+ | ALS-Ox | NA |
| chr9\|84760109\|84760280\|MIRb:MIR:SINE\|352\|+ | ALS-Ox | ALS-Ox |
| chr9\|85024426\|85024610\|MIRb:MIR:SINE\|289\|− | ALS-Ox | ALS-Ox |
| CHRM4 | ALS-Ox | ALS-Ox |
| CHR2 | ALS-Glia | ALS-Ox |
| chrX\|101554375\|101554585\|Mam_R4:Dong-R4:LINE\|291\|− | ALS-Ox | ALS-Ox |
| chrX\|10235000\|10235355\|MLT1J:ERVL-MaLR:LTR\|298\|− | ALS-Ox | NA |
| chrX\|102657424\|102657499\|AmnSINE2:tR-Deu:SINE\|370\|− | ALS-Ox | ALS-Ox |
| chrX\|103062725\|103062791\|L1M5:L1:LINE\|234\|+ | ALS-Ox | ALS-Ox |
| chrX\|103377819\|103378035\|L1M5:L1:LINE\|345\|− | ALS-Ox | ALS-Ox |
| chrX\|111223842\|111223996\|L1M E4c:L1:LINE\|123\|+ | ALS-Ox | ALS-Ox |
| chrX\|123478917\|123478996\|MIRc:MIR:SINE\|246\|− | ALS-Ox | ALS-TD |
| chrX\|129447597\|129447769\|MER 104:TcMar-Tc2:D\|186\|+ | ALS-Ox | ALS-Ox |
| chrX\|135963017\|135963318\|L1MB8:L1:LINE\|139\|+ | ALS-Ox | NA |
| chrX\|140774736\|140774891\|Tigger20a:TcMar-Tigger:D\|286\|+ | ALS-Ox | ALS-TD |
| chrX\|140781721\|140781935\|MER58A:hAT-Charlie:D\|201\|+ | ALS-Ox | ALS-Ox |
| chrX\|17150666\|17150837\|MIR3:MIR:SINE\|358\|− | ALS-Ox | NA |
| chrX\|24545754\|24545941\|L2a:L2:LINE\|267\|− | ALS-Ox | NA |
| chrX\|46602445\|46602619\|MER5B:hAT-Charlie:D\|301\|+ | ALS-Ox | NA |
| chrX\|51902142\|51902279\|MER117:hAT-Charlie:D\|285\|− | ALS-Ox | ALS-Ox |
| chrX\|54815877\|54816014\|MER117:hAT-Charlie:D\|248\|− | ALS-Ox | NA |
| chrX\|74589722\|74589950\|MamSINE1:tR-RALS-TE:SINE\|303\|+ | ALS-Ox | NA |
| chrX\|9948055\|9948253\|L1MD:L1:LINE\|217\|+ | ALS-Ox | NA |
| CICP18 | ALS-TD | ALS-TD |
| CICP4 | ALS-TD | ALS-TD |
| CKS2 | ALS-Ox | ALS-Ox |
| CLCA2 | ALS-Ox | ALS-Glia |
| CLCA4 | ALS-TD | ALS-Glia |
| CLCF1 | ALS-Glia | NA |
| CLEC17A | ALS-Glia | ALS-Glia |
| CLEC18A | ALS-TD | ALS-TD |
| CLEC18C | ALS-TD | ALS-TD |
| CLEC7A | ALS-Glia | ALS-Ox |
| CLGN | ALS-Ox | ALS-Ox |
| CLIC6 | ALS-Glia | ALS-Ox |
| CMTM5 | ALS-TD | ALS-Glia |
| CNGB1 | ALS-Ox | ALS-Ox |
| CNGB3 | ALS-Ox | ALS-Ox |
| CNN1 | ALS-Glia | NA |
| CNN2 | ALS-Glia | ALS-Glia |
| CNTN6 | ALS-Ox | ALS-Ox |
| COL14A1 | ALS-Glia | ALS-Glia |
| COL18A1 | ALS-Glia | ALS-Glia |
| COL1A1 | ALS-Glia | ALS-Ox |
| COL1A2 | ALS-Glia | ALS-TD |
| COL2A1 | ALS-Glia | NA |
| COL3A1 | ALS-Glia | ALS-Ox |
| COL4A6 | ALS-Glia | ALS-Glia |
| COL6A3 | ALS-Glia | ALS-Ox |
| COL8A1 | ALS-Glia | ALS-Glia |
| CALS-Ox412 | ALS-Glia | ALS-TD |
| CALS-Ox7A2 | ALS-Ox | ALS-Ox |
| CP | ALS-TD | ALS-Glia |
| CPA2 | ALS-TD | ALS-Glia |
| CPAMD8 | ALS-Glia | ALS-Glia |
| CPB1 | ALS-TD | ALS-Glia |
| CPXM1 | ALS-Glia | ALS-Ox |
| CPZ | ALS-Glia | ALS-Ox |
| CR1 | ALS-TD | ALS-Glia |
| CR2 | ALS-Glia | ALS-Glia |
| CRABP1 | ALS-Glia | ALS-Ox |
| CRACR2B | ALS-Glia | NA |
| CRH | ALS-Glia | ALS-Ox |
| CRHBP | ALS-Ox | ALS-Ox |
| CRIP1 | ALS-Glia | ALS-TD |
| CRYM | ALS-Ox | ALS-Ox |
| CSF3 | ALS-TD | ALS-Glia |
| CSPG4P5 | ALS-Ox | ALS-Ox |
| CSTA | ALS-Glia | ALS-Glia |
| CTXN2 | ALS-Ox | NA |
| CTXN3 | ALS-Ox | ALS-Ox |
| CX3CR1 | ALS-Glia | ALS-Ox |
| CXCL1 | ALS-Glia | ALS-TD |
| CYB5R2 | ALS-Glia | ALS-Glia |
| CYP19A1 | ALS-Glia | ALS-Glia |
| CYP4F12 | ALS-TD | ALS-Glia |
| DACH2 | ALS-Ox | ALS-Ox |
| DAO | ALS-TD | ALS-Glia |
| DAPP1 | ALS-Glia | ALS-Glia |
| DAW1 | ALS-Ox | ALS-Ox |
| DCLK3 | ALS-Ox | ALS-Ox |
| DDX12P | ALS-TD | ALS-Glia |
| DES | ALS-Glia | ALS-Ox |
| DKK2 | ALS-Glia | ALS-Ox |
| DLGAP1-AS4 | ALS-Ox | ALS-Ox |
| DLX1 | ALS-Glia | ALS-Ox |
| DLX5 | NA | ALS-Ox |
| DLX6 | NA | ALS-Ox |
| DMBT1 | ALS-TD | ALS-Glia |
| DMRT2 | ALS-TD | ALS-Glia |
| DMRT3 | ALS-Ox | ALS-Ox |
| DH17 | ALS-TD | ALS-Glia |
| DH17-AS1 | ALS-TD | ALS-Glia |
| DJC5G | ALS-Ox | ALS-TD |
| DSE1L2 | ALS-TD | ALS-TD |
| DNM1P47 | ALS-TD | ALS-Glia |
| DOCK6 | ALS-TD | ALS-TD |
| DRGX | ALS-Ox | ALS-Ox |
| DSC1 | ALS-Ox | ALS-Ox |
| DSG2 | ALS-Glia | ALS-Ox |
| DSG3 | ALS-Ox | ALS-Ox |
| DSP | ALS-Glia | ALS-Ox |
| DTHD1 | ALS-TD | ALS-Glia |
| DUALS-Ox1 | ALS-TD | ALS-Glia |
| DUALS-OxA1 | ALS-TD | ALS-Glia |
| DYDC2 | ALS-Ox | ALS-Ox |
| DYNLT3 | ALS-Ox | ALS-Ox |
| EEF1E1 | ALS-Ox | ALS-Ox |
| EGFL6 | ALS-Glia | ALS-Ox |
| EGLN1P1 | ALS-TD | ALS-TD |
| EGR4 | ALS-Glia | ALS-Ox |
| EIF5A2 | ALS-Ox | ALS-Ox |
| ELOVL4 | ALS-Ox | ALS-Ox |
| ENSG00000188897 | ALS-TD | ALS-Glia |
| ENSG00000197320 | ALS-Glia | ALS-Glia |
| ENSG00000204584 | ALS-TD | ALS-TD |
| ENSG00000205041 | ALS-TD | ALS-TD |
| ENSG00000205562 | NA | ALS-Ox |
| ENSG00000213197 | ALS-TD | ALS-Glia |
| ENSG00000214100 | ALS-Glia | ALS-Glia |
| ENSG00000214265 | NA | ALS-Ox |
| ENSG00000215068 | ALS-TD | ALS-Glia |
| ENSG00000216285 | ALS-Ox | ALS-Ox |
| ENSG00000223379 | ALS-TD | ALS-TD |
| ENSG00000223522 | ALS-TD | ALS-Glia |
| ENSG00000223812 | ALS-Ox | ALS-Ox |
| ENSG00000223855 | ALS-TD | ALS-TD |
| ENSG00000223930 | ALS-Ox | ALS-Ox |
| ENSG00000224153 | ALS-TD | ALS-TD |
| ENSG00000224415 | ALS-TD | ALS-TD |
| ENSG00000225032 | ALS-TD | ALS-TD |
| ENSG00000225140 | ALS-TD | ALS-Glia |
| ENSG00000225539 | ALS-Ox | ALS-Ox |
| ENSG00000225877 | ALS-TD | ALS-Glia |
| ENSG00000226070 | ALS-TD | ALS-TD |
| ENSG00000226454 | ALS-TD | ALS-TD |
| ENSG00000226994 | ALS-TD | ALS-Glia |
| ENSG00000227544 | ALS-Ox | ALS-Glia |
| ENSG00000228242 | ALS-TD | ALS-TD |
| ENSG00000228392 | ALS-TD | ALS-Glia |
| ENSG00000228434 | ALS-TD | ALS-TD |
| ENSG00000228477 | ALS-TD | ALS-TD |
| ENSG00000228496 | ALS-TD | ALS-TD |
| ENSG00000228510 | ALS-TD | ALS-Glia |
| ENSG00000228543 | ALS-Ox | ALS-Ox |
| ENSG00000228741 | ALS-TD | ALS-Glia |
| ENSG00000228971 | ALS-Ox | ALS-Ox |
| ENSG00000228998 | ALS-TD | NA |
| ENSG00000228999 | ALS-Ox | ALS-Ox |
| ENSG00000229425 | ALS-Ox | ALS-Ox |
| ENSG00000229492 | ALS-TD | ALS-Glia |
| ENSG00000229569 | ALS-TD | ALS-TD |
| ENSG00000229618 | ALS-Ox | ALS-Ox |
| ENSG00000229657 | ALS-TD | ALS-TD |
| ENSG00000230084 | ALS-TD | ALS-Glia |
| ENSG00000230387 | ALS-Ox | ALS-TD |
| ENSG00000230715 | ALS-TD | ALS-TD |
| ENSG00000230852 | ALS-Ox | ALS-Ox |
| ENSG00000231013 | ALS-Ox | ALS-Ox |
| ENSG00000231449 | NA | ALS-TD |
| ENSG00000231536 | NA | ALS-Ox |
| ENSG00000231840 | ALS-TD | ALS-Glia |
| ENSG00000232310 | ALS-TD | ALS-Glia |
| ENSG00000232748 | ALS-TD | ALS-TD |
| ENSG00000233508 | ALS-Ox | ALS-Ox |
| ENSG00000234111 | ALS-Ox | ALS-Ox |
| ENSG00000234292 | ALS-TD | ALS-Glia |
| ENSG00000234394 | ALS-Ox | ALS-TD |
| ENSG00000234810 | ALS-TD | ALS-Glia |
| ENSG00000234913 | ALS-TD | ALS-TD |
| ENSG00000235012 | ALS-TD | ALS-TD |
| ENSG00000235390 | ALS-TD | ALS-Glia |
| ENSG00000235672 | ALS-TD | ALS-TD |
| ENSG00000235683 | ALS-TD | ALS-TD |
| ENSG00000236841 | ALS-Ox | ALS-Ox |
| ENSG00000237166 | ALS-TD | ALS-Glia |
| ENSG00000237250 | ALS-Ox | ALS-Ox |
| ENSG00000237268 | ALS-TD | ALS-Glia |
| ENSG00000238035 | ALS-TD | ALS-TD |
| ENSG00000238194 | ALS-TD | ALS-Glia |
| ENSG00000238217 | ALS-TD | ALS-Glia |
| ENSG00000239828 | ALS-TD | ALS-Glia |
| ENSG00000239922 | ALS-TD | ALS-Glia |
| ENSG00000240265 | ALS-TD | ALS-Glia |
| ENSG00000240687 | ALS-TD | ALS-Glia |
| ENSG00000240695 | ALS-TD | ALS-TD |
| ENSG00000241218 | ALS-TD | ALS-Glia |
| ENSG00000241345 | ALS-TD | ALS-Glia |
| ENSG00000241956 | ALS-Ox | ALS-TD |
| ENSG00000243302 | ALS-TD | ALS-TD |
| ENSG00000243679 | ALS-TD | ALS-TD |
| ENSG00000243696 | ALS-TD | ALS-TD |
| ENSG00000244198 | ALS-TD | ALS-Glia |
| ENSG00000244427 | ALS-TD | ALS-TD |
| ENSG00000244738 | ALS-TD | ALS-Glia |
| ENSG00000246363 | ALS-Ox | ALS-Ox |
| ENSG00000248015 | ALS-TD | ALS-Glia |
| ENSG00000248115 | NA | ALS-Ox |
| ENSG00000248710 | ALS-TD | ALS-Glia |
| ENSG00000248713 | ALS-Ox | ALS-Ox |
| ENSG00000248738 | ALS-Ox | ALS-TD |
| ENSG00000249436 | ALS-Ox | ALS-Ox |
| ENSG00000249604 | ALS-TD | ALS-Glia |
| ENSG00000250072 | ALS-TD | ALS-Glia |
| ENSG00000250135 | ALS-Glia | NA |
| ENSG00000250198 | ALS-TD | ALS-Glia |
| ENSG00000250397 | ALS-TD | ALS-TD |
| ENSG00000250575 | ALS-TD | ALS-TD |
| ENSG00000251226 | ALS-Ox | ALS-Ox |
| ENSG00000251423 | ALS-Ox | ALS-Ox |
| ENSG00000253547 | ALS-Ox | ALS-TD |
| ENSG00000254187 | ALS-Ox | ALS-Ox |
| ENSG00000254491 | ALS-TD | ALS-Glia |
| ENSG00000255087 | ALS-Ox | ALS-Ox |
| ENSG00000255176 | ALS-TD | ALS-Glia |
| ENSG00000255521 | ALS-TD | ALS-Glia |
| ENSG00000256422 | NA | ALS-Ox |
| ENSG00000256443 | ALS-TD | ALS-TD |
| ENSG00000256500 | ALS-Ox | ALS-Ox |
| ENSG00000256538 | NA | ALS-Ox |
| ENSG00000256966 | ALS-Ox | ALS-Ox |
| ENSG00000257501 | ALS-Ox | ALS-Ox |
| ENSG00000257522 | NA | ALS-Ox |
| ENSG00000257607 | ALS-TD | ALS-TD |
| ENSG00000257894 | ALS-Glia | ALS-Ox |
| ENSG00000258342 | ALS-TD | ALS-Glia |
| ENSG00000258483 | NA | ALS-Glia |
| ENSG00000258559 | ALS-TD | ALS-TD |
| ENSG00000258611 | ALS-TD | ALS-TD |
| ENSG00000258674 | ALS-TD | ALS-TD |
| ENSG00000258811 | NA | ALS-TD |
| ENSG00000258844 | ALS-TD | ALS-Glia |
| ENSG00000259288 | ALS-Glia | ALS-Ox |
| ENSG00000259433 | ALS-TD | ALS-Glia |
| ENSG00000259604 | ALS-TD | ALS-Glia |
| ENSG00000259605 | ALS-Glia | NA |
| ENSG00000259707 | ALS-TD | ALS-TD |
| ENSG00000259780 | NA | ALS-Glia |
| ENSG00000259804 | ALS-TD | ALS-TD |
| ENSG00000259807 | ALS-TD | ALS-Glia |
| ENSG00000259834 | ALS-Ox | ALS-Ox |
| ENSG00000259895 | ALS-TD | ALS-TD |
| ENSG00000259914 | ALS-TD | ALS-Glia |
| ENSG00000260157 | ALS-Ox | ALS-Ox |
| ENSG00000260179 | ALS-TD | ALS-TD |
| ENSG00000260198 | ALS-TD | ALS-TD |
| ENSG00000260276 | ALS-TD | ALS-TD |
| ENSG00000260316 | ALS-TD | ALS-TD |
| ENSG00000260328 | ALS-Ox | ALS-Ox |
| ENSG00000260401 | ALS-TD | ALS-Glia |
| ENSG00000260412 | ALS-Ox | ALS-Ox |
| ENSG00000260417 | ALS-TD | ALS-TD |
| ENSG00000260426 | ALS-TD | ALS-Glia |
| ENSG00000260515 | ALS-TD | ALS-TD |
| ENSG00000260585 | ALS-Ox | ALS-Ox |
| ENSG00000260604 | ALS-TD | ALS-Glia |
| ENSG00000260648 | ALS-TD | ALS-TD |
| ENSG00000260788 | ALS-TD | ALS-Glia |
| ENSG00000260838 | ALS-Ox | ALS-Ox |
| ENSG00000260878 | ALS-Ox | ALS-Ox |
| ENSG00000261026 | ALS-Ox | ALS-TD |
| ENSG00000261037 | ALS-Ox | ALS-Ox |
| ENSG00000261067 | ALS-TD | ALS-TD |
| ENSG00000261121 | ALS-TD | ALS-Glia |
| ENSG00000261292 | ALS-Ox | ALS-Ox |
| ENSG00000261325 | ALS-Ox | ALS-Ox |
| ENSG00000261327 | ALS-TD | ALS-Glia |
| ENSG00000261329 | ALS-TD | ALS-Glia |
| ENSG00000261542 | ALS-Ox | ALS-Ox |
| ENSG00000261634 | ALS-Glia | ALS-TD |
| ENSG00000261710 | ALS-TD | ALS-Glia |
| ENSG00000261728 | ALS-Ox | ALS-Ox |
| ENSG00000261795 | ALS-TD | ALS-Glia |
| ENSG00000262188 | ALS-TD | ALS-Glia |
| ENSG00000262304 | ALS-TD | ALS-TD |
| ENSG00000262312 | ALS-TD | ALS-TD |
| ENSG00000262528 | ALS-TD | ALS-Glia |
| ENSG00000262580 | ALS-TD | ALS-TD |
| ENSG00000262668 | ALS-Ox | ALS-Ox |
| ENSG00000262877 | ALS-TD | ALS-Glia |
| ENSG00000262903 | ALS-TD | ALS-TD |
| ENSG00000263020 | ALS-TD | ALS-TD |
| ENSG00000263278 | ALS-TD | ALS-TD |
| ENSG00000263603 | ALS-TD | ALS-TD |
| ENSG00000263715 | ALS-Ox | ALS-Ox |
| ENSG00000264324 | ALS-TD | ALS-Glia |
| ENSG00000265118 | ALS-Glia | ALS-Glia |
| ENSG00000265179 | ALS-Ox | ALS-Ox |
| ENSG00000265579 | ALS-Ox | ALS-Ox |
| ENSG00000265690 | ALS-TD | ALS-TD |
| ENSG00000266498 | ALS-TD | ALS-TD |
| ENSG00000266844 | ALS-TD | ALS-Glia |
| ENSG00000267034 | ALS-Ox | ALS-Ox |
| ENSG00000267065 | ALS-TD | ALS-TD |
| ENSG00000267077 | ALS-TD | NA |
| ENSG00000267152 | ALS-Ox | ALS-Ox |
| ENSG00000267160 | ALS-Ox | ALS-Ox |
| ENSG00000267283 | ALS-TD | ALS-TD |
| ENSG00000267385 | ALS-TD | ALS-TD |
| ENSG00000267412 | ALS-TD | ALS-TD |
| ENSG00000267605 | ALS-TD | ALS-TD |
| ENSG00000267629 | ALS-Ox | ALS-Ox |
| ENSG00000267740 | ALS-Glia | ALS-Ox |
| ENSG00000267749 | ALS-TD | ALS-TD |
| ENSG00000267801 | ALS-TD | ALS-Glia |
| ENSG00000268170 | ALS-TD | ALS-Glia |
| ENSG00000268173 | ALS-Glia | NA |
| ENSG00000268518 | ALS-TD | ALS-Glia |
| ENSG00000268670 | ALS-TD | ALS-TD |
| ENSG00000269242 | ALS-Glia | ALS-Glia |
| ENSG00000269318 | ALS-TD | ALS-TD |
| ENSG00000269653 | ALS-TD | ALS-TD |
| ENSG00000270020 | ALS-TD | ALS-TD |
| ENSG00000270111 | NA | ALS-Ox |
| ENSG00000271653 | ALS-TD | ALS-TD |
| ENSG00000271894 | ALS-Ox | ALS-Ox |
| ENSG00000271959 | ALS-TD | ALS-TD |
| ENSG00000272010 | ALS-Ox | ALS-Ox |
| ENSG00000272078 | ALS-TD | ALS-TD |
| ENSG00000272219 | ALS-TD | ALS-TD |
| ENSG00000272321 | ALS-Ox | ALS-Ox |
| ENSG00000272512 | ALS-TD | ALS-TD |
| ENSG00000272807 | ALS-Ox | ALS-Ox |
| ENSG00000272821 | ALS-TD | ALS-TD |
| ENSG00000272884 | ALS-TD | ALS-TD |
| ENSG00000272941 | ALS-TD | ALS-Glia |
| ENSG00000272944 | NA | ALS-Ox |
| ENSG00000273142 | ALS-TD | ALS-TD |
| ENSG00000273151 | ALS-TD | ALS-Glia |
| ENSG00000273259 | ALS-TD | ALS-Glia |
| ENSG00000273301 | ALS-Ox | ALS-Ox |
| ENSG00000273409 | ALS-Glia | ALS-Ox |
| ENSG00000273580 | ALS-TD | ALS-TD |
| ENSG00000273597 | ALS-TD | ALS-TD |
| ENSG00000273618 | ALS-TD | ALS-TD |
| ENSG00000274080 | ALS-TD | ALS-TD |
| ENSG00000274184 | ALS-TD | ALS-Glia |
| ENSG00000274251 | ALS-TD | ALS-TD |
| ENSG00000274272 | ALS-TD | ALS-Glia |
| ENSG00000274330 | ALS-Ox | ALS-Ox |
| ENSG00000274565 | ALS-Glia | ALS-Glia |
| ENSG00000274628 | ALS-TD | ALS-TD |
| ENSG00000275286 | ALS-Ox | ALS-Ox |
| ENSG00000275426 | ALS-Ox | ALS-Ox |
| ENSG00000275468 | ALS-TD | ALS-TD |
| ENSG00000275484 | ALS-TD | ALS-TD |
| ENSG00000275850 | ALS-TD | ALS-TD |
| ENSG00000276127 | ALS-TD | ALS-Glia |
| ENSG00000276232 | ALS-Glia | ALS-Glia |
| ENSG00000276462 | ALS-Ox | ALS-TD |
| ENSG00000276690 | ALS-TD | ALS-Glia |
| ENSG00000278434 | ALS-TD | ALS-TD |
| ENSG00000278716 | ALS-TD | ALS-TD |
| ENSG00000278727 | NA | ALS-Ox |
| ENSG00000278803 | ALS-Glia | ALS-Ox |
| ENSG00000278872 | ALS-TD | ALS-TD |
| ENSG00000278911 | ALS-Ox | ALS-Ox |
| ENSG00000278962 | ALS-Ox | ALS-Ox |
| ENSG00000279013 | ALS-Ox | ALS-Ox |
| ENSG00000279042 | ALS-Ox | ALS-TD |
| ENSG00000279044 | ALS-TD | ALS-TD |
| ENSG00000279057 | ALS-TD | ALS-TD |
| ENSG00000279094 | ALS-Glia | ALS-Glia |
| ENSG00000279146 | ALS-TD | ALS-TD |
| ENSG00000279147 | ALS-TD | ALS-TD |
| ENSG00000279161 | ALS-TD | ALS-Glia |
| ENSG00000279179 | ALS-TD | ALS-TD |
| ENSG00000279189 | ALS-Ox | ALS-TD |
| ENSG00000279191 | ALS-TD | ALS-TD |
| ENSG00000279196 | ALS-TD | ALS-TD |
| ENSG00000279233 | ALS-TD | ALS-TD |
| ENSG00000279283 | ALS-TD | ALS-TD |
| ENSG00000279305 | ALS-Ox | ALS-Ox |
| ENSG00000279328 | ALS-TD | ALS-TD |
| ENSG00000279360 | ALS-TD | ALS-Glia |
| ENSG00000279387 | ALS-TD | ALS-TD |
| ENSG00000279419 | ALS-Ox | ALS-Glia |
| ENSG00000279462 | ALS-TD | ALS-TD |
| ENSG00000279474 | ALS-TD | ALS-TD |
| ENSG00000279476 | ALS-TD | ALS-TD |
| ENSG00000279495 | ALS-TD | ALS-Glia |
| ENSG00000279513 | ALS-Ox | ALS-Ox |
| ENSG00000279537 | ALS-TD | ALS-TD |
| ENSG00000279543 | ALS-TD | ALS-TD |
| ENSG00000279573 | ALS-TD | ALS-TD |
| ENSG00000279583 | ALS-TD | ALS-TD |
| ENSG00000279586 | ALS-TD | ALS-TD |
| ENSG00000279620 | ALS-TD | ALS-TD |
| ENSG00000279675 | ALS-Ox | ALS-Ox |
| ENSG00000279678 | ALS-TD | ALS-TD |
| ENSG00000279693 | ALS-TD | ALS-TD |
| ENSG00000279700 | ALS-TD | ALS-TD |
| ENSG00000279712 | ALS-TD | ALS-TD |
| ENSG00000279744 | ALS-TD | ALS-TD |
| ENSG00000279759 | ALS-TD | ALS-TD |
| ENSG00000279764 | ALS-TD | ALS-TD |
| ENSG00000279801 | ALS-TD | ALS-TD |
| ENSG00000279803 | ALS-TD | ALS-Glia |
| ENSG00000279819 | ALS-TD | ALS-Glia |
| ENSG00000279821 | ALS-TD | ALS-TD |
| ENSG00000279839 | ALS-TD | ALS-TD |
| ENSG00000279861 | ALS-TD | ALS-TD |
| ENSG00000279878 | ALS-TD | ALS-TD |
| ENSG00000279917 | ALS-TD | ALS-TD |
| ENSG00000279952 | ALS-Ox | ALS-Ox |
| ENSG00000279980 | ALS-TD | ALS-TD |
| ENSG00000279981 | ALS-TD | ALS-Glia |
| ENSG00000279996 | ALS-TD | ALS-Glia |
| ENSG00000280033 | ALS-TD | ALS-TD |
| ENSG00000280063 | ALS-TD | ALS-TD |
| ENSG00000280073 | ALS-TD | ALS-TD |
| ENSG00000280105 | ALS-Ox | ALS-Ox |
| ENSG00000280143 | ALS-Glia | ALS-Glia |
| ENSG00000280152 | ALS-TD | ALS-TD |
| ENSG00000280163 | ALS-TD | ALS-TD |
| ENSG00000280205 | ALS-TD | ALS-TD |
| ENSG00000280206 | ALS-TD | ALS-Glia |
| ENSG00000280214 | ALS-TD | ALS-TD |
| ENSG00000280222 | ALS-TD | ALS-TD |
| ENSG00000280245 | ALS-TD | ALS-TD |
| ENSG00000280248 | ALS-TD | ALS-Glia |
| ENSG00000280287 | ALS-TD | ALS-TD |
| ENSG00000280332 | ALS-TD | ALS-TD |
| ENSG00000280388 | ALS-TD | ALS-TD |
| ENSG00000280486 | ALS-TD | ALS-Glia |
| ENSG00000280571 | ALS-TD | ALS-Glia |
| ENSG00000280893 | ALS-TD | ALS-TD |
| ENSG00000281160 | ALS-Ox | ALS-Ox |
| ENSG00000281969 | ALS-TD | ALS-Glia |
| ENSG00000282033 | NA | ALS-Ox |
| ENSG00000283025 | ALS-Ox | ALS-Ox |
| ENSG00000283088 | ALS-TD | ALS-Glia |
| ENSG00000283149 | ALS-Glia | NA |
| ENSG00000283178 | ALS-Glia | NA |
| ENSG00000283199 | ALS-TD | ALS-TD |
| ENSG00000283239 | ALS-TD | ALS-Glia |
| ENSG00000283462 | ALS-TD | ALS-Glia |
| ENSG00000283486 | ALS-TD | ALS-Glia |
| ENSG00000283633 | ALS-TD | ALS-Glia |
| ENSG00000283646 | ALS-Glia | ALS-Ox |
| ENSG00000283886 | ALS-Ox | ALS-Ox |
| ENSG00000283914 | ALS-TD | ALS-Glia |
| ENSG00000284240 | NA | ALS-Ox |
| ENSG00000284377 | ALS-TD | ALS-Glia |
| ENSG00000284657 | ALS-TD | ALS-Glia |
| ENSG00000284703 | ALS-Ox | ALS-Ox |
| ENSG00000284762 | ALS-TD | ALS-Glia |
| ENSG00000284779 | ALS-Glia | ALS-TD |
| ENSG00000284848 | ALS-Ox | ALS-Ox |
| ENSG00000284981 | ALS-TD | ALS-TD |
| ENSG00000285051 | ALS-TD | ALS-Glia |
| ENSG00000285188 | ALS-Ox | ALS-TD |
| ENSG00000285238 | ALS-TD | NA |
| ENSG00000285269 | ALS-TD | ALS-Glia |
| ENSG00000285292 | ALS-Ox | ALS-Ox |
| ENSG00000285492 | ALS-TD | ALS-Glia |
| ENSG00000285561 | ALS-Ox | ALS-Ox |
| ENSG00000285565 | ALS-Glia | ALS-Ox |
| ENSG00000285578 | ALS-Ox | ALS-Ox |
| ENSG00000285581 | ALS-TD | ALS-TD |
| ENSG00000285596 | ALS-TD | ALS-TD |
| ENSG00000285634 | ALS-Ox | ALS-Ox |
| ENSG00000285839 | ALS-Glia | ALS-Glia |
| ENSG00000285897 | NA | ALS-Ox |
| ENSG00000285939 | ALS-Ox | ALS-Ox |
| ENSG00000285942 | ALS-Glia | ALS-TD |
| ENSG00000285952 | ALS-TD | ALS-Glia |
| ENSG00000285972 | ALS-Glia | ALS-Glia |
| ENSG00000286015 | ALS-Ox | ALS-Ox |
| ENSG00000286044 | ALS-TD | ALS-TD |
| ENSG00000286069 | NA | ALS-Ox |
| ENSG00000286084 | ALS-Ox | ALS-Ox |
| ENSG00000286159 | ALS-TD | ALS-Glia |
| ENSG00000286198 | ALS-Ox | ALS-Ox |
| ENSG00000286220 | ALS-TD | ALS-Glia |
| ENSG00000286235 | ALS-Ox | ALS-Glia |
| ENSG00000286269 | ALS-Ox | ALS-Ox |
| ENTPD3 | NA | ALS-Ox |
| EPHX4 | ALS-Ox | ALS-Ox |
| EPN3 | ALS-Ox | ALS-TD |
| ERFE | ALS-Ox | ALS-Ox |
| ESM1 | ALS-Glia | ALS-Ox |
| ESRP1 | ALS-Ox | ALS-Ox |
| EVA1B | ALS-Glia | ALS-Glia |
| EVI2A | ALS-Ox | ALS-Glia |
| F10 | ALS-Glia | NA |
| FAM153B | ALS-TD | ALS-TD |
| FAM162B | ALS-Ox | ALS-Ox |
| FAM163A | ALS-Glia | ALS-Ox |
| FAM178B | ALS-TD | ALS-Glia |
| FAM19A1 | ALS-Ox | ALS-Ox |
| FAM19A2 | ALS-Ox | ALS-Ox |
| FAM220A | ALS-Glia | ALS-Ox |
| FAM225A | ALS-TD | ALS-Glia |
| FAM27E4 | ALS-TD | ALS-TD |
| FAM3C | ALS-Ox | ALS-Ox |
| FAM3C2 | ALS-Ox | ALS-Ox |
| FAM65C | ALS-TD | ALS-TD |
| FAM95C | ALS-TD | ALS-Glia |
| FBLIM1 | ALS-Glia | ALS-Ox |
| FBLN7 | ALS-Ox | ALS-Ox |
| FBXO40 | NA | ALS-Ox |
| FBXW4P1 | ALS-TD | ALS-TD |
| FCER1G | ALS-Glia | ALS-Glia |
| FCGR1A | ALS-Glia | ALS-Glia |
| FCGR1B | ALS-Glia | ALS-Ox |
| FCGR1CP | ALS-Glia | ALS-Glia |
| FCGR2B | ALS-Glia | ALS-Glia |
| FCGR2C | ALS-Glia | ALS-Glia |
| FCGR3A | ALS-Glia | ALS-Glia |
| FER1L6 | ALS-Glia | ALS-Ox |
| FFAR1 | ALS-TD | ALS-Glia |
| FGF17 | ALS-TD | ALS-TD |
| FGF9 | ALS-Ox | ALS-Ox |
| FGR | ALS-Glia | ALS-Glia |
| FLRT3 | ALS-Ox | ALS-Ox |
| FLT4 | ALS-Glia | ALS-TD |
| FMOD | ALS-Glia | ALS-Ox |
| FNDC9 | ALS-Ox | ALS-Ox |
| FOLH1 | ALS-TD | ALS-Glia |
| FALS-OxC2 | ALS-Glia | ALS-TD |
| FALS-OxD2 | ALS-Glia | ALS-Ox |
| FALS-OxS1 | ALS-Glia | NA |
| FPR1 | ALS-Glia | ALS-Glia |
| FPR2 | ALS-Glia | ALS-Glia |
| FPR3 | ALS-Glia | ALS-Glia |
| FREM3 | ALS-Ox | ALS-Ox |
| FRZB | ALS-Glia | ALS-Ox |
| FSHR | ALS-Ox | ALS-Ox |
| FXYD5 | ALS-Glia | ALS-Glia |
| FZD10 | ALS-Glia | NA |
| GABRA1 | ALS-Ox | ALS-Ox |
| GABRA6 | ALS-Ox | ALS-Ox |
| GAD2 | ALS-Ox | ALS-Ox |
| GAPDHP32 | ALS-Ox | ALS-TD |
| GAPT | ALS-Glia | ALS-Ox |
| GAS6-AS1 | ALS-TD | ALS-Glia |
| GATA2 | ALS-Glia | ALS-TD |
| GATA2-AS1 | ALS-TD | ALS-TD |
| GATA6 | ALS-Glia | NA |
| GDA | ALS-Ox | ALS-Ox |
| GGH | ALS-Ox | ALS-Ox |
| GGT5 | ALS-Glia | ALS-Glia |
| GJB1 | ALS-TD | ALS-Glia |
| GJB2 | ALS-Glia | ALS-Ox |
| GJD2 | ALS-Ox | ALS-Ox |
| GLIPR2 | ALS-Glia | ALS-Glia |
| GLP2R | ALS-Ox | ALS-Ox |
| GLRA2 | ALS-Ox | ALS-Ox |
| GLRA3 | ALS-Ox | ALS-Ox |
| GNB3 | ALS-TD | ALS-TD |
| GNG13 | ALS-Ox | ALS-Ox |
| GOLGA80 | ALS-TD | ALS-TD |
| GPIHBP1 | ALS-TD | ALS-Glia |
| GPR149 | NA | ALS-Ox |
| GPR183 | ALS-Glia | ALS-Ox |
| GPR22 | ALS-Ox | ALS-Ox |
| GPR26 | ALS-Ox | ALS-Ox |
| GPR34 | ALS-Glia | ALS-Ox |
| GPR35 | ALS-TD | ALS-TD |
| GPR4 | ALS-Glia | ALS-TD |
| GPR6 | ALS-Ox | ALS-Ox |
| GPR62 | ALS-TD | ALS-Glia |
| GPR68 | ALS-Ox | ALS-Ox |
| GPR83 | ALS-Ox | ALS-Ox |
| GPRC5A | ALS-Glia | ALS-Glia |
| GPX8 | ALS-Glia | ALS-Glia |
| GRAPL | ALS-TD | NA |
| GRHL3 | ALS-TD | ALS-Glia |
| GSTM1 | ALS-Ox | NA |
| GTF2IP13 | ALS-TD | ALS-Glia |
| GULP1 | ALS-Ox | ALS-Ox |
| GUSBP5 | ALS-Ox | ALS-Ox |
| GYPC | ALS-Glia | ALS-Glia |
| HAGLR | ALS-TD | ALS-Glia |
| HAMP | ALS-Glia | ALS-Glia |
| HAPLN1 | ALS-Ox | ALS-Ox |
| HCG4P3 | ALS-TD | ALS-TD |
| HDGFL1 | ALS-TD | ALS-TD |
| HEYL | ALS-Glia | ALS-Glia |
| HIC1 | ALS-Glia | ALS-TD |
| HILS1 | NA | ALS-Glia |
| HIST1H1B | ALS-Glia | ALS-Glia |
| HIST1H1T | ALS-TD | ALS-Glia |
| HIST2H3A | ALS-Glia | ALS-Ox |
| HIST2H3C | ALS-Glia | ALS-Ox |
| HK3 | ALS-Glia | ALS-Glia |
| HLA-DOA | ALS-Glia | ALS-Glia |
| HLA-DQA1 | ALS-Glia | ALS-Glia |
| HLA-DQA2 | ALS-Glia | ALS-Ox |
| HLA-DRA | ALS-Glia | ALS-Glia |
| HLA-DRB1 | ALS-Glia | ALS-Glia |
| HLA-DRB5 | ALS-Glia | ALS-Glia |
| HMGB1P35 | ALS-TD | ALS-Glia |
| HMSD | ALS-Glia | ALS-Glia |
| HNF4A | ALS-Ox | ALS-Ox |
| HALS-OxD1 | ALS-TD | ALS-Glia |
| HPN-AS1 | ALS-TD | ALS-Glia |
| HPRT1 | ALS-Ox | ALS-Ox |
| HPYR1 | ALS-Ox | ALS-TD |
| HS6ST2 | ALS-Ox | ALS-Ox |
| HSD17B1 | ALS-TD | ALS-Glia |
| HSD17B1P1 | ALS-TD | ALS-Glia |
| HSD17B3 | ALS-TD | ALS-Glia |
| HSFX2 | ALS-TD | ALS-Ox |
| HSP90AB4P | ALS-TD | ALS-TD |
| HSPA7 | ALS-Glia | ALS-Glia |
| HSPB3 | ALS-Ox | ALS-Ox |
| HSPB7 | ALS-Glia | ALS-Glia |
| HTR2A | ALS-Ox | ALS-Ox |
| HTR2C | ALS-Ox | ALS-Ox |
| HTR3B | ALS-Ox | ALS-Ox |
| HTR5A-AS1 | NA | ALS-Ox |
| IFI30 | ALS-Glia | ALS-Glia |
| IGF1 | ALS-Ox | ALS-Ox |
| IGF2 | ALS-Glia | ALS-Ox |
| IGF2BP2 | ALS-Glia | ALS-Glia |
| IGSF6 | ALS-Glia | ALS-Glia |
| IKBKGP1 | ALS-Glia | NA |
| IL12A-AS1 | ALS-Ox | ALS-TD |
| IL1B | ALS-Glia | ALS-Ox |
| IL1RAPL2 | ALS-Ox | ALS-Ox |
| IL21R | ALS-Glia | ALS-Glia |
| IL4R | ALS-Glia | ALS-Glia |
| IMPG1 | ALS-Ox | ALS-Ox |
| INHBA-AS1 | ALS-Ox | ALS-Ox |
| INMT | ALS-Glia | ALS-Glia |
| INSRR | ALS-Glia | NA |
| IP6K3 | ALS-TD | ALS-Glia |
| IRF7 | ALS-TD | ALS-Glia |
| ISG20 | ALS-TD | ALS-Glia |
| ITGA10 | ALS-Glia | ALS-TD |
| ITGA2 | ALS-TD | ALS-Glia |
| ITGA5 | ALS-Glia | ALS-Glia |
| ITGB3 | ALS-Glia | ALS-Glia |
| ITGBL1 | ALS-Glia | ALS-Glia |
| ITIH2 | ALS-Glia | ALS-Ox |
| ITIH3 | ALS-Glia | NA |
| ITPKB-IT1 | ALS-TD | ALS-Glia |
| ITPR3 | ALS-Glia | ALS-TD |
| JAML | ALS-Glia | ALS-Glia |
| KANK3 | ALS-Glia | ALS-TD |
| KCNB2 | ALS-Ox | ALS-Ox |
| KCNE4 | ALS-Glia | ALS-Glia |
| KCNG3 | ALS-Ox | ALS-Ox |
| KCNIP4 | ALS-Ox | ALS-Ox |
| KCNJ13 | ALS-Glia | ALS-Ox |
| KCNMB1 | ALS-Glia | NA |
| KCNQ1DN | ALS-TD | ALS-Glia |
| KCNV1 | ALS-Ox | ALS-Ox |
| KEL | ALS-TD | ALS-Glia |
| KIAA0408 | ALS-Ox | ALS-Ox |
| KIAA1210 | ALS-Glia | ALS-Ox |
| KIF12 | ALS-Ox | ALS-Ox |
| KIF19 | ALS-TD | ALS-Glia |
| KITLG | ALS-Ox | ALS-Ox |
| KLF2 | ALS-Glia | ALS-TD |
| KLHDC7A | ALS-Glia | ALS-Ox |
| KLHL14 | ALS-Glia | ALS-Ox |
| KLK5 | ALS-Ox | ALS-Ox |
| KLK6 | ALS-TD | ALS-Glia |
| KLK7 | ALS-Ox | ALS-Ox |
| KNG1 | ALS-Ox | ALS-Ox |
| KNOP1P4 | ALS-TD | ALS-TD |
| KRT18P4 | ALS-TD | ALS-TD |
| KRT18P5 | ALS-TD | ALS-TD |
| KRT19 | ALS-Glia | ALS-TD |
| KRT222 | ALS-Ox | NA |
| KRT5 | ALS-Ox | ALS-Ox |
| KRT8P13 | ALS-TD | ALS-Glia |
| KRT8P39 | ALS-TD | ALS-TD |
| KRT8P42 | ALS-TD | ALS-Glia |
| LAMC3 | ALS-Glia | ALS-Glia |
| LAMP5 | ALS-Ox | ALS-Ox |
| LANCL3 | ALS-Ox | ALS-Ox |
| LBX2-AS1 | ALS-TD | ALS-Glia |
| LGR5 | ALS-TD | ALS-Glia |
| LIF | ALS-Glia | ALS-Glia |
| LILRA4 | ALS-Glia | ALS-Glia |
| LILRA6 | ALS-Glia | ALS-Glia |
| LILRB2 | ALS-Glia | ALS-Glia |
| LILRB3 | ALS-Glia | ALS-Glia |
| LILRB5 | ALS-Glia | ALS-Ox |
| LIMS2 | ALS-Glia | ALS-TD |
| LIN28B | ALS-Ox | ALS-Ox |
| LINC00176 | ALS-TD | ALS-TD |
| LINC00283 | ALS-Ox | ALS-Ox |
| LINC00323 | ALS-TD | ALS-Glia |
| LINC00326 | ALS-Ox | ALS-TD |
| LINC00460 | ALS-Ox | ALS-Ox |
| LINC00482 | ALS-TD | ALS-Glia |
| LINC00488 | ALS-Ox | ALS-Ox |
| LINC00507 | ALS-Ox | ALS-Ox |
| LINC00601 | ALS-TD | ALS-Glia |
| LINC00609 | ALS-TD | ALS-Glia |
| LINC00638 | ALS-TD | ALS-Glia |
| LINC00639 | ALS-TD | ALS-Glia |
| LINC00643 | ALS-Ox | ALS-Ox |
| LINC00862 | ALS-TD | ALS-Glia |
| LINC00877 | ALS-TD | ALS-Glia |
| LINC00898 | ALS-Ox | ALS-Ox |
| LINC00930 | ALS-TD | ALS-TD |
| LINC00940 | ALS-TD | ALS-Glia |
| LINC01007 | ALS-Ox | ALS-Ox |
| LINC01010 | ALS-TD | ALS-Glia |
| LINC01088 | ALS-Glia | ALS-Glia |
| LINC01139 | ALS-Glia | ALS-TD |
| LINC01140 | ALS-Ox | ALS-Ox |
| LINC01164 | ALS-Ox | ALS-TD |
| LINC01202 | ALS-Ox | ALS-Ox |
| LINC01222 | ALS-TD | ALS-Glia |
| LINC01235 | ALS-Glia | ALS-Glia |
| LINC01250 | ALS-Ox | ALS-TD |
| LINC01289 | NA | ALS-Ox |
| LINC01347 | ALS-TD | ALS-TD |
| LINC01361 | NA | ALS-Ox |
| LINC01378 | ALS-Ox | ALS-Ox |
| LINC01445 | ALS-TD | ALS-Glia |
| LINC01476 | ALS-Ox | ALS-Ox |
| LINC01511 | ALS-Ox | ALS-Ox |
| LINC01549 | ALS-TD | ALS-Glia |
| LINC01616 | ALS-Ox | ALS-Ox |
| LMAN1L | ALS-TD | ALS-Glia |
| LMF1-AS1 | ALS-TD | ALS-Glia |
| LPAR4 | ALS-Glia | ALS-Glia |
| LRRC2 | ALS-Ox | ALS-Ox |
| LRRC32 | ALS-Glia | ALS-TD |
| LRRC53 | NA | ALS-Ox |
| LRRC63 | ALS-TD | ALS-Glia |
| LRRN4CL | ALS-Glia | NA |
| LSP1 | ALS-Glia | ALS-Glia |
| LSR | ALS-Glia | ALS-TD |
| LTF | ALS-TD | ALS-Glia |
| LTK | ALS-Ox | ALS-TD |
| LUCAT1 | ALS-TD | ALS-Glia |
| LURAP1L-AS1 | ALS-TD | ALS-Glia |
| LY86 | ALS-Glia | ALS-Glia |
| LY86-AS1 | ALS-Ox | ALS-Ox |
| LY96 | ALS-Glia | ALS-Glia |
| MAD2L1 | ALS-Ox | ALS-Ox |
| MAGEC3 | ALS-TD | ALS-Glia |
| MAL2 | ALS-Ox | ALS-Ox |
| MARCO | ALS-Glia | ALS-Ox |
| MAS1 | ALS-Ox | ALS-Ox |
| MBOAT1 | ALS-Glia | ALS-Glia |
| MCHR1 | ALS-Ox | ALS-Ox |
| MCHR2 | ALS-Ox | ALS-Ox |
| MCUB | ALS-Glia | ALS-Ox |
| MEG9 | ALS-TD | ALS-TD |
| MEPE | ALS-Ox | ALS-Ox |
| METTL21C | ALS-Ox | ALS-Ox |
| MFAP2 | ALS-Glia | NA |
| MFAP4 | ALS-Glia | ALS-Ox |
| MIR133A1HG | NA | ALS-Ox |
| MIR219A2 | ALS-TD | ALS-Glia |
| MIR24-2 | ALS-TD | ALS-Glia |
| MIR3648-1 | ALS-TD | ALS-TD |
| MIR3648-2 | ALS-TD | ALS-TD |
| MIR7-3HG | ALS-Ox | ALS-Ox |
| MIRLET7BHG | ALS-TD | ALS-TD |
| MLKL | ALS-TD | ALS-Glia |
| MLPH | ALS-Glia | ALS-Glia |
| MMP25 | ALS-TD | ALS-Glia |
| MMP8 | NA | ALS-Glia |
| MMP9 | ALS-TD | ALS-Glia |
| MS4A4A | ALS-Glia | ALS-Glia |
| MS4A8 | ALS-Ox | ALS-Ox |
| MSANTD3-TMEFF1 | ALS-Ox | ALS-Ox |
| MSH5-SAPCD1 | ALS-TD | ALS-Glia |
| MSL3P1 | ALS-Ox | ALS-Ox |
| MSR1 | ALS-Glia | ALS-Glia |
| MSX2 | ALS-Glia | ALS-Ox |
| MTMR9LP | ALS-TD | ALS-Glia |
| MUC20P1 | ALS-TD | ALS-TD |
| MUC4 | ALS-Glia | ALS-TD |
| MUC5B | ALS-Ox | ALS-TD |
| MUCL1 | NA | ALS-Ox |
| MYH11 | ALS-Glia | ALS-TD |
| MYL9 | ALS-Glia | ALS-TD |
| MYOZ3 | NA | ALS-Ox |
| MZT1 | ALS-Ox | ALS-Ox |
| NOGP4 | ALS-TD | ALS-Glia |
| PSB | ALS-Glia | ALS-Glia |
| NCF2 | ALS-Glia | ALS-Glia |
| NDNF | ALS-Glia | ALS-Ox |
| NDST4 | ALS-Ox | ALS-Ox |
| NDUFA4L2 | ALS-Glia | ALS-TD |
| NDUFAB1 | ALS-Ox | ALS-Ox |
| NEK2 | ALS-Ox | ALS-Ox |
| NEUROD1 | ALS-Ox | ALS-Ox |
| NEUROD6 | ALS-Ox | ALS-Ox |
| NFE2L3 | ALS-Glia | ALS-Glia |
| NGFR | ALS-TD | ALS-Glia |
| NINJ2 | ALS-Glia | ALS-Glia |
| NIPAL4 | ALS-TD | ALS-Glia |
| NKX6-2 | ALS-TD | ALS-Glia |
| NME5 | NA | ALS-Ox |
| NMU | ALS-Ox | ALS-Ox |
| NOS2P3 | ALS-TD | ALS-TD |
| NOS3 | ALS-Glia | ALS-TD |
| NOTCH3 | ALS-Glia | ALS-TD |
| NPC1L1 | ALS-TD | ALS-TD |
| NPIPA2 | ALS-TD | ALS-TD |
| NPIPA8 | ALS-Ox | ALS-TD |
| NPR1 | ALS-Glia | ALS-TD |
| NPR3 | ALS-Ox | ALS-Ox |
| NPY2R | ALS-Ox | ALS-Ox |
| NPY5R | ALS-Ox | ALS-Ox |
| NRG4 | ALS-Ox | ALS-Ox |
| NSRP1P1 | ALS-TD | ALS-TD |
| NTM-AS1 | ALS-TD | ALS-Glia |
| NUDT4P1 | ALS-Ox | ALS-Ox |
| NWD2 | ALS-Ox | ALS-Ox |
| NXPH2 | ALS-Ox | ALS-Ox |
| ODF3B | ALS-Glia | ALS-Glia |
| OGN | ALS-Glia | ALS-Ox |
| OLFM3 | ALS-Ox | ALS-Ox |
| OLFM4 | ALS-Ox | ALS-Ox |
| OLFML1 | ALS-Glia | ALS-TD |
| OLR1 | ALS-Glia | ALS-Glia |
| OPCML-IT1 | ALS-TD | ALS-TD |
| OR1411 | ALS-Ox | ALS-TD |
| OR2A1-AS1 | ALS-Glia | NA |
| OR6W1P | ALS-TD | ALS-Glia |
| OR7A5 | ALS-TD | ALS-Glia |
| OR7C1 | ALS-TD | ALS-Glia |
| OR7E125P | ALS-TD | ALS-TD |
| OR9A2 | ALS-TD | ALS-Glia |
| OSR1 | ALS-Glia | ALS-TD |
| OSTN | ALS-Ox | ALS-Ox |
| OTOGL | ALS-Ox | ALS-Ox |
| ALS-OxGR1 | ALS-Ox | ALS-Ox |
| P2RX1 | ALS-Glia | ALS-Glia |
| P2RY12 | ALS-Ox | ALS-Ox |
| P2RY13 | ALS-Glia | ALS-Ox |
| PACRG-AS1 | ALS-TD | ALS-Glia |
| PACRG-AS3 | ALS-TD | ALS-Glia |
| PCBP3-OT1 | ALS-TD | ALS-TD |
| PCDH8 | ALS-Glia | ALS-Ox |
| PCDHA14 | ALS-TD | ALS-TD |
| PCP4 | ALS-Ox | ALS-Ox |
| PCP4L1 | ALS-Ox | ALS-Ox |
| PCSK1 | ALS-Ox | ALS-Ox |
| PDE4C | ALS-Glia | ALS-TD |
| PDGFD | ALS-Glia | ALS-Ox |
| PDLIM1 | ALS-Glia | ALS-Ox |
| PEAR1 | ALS-Glia | ALS-TD |
| PENK | ALS-Glia | ALS-Ox |
| PES1P2 | NA | ALS-TD |
| PFDN4 | ALS-Ox | ALS-Ox |
| PGA3 | ALS-Glia | ALS-Ox |
| PGAM2 | ALS-Glia | ALS-Glia |
| PGGHG | ALS-TD | ALS-Glia |
| PHBP7 | ALS-TD | ALS-TD |
| PIRT | ALS-Glia | ALS-Glia |
| PKD2L1 | ALS-Ox | ALS-TD |
| PKHD1L1 | ALS-Glia | ALS-Ox |
| PKIB | ALS-Ox | ALS-Ox |
| PKN3 | ALS-Glia | ALS-TD |
| PLA1A | ALS-Glia | ALS-Glia |
| PLA2G4B | ALS-TD | ALS-TD |
| PLAC8 | ALS-Glia | ALS-Glia |
| PLEKHA4 | ALS-Glia | ALS-Glia |
| PLEKHG2 | ALS-Glia | ALS-Glia |
| PLN | ALS-Glia | ALS-Ox |
| PLS1 | ALS-Ox | ALS-Ox |
| PNMA5 | ALS-Ox | ALS-Ox |
| PNOC | ALS-Glia | ALS-Ox |
| PODN | ALS-Glia | ALS-TD |
| POLR3DP1 | ALS-TD | ALS-TD |
| POM121L9P | ALS-TD | ALS-Glia |
| POU5F1P5 | ALS-TD | ALS-TD |
| PPP1R14A | ALS-Glia | ALS-Glia |
| PPP1R14BP3 | ALS-TD | NA |
| PPP1R17 | ALS-Ox | ALS-Ox |
| PRB2 | ALS-TD | ALS-TD |
| PRF1 | ALS-Glia | NA |
| PRICKLE4 | ALS-TD | ALS-TD |
| PRIMA1 | ALS-TD | ALS-Glia |
| PRKAG2-AS1 | NA | ALS-Ox |
| PRLHR | ALS-Ox | ALS-Ox |
| PRMT8 | NA | ALS-Ox |
| PRR26 | ALS-TD | ALS-Glia |
| PRR5L | ALS-TD | ALS-Glia |
| PRRX2 | ALS-Glia | NA |
| PRSS12 | ALS-Ox | ALS-Ox |
| PRSS53 | ALS-TD | ALS-TD |
| PRTN3 | ALS-Glia | ALS-Ox |
| PSORS1C1 | ALS-TD | ALS-Glia |
| PTGDR | ALS-Glia | ALS-Ox |
| PTGER3 | ALS-Ox | ALS-Ox |
| PTGER4 | ALS-Glia | ALS-Glia |
| PTGFR | ALS-Glia | ALS-Ox |
| PTH2R | NA | ALS-Ox |
| PTOV1-AS2 | ALS-TD | NA |
| PTPN3 | ALS-Ox | ALS-Ox |
| PTPN7 | ALS-Glia | ALS-Glia |
| PTPRCAP | ALS-Glia | ALS-Glia |
| PTPRD-AS2 | NA | ALS-Ox |
| PTPRH | ALS-TD | ALS-Glia |
| PVALB | ALS-Ox | ALS-Ox |
| PWAR5 | ALS-Ox | ALS-Ox |
| QPCT | ALS-Ox | ALS-Ox |
| RAB27B | ALS-Ox | ALS-Ox |
| RAB3C | NA | ALS-Ox |
| RAB42 | ALS-Glia | ALS-Glia |
| RASL11B | ALS-Ox | ALS-Ox |
| RASL12 | ALS-Glia | ALS-Glia |
| RASSF9 | ALS-Glia | ALS-Glia |
| RBM43P1 | ALS-TD | ALS-Glia |
| RBP4 | ALS-Ox | ALS-Ox |
| RBP7 | ALS-Glia | ALS-Glia |
| RBPMS | ALS-Glia | ALS-Glia |
| REM1 | ALS-Glia | NA |
| RFK | ALS-Ox | ALS-Ox |
| RGL3 | ALS-TD | ALS-TD |
| RGS1 | ALS-Glia | ALS-Glia |
| RGS18 | ALS-Glia | ALS-Ox |
| RGS4 | ALS-Ox | ALS-Ox |
| RHBDL2 | ALS-TD | ALS-Glia |
| RHEBL1 | ALS-Ox | ALS-Ox |
| RHEBP1 | ALS-Ox | ALS-Ox |
| RHOV | ALS-Ox | ALS-Ox |
| RN7SKP23 | ALS-TD | ALS-TD |
| RSE2 | ALS-Glia | ALS-Ox |
| RSE6 | ALS-Glia | ALS-Glia |
| RNF103-CHMP3 | ALS-Ox | ALS-Ox |
| RPE65 | ALS-Glia | ALS-Ox |
| RPL17-C18orf32 | ALS-Glia | ALS-Ox |
| RPL3P5 | ALS-TD | ALS-TD |
| RPS20P22 | ALS-TD | ALS-TD |
| RPS20P33 | ALS-TD | ALS-Glia |
| RPSAP69 | NA | ALS-Ox |
| RRAD | ALS-Glia | ALS-Glia |
| RTBDN | ALS-Ox | ALS-Ox |
| RTKN2 | ALS-Ox | ALS-Ox |
| RTP1 | ALS-Ox | ALS-Ox |
| RUNX3 | ALS-Glia | ALS-Glia |
| RXFP3 | ALS-Ox | ALS-Ox |
| S100A3 | ALS-Glia | ALS-Glia |
| S100A4 | ALS-Glia | ALS-Glia |
| S100A9 | ALS-Glia | ALS-Glia |
| S1PR4 | ALS-Glia | ALS-Glia |
| S1PR5 | ALS-TD | ALS-Glia |
| SAMD11 | ALS-Glia | ALS-Glia |
| SAP25 | ALS-TD | ALS-Glia |
| SCARA5 | ALS-Glia | ALS-Ox |
| SCIMP | ALS-Glia | ALS-Glia |
| SCIN | ALS-Glia | ALS-Glia |
| SCN5A | ALS-Glia | ALS-Ox |
| SCNN1A | ALS-Glia | ALS-Ox |
| SDR16C5 | NA | ALS-Ox |
| SELL | ALS-Glia | ALS-Glia |
| SEPHS1P6 | ALS-TD | ALS-Glia |
| SERINC2 | ALS-Glia | NA |
| SERPI1 | ALS-Glia | ALS-Glia |
| SERPI3 | ALS-TD | ALS-Glia |
| SERPI5 | ALS-TD | ALS-Glia |
| SERPINB2 | ALS-Ox | ALS-Ox |
| SERPIND1 | ALS-Glia | ALS-Ox |
| SERPINE1 | ALS-Glia | ALS-Glia |
| SERPINI1 | ALS-Ox | ALS-Ox |
| SERTM1 | ALS-Ox | ALS-Ox |
| SFN | ALS-TD | ALS-Glia |
| SFRP2 | ALS-Glia | ALS-Ox |
| SGK2 | ALS-TD | ALS-Glia |
| SH2D2A | ALS-Glia | NA |
| SH2D6 | ALS-TD | ALS-Glia |
| SH3GL1P1 | ALS-TD | ALS-TD |
| SIGLEC5 | ALS-Glia | ALS-Glia |
| SIGLEC7 | ALS-Glia | ALS-Glia |
| SIGLEC9 | ALS-Glia | ALS-Glia |
| SIX1 | ALS-Glia | NA |
| SIX2 | ALS-Glia | ALS-Ox |
| SLAMF8 | ALS-Glia | ALS-Glia |
| SLC13A4 | ALS-Glia | ALS-Ox |
| SLC16A12 | ALS-Glia | ALS-Ox |
| SLC17A6 | ALS-Ox | ALS-Ox |
| SLC17A8 | NA | ALS-Ox |
| SLC1A7 | ALS-Glia | ALS-Glia |
| SLC22A13 | ALS-TD | ALS-TD |
| SLC22A2 | ALS-Glia | NA |
| SLC22A6 | ALS-Glia | ALS-Ox |
| SLC22A8 | ALS-Glia | NA |
| SLC22A9 | ALS-Ox | ALS-Ox |
| SLC25A5P3 | ALS-TD | ALS-Glia |
| SLC26A4-AS1 | ALS-Ox | ALS-Ox |
| SLC26A7 | ALS-Glia | ALS-Ox |
| SLC26A9 | ALS-TD | ALS-Glia |
| SLC27A2 | NA | ALS-Ox |
| SLC27A6 | ALS-TD | ALS-Glia |
| SLC2A3P1 | ALS-Glia | ALS-Glia |
| SLC30A3 | ALS-Ox | ALS-Ox |
| SLC32A1 | ALS-Ox | ALS-Ox |
| SLC45A3 | ALS-TD | ALS-Glia |
| SLC47A2 | ALS-Glia | ALS-Glia |
| SLC4A9 | ALS-TD | ALS-Glia |
| SLC52A3 | ALS-TD | ALS-TD |
| SLC5A11 | ALS-TD | ALS-Glia |
| SLC5A5 | ALS-Glia | ALS-Ox |
| SLC6A13 | ALS-Glia | ALS-TD |
| SLC6A20 | ALS-Glia | ALS-Ox |
| SLC6A7 | NA | ALS-Ox |
| SLC7A5P2 | ALS-TD | NA |
| SLC7A7 | ALS-Glia | ALS-Glia |
| SLCO4A1-AS1 | ALS-TD | ALS-Glia |
| SLFNL1 | ALS-TD | ALS-TD |
| SLN | ALS-Ox | ALS-Ox |
| SLPI | ALS-Glia | NA |
| SLX1B-SULT1A4 | ALS-TD | ALS-TD |
| SMIM17 | ALS-Ox | ALS-Ox |
| SMIM18 | NA | ALS-Ox |
| SMIM5 | ALS-TD | ALS-Glia |
| SMIM6 | ALS-Glia | ALS-Glia |
| SMOC2 | ALS-Glia | ALS-TD |
| SMPX | ALS-Ox | ALS-Ox |
| SMTN | ALS-TD | ALS-TD |
| SMTNL2 | ALS-Glia | ALS-Ox |
| SI1P1 | ALS-TD | ALS-TD |
| SNHG18 | ALS-Glia | ALS-Glia |
| SNX10 | ALS-Ox | ALS-Ox |
| SNX18P3 | ALS-TD | ALS-Glia |
| SNX18P7 | ALS-TD | ALS-TD |
| SNX18P9 | ALS-TD | ALS-TD |
| SNX20 | ALS-Glia | ALS-Glia |
| SOCS3 | ALS-TD | ALS-Glia |
| SOSTDC1 | ALS-Ox | ALS-Ox |
| SOWAHB | ALS-Ox | ALS-Ox |
| SALS-Ox17 | ALS-Glia | ALS-TD |
| SALS-Ox18 | ALS-Glia | ALS-TD |
| SPATA31C1 | ALS-Ox | ALS-Ox |
| SPOCD1 | ALS-TD | ALS-Glia |
| SPTSSB | ALS-Ox | ALS-Ox |
| SRPK3 | ALS-TD | ALS-Glia |
| SRRM5 | ALS-TD | ALS-TD |
| SST | ALS-Glia | ALS-Ox |
| ST6GALC2 | ALS-Glia | ALS-Glia |
| ST6GALC5 | ALS-Ox | ALS-Ox |
| ST8SIA2 | ALS-Glia | ALS-Ox |
| STAT4 | NA | ALS-Ox |
| STH | ALS-TD | ALS-TD |
| STRA6 | ALS-Glia | ALS-TD |
| STYK1 | ALS-Ox | ALS-Ox |
| SUCNR1 | ALS-Glia | ALS-Ox |
| SYNJ2BP-CALS-Ox16 | ALS-Ox | ALS-Ox |
| SYNPR | ALS-Ox | ALS-Ox |
| SYT10 | ALS-Ox | ALS-Ox |
| SYT4 | ALS-Ox | ALS-Ox |
| SYTL5 | ALS-Ox | ALS-Ox |
| TAC1 | ALS-Ox | ALS-Ox |
| TAC3 | ALS-Ox | ALS-Ox |
| TAF7L | ALS-Ox | ALS-Ox |
| TAGLN | ALS-Glia | ALS-Glia |
| TBX15 | ALS-Glia | ALS-TD |
| TBX2-AS1 | ALS-Glia | NA |
| TCIRG1 | ALS-Glia | ALS-Glia |
| TDO2 | ALS-Ox | ALS-Ox |
| TDRD10 | ALS-Glia | NA |
| ALS-TEKT4P2 | ALS-TD | ALS-Glia |
| ALS-TESPA1 | ALS-Ox | ALS-Ox |
| TGFB111 | ALS-Glia | ALS-Glia |
| TGFB2-OT1 | ALS-Ox | ALS-Ox |
| TGM1 | ALS-TD | ALS-TD |
| TIGL1 | ALS-Glia | ALS-TD |
| TLR7 | ALS-Glia | ALS-Glia |
| TLR8 | ALS-Glia | ALS-Glia |
| TLR9 | ALS-TD | ALS-TD |
| TMEM125 | ALS-Glia | ALS-Glia |
| TMEM126A | ALS-Ox | NA |
| TMEM139 | ALS-TD | ALS-Glia |
| TMEM140 | ALS-TD | ALS-Glia |
| TMEM14A | ALS-Ox | ALS-Ox |
| TMEM155 | NA | ALS-Ox |
| TMEM196 | ALS-Ox | ALS-Ox |
| TMEM200A | ALS-Ox | ALS-Ox |
| TMEM204 | ALS-Glia | ALS-TD |
| TMEM215 | ALS-Ox | ALS-Ox |
| TMEM235 | ALS-TD | ALS-Glia |
| TMEM30B | ALS-Glia | ALS-Ox |
| TMEM88B | ALS-TD | ALS-Glia |
| TNC | ALS-Glia | ALS-Glia |
| TNFRSF12A | ALS-Glia | ALS-Glia |
| TNFRSF25 | ALS-TD | ALS-TD |
| TNFRSF6B | ALS-TD | ALS-Glia |
| TNFSF15 | ALS-Ox | ALS-Ox |
| TNRC6C-AS1 | ALS-TD | ALS-Glia |
| TOP2A | ALS-Glia | ALS-Ox |
| TOR4A | ALS-Glia | ALS-Glia |
| TP53TG5 | ALS-TD | ALS-Glia |
| TP63 | ALS-Glia | NA |
| TPH2 | ALS-Ox | ALS-Ox |
| TPI1P1 | ALS-Ox | NA |
| TPALS-TEP1 | ALS-TD | ALS-Glia |
| TRBC2 | ALS-Ox | ALS-Ox |
| TREM1 | ALS-Glia | ALS-Glia |
| TREM2 | ALS-Glia | ALS-Glia |
| TRIM54 | ALS-TD | ALS-TD |
| TRIM59 | ALS-TD | ALS-Glia |
| TRPA1 | ALS-Ox | ALS-Ox |
| TRPV5 | ALS-TD | ALS-Glia |
| TRPV6 | ALS-TD | ALS-Glia |
| TSX-DISC1 | ALS-TD | ALS-Glia |
| TSPAN13 | ALS-Ox | ALS-Ox |
| TSPAN8 | ALS-Glia | ALS-Glia |
| TTC6 | ALS-Ox | ALS-Ox |
| TUB-AS1 | ALS-TD | ALS-TD |
| TUBB6 | ALS-Glia | ALS-Glia |
| TUBBP10 | ALS-TD | ALS-TD |
| TUR | ALS-Ox | ALS-Ox |
| TWF1P1 | ALS-TD | ALS-Glia |
| TYMP | ALS-Glia | ALS-Glia |
| TYMS | ALS-Glia | ALS-Glia |
| TYMSOS | ALS-Glia | ALS-Glia |
| TYROBP | ALS-Glia | ALS-Glia |
| TYRP1 | ALS-Ox | ALS-Ox |
| UCP3 | ALS-TD | ALS-TD |
| UGT3A2 | ALS-Glia | ALS-Ox |
| UMODL1-AS1 | NA | ALS-Ox |
| UNC13D | ALS-TD | ALS-Glia |
| USH1C | ALS-Glia | ALS-Glia |
| USHBP1 | ALS-Glia | ALS-TD |
| USMG5 | ALS-Ox | ALS-Ox |
| VCAM1 | ALS-Glia | ALS-Glia |
| VIP | ALS-Ox | ALS-Ox |
| VRK2 | ALS-TD | ALS-Glia |
| VSTM2A | ALS-Ox | ALS-Ox |
| VSTM2A-OT1 | ALS-Ox | ALS-Ox |
| VWA7 | NA | ALS-TD |
| WBP11P1 | ALS-Ox | ALS-TD |
| WDR86-AS1 | ALS-Glia | NA |
| WFIKKN2 | ALS-Glia | ALS-Glia |
| WNK4 | ALS-Glia | ALS-TD |
| WNT16 | ALS-Ox | ALS-Ox |
| WNT2 | ALS-Ox | ALS-Ox |
| ZAP70 | ALS-TD | NA |
| ZCCHC12 | ALS-Ox | ALS-Ox |
| ZGLP1 | ALS-TD | ALS-TD |
| ZNF602P | ALS-TD | ALS-Glia |
| ZNF702P | ALS-Ox | ALS-Ox |
| ZRSR1 | ALS-TD | ALS-Glia |

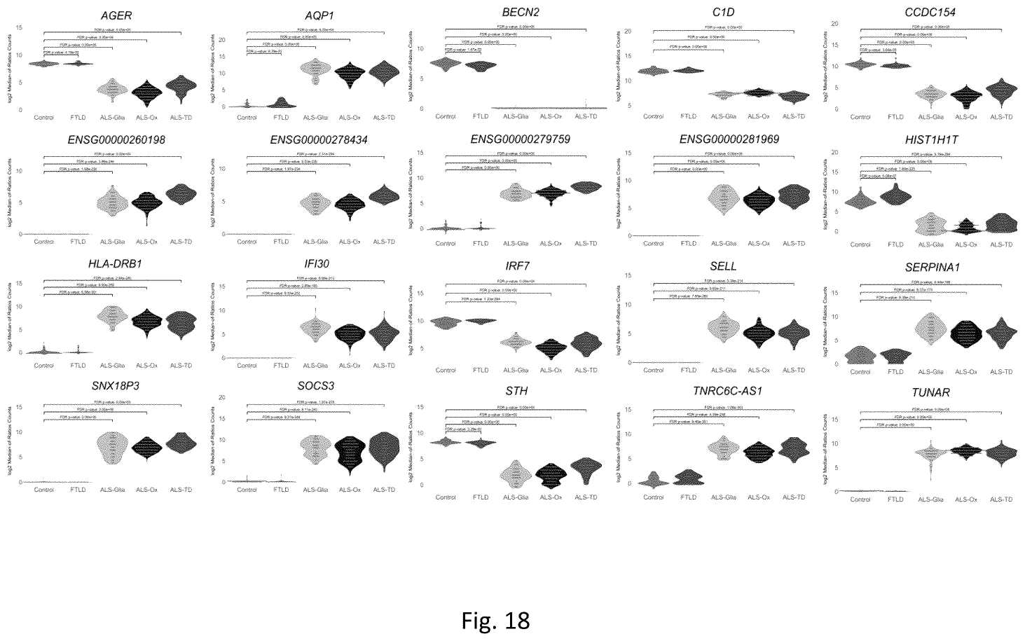

Out of the 36 transcripts selected to support the characterization of these distinct ALS phenotypes (FIG. 9), 34 were found to have distinctive expression for a single subtype, independent of the RNA-seq platform used for analysis (Table 3). Given the significant differences in patient survival (FIG. 4A), subtype-specific gene expression (FIG. 9) represents the first known prognostic transcripts for ALS. A few features show rather extreme differences in normalized expression between control and ALS groups, which may suggest simple thresholding could be used to distinguish the two cohorts (FIG. 18). Many of these genes and transcripts have not been previously associated with ALS neurodegeneration, offering new insight into disease pathologies and potential targets for diagnostic or therapeutic development.

### ALS-Glia

In the ALS-Glia subtype, significantly elevated expression of microglia, astrocyte, and oligodendrocyte marker genes were noted AIF1, CCR5, CD44, CD68, CHI3L2, CR1, CX3CR1, HLA-DRA, MSR1 (FIG. 14), TLR7, TMEM125, TNC, TREM2, and TYROBP (FIG. 9A). ALS-Glia upregulation of CHI3L2, CX3CR1, FOLH1, HLA-DRA, ALOX5AP, CCR5, CR1, FPR3, NCF2, TLR8, and TNFRSF25 generally indicates a pro-neuroinflammatory and pro-apoptotic disease phenotype (FIG. 9A and FIG. 14). ALS-Glia negative enrichment for PI3K/AKT signaling further supports a pro-apoptotic disease phenotype (FIG. 10A).

Elevated expression of TREM2, TYROBP, and CLEC7A (FIG. 9A, FIG. 14) may suggest a compensatory neuroprotective mechanism, where the activated (DAM) microglia state enhances phagocytic clearance and slows neurodegeneration. The DAM phenotype also known to promote ROS generation and neuroinflammation, obscuring the relationship between disease-associated microglia and ALS-Glia pathogenesis. Alterations to lipid metabolism in the ALS-Glia subtype are evidenced by APOBR, APOC1, and APOC2 overexpression compared to ALS-Ox and ALS-TD patients (FIG. 9A, FIG. 14), and may further reflect the elevated APOE and LPL expression seen in disease-associated microglia. Interestingly, there was upregulated expression of transcripts CX3CR1, TYROBP and TREM2 in this subtype, possibly suggesting dysregulation or competition between homeostatic and activated microglia phenotypes (FIG. 9A).

The expression of many Fc-gamma receptors and MHC Class II molecules were consistent with the ALS-Glia subtype. (FIG. 10E-10I, Table 3). Heightened VRK2 expression suggests some anti-apoptotic regulation occurs in ALS Glia patients (FIG. 14). Overexpression of FOLH1 may provide evidence for glutamate excitotoxicity susceptibility in the ALS-Glia subtype (FIG. 9A). Elevated transcription of ST6GALNAC2 suggests alterations to post-translational protein O-glycosylation (FIG. 14), while NINJ2 expression may support the proclivity for neuronal damage and death (FIG. 14). Although additional work is needed to better understand the consequences of the apparently dichotomous microglial phenotypes in the ALS-Glia frontal and motor cortex, these results clearly demonstrate that a subset of ALS patients are defined by glial activation and elevated inflammatory signaling.

### ALS-Ox

The ALS-Ox subtype is defined by oxidative stress, evidenced by upregulated expression of OXR1 and SOD1 and downregulation of CP (ceruloplasmin), UCP2, and oxidative phosphorylation genes NDUFA4L2, TCIRG1, and COX412 (FIG. 9B, FIG. 17). NDUFA4L2 and BECN1 expression further implicate impaired autophagy in ALS-Ox pathology. Subtype-specific expression of many synaptic signaling associated genes was observed, including: GABRA1 (GABA receptor), GABRA6, GAD2 (catalyzes production of GABA), GLRA2 (glycine receptor), GLRA3, HTR2A (serotonin receptor), KCNV1 (voltage-gated ion channel), KCNMB1, PCSK1, SLC6A13 (GABA transporter), SLC17A6 (glutamate transporter), SLC17A8 (glutamate transporter), and TCIRG1 (proton transporter associated with synaptic vesicle formation) (FIG. 9B, FIG. 15, Table 3). Together, the upregulated transcription of GABRA1, GABRA6, GAD2, GLRA2, and GLRA3 and downregulation of SLC6A13 strongly suggest increased inhibition in the ALS-Ox frontal and motor cortex. Increased expression of SLC17A6 and SLC17A8 may reflect a neuronal process to alleviate reduced excitability. Elevated transcription of BECN1, PFDN4, SERPINI1 (neuroserpin), UBQLN1, and UBQLN2 suggests proteotoxic stress is also a defining characteristic of this ALS subtype (FIG. 9B, FIG. 15, Table 3).

Downregulation of NOS3, NOTCH3, MYH11, MYL9, and TAGLN may implicate pericyte and vascular smooth muscle cell dysfunction and alterations to the blood-brain barrier in ALS-Ox patients (FIG. 15).

Similar to the ALS-Glia subtype, B4GALT6 overexpression suggests changes to the O-glycosylated proteome (FIG. 15). Evidence for alterations to the extracellular matrix in the frontal and motor cortex of ALS-Ox patients is observed in the downregulated expression of ADAMTSL4, ADAMTS7, ADAMTS14, COLIA1, COLIA2, COL2A1, COL3A1, COL4A6, COL6A3, COL8A1, COL14A1, COL18A1, and TAGLN (FIG. 9B, FIG. 15, Table 3). The common disease themes between Alzheimer's disease and the ALS-Ox subtype are the expression of oxidation associated transcripts COX412, NDUFA4L2, and OXR1, although CP is known to be upregulated in Alzheimer's (FIG. 10A, 9B). Interestingly, upregulated transcription of GABRA1, GAD2, HTR2A, and PCSK1 was observed in ALS-Ox patients, which have been previously reported to be downregulated in Alzheimer's patients, suggesting distinct synaptic signaling pathological mechanisms (FIG. 10A, 9B). Taken together, these results generally suggest ALS-Ox patients reflect that of more traditional neurodegenerative themes, such as oxidative and proteotoxic stress, impaired blood-brain barrier function, and alterations to synaptic signaling.

### ALS-TD

The defining characteristic of ALS-TD patients is the dysregulation of transcription, evident by the overexpression of pseudogenes (EGLNIP1, ENSG00000213197, HSP90AB4P, KRT8P13, NANOGP4, RPS20P22), intronic and antisense transcripts (AGPAT4-IT1, GATA2-AS1, TUB-AS1, ENSG00000205041, ENSG00000263278, ENSG00000268670, and ENSG00000273151), long non-coding RNA (LINC00176, LINC00638, LINC01347), and nonsense-mediated decay mRNA (ARHGAP19-SLIT1, C1QTNF3-AMACR, CHKB-CPT1B, and SLX1B-SULTIA4) (FIG. 9C, FIG. 16, Table 3). Upregulated expression of microRNAs miR24-2, miR219A2, miR3648-1, and MIRLET7BHG, relative to the other ALS subtypes, provides additional support for transcriptional and translational dysregulation in ALS-TD patients (FIG. 9C, FIG. 16, Table 3). miR24-2 has been previously shown to participate in many diseases, including neurodegeneration, serving to regulate cellular proliferation, differentiation, and apoptosis. miR219A2 is known to modulate oligodendrocyte differentiation and remyelination and has been previously reported to be downregulated in the brains of Alzheimer's patients.

MIRLET7BHG (LET-7B host gene) is also known to regulate gene expression and has been shown to interact with glial receptor TLR7 to promote neurodegeneration. Therefore, downregulation of TLR7 in the ALS-TD subtype (FIG. 9A) may reflect a neuroprotective state. Altered expression of transcription factors NKX6-2 and RUNX3, relative to controls, further emphasizes transcription as a central pathological mechanism in ALS-TD patients (FIG. 16, Table 3).

Downregulation of transcripts encoding extracellular matrix proteins (FIG. 9C, FIG. 16, Table 3) and characteristic expression of some transposable elements were observed (FIG. 17) in the ALS-TD subgroup. Surprisingly, TARDBP (encoding TDP-43) transcription was not a defining feature of ALS-TD patients and expression was relatively conserved across ALS subtypes, with only moderate upregulation observed compared to healthy controls (FIG. 7B). Transcription of ADAT3 in ALS-TD patients suggests that the pathological dysregulation of transcription and translation extends to tRNAs (FIG. 16). Consistent with the ALS-TD phenotype, elevated expression of many novel mRNA transcripts was observed, with some examples being ENSG00000258674, ENSG00000279233, ENSG00000279712, ENSG00000228434, ENSG00000234913, and ENSG00000250397 (FIG. 9C, FIG. 16, Table 3). Downregulation of TP63 suggests alterations to TP53 signaling and an anti-apoptotic phenotypic state in the ALS-TD subtype (FIG. 16). This interpretation is further supported by the survival analysis (FIG. 12A), given that ALS-TD patients demonstrated the longest median disease duration. Taken together, these results suggest poor control of gene transcription and translation in ALS-TD frontal and motor cortices and provide additional insight into the role of TEs in this subtype.

### Example 2: Elevated Expression of B4GALT6, GABRA1, GAD2, GLRA3, HTR2A, PCSK1, and SLC17A6 are Postmortem Markers for the ALS-Ox Subtype

Variable onset and progression of ALS has limited clinical trial success and slowed the development of effective therapeutics. This study builds on previous work (Eshima, J. et al. 2023. Nature Communications, 14, 95) to show this large cohort of ALS patients (Prudencio, M. et al. 2020. J. Clin. Investig. 130, e139741) can be stratified into three distinct molecular subtypes in both the postmortem cortex and spinal cord, with subtype-specific enrichment pathologies remaining mostly consistent. These findings lend additional support to the biological role of these subtypes in disease progression and offer a promising foundation for development of more effective and personalized therapeutics. Conversely, the survival analysis indicates additional factors other than molecular subtype likely play a significant role in the variability in patient survival after adjustment for repeat patient measures but continue to support the relevance of these subtypes in the context of clinical heterogeneity. Drawing on other works involving cell and animal models, more aggressive ALS progression is broadly linked to neuroinflammation (Beers, D. R. et al. 2006. Proc. Natl Acad. Sci. USA 103, 16021-16026; Boillée, S. et al. 2006. Science 312, 1389-1392; Yamanaka, K. et al. 2008. Nat. Neurosci. 11, 251-253), in agreement with these observed differences in subtype survival and hazard ratio. Additional work is needed to better understand how these phenotypes progress and interact with other factors like sex and disease comorbidity to drive variability in patient clinical parameters.

Comparing phenotypes assigned in the postmortem cortex and spinal cord, the lumbar region of the spinal cord was found to be most concordant with the cortical phenotype and most closely reflects the proportion of patients allocated to each subtype in the cortex (Eshima, J. et al. 2023. Nature Communications, 14, 95). To understand why, the work from Humphrey et al (Humphrey, J. et al. Nature Neuroscience, 1-13) was considered, which demonstrates cell type composition in the spinal cord acts as a major driver in perceived gene expression differences. Keeping the limitations of bulk tissue RNA-seq in mind, the lumbar region of the spinal cord is reported to have the highest percentage of neuronal cells relative to glia (Bahney J, von Bartheld C S. 2018. The Anatomical Record. 301 (4): 697-710), allowing for a more detectable neuronal expression signature in the bulk tissue profile. Many of the altered pathways in the ALS-Ox subtype implicate neurons, leading to the conclusion that the higher percentage of these cell types in the lumbar region allows for improved stratification of the ALS cohort. As ALS patient stratification matures and clinical translation begins to take shape, the authors recommend biomarker sampling take place in the lumbar region of the spinal cord to limit cell type composition influences.

In the ALS-Glia and ALS-TD spinal cord, many neuroinflammatory genes from the cortex (Eshima, J. et al. 2023. Nature Communications, 14, 95) were similarly elevated, likely reflecting cell type composition and stringent filtering of covariate-dependent gene expression. Enrichment analysis further supports shared phenotype themes between the two subtypes, suggesting the spinal cord may be less suited for stratification of ALS-Glia and ALS-TD patients, when using bulk tissue expression. Conversely, some of the most differentially expression transcripts in each subtype broadly captured pathological themes observed in the postmortem cortex (Eshima, J. et al. 2023. Nature Communications, 14, 95). ALS-Glia samples maintain the most elevated expression of inflammatory genes, while ALS-TD samples primarily show the highest expression of non-coding transcripts, including pseudogenes, transcription factors, and long intergenic non-coding RNA. Further, expression of transcripts MYL9, ST6GALNAC2, and TAGLN were elevated in both the postmortem cortex and spinal cord of ALS-Glia patients, relative to the other subtypes and controls, providing a foundation for complete stratification using spinal cord expression. Interestingly, these marker genes were not directly involved in neuroinflammation and instead related to muscle contraction and protein glycosylation-indicating neuroinflammation is not a unique facet of the ALS-Glia spinal cord. In addition, seven transcripts (‘ALS-Ox marker genes’) were observed uniquely elevated in the postmortem cortex and spinal cord of ALS-Ox patients, supporting an ALS-Ox-specific pathology involving dysregulated and altered synaptic signaling. Identification of shared marker genes between the cortex and spinal cord lends strength to a generalized and coherent subtype presented at the patient level, although the unsupervised clustering and concordance analyses reveal the challenges surrounding the assignment of a single subtype to a patient. Again, additional work is necessary to address this gap, potentially including examination of time-dependent phenotype progression and association of subtype with other relevant clinical measures like MUNE, MRI imaging, dendritic density, electrophysiological recordings, and the qualitative ALSFRS-R score.

Demonstrating the utility of the ALS-Ox marker genes, a variety of different machine learning classifiers that achieve impressive stratification accuracy in three unique holdout cohorts were constructed. While a similar analytical interpretation is obtained from the cortex and spinal cord validation cohorts. Both cohorts were specifically constructed to illustrate the global nature of elevated transcript expression and emphasize the capacity to use either region to predict the other. From a patient perspective, there is likely more benefit in the ability to use the spinal cord to predict the phenotype in the cortex—although as research grows the authors anticipate both directions may be relevant. The sequencing platform (HiSeq) holdout may best estimate predictive performance of these classifiers when applied to new patient cohorts with inconsistent batch effects and confounding factors. Lending strength to the biological relevance of the ALS-Ox marker genes, these classifiers continue to demonstrate high predictive accuracy when trained on NovaSeq samples and tested on HiSeq, and greatly outperform the ˜300 gene classifiers previously developed (Eshima, J. et al. 2023. Nature Communications, 14, 95). Finally, the demonstrated ability to refine the set of seven ALS-Ox marker genes to achieve mostly equivalent predictive performances reduces barriers to clinical implementation and diagnostic burden. Looking forward, additional patient cohorts are needed to validate the utility of the ALS-Ox marker genes, including a consideration of expression from living individuals. The newfound ability to predict, with reasonable accuracy, the cortical phenotype from spinal tissue expression reduces the invasiveness of stratification procedures and provides an important foundation to validate the relevance of ALS-Ox marker genes in vivo.

### Study Approval

The NYGC ALS Consortium samples presented in this work were acquired through various IRB protocols from member sites and the Target ALS postmortem tissue core and transferred to the NYGC in accordance with all applicable foreign, domestic, federal, state, and local laws and regulations for processing, sequencing, and analyses (Prudencio, M. et al. 2020. J. Clin. Investig. 130, e139741).

Postmortem brain tissues from cognitively normal individuals were obtained from the Mayo Clinical Florida Brain Bank. Diagnosis was independently ascertained by trained neurologists and neuropathologists upon neurological and pathological examinations, respectively. Written informed consent was given by all participants or authorized family members, and all protocols were approved by the IRB and ethics committee of the Mayo Clinic (Prudencio, M. et al. 2020. J. Clin. Investig. 130, e139741).

### Data Sources

GSE153960 contains RNA-seq data from 1659 tissue samples, spanning 11 regions of the CNS, from 439 patients with ALS, frontotemporal lobar degeneration (FTLD), or comorbidities for ALS-Alzheimer's (ALS/AD) or ALS-FTLD. Patients were filtered such that only the individuals belonging to the groups ALS-TDP, ALS/FTLD, ALS/AD, and ALS-SOD1 were included in the ALS disease cohort. Patient samples were further filtered to consider the postmortem spinal cord exclusively, yielding 428 unique tissue transcriptomes from the cervical, thoracic, and lumbar regions. Tissue-matched control samples were obtained from the same publicly available dataset, totaling 91 tissue transcriptomes from 56 non-neurological control patients. Cohort demographics for this analysis are included in Table 4. Four samples that passed the inclusion criteria were excluded from the analysis due to consistent file transfer issues (SRR12166443, SRR12166526, SRR12166549, SRR12166553).

### Quantification

Quantification of gene expression was performed using STAR alignment (Dobin, A. et al. 2013. Bioinformatics 29, 15-21) and RSEM (Li, B. & Dewey, C. N. 2011. BMC Bioinforma. 12, 1-6), as detailed by Prudencio et al (Prudencio, M. et al. 2020. J. Clin. Investig. 130, e139741), and the processed gene count matrix was accessed directly from the GEO Accession (GSE153960). For quantification of transposable elements using SQUIRE, raw paired-end FASTQ files for all ALS and non-neurological control patient samples were downloaded from the European Bioinformatics Institute data repository (NCBI mirror) using Globus software. SQUIRE's Fetch, Clean, Map, and Count functions were utilized as indicated (Yang, W. R. et al. 2019. Nucleic acids Res. 47, e27-e27) to quantify locus-specific transposable elements. The expectation maximized ‘tot_counts’ values were selected as the estimate for sequencing reads attributed to each transposable element with gene locus resolution. The hg38 build was used during mapping, with default trim and EM parameters, and a read length of 100 or 125 base pairs depending on the sequencing platform specified. A scoring threshold of ≥99 was used to restrict the number of false positive TEs with few uniquely mapping reads. Stringent filtering was then applied to ensure all TEs included in the downstream analysis had at least one count for all available ALS samples (n=428), resulting in 475 unique TE features.

### Clustering

Estimation of factorization rank was determined using the NMF package (Gaujoux, R. & Seoighe, C. 2010. BMC Bioinforma. 11, 1-9) in R, Version 4.0.3 (The R Foundation for Statistical Computing, Vienna, Austria). Quality measures were determined for ranks spanning 2 to 6, using 50 iterations at each rank and the default seeding method. The nsNMF (non-smooth non-negative matrix factorization) method was used for all NMF clustering.

Given previous work (Humphrey, J. et al. 2023. Nature Neuroscience, 1-13), it was understood that cell type composition strongly influences bulk tissue expression in the spinal cord and used marker genes defined by the same study to remove these tissue-dependent features. Glial marker genes were obtained from Humphrey et al. (Humphrey, J. et al. 2023. Nature Neuroscience, 1-13). Using DESeq2 (Love, M. I. et al. 2014. Genome Biol. 15, 1-21), a cumulative of 22,563 genes were found in the NovaSeq cohort and 17,804 genes in the HiSeq that were differentially expressed due to (i) sex, (ii) site of collection (NYGC versus Target ALS), (iii) RIN, and (iv) tissue region with an FDR adjusted p-value less than 0.05. After identifying dependent gene expression, patient transcriptomes were first subject to a variance stabilizing transformation, covariate-dependent genes were then removed, and filtering was applied such that the top 5,000 most variable genes (by median absolute deviation) were selected for clustering. Non-negative matrix factorization and visualization was performed in SAKE (Ho, Y. J. et al. 2018. Genome Res. 28, 1353-1363) (Version 0.4.0). No samples were removed during the quality control step and further data transformations were not necessary. To robustly assign ALS sample subtype, 11 rounds of NMF clustering were performed in SAKE for both sequencing platform cohorts. A rank of three was used for each independent round of clustering, with 100 iterations per round, and the nsNMF algorithm. All software package versions have been detailed previously (Eshima, J. et al. 2023. Nature Communications, 14, 95).

### Enrichment

After each replicate of NMF clustering, gene and TE feature scores were calculated for all 5,000 MAD transcripts (Kim, H. & Park, H. 2007. Bioinformatics 23, 1495-1502). Feature scores were averaged across nsNMF clustering replicates and reordered. All features from both sequencing platform cohorts were combined, and after the removal of duplicates, 8163 transcripts remained for enrichment, corresponding to 5438 gene symbols.

For GSEA (Subramanian, A. et al. 2005. Proc. Natl Acad. Sci. USA 102, 15545-15550), transcript expression was normalized to the DESeq2 median-of-ratios scale. Default parameters were maintained, aside from lowering the minimum gene set size to 5 and maximum to 150. The canonical pathways contained in the Reactome database (Jassal, B. et al. 2020. Nucleic acids Res. 48, D498-D503) and present pathway-level normalized enrichment scores for each ALS subtype. Non-neurological controls were specified as the reference level for subtype enrichment. To further support subtype-specific pathway enrichment observed in GSEA, hypergeometric enrichment analysis was performed using Enrichr (Kuleshov, M. V. et al. 2016. Nucleic acids Res. 44, W90-W97), the Reactome 2022 database, and the feature assignment approach detailed previously (Eshima, J. et al. Nature Communications, 14, 95 (2023). Enrichment p-values are determined by Fisher's exact test, and presented as −log 10 transformed values after FDR adjustment. The p-value heatmap is color-coded to indicate upregulation or downregulation relative to the other subtypes, and blank cells indicate an FDR adjusted p-value >0.05.

### Clinical Parameters

The majority of patients in this cohort have more than one observation from the postmortem spinal cord. As a consequence, patient clinical parameters are considered using the majority agreement approach detailed previously (Tam, O. H. et al. 2019. Cell Rep. 29, 1164-1177; Eshima, J. et al. 2023. Nature Communications, 14, 95). In brief, patients were assigned a label only if there was a majority consensus among their cervical, thoracic, and lumbar samples, or if there was a single sample characterized, and labeled ‘Discordant’ in all other cases. Using this approach, differences in ALS patient survival were assessed using the Kaplan-Meier analysis (Kaplan, E. L. & Meier, P. 1958. J. Am. Stat. Assoc. 53, 457-481) with application of the log-rank statistical test. Subtype-level differences in age at symptom onset and death were analyzed by ANOVA with post hoc students' t-tests (two-sided, unequal variance) and FDR p-value adjustment. Chi-squared tests of independence were applied to assess subtype-specificity for FTLD and Alzheimer's comorbidity.

### Cox Proportional Hazard Regression

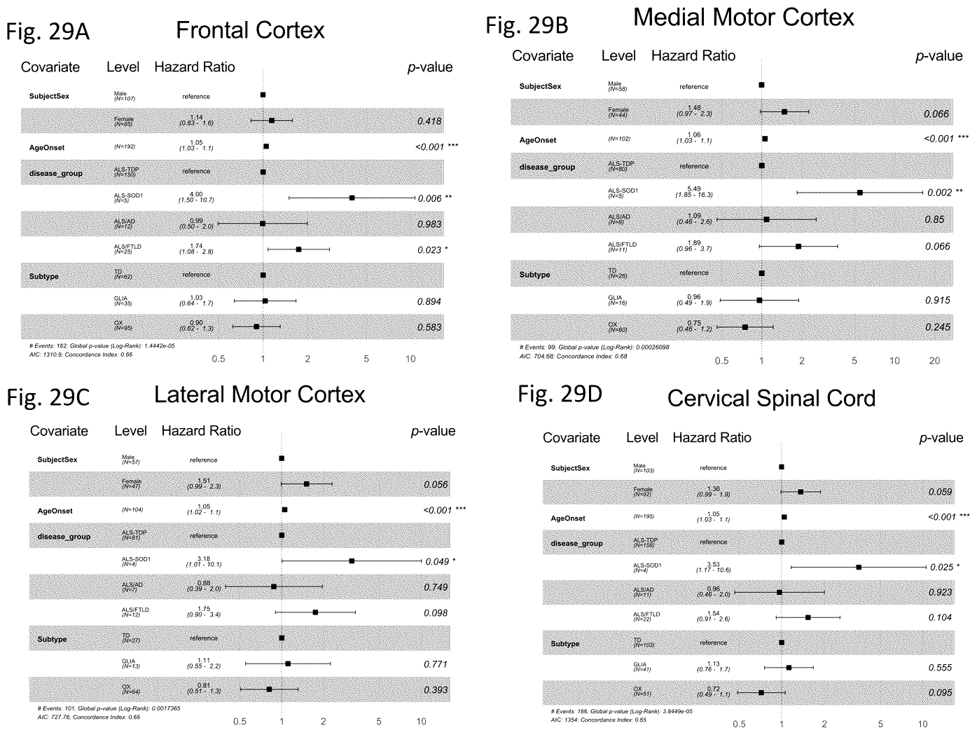

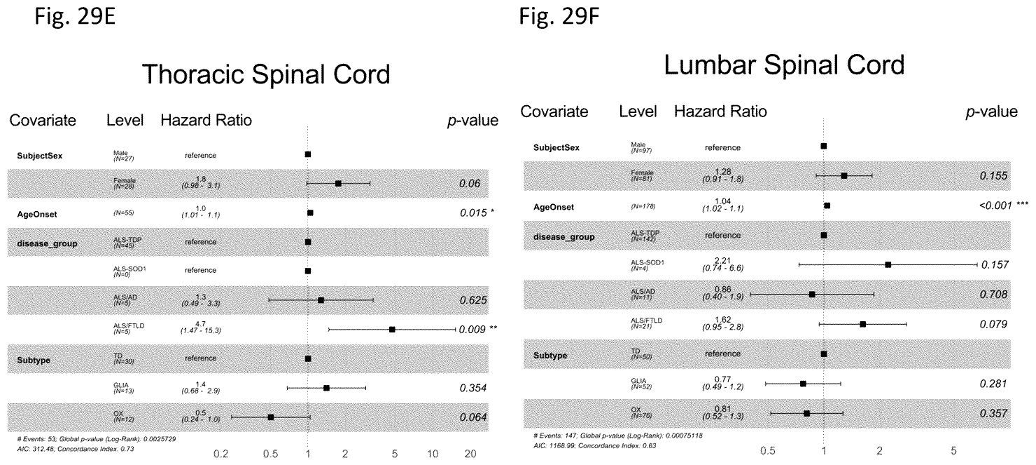

To address sample dependence due to repeat patient measures, a Cox proportional hazard regression model (Therneau, T. M. & Lumley, T. 2015. R. Top. Doc. 128, 28-33) was constructed in R to assess multivariate contribution to patient survival. Sex, site, subtype, age at symptom onset, and disease group covariates are included as fixed effects and obtain hazard ratios from the exponential of the beta coefficient. The patient-specific random effects were incorporated in the proportional hazard model by setting the ‘cluster’ parameter equal to patient ID. Sample observations were split at month 20 to create two non-overlapping time intervals, allowing approximation of time-dependent covariate hazards, and ensuring the proportional hazard assumption is met for nearly all model terms (FIG. 29). The R function call for the proportional hazard regression model is provided in the Supporting Information Text and requires the ‘survival’ R library (Therneau, T. M. & Lumley, T. 2015. R. Top. Doc. 128, 28-33). Model term p-values were calculated from the coefficient z-scores, while testing of the proportional hazard assumption at the covariate level was performed using the score test, with p <0.05 indicating time-dependent hazard and assumption violation (Therneau, T. M. & Lumley, T. 2015. R. Top. Doc. 128, 28-33).

### Concordance Analysis

Postmortem cortex subtype labels were previously determined (Eshima, J. et al. 2023. Nature Communications, 14, 95) and utilized in the present study to assess concordance with the molecular phenotype presented in the spinal cord of the same patients. Agreement is considered at the tissue-level rather than CNS level (i.e. cortex, spinal cord), to avoid sample dependence concerns with the majority agreement approach. Subtype discordance between the cortex and spinal is color-coded using a previous scheme (Eshima, J. et al. 2023. Nature Communications, 14, 95), to illustrate which reassignment was more common, given the postmortem cortex observation.

### Differential Expression

Differential transcript expression between ALS subtypes was considered using DESeq2 (Love, M. I., Huber, W. & Anders. 2014. Genome Biol. 15, 1-21) with counts presented on the log 2 scale, following size factor normalization (median-of-ratios). All patient samples were used to estimate size factors for normalization (n=519 samples). A multifactor design equation was implemented, which included platform, site, RIN, tissue, and subtype covariates. Pairwise comparisons were performed using the contrast ( ) argument, and FDR-adjusted p-values <0.05 were considered to be significant. For presentation as a heatmap, transcript expression was z-score normalized, and observations that fell outside four standard deviations were adjusted to +4 for plotting purposes only. FDR-adjusted p-values were −log₁₀ transformed prior to plotting.

Given relevant work from Tam et al (Tam, O. H. et al. 2019. Cell Rep. 29, 1164-1177), Prudencio et al (Prudencio, M. et al. 2020. J. Clin. Investig. 130, e139741), and Humphrey et al (Humphrey, J. et al. 2023. Nature Neuroscience, 1-13)—which consider TARDBP, truncated stathmin-2, and cell type composition from the same cohort—these features were reexamined in the context of the stratified ALS cohort. The DESeq2 was utilized to assess whether TARDBP expression was specific to a single subtype, and include all available observations from the postmortem cortex of the same patients for reference (Eshima, J. et al. 2023. Nature Communications, 14, 95). The normal length and truncated form of STMN2 were determined previously by Prudencio et al. (Prudencio, M. et al. 2020. J. Clin. Investig. 130, e139741) and provided by the NYGC ALS Consortium. Similarly, cell type composition-estimated from cell deconvolution-were previously determined by Humphrey et al (Humphrey, J. et al. 2023. Nature Neuroscience, 1-13).

### Classification

Classifiers to stratify ALS-Ox from all other patients (‘NotOx’) were developed using an 80/20 train/test split and three unique holdout cohorts comprised of (i) all cortical transcriptomes, (ii) all spinal cord transcriptomes, and (iii) all samples analyzed by HiSeq. 100-fold cross-validation was used to estimate F1 scores, with predictions made using the max distance metric and the first component. PLS-DA was performed using the ‘Mixomics’ library in R (Rohart F et al. 2017. PLoS computational biology. 13 (11): e1005752).

Using the same train/test split, cross-validation, and holdout cohorts additional classifiers were developed in Python (Version 3.9.10, Python Software Foundation, Wilmington, DE) using the scikit-learn framework (Pedregosa, F. et al. 2011. J. Mach. Learn. Res. 12, 2825-2830) (Version 1.3.0). Five different models were considered, which included k-nearest neighbors (KNN), linear discriminant analysis (LDA), multilayer perceptron (MLP), random forest (RF), and support vector machine classification (SVM). Default parameters were maintained unless otherwise noted. For the k-nearest neighbor classifier the number of neighbors was set to 8. For the SVM, a linear kernel was used with the regularization parameter, ‘C’ set to 0.025. Finally, the multilayer perceptron classifier was built using three hidden layers, with 100 ‘neurons’ comprising each hidden layer. The learning rate was set to 0.0001, while alpha was set equal to 1E-5.

### Clustering of Spinal Cord Transcriptomes Recaptures Cortical Phenotypes

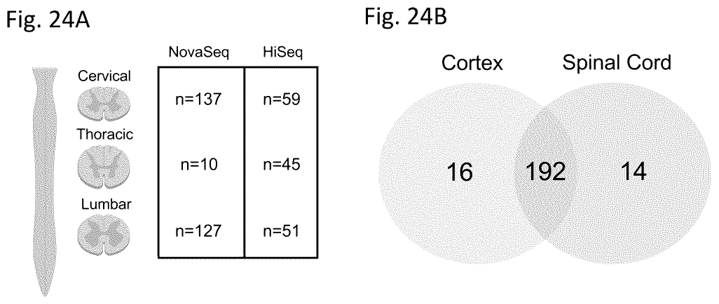

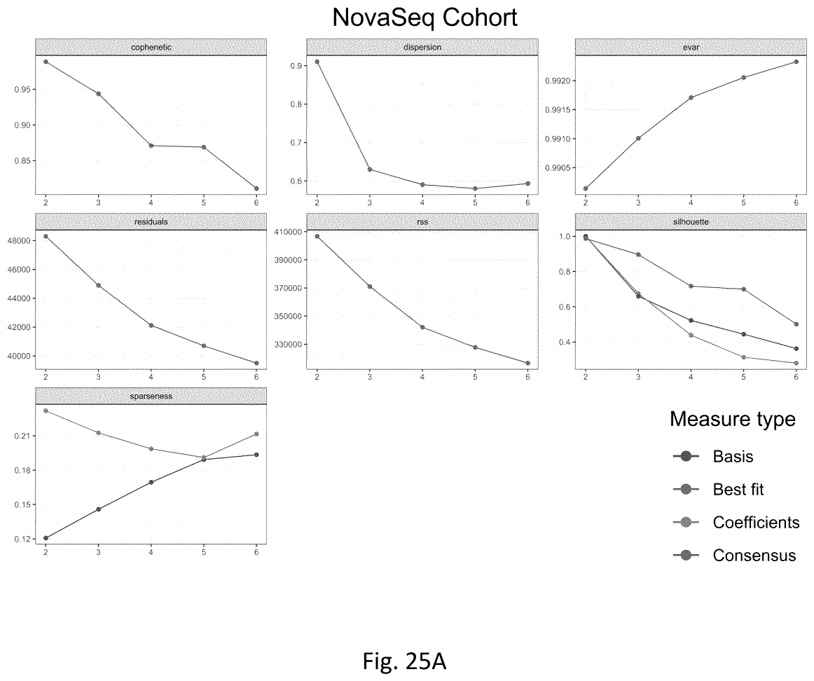

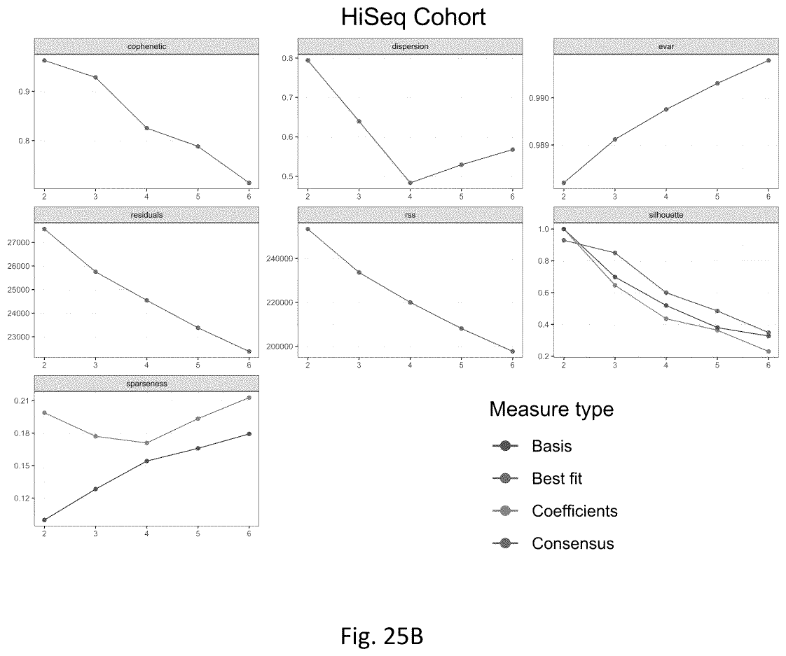

To ascertain concordance between molecular subtypes presented in the ALS cortex with those presented in the spinal cord, unsupervised clustering was performed using 428 transcriptomes derived from cervical, thoracic and lumbar postmortem tissue, corresponding to 206 unique ALS patients (FIG. 24; Table 4). Transposable elements (TEs) were quantified for all patient samples using SQUIRE (Yang, W. R. et al. 2019. Nucleic acids Res. 47, e27-e27), with transcript filtering criteria detailed previously (Eshima, J. et al. 2023. Nature Communications, 14, 95) (see Methods). Covariate-dependent gene expression was determined using DESeq2 (Love, M. I. et al. 2014. Genome Biol. 15, 1-21) and all features differentially expressed due to (i) sex, (ii) site of collection, (iii) RIN, and (iv) tissue region were removed prior to clustering. To help address bulk tissue effects during clustering, oligodendrocyte, microglia, astrocyte, and endothelial cell marker genes (n=1282) presented by Humphrey et al, were further removed given their findings show differential gene expression in the ALS spinal cord is partially driven by cell type composition (Humphrey, J. et al. 2023. Nature Neuroscience, 1-13). The majority of these features showed gene expression dependent on one of the four covariates previously addressed for both the NovaSeq (1061/1282) and HiSeq (855/1282) cohorts. The sequencing platform covariate was further addressed by splitting the cohort according to analytical platform (HiSeq 2500 and NovaSeq 6000, Illumina, San Diego, CA) and ran the stratification analyses separately. Factorization rank was estimated from clustering metrics (FIG. 25), and a rank of three was used in both cohorts.

TABLE 4
Cohort Demographics. ALS patients and healthy controls considered
in this analysis. Disease duration, age of onset, and age of
death statistics are provided as mean ± standard error.

Cohort Demographics

| A-L: Axial and Limb | | | | |
|---|---|---|---|---|
| Onset | | | | |
| B-L: Bulbar and Limb | | | | |
| Onset | | | Healthy Control | |
| A-B: Axial and Bulbar | ALS Spectrum | | Donors | |
| Onset | (n = 206) | | (n = 56) | |
| Sex | | | | |
| Female | 97 | (47.1%) | 31 | (55.4%) |
| Male | 109 | (52.9%) | 25 | (44.6%) |
| Tissue Site | n = 428 | | n = 91 | |
| Cervical Spinal Cord | 195 | (45.6%) | 40 | (44.0%) |
| Thoracic Spinal Cord | 55 | (12.9%) | 9 | (9.9%) |
| Lumbar Spinal Cord | 178 | (41.6%) | 42 | (46.2%) |

| ALS Subtype | | | NA |
|---|---|---|---|
| ALS-Glia | 34 | (16.5%) | — |
| ALS-TD | 68 | (33.0%) | — |
| ALS-Ox | 45 | (21.8%) | — |
| ALS-Discordant | 59 | (28.6%) | — |
| Disease Duration | | | NA |
| (months) | | | |
| ALS-Glia | 31.3 ± 3.97 | | — |
| ALS-TD | 36.0 ± 3.01 | | — |
| ALS-Ox | 45.6 ± 4.87 | | — |
| ALS-Discordant | 43.2 ± 3.98 | | — |
| Age of Onset (years) | | | NA |
| ALS-Glia | 64.7 ± 1.50 | | — |
| ALS-TD | 61.6 ± 1.58 | | — |
| ALS-Ox | 62.6 ± 1.63 | | — |
| ALS-Discordant | 58.4 ± 1.41 | | — |
| Site of Onset | | | NA |
| ALS-Glia | A-B: 1; A-L: 1; Bulbar: 8; | | — |
| | B-L: 1 Limb: 23 | | |
| ALS-TD | A-L: 1; Bulbar: 21; B-L: 1; | | — |
| | Limb: 37; Unknown: 8 | | |
| ALS-Ox | Axial: 2; Bulbar: 12; B-L: 2; | | — |
| | Limb: 26; Generalized: 1; | | |
| | Unknown: 2 | | |
| ALS-Discordant | Axial: 1; A-B: 1; Bulbar: 16; | | — |
| | B-L: 1 Limb: 39; Unknown: 1 | | |
| Age of Death (years) | | | 63.8 ± 2.16 * |
| ALS-Glia | 67.4 ± 1.48 | | — |
| ALS-TD | 65.5 ± 1.36 | | — |
| ALS-Ox | 66.1 ± 1.15 | | — |
| ALS-Discordant | 62.7 ± 1.29 | | — |
| FTLD Comorbidity | 23/206 | (11.2%) | NA |
| ALS-Glia | 6/34 | (17.6%) | — |
| ALS-TD | 6/68 | (8.8%) | — |
| ALS-Ox | 3/45 | (6.7%) | — |
| ALS-Discordant | 8/59 | (13.6%) | — |

* Two non-neurological control donors had an age of death listed as “90 or Older”. A conservative estimate of 90 years was used for all samples listed as such.

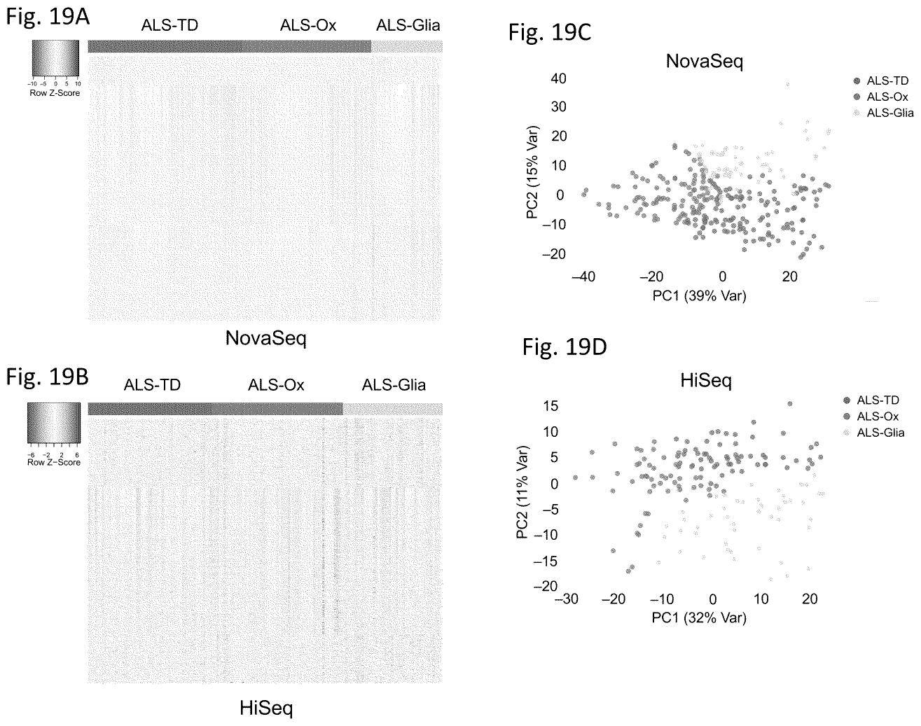

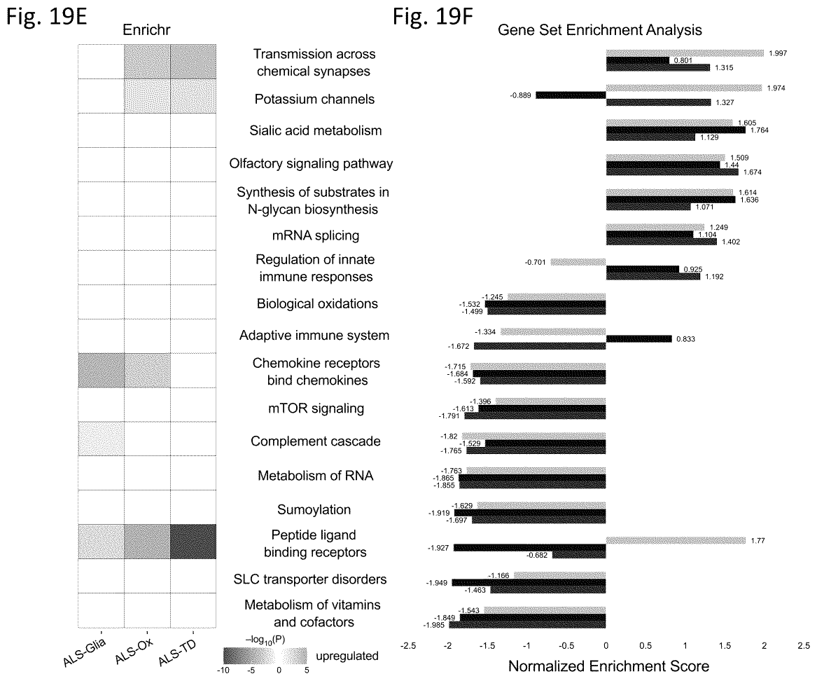

After applying a variance stabilizing transformation (VST) (Love, M. I., Huber, W. & Anders, S. 2014. Genome Biol. 15, 1-21), the top 5000 most variably expressed transcripts were selected for non-smooth non-negative matrix factorization (nsNMF) (Pascual-Montano, A. et al. 2006. IEEE Trans. Pattern Anal. Mach. Intell. 28, 403-415) using SAKE (Ho, Y. J. et al. 2018. Genome Res. 28, 1353-1363). These results capture three distinct groups in both the NovaSeq and HiSeq cohorts. These groups show distinct expression profiles (FIGS. 19A and 19B) and yield three clusters following application of principal component analysis (FIGS. 19C and 19D). To better understand biological context of these stratified groups, enrichment was performed using all 5000 features from the NovaSeq and HiSeq cohorts, and combined them to yield 8163 non-duplicate transcripts (corresponding to 5438 gene symbols). The resulting feature set was used to perform hypergeometric enrichment analysis with Enrichr (Kuleshov, M. V. et al. 2016. Nucleic acids Res. 44, W90-W97) and the Reactome (Jassal, B. et al. 2020. Nucleic acids Res. 48, D498-D503) pathway database, according to the approach described previously (Eshima, J. et al. 2023. Nature Communications, 14, 95). GSEA (Subramanian, A. et al. 2005. Proc. Natl Acad. Sci. USA 102, 15545-15550) was further leveraged to enrich each stratified group against a non-neurological control cohort comprised of 91 donor samples from the cervical, thoracic, and lumbar regions of the spinal cord (FIGS. 19E and 19F and Table 4). In agreement with the phenotypes identified in the ALS postmortem cortex (Eshima, J. et al. 2023. Nature Communications, 14, 95), results showed significant enrichment for neuroinflammatory signatures in ALS-Glia patients when compared to the other two subtypes. Interestingly, normalized enrichment scores indicate negative enrichment for neuroinflammatory pathways in the ALS spinal cord, relative to controls, whereas positive enrichment is observed in the cortex (Eshima, J. et al. 2023. Nature Communications, 14, 95)—findings which may be linked to cell type composition (Humphrey, J. et al. 2023. Nature Neuroscience, 1-13) and the stringent filtering of glial marker genes. In ALS-Ox patients, results showed significant positive enrichment for genes associated with synaptic signaling, mirroring the phenotype observed in the postmortem cortex. Pathways associated with RNA metabolism and processing were the most strongly enriched in the ALS-TD subtype when compared to controls using GSEA, although significant associations were not observed at an adjusted p-value <0.05 by either enrichment approach. Collectively, these findings demonstrate that unsupervised clustering of the ALS spinal cord recapitulates many of the same subtype signatures observed in the postmortem cortex. Conversely, phenotypic differences between the groups are less pronounced than in the cortex, evident in the magnitude of GSEA normalized enrichment scores, likely reflecting differences in cell type composition between the two regions of the central nervous system and the filtering of covariate-dependent gene expression.

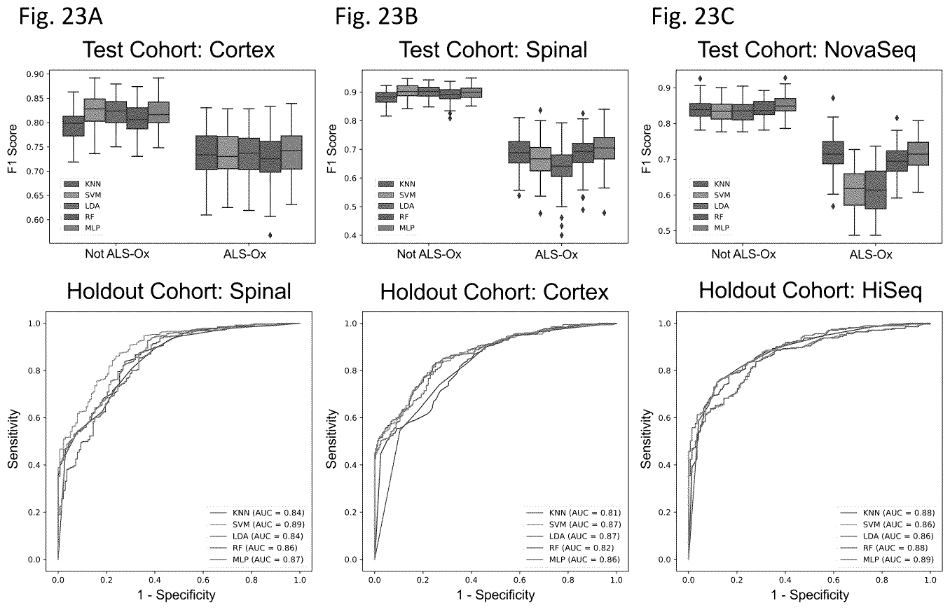

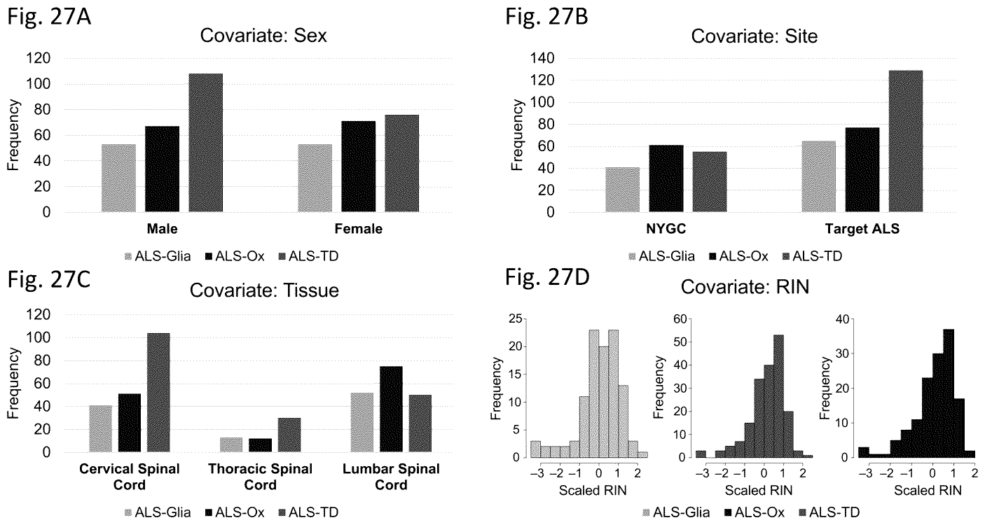

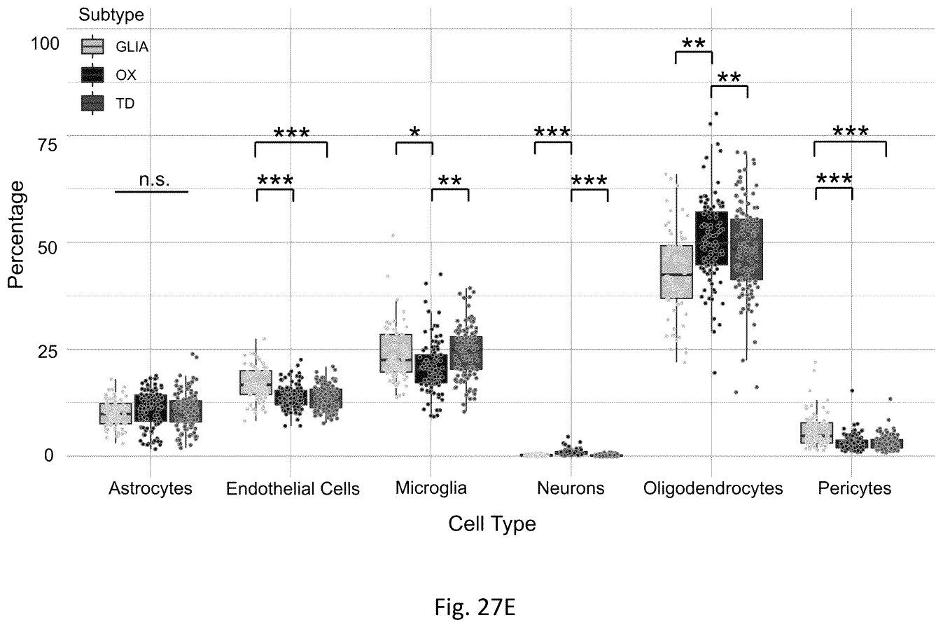

Allocation to each of the three subtypes in the NovaSeq cohort more closely agrees with findings from the frontal and motor cortex (Eshima, J. et al. 2023. Nature Communications, 14, 95), with ALS-Glia representing the rarest subtype (21.9% of spinal transcriptomes compared to 19.2% in the cortex (Eshima, J. et al. 2023. Nature Communications, 14, 95) corresponding to a Glia:Ox:TD ratio of 1:1.5:2 (FIG. 23). In the HiSeq cohort, a Glia:Ox:TD subtype ratio of 1:1:1.4 was observed, corresponding to ˜30% of patients classified as ALS-Glia, indicating the selected sequencing platform influences the detectable subtype expression signature (FIG. 23). In both cases, the transcriptional dysregulation (TD) subtype was the most commonly assigned—as compared to the oxidative stress subtype in the postmortem cortex (Eshima, J. et al. 2023. Nature Communications, 14, 95)—potentially a consequence of weak dependency on the removed covariates (FIG. 27A-D), weak neuronal expression associated with cell type composition in the spinal cord (FIG. 27E), and stringent filtering of covariate-dependent genes.

### Differences in Survival Persist but are Primarily Driven by Repeat Patient Measures

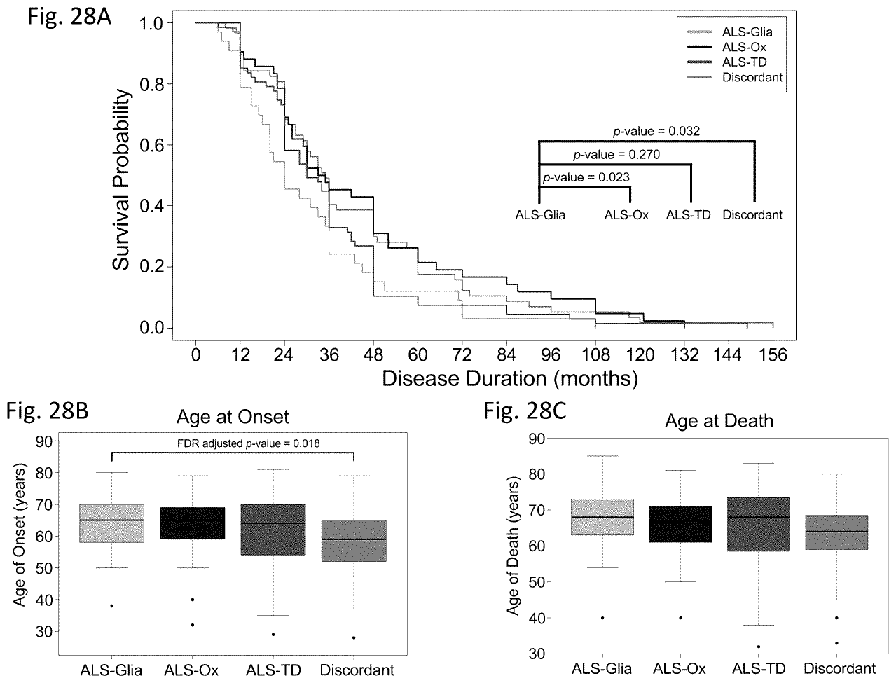

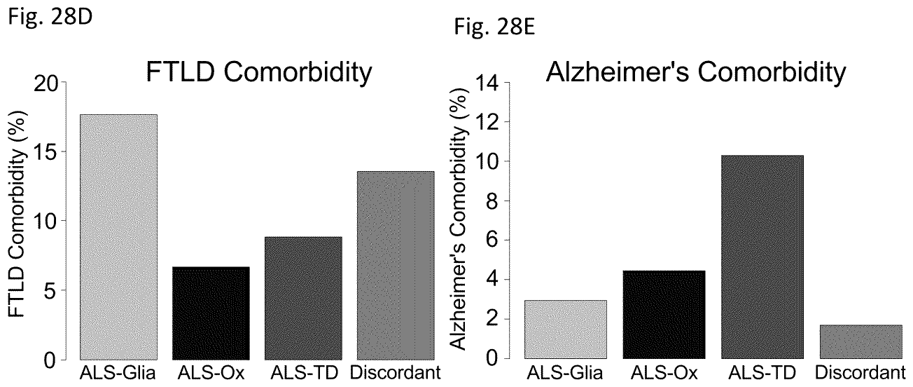

After stratification of the spinal cord cohort, patient clinical parameters were examined to determine if subtype level differences in survival are maintained. A Kaplan-Meier survival analysis (Kaplan, E. L. & Meier, P. 1958. J. Am. Stat. Assoc. 53, 457-481) was performed after assigning patient-level subtype using majority consensus between all available regions of the spinal cord or if a single tissue sample was characterized for a given patient (26/206; 12.6%). Similar to the postmortem cortex, results showed a significantly shorter survival duration in the ALS-Glia subtype when compared to ALS-Ox (p=0.032) and Discordant (p=0.023) groups but not the ALS-TD subtype (p=0.27) (FIG. 28A). The mean survival duration for ALS-Glia patients was found to be 31.3±3.97 months (mean±SE), while ALS-Ox patients were found to have the longest mean survival duration at 45.6±4.87 months (Table 4). Interestingly, results showed a significant difference in the age of disease onset between ALS-Glia and discordant patients after a false discovery rate (Benjamini, Y. & Hochberg Y. 1995. Journal of the Royal statistical society: series B (Methodological) 57, 289-300) (FDR) correction (FDR p=0.018), strengthening the trend seen in the postmortem cortex (Eshima, J. et al. 2023. Nature Communications, 14, 95) (FIG. 28B). Differences in age at death were not significant after FDR correction (FIG. 28C). No significant relationships were found between disease comorbidity and ALS subtype in the spinal cord using Chi-squared tests of independence (FIGS. 28D and 28E).

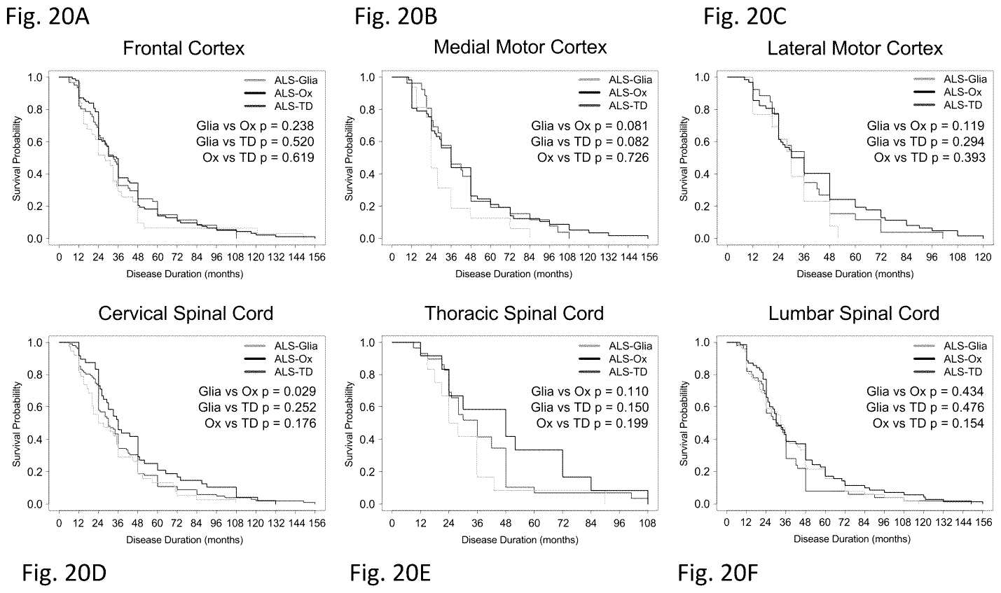

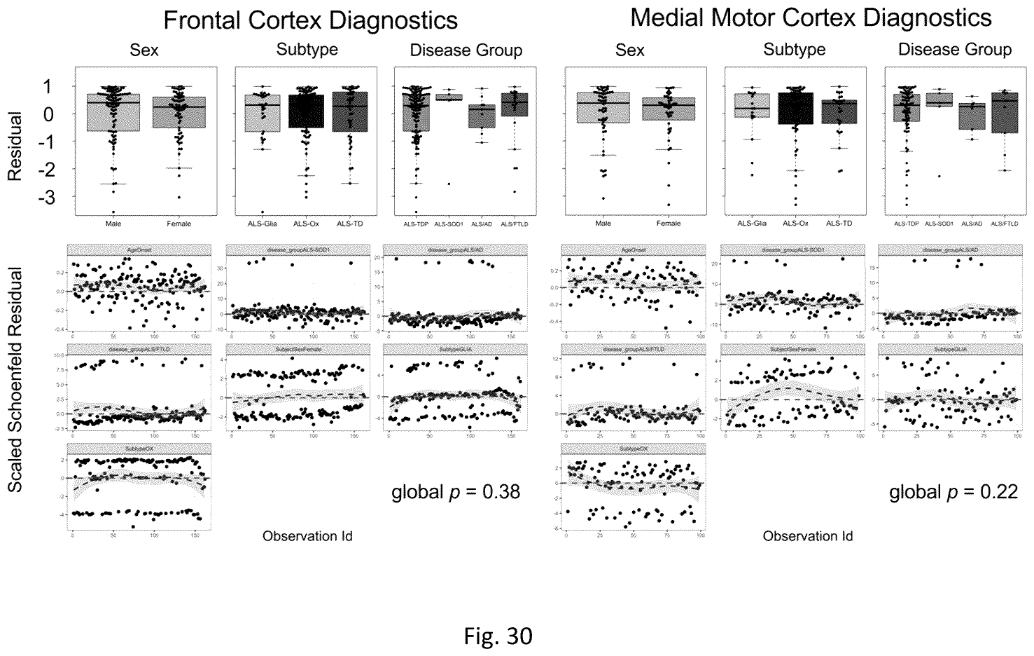

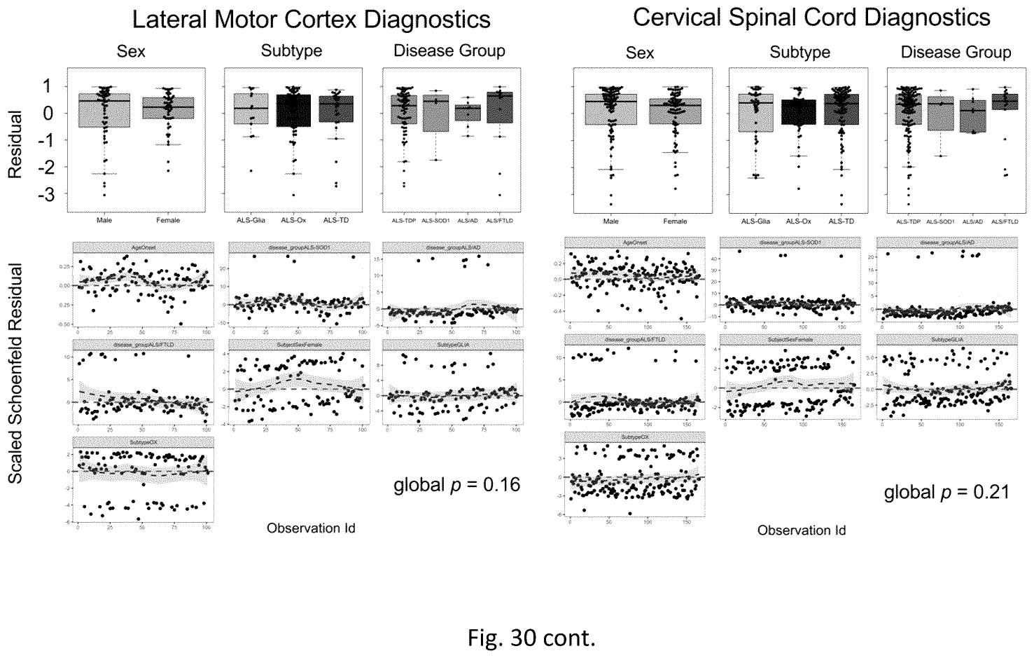

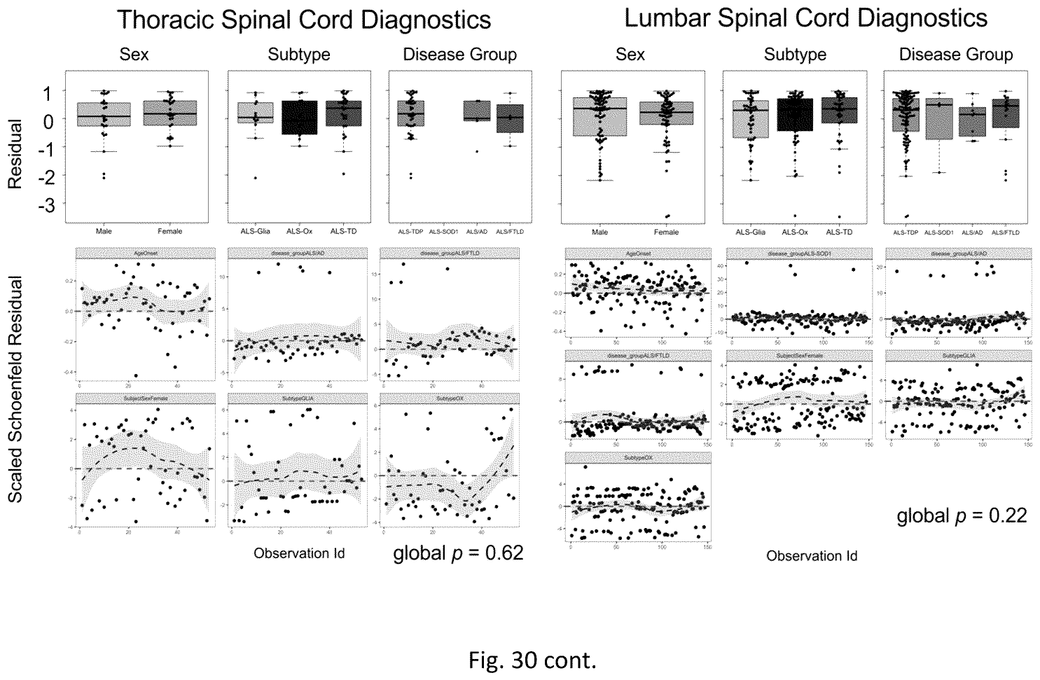

To better understand how repeat patient measures and the majority agreement approach (Tam, O. H. et al. 2019. Cell Rep. 29, 1164-1177; Eshima, J. et al. 2023. Nature Communications, 14, 95) influences observed differences in survival, additional survival analyses were performed with independent measures using (i) tissue-region specific survival and (ii) a Cox proportional hazard regression model accounting for repeat patient measures at time of death by incorporating subject-specific random effects (“frailty model”) (Austin, P. C. 2017. International Statistical Review 85, 185-203; Andersen, P. & Gill. 1982. Annals of Statistics 10, 1100-1120; Therneau, T. M. et al. 2003. Journal of computational and graphical statistics 12, 156-175; Therneau, T. M. & Lumley, T. 2015. R. Top. Doc. 128, 28-33; Kassambara A., Kosinski M., & Biecek, P. 2021. Survminer: Drawing Survival Curves using ‘ggplot2’ R package version 0.4.9; Kleinbaum D G. 1996. Springer). Survival analysis in each region of the CNS shows weaker differences in disease duration dependent on subtype, revealing that repeat patient measures explain a portion of the observed differences in subtype survival, and significance is often, but not always, lost after establishing sample independence (FIG. 20). These findings are generally supported by the frailty regression model, which show that the random effect due to repeat sampling of patients and the fixed effects due to sex and disease group drive differences in survival, with patient age, site, and subtype having weaker contributions (FIG. 29). Model diagnostics show the proportional hazard assumption is met for nearly all covariates, excluding disease groups with neurodegenerative comorbidity between 0-20 months (FIG. 30). Survival effects due to the ALS-Ox subtype covariate remained statistically significant (p=0.034), indicating repeat patient measures influence the observed differences in survival when defining patients by the majority agreement approach (Tam, O. H. et al. 2019. Cell Rep. 29, 1164-1177; Eshima, J. et al. 2023. Nature Communications, 14, 95), but show ALS molecular subtypes still capture heterogeneity in disease progression. The ALS-Ox hazard ratio was found to be 0.907 relative to the ALS-TD subtype, demonstrating the oxidative stress subtype is associated with a ˜9% reduction in risk for death. Similar hazard ratios are seen for the ALS-Glia and ALS-TD subtypes, indicating the ALS-Ox subtype is weakly associated with a better patient prognosis. Large confidence intervals seen in the disease group covariate likely reflect low sample number, although it is notable to see the greatest difference in hazard ratio in this covariate and shows disease comorbidity negatively affects risk of death. Interestingly, sex-dependent differences in risk are seen, with males showing roughly 33% lower risk for death during the first 20 months. Taken together, the consideration of repeat patient measures shows the majority agreement approach is limited due to sample-dependence on patient and differences in subtype stratified survival are generally weaker, yet remain relevant, when maintaining observational independence.

### Concordance Between Cortical and Spinal Subtypes is Dependent on Tissue and Sequencing Platform

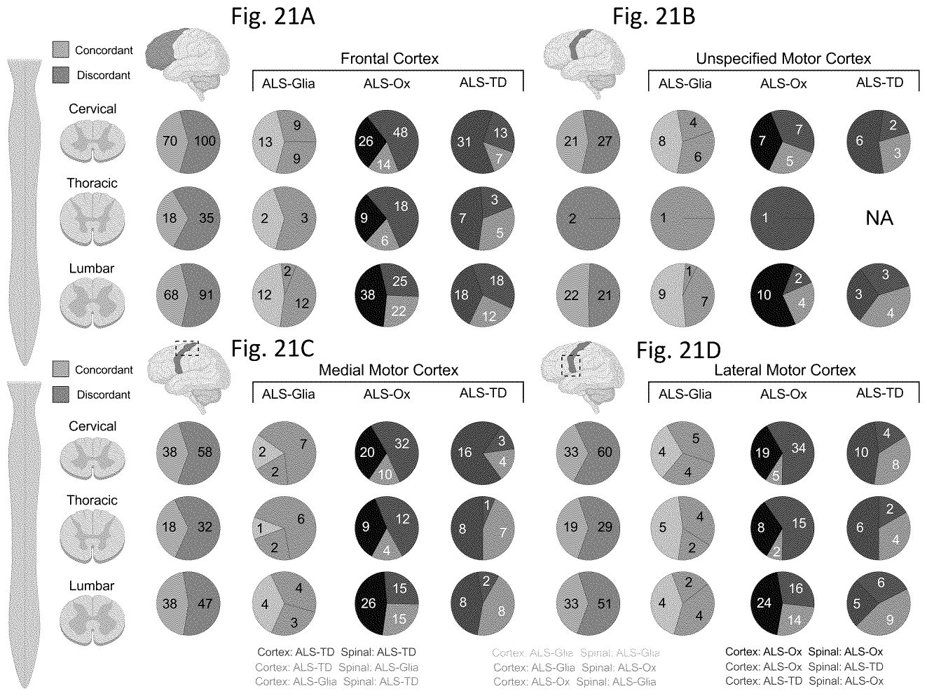

Given the high degree of overlap between the patients considered previously (Eshima, J. et al. 2023. Nature Communications, 14, 95) and those included in this analysis (FIG. 24B), this study was employed to better understand the agreement between the postmortem cortex (frontal, lateral motor, and medial motor regions) and spinal cord in presentation of ALS subtype. Concordance was considered at the sample level, to ensure independence, and presented as a matrix of pie charts, with spinal cord region along the rows and cortical region along the columns (FIG. 21). Excluding the unspecified motor cortex samples, results showed the highest concordance between the frontal cortex and lumbar region of the spinal cord for the ALS-Glia subtype (46.2%), the medial motor cortex and lumbar region for the ALS-Ox subtype (46.4%), and the medial motor cortex and cervical spinal cord for the ALS-TD subtype (69.6%). The higher concordance observed in the ALS-TD subtype, and generally lower overall agreement, likely stems from bias towards the ALS-TD subtype during unsupervised clustering, despite removal of covariate dependent genes (FIGS. 27A-D), and differences in cell type composition as compared to the cortex (FIG. 27E). Similarly, the lumbar region is seen to more closely reflect the proportion of subtypes observed in the cortex (Eshima, J. et al. 2023. Nature Communications, 14, 95) (FIG. 27C), generally corresponding to a noticeable improvement in concordance between the cortex and lumbar spinal cord, relative to other spinal cord regions, for ALS-Ox and ALS-Glia patients (FIG. 21).

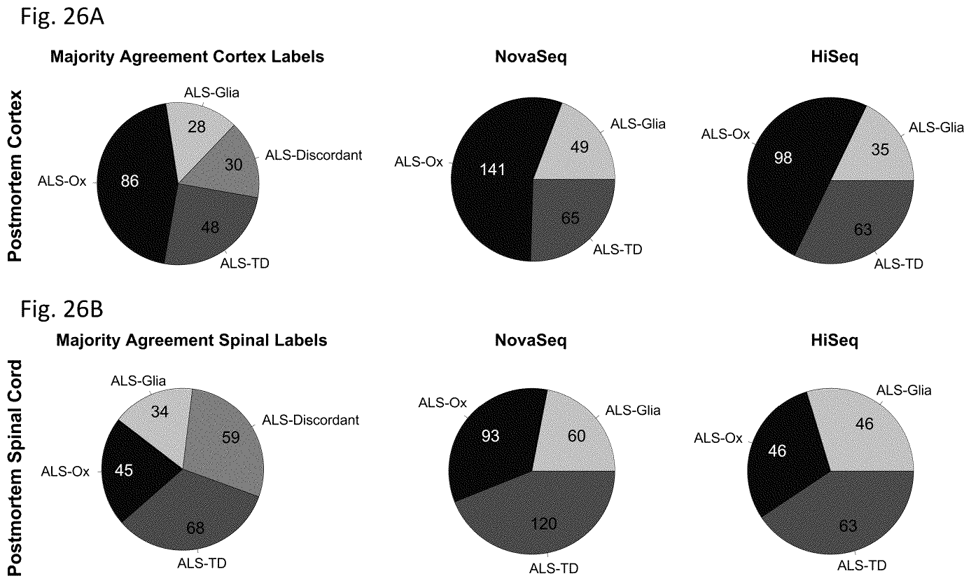

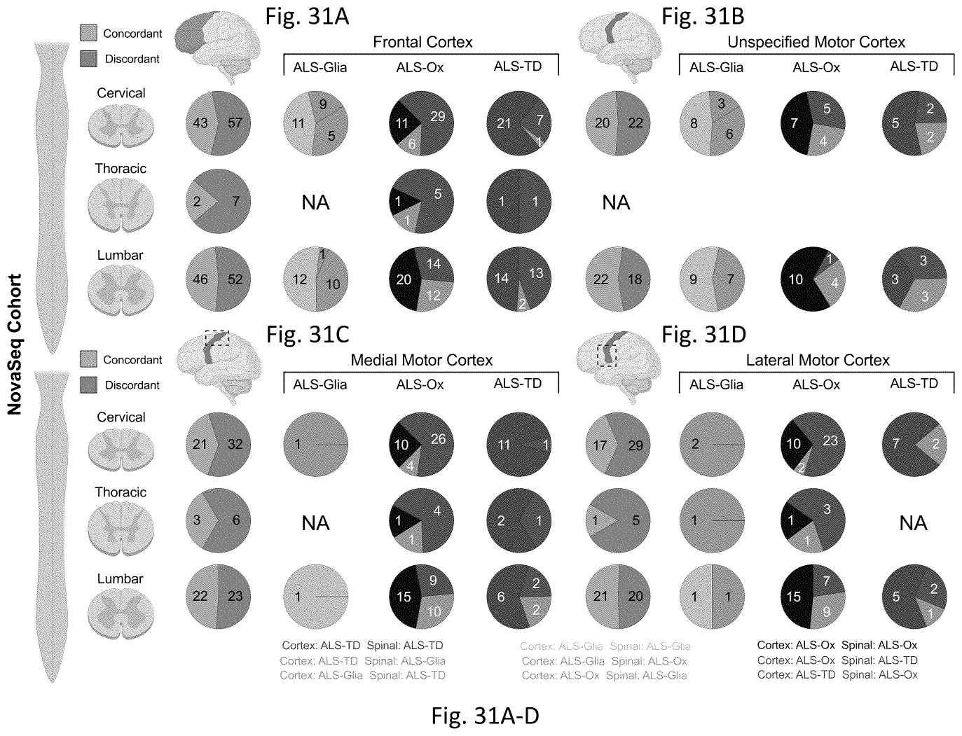

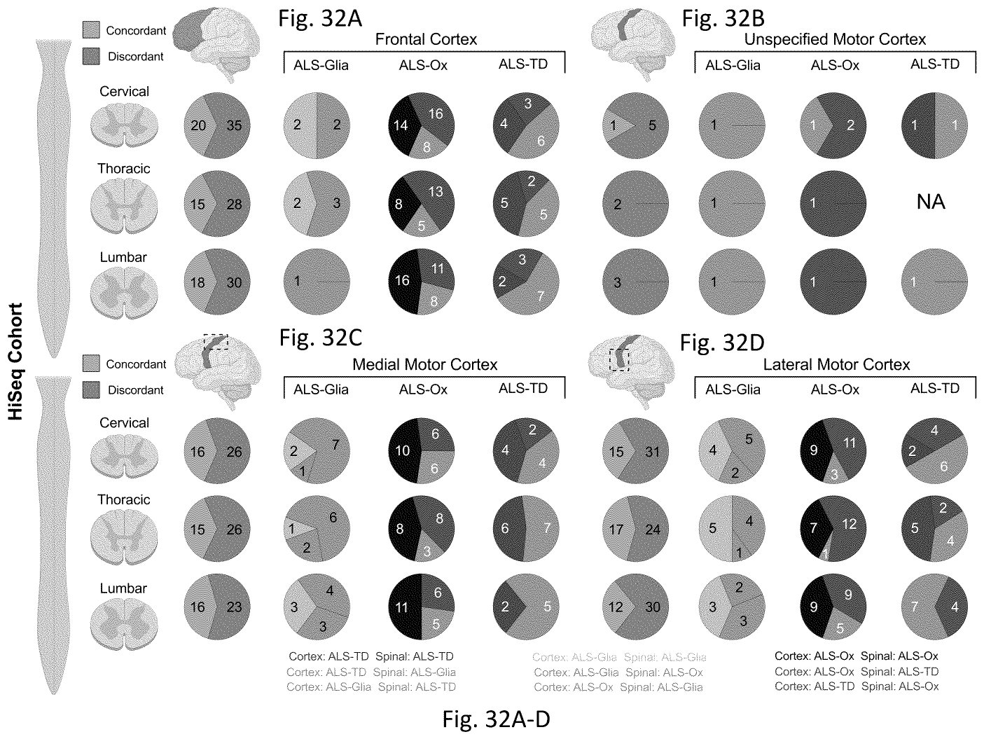

Next, the concordance analysis was extended by further separating patients by sequencing platform to assess dependence on instrumentation (FIGS. 26, 31, and 32). Results showed that the NovaSeq 6000 sequencing platform outperforms the HiSeq 2500 sequencing platform in the cervical (41.9% vs 34.9%) and lumbar spinal cord (49.6% vs 34.8%) but not the thoracic spinal cord (25.0% vs 37.0%). In the NovaSeq cohort, the highest concordance for ALS-Glia remains the same tissue pairing at 52.2%, and for ALS-Ox the highest agreement was seen between the lateral motor cortex and lumbar spinal cord at 48.4% (excluding the unspecified motor cortex and pairings with a single observation)—although lower sample numbers may partially explain these differences. Collectively this analysis shows weak to moderate agreement in subtype presented throughout the ALS postmortem cortex and spinal cord, despite differences in cell type composition, after removal of covariate-dependent genes. Results further showed concordance between the cortex and spinal cord phenotype is dependent on tissue and sequencing platform and find the lumbar region of the spinal cord shows the highest overall concordance with the cortical phenotype in this cohort.

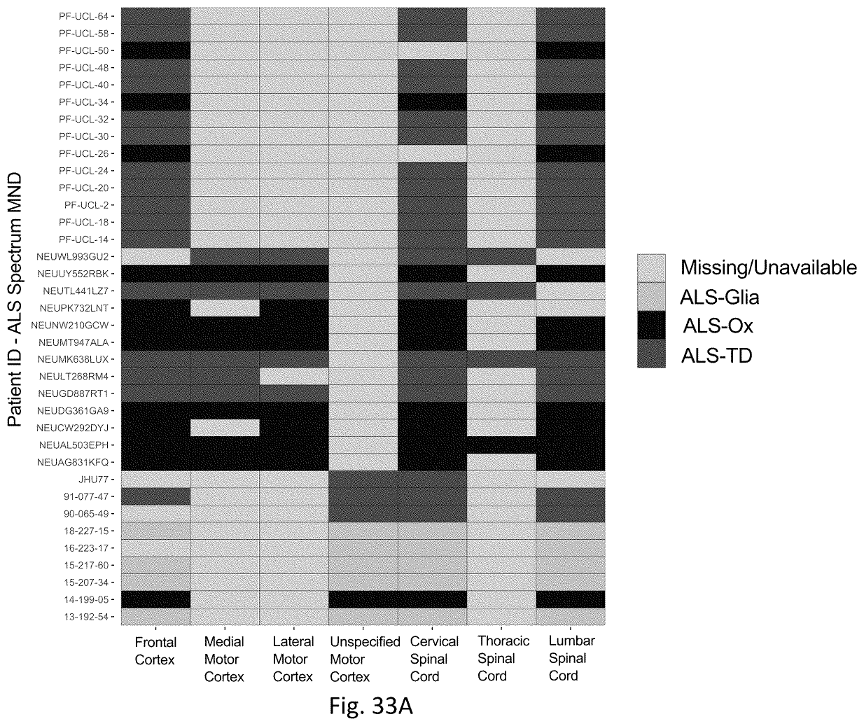

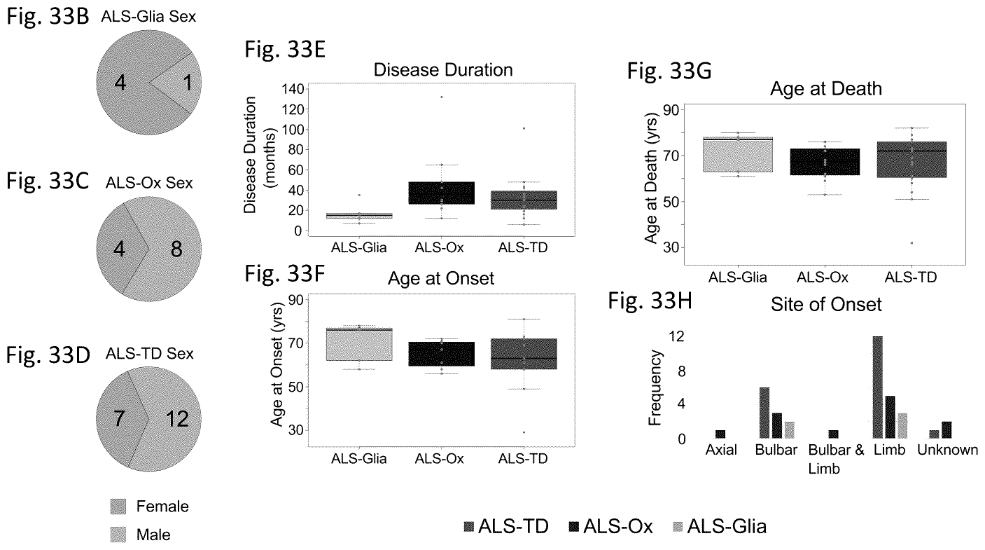

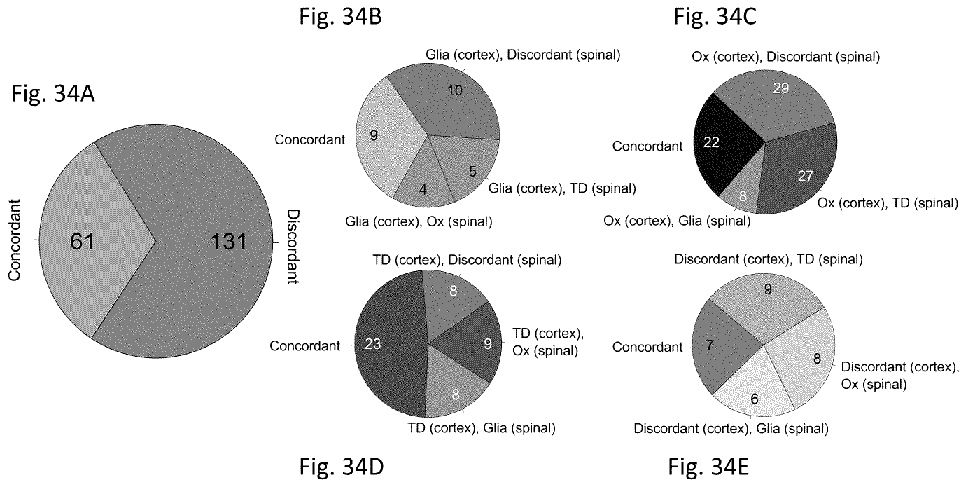

The patient concordance was further considered by screening for patients assigned the same subtype in every sample considered in this study and the previous (Eshima, J. et al. 2023. Nature Communications, 14, 95). Results showed that a total of 45 patients pass this criterion (45/222; 20.3%) and filtering these patients further to shows that 5 ALS-Glia patients, 12 ALS-Ox patients, and 19 ALS-TD patients coherently assigned a single subtype in both the cortex and spinal cord (FIG. 33). While the lower patient number limits the extrapolation of these findings, results showed a surprising association with sex and a stark difference in disease duration in this concordant patient subset (FIG. 33). Despite the statistical limitations of assigning patient subtype using a majority agreement between samples, concordance was considered between the postmortem cortex and spinal cord using this method. Results showed the majority of patients are discordant (68.2%), which likely reflects differences in cell type composition between the cortex and spinal cord, and further highlights the challenges of linking patient phenotype between the two regions (FIG. 34).

### Differential Expression Reveals Transcripts B4GALT6, GABRA1, GAD2, GLRA3, HTR2A, PCSK1, and SLC17A6 are Elevated in the ALS-Ox Cortex and Spinal Cord

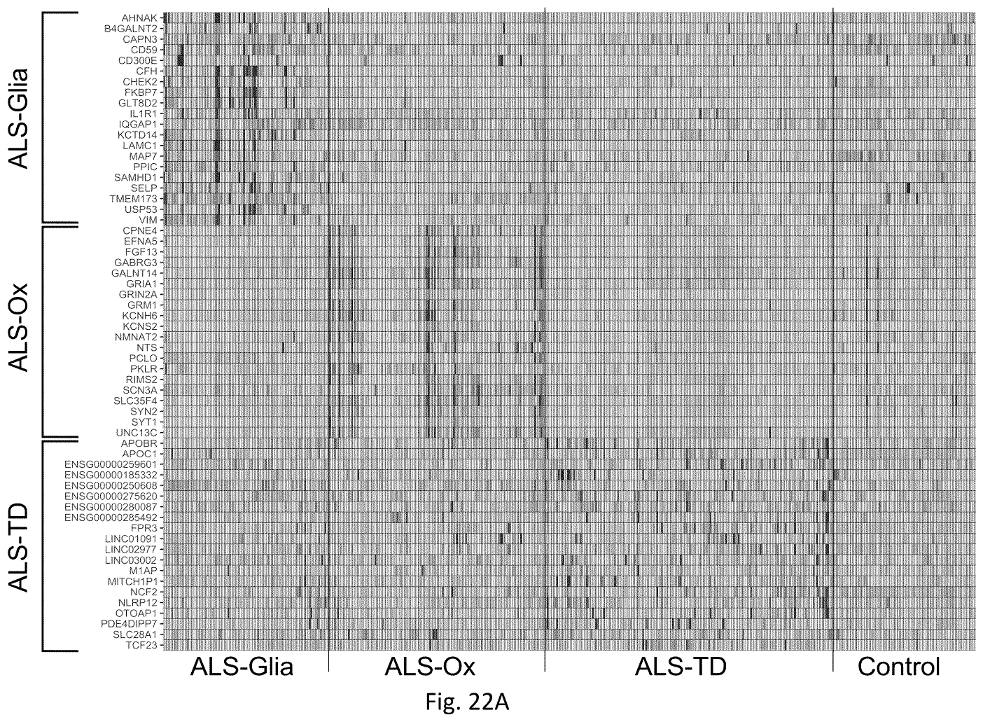

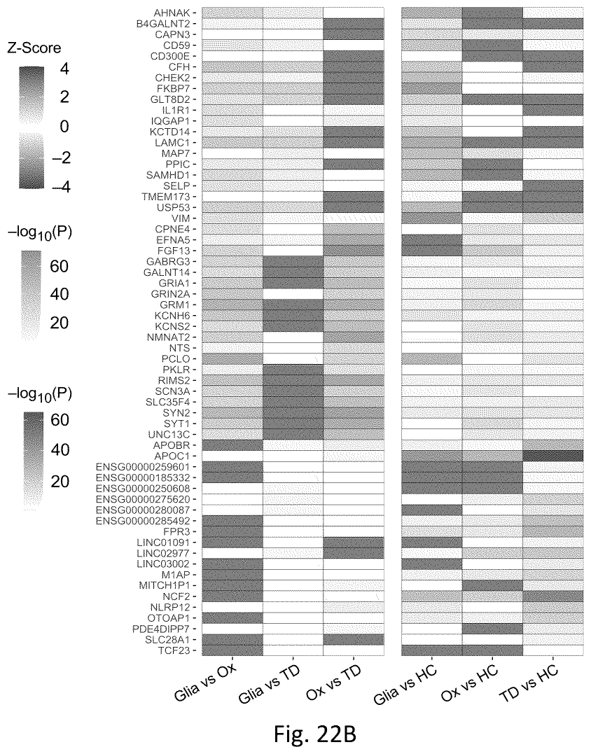

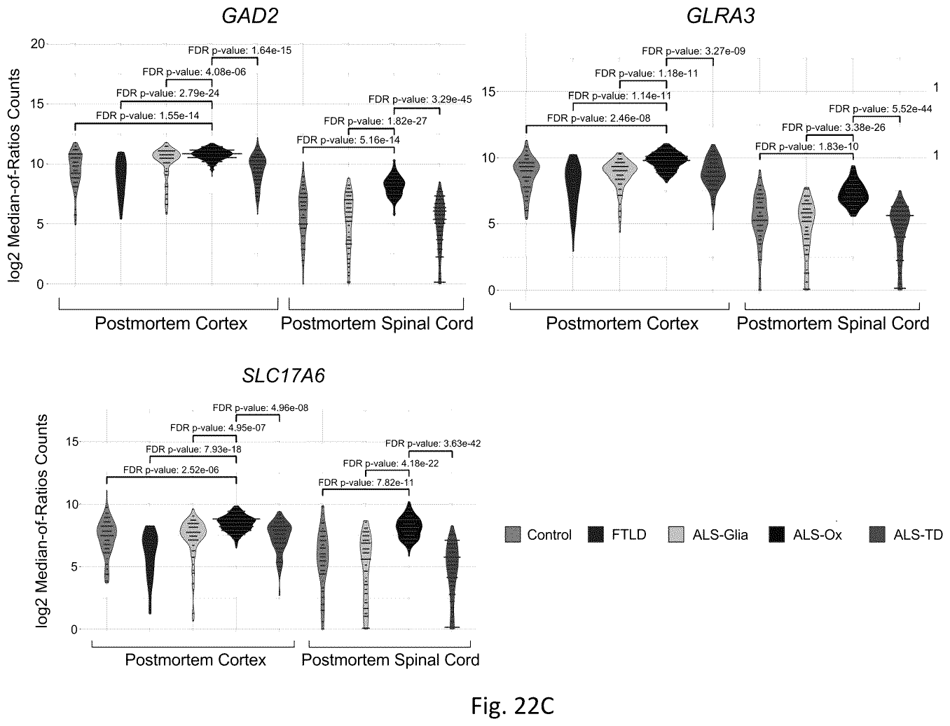

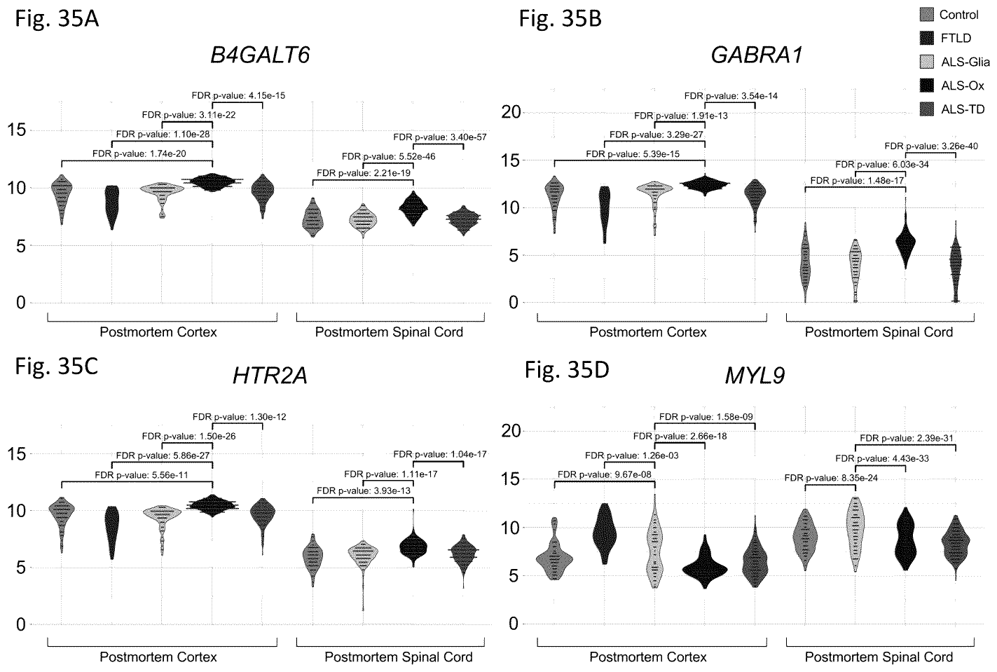

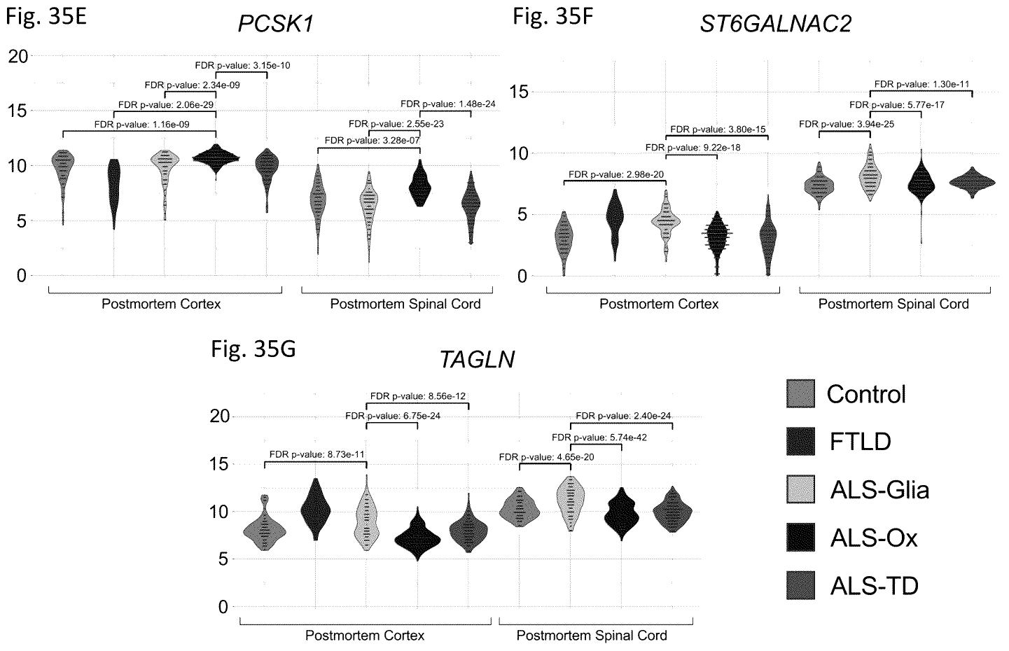

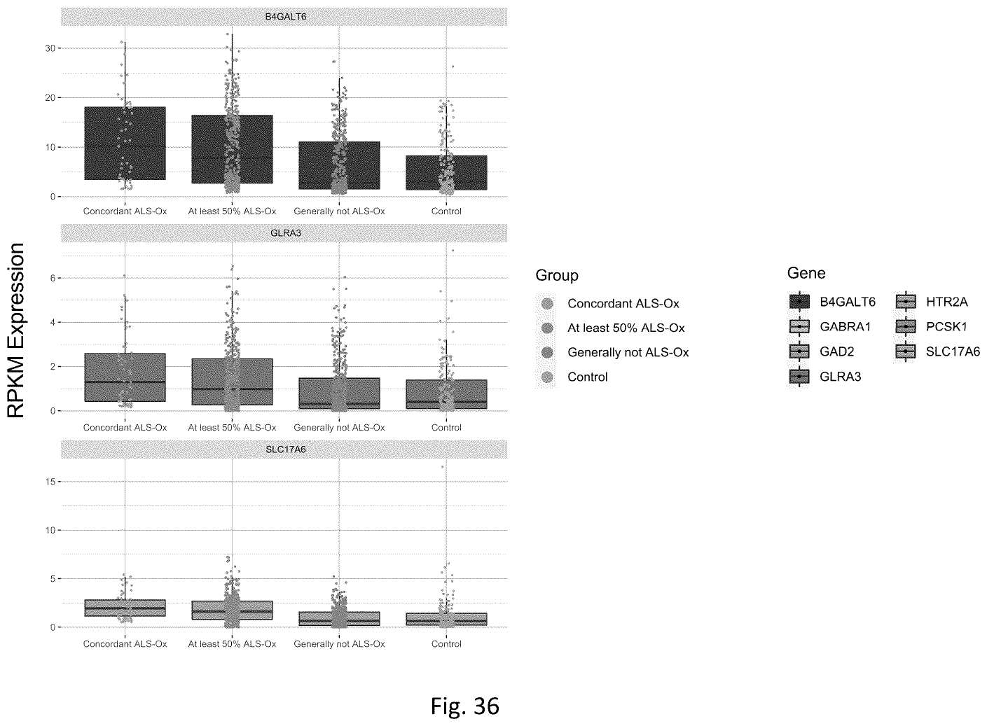

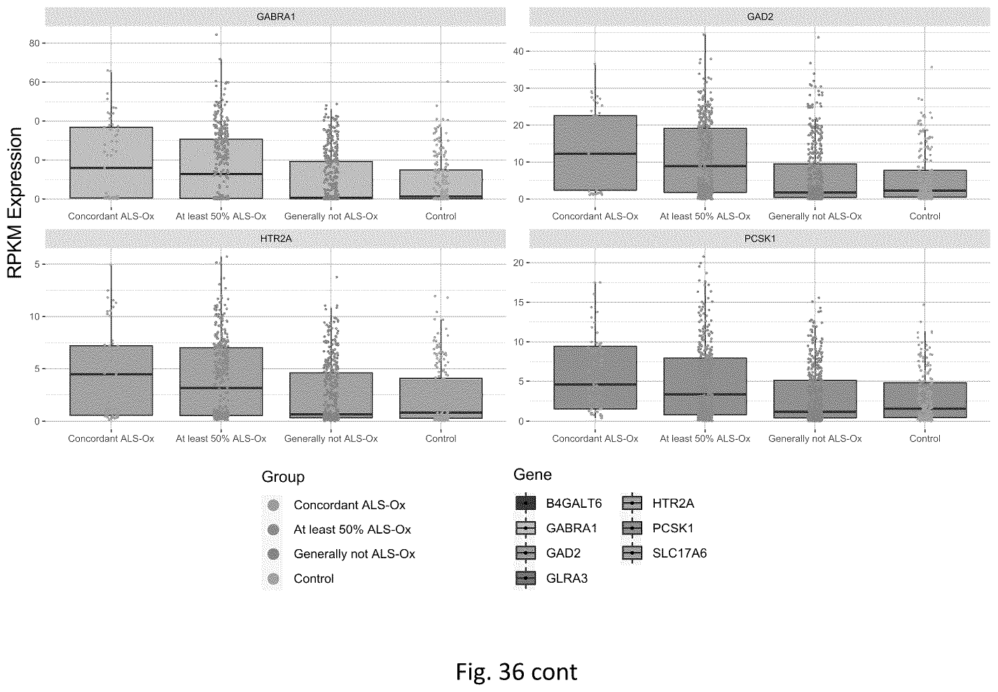

Differential expression (Love, M. I., Huber, W. & Anders, S. 2014. Genome Biol. 15, 1-21) was applied to identify subtype-specific transcript expression in the full spinal cord cohort. After adjusting for sex, site of collection, RIN, tissue, and sequencing platform covariates, results showed transcript expression uniquely defines each subtype regardless of analytical platform (FIG. 22A). Differential expression p-values, after FDR adjustment and—log 10 transformation, are presented as heatmaps using pairwise comparisons for all group combinations (FIG. 22B). Expression of transcripts stratifying ALS-Glia and ALS-TD subtypes in the spinal cord is weaker, evident in the heatmap and the differential expression p-values relative to the cortex (Eshima, J. et al. 2023. Nature Communications, 14, 95), and likely reflects differences in cell type composition in the spinal cord relative to the cortex (Eshima, J. et al. 2023. Nature Communications, 14, 95) (FIG. 27E). Most notably, results showed a total of ten transcripts with consistently elevated expression, irrespective of tissue region in the postmortem ALS central nervous system, relative to the other subtypes and non-neurological controls in this cohort (FIG. 22C and FIG. 35). Seven of these transcripts were specific for the ALS-Ox subtype, while the remaining three were specific for ALS-Glia. A total of 1,104 unique transcriptomes were considered, from 5 distinct regions of the central nervous system, corresponding to 222 ALS patients, 88 non-neurological controls, and 42 frontotemporal dementia (FTLD) patients. ALS-Ox marker genes GABRA1, GAD2, GLRA3, HTR2A, PCSK1, and SLC17A6 collectively implicate changes to synaptic signaling, with elevation of inhibitory receptors and enzymes involved in the biosynthesis of inhibitory neurotransmitters. Upregulation of B4GALT6 in ALS-Glia samples and ST6GALNAC2 in ALS-Ox suggests protein glycosylation may play a surprising role in the progression of ALS phenotype. Marker gene expression was further considered following RPKM normalization in a refined set of ALS patients with observations available from both the postmortem cortex and spinal cord (n=206 ALS patients, 88 non-neurological controls) (FIG. 36). ALS patient samples were binned into one of three categories in an effort to capture the spectrum of phenotypes typically observed in most patients, which included: “Concordant ALS-Ox” (100% of tissue samples are ALS-Ox), “At least 50% ALS-Ox”, and “Generally not ALS-Ox” (<50% of tissue samples are ALS-Ox). All ALS-Ox marker genes show a decreasing trend in the median expression as the percentage of patient samples defined as ALS-Ox decreases. Further, patients that generally do not present as ALS-Ox demonstrate similar marker gene expression as non-neurological controls. Collectively, these findings show the ALS-Ox marker genes defined in this work provide a foundation to stratify ALS patients despite the weak to moderate intra-patient concordance observed.

As may be expected, expression of the subtype marker genes was generally different in the cortex and spinal cord regions. Notably, results showed that these genes better stratify this patient cohort when considering spinal cord expression, evident in the FDR-adjusted p-values (FIG. 22C and FIG. 35), offering promise for clinical translation. Yet, modest concordance between the two regions may limit the practicality of patient subtyping using these marker transcripts. However, considering the influence of technical covariates, concordance for ALS-Ox patients between the “unspecified motor cortex” (85% of samples with NYGC as the site of collection) and lumbar spinal cord, excluding a single HiSeq pairing, reaches 66.7% (10/15) with all subtypes showing >55% agreement in at least one region of the cortex when paired with the lumbar spinal cord in the NovaSeq cohort (FIGS. 31, 27, 31, and 32).

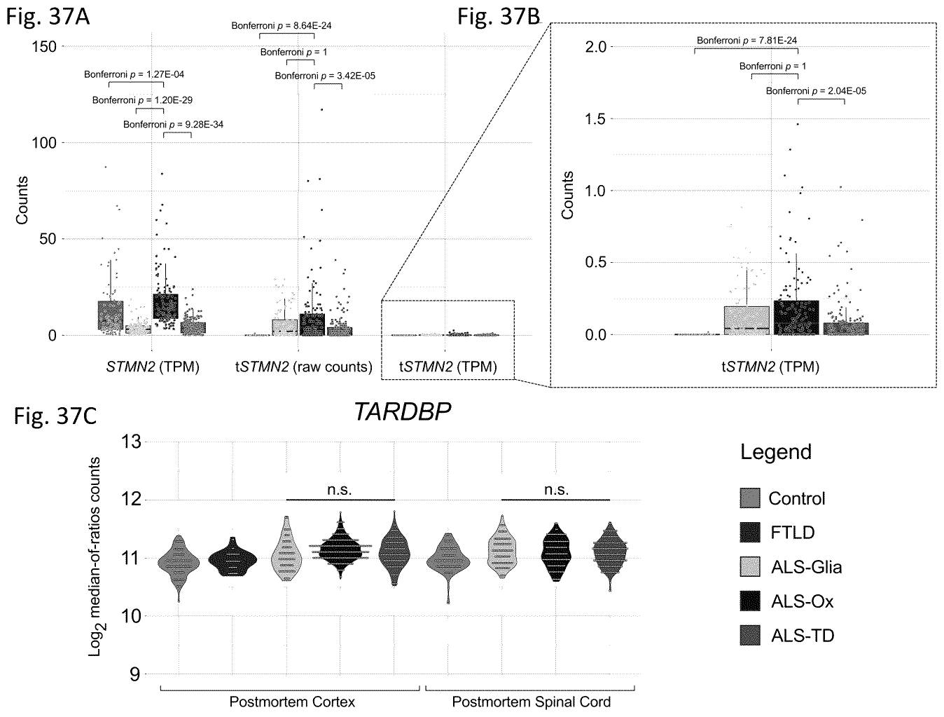

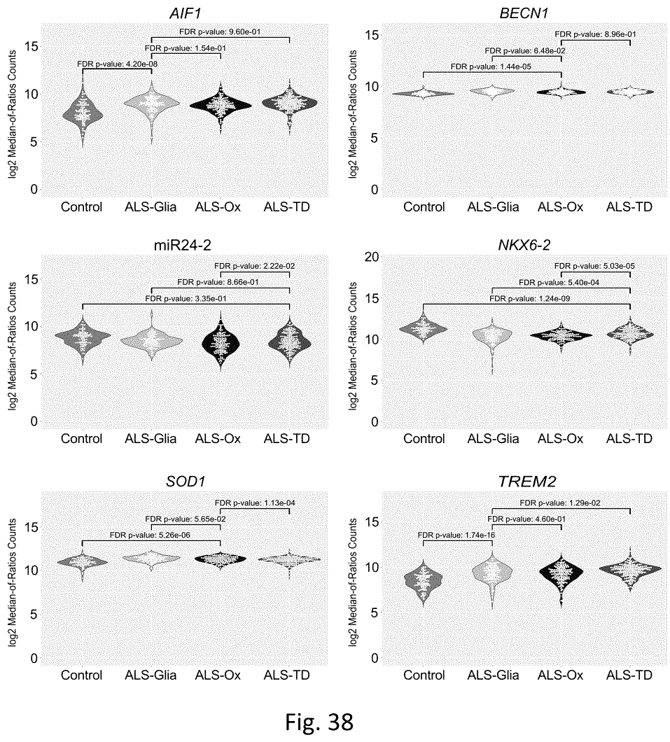

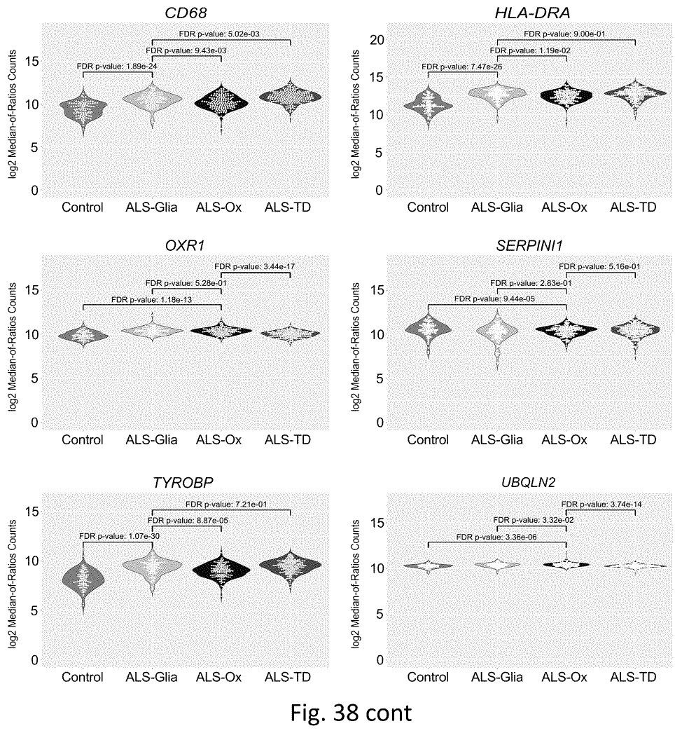

The differential expression analysis was extended by considering other relevant transcripts, including those found to stratify this cohort when considering postmortem cortex transcriptomes (Eshima, J. et al. 2023. Nature Communications, 14, 95). In the ALS-Ox spinal cord, results showed statistically significant upregulation of the STMN2 transcript and the truncated pathological form associated with TDP-43 cryptic exon splicing when compared to the ALS-TD subtype, previously determined by Prudencio et al (Prudencio, M. et al. 2020. J. Clin. Investig. 130, e13974) (FIG. 37A-B). No significant differences in TARDBP, encoding TDP-43, were observed between the ALS subtypes in either the postmortem cortex or the spinal cord (FIG. 37C). Neuroinflammatory genes AIF1, CD68, HLA-DRA, TREM2, and TYROBP were among the most elevated transcripts in the cortex of ALS-Glia patients (Eshima, J. et al. 2023. Nature Communications, 14, 95) but not the spinal cord, likely reflecting regional differences in cell type populations (FIG. 37E and FIG. 38). Further these transcripts were included in the 1282 glial marker genes removed prior to clustering-which may partially explain the similar expression of these transcripts in ALS-Glia and ALS-TD subtypes. Oxidative and proteotoxic stress genes BECN1, OXR1, SERPINI1, SOD1, and UBQLN2 generally show weaker differences in spinal cord expression when compared to the other two subtypes (FIG. 38). Similarly, transcriptional regulators miR24-2 and NKX6-2 show specificity for the postmortem cortex, although NKX6-2 expression is most elevated in the spinal cord of ALS-TD patients, relative to the other two subtypes (FIG. 38).

### Translation of ALS-Ox Marker Genes

ALS-Ox marker genes B4GALT6, GABRA1, GAD2, GLRA3, HTR2A, PCSK1, and SLC17A6 (FIG. 22C, FIG. 35) were utilized develop multiple classifiers of varying complexity (FIGS. 23, 39, 40, and 41). In each case, classifier performance was assessed using RPKM normalized expression, an 80/20 train-test split, 100-fold cross validation, two classes (“Ox” and “NotOx”), and three different holdout (validation) cohorts comprised of all (i) postmortem spinal cord samples (ii) postmortem cortex samples and (iii) HiSeq samples. The first holdout cohort estimates predictive accuracy when assigning spinal cord subtype using cortex expression, while the opposite is true in the second holdout cohort. The final holdout cohort is designed to better estimate predictive accuracy when applied to new patient cohorts accounting for instrument-dependent expression. While it may be reasonable to assume that predicting the cortex phenotype using spinal cord expression is more clinically useful as it limits diagnostic invasiveness, models were constructed for both cases to demonstrate the capability of these subtype-specific transcripts to stratify the cohort regardless of region-dependent expression differences.

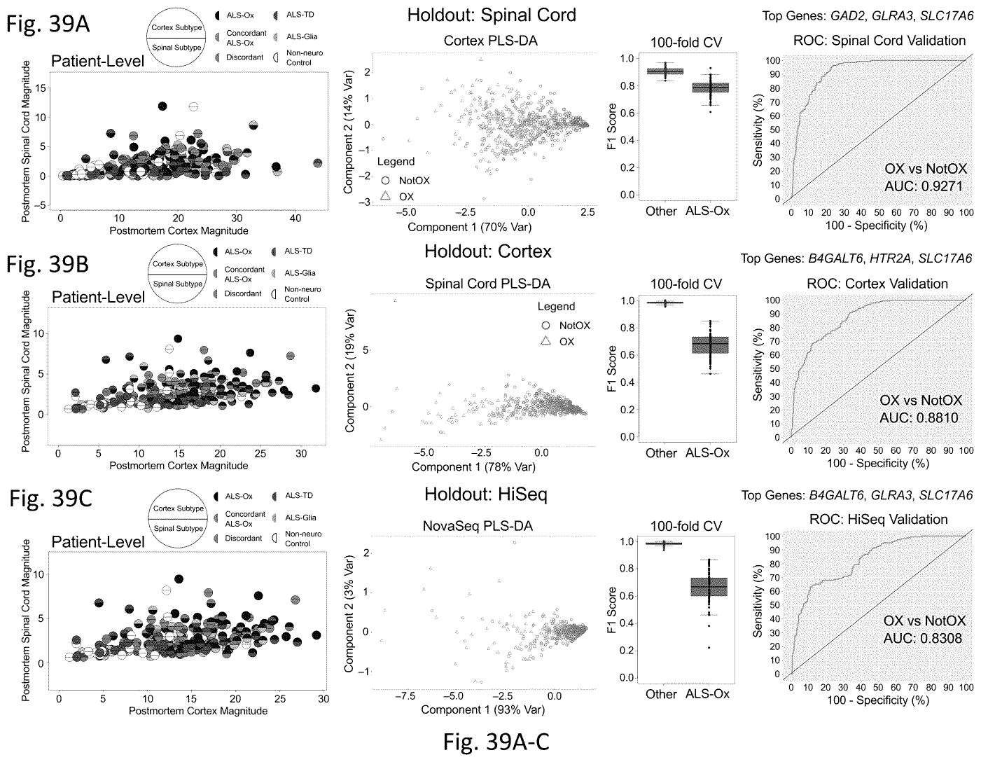

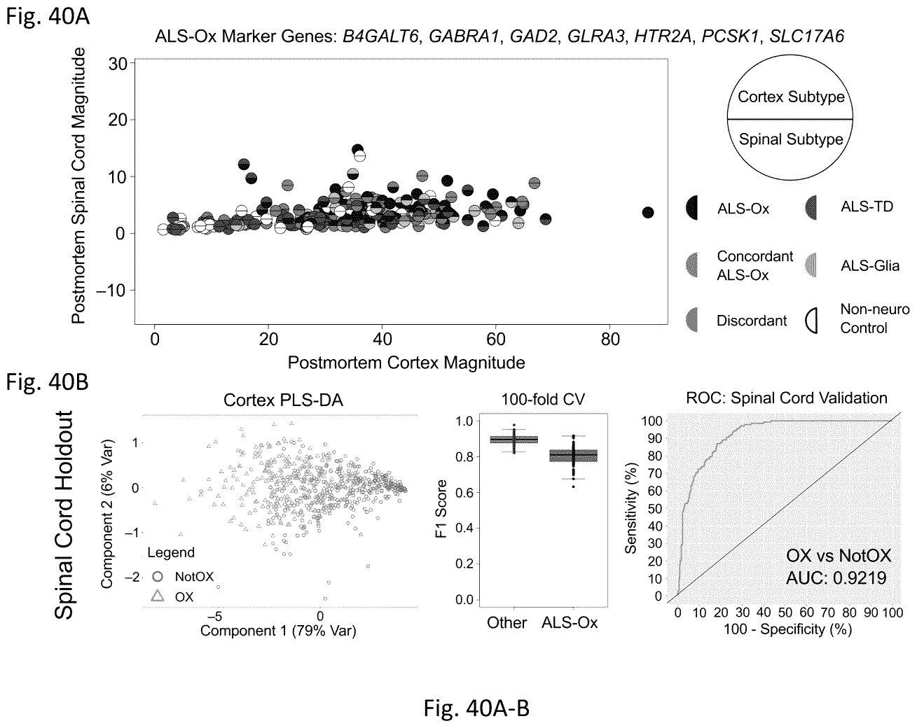

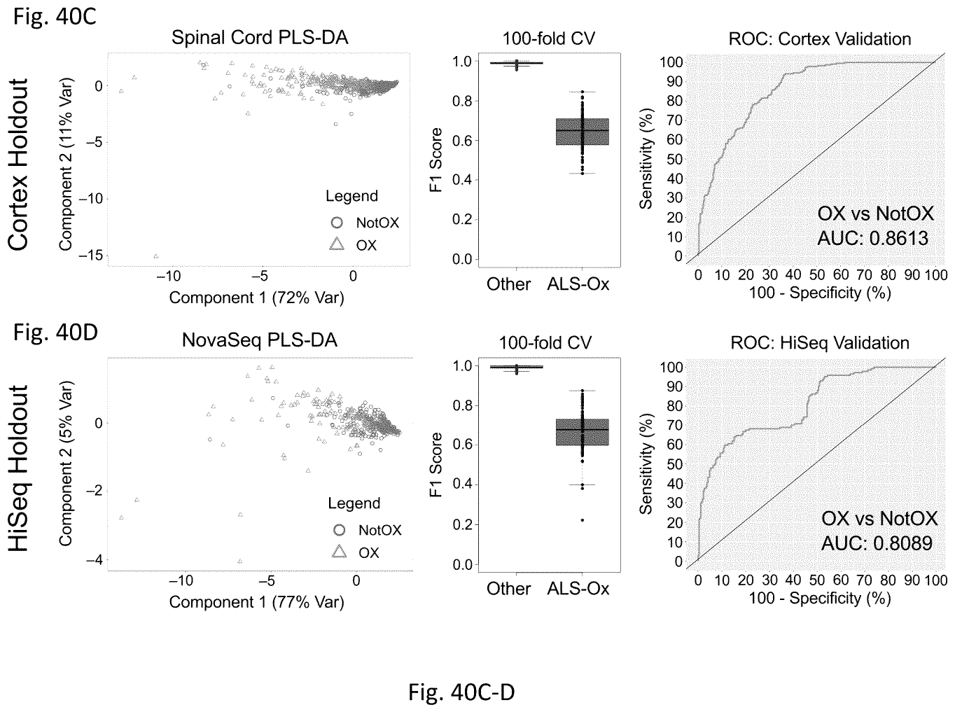

With the aim of reducing clinical diagnostic burden, classifiers were constructed from all three-gene combinations of ALS-Ox marker genes using partial least squares discriminate analysis (Rohart F. et al. 2017. PLOS computational biology. 13 (11): e1005752) (PLS-DA). After training and testing these classifiers, results showed that the three-gene combination of GAD2, GLRA3, and SLC17A6 slightly outperforms other gene combinations when predicting subtype in the spinal cord validation cohort (AUC=0.927) (FIG. 39A). Conversely, when training on the spinal cord cohort, HTR2A, SLC17A6, and B4GALT6 gene set showed the highest predictive accuracy after application to the cortex validation cohort (AUC=0.881) (FIG. 39B). In the HiSeq validation cohort, B4GALT6, GLRA3, SLC17A6, and demonstrated the highest predictive accuracy (AUC=0.831), suggesting these transcripts may be more invariant to differences in sample preparation and instrumentation (FIG. 39C). Furthermore, the same gene combination of B4GALT6, GLRA3, and SLC17A6 demonstrated the highest average AUC across all three validation cohorts (AUC=0.873), indicating these genes may be most robust for assigning ALS-Ox patient subtype. When compared to the PLS-DA classifier using all seven ALS-Ox marker genes (FIG. 40A), a decrease in predictive power is observed in the spinal cord holdout (AUC=0.922) (FIG. 40B), the cortex holdout (AUC=0.861) (FIG. 40C), and the HiSeq holdout (AUC=0.809) (FIG. 40D).

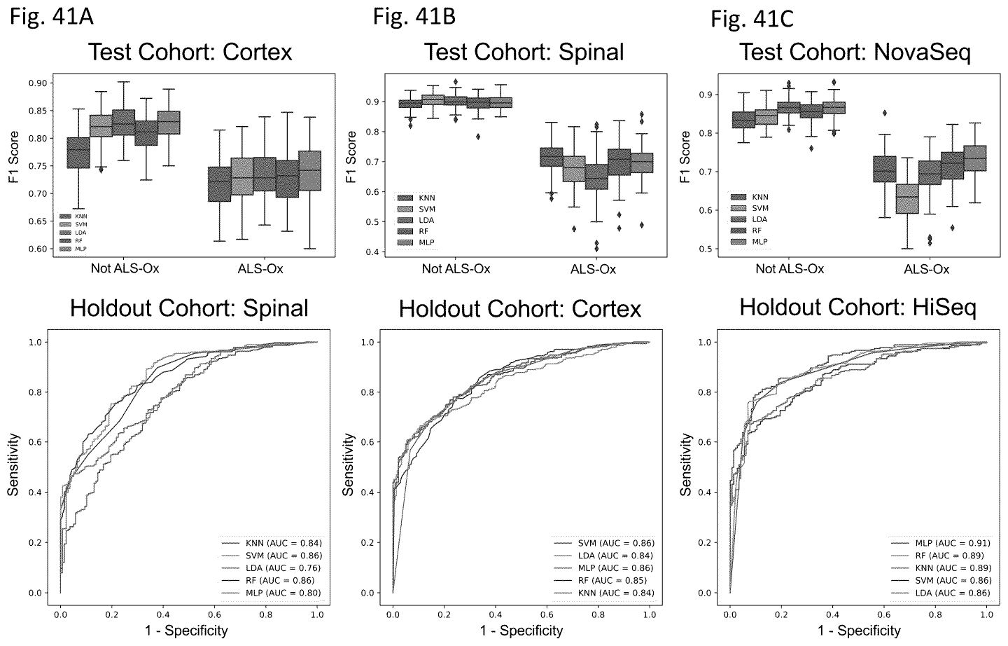

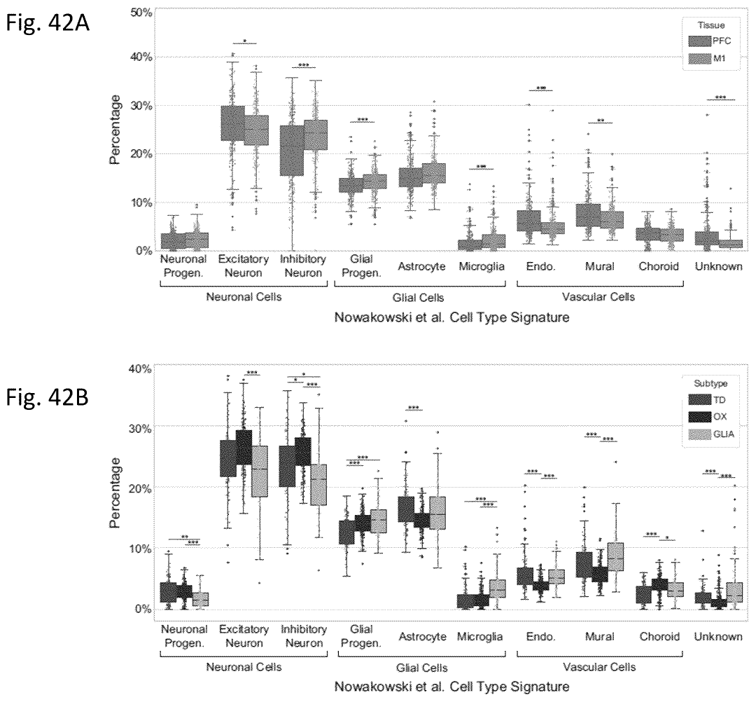

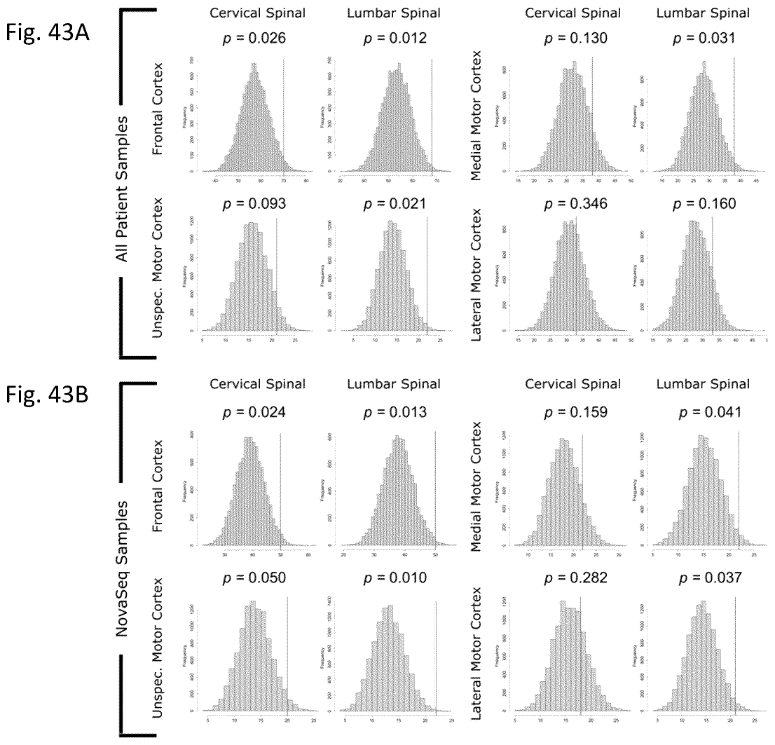

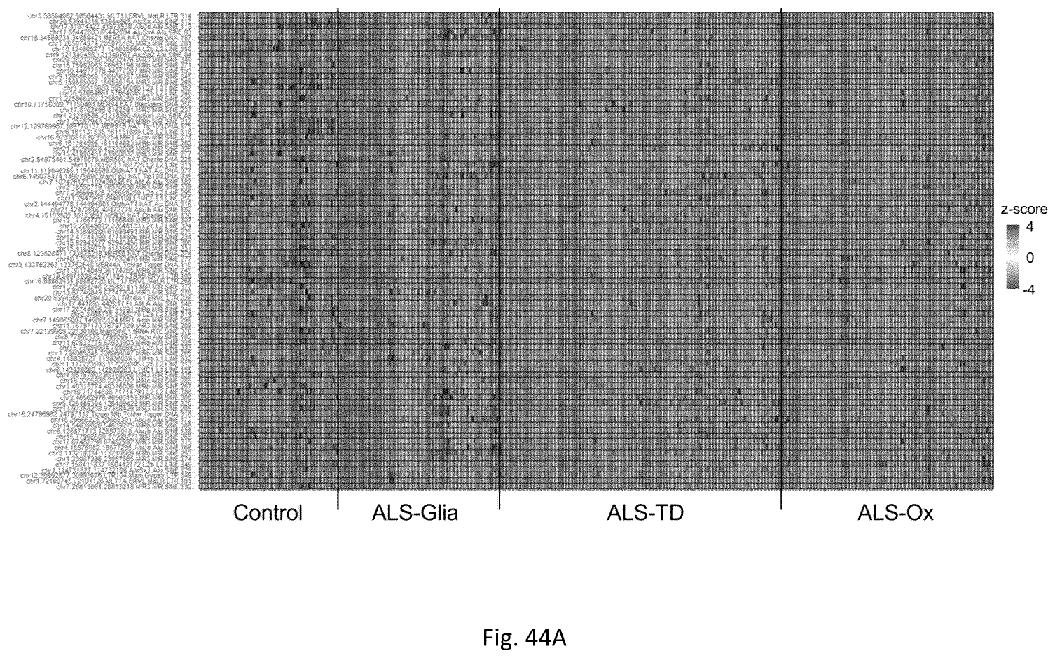

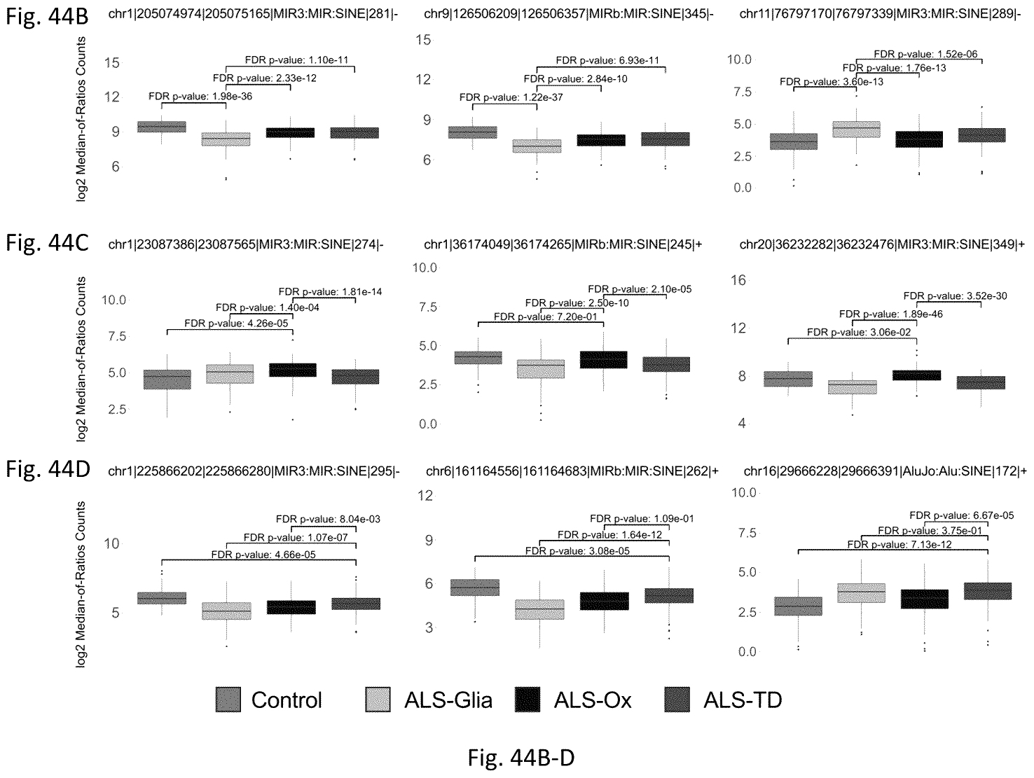

Building on promising results from PLS-DA, this analysis was extended by performing supervised machine learning using k-nearest neighbor (KNN), linear discriminant analysis (LDA), random forest (RF), support vector machine classifier (SVM), and multilayer perceptron (MLP) classification frameworks (Pedregosa, F. et al. 2011. J. Mach. Learn. Res. 12, 2825-2830). Classifiers were constructed using RPKM normalized expression of (i) the best three gene combination from PLS-DA (B4GALT6, GLRA3, SLC17A6) (FIG. 23) and (ii) all seven ALS-Ox marker genes (FIG. 41). Using the top three discriminatory genes, the SVM classifier demonstrates the highest overall predictive accuracy when stratifying ALS-Ox and “not ALS-Ox” with median F1 scores from the test cohort ranging between 0.62-0.73 for ALS-Ox and 0.83-0.90 for ‘not ALS-Ox’, and holdout cohort AUCs ranging from 0.86-0.89 (FIG. 23). Similar performance is observed in the MLP classifier. In agreement with results observed during PLS-DA, the seven gene classifier generally demonstrated worse predictive accuracy in the cortex (AUCs=0.84-0.86) and spinal cord (AUCs=0.76-0.86) holdout cohorts (FIGS. 41A and 41B). However, improved predictive accuracy was seen when the seven-gene classifiers were applied to the HiSeq holdout cohort, with AUCs ranging from 0.86-0.91 (FIG. 41C), suggesting the seven-gene classifier may outperform the three-gene as the ‘strength’ of batch effects and confounding covariates increases. Collectively, these classification results demonstrate that the set of ALS-Ox marker genes established in this work can achieve appreciable stratification accuracy when predicting patient phenotype between regions of the central nervous system—with different cell type composition—or when using different instrumentation for quantification.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of diagnosing a subject as having ALS or a specific ALS subtype, or an increased or decreased risk of ALS or a specific ALS subtype the method comprising:
a) detecting the level or activity of at least two biomarkers selected from the biomarkers listed in Table 3 in a sample from the subject;
b) comparing the level or activity of the biomarker in the sample to the level or activity of the biomarker in a comparator control; and
c) diagnosing the subject as having ALS or a specific ALS subtype when the level or activity of the biomarker is significantly increased or decreased as compared to the comparator control.

2. The method of claim 1, wherein the subtype of ALS is selected from the group consisting of a subset of ALS associated with activated glial cells (ALS-Glia), a subset of ALS associated with oxidative stress (ALS-Ox) and a subset of ALS associated with transcriptional dysregulation (ALS-TD).

3. The method of claim 2, wherein the subtype of ALS is ALS-Glia, and further wherein the subject is diagnosed with ALS-Glia when the level or activity of one or more of APOBR and TNFRSF25 is decreased as compared to the comparator control.

4. The method of claim 2, wherein the subtype of ALS is ALS-Glia, and further wherein the subject is diagnosed with ALS-Glia when the level or activity of one or more of AIF1, APOC2, CD44, CHI3L2, CX3CR1, FOLH1, HLA-DRA, TLR7, TNC, TREM2, TYROBP, ALOX5AP, APOC1, CCR5, CD68, CLEC7A, CR1, MSR1, MYL9, NCF2, NINJ2, ST6GALNAC2, TAGLN, TLR8 or VRK2 is increased as compared to the comparator control.

5. The method of claim 2, wherein the subtype of ALS is ALS-Ox, and further wherein the subject is diagnosed with ALS-Ox when the level or activity of one or more of COL18A1, SLC6A13, TCIRG1, CP, NDUFA4L2, NOS3, NOTCH3, and TAGLN is decreased as compared to the comparator control.

6. The method of claim 2, wherein the subtype of ALS is ALS-Ox, and further wherein the subject is diagnosed with ALS-Ox when the level or activity of one or more of GABRA1, GAD2, GLRA3, HTR2A, OXR1, SERPINI1, SLC17A6, UBQLN2, B4GALT6, BECN1, GABRA6, GPR22, PCSK1, and UBQLN1 is increased as compared to the comparator control.

7. The method of claim 2, wherein the subtype of ALS is ALS-TD, and further wherein the subject is diagnosed with ALS-TD when the level or activity of one or more of COL3A1, ENSG00000273151, MIRLET7BHG, and TUB-AS1 is decreased as compared to the comparator control.

8. The method of claim 2, wherein the subtype of ALS is ALS-TD, and further wherein the subject is diagnosed with ALS-TD when the level or activity of one or more of AGPAT4-IT1, CHKB-CPT1B, ENSG00000205041, ENSG00000258674, HSP90AB4P, LINC01347, MIR24-2, ADAT3, EGLNIP1, ENSG00000263278, ENSG00000268670, ENSG00000279233, LINC00176, MIR219A2, RPS20P22, and SLX1B-SULT1A4 is increased as compared to the comparator control.

9. The method of claim 1, further comprising a step of administering a therapeutic agent for the treatment of the diagnosed ALS or ALS-subtype.

10. The method of claim 9, wherein the treatment comprises administering a modulator of one or more biomarker of Table 3.

11. The method of claim 10, wherein the modulator is selected from the group consisting of a nucleic acid, a peptide, a small molecule chemical compound, an siRNA, a ribozyme, an antisense nucleic acid, an aptamer, a peptidomimetic, an antibody, an antibody fragment.

12. The method of claim 11, wherein the subtype of ALS is ALS-Glia, and further wherein the subject diagnosed with ALS-Glia is administered an activator of one or more of APOBR and TNFRSF25.

13. The method of claim 11, wherein the subtype of ALS is ALS-Glia, and further wherein the subject diagnosed with ALS-Glia is administered an inhibitor of one or more of AIF1, APOC2, CD44, CHI3L2, CX3CR1, FOLH1, HLA-DRA, TLR7, TNC, TREM2, TYROBP, ALOX5AP, APOC1, CCR5, CD68, CLEC7A, CR1, MSR1, MYL9, NCF2, NINJ2, ST6GALNAC2, TAGLN, TLR8 or VRK2.

14. The method of claim 11, wherein the subtype of ALS is ALS-Ox, and further wherein the subject diagnosed with ALS-Ox is administered an activator of one or more of COL18A1, SLC6A13, TCIRG1, CP, NDUFA4L2, NOS3, NOTCH3, and TAGLN.

15. The method of claim 11, wherein the subtype of ALS is ALS-Ox, and further wherein the subject diagnosed with ALS-Ox is administered an inhibitor of one or more of GABRA1, GAD2, GLRA3, HTR2A, OXR1, SERPINI1, SLC17A6, UBQLN2, B4GALT6, BECN1, GABRA6, GPR22, PCSK1, and UBQLN1.

16. The method of claim 11, wherein the subtype of ALS is ALS-TD, and further wherein the subject diagnosed with ALS-TD is administered an activator of one or more of COL3A1, ENSG00000273151, MIRLET7BHG, and TUB-AS1.

17. The method of claim 2, wherein the subtype of ALS is ALS-TD, and further wherein the subject diagnosed with ALS-TD is administered an inhibitor of one or more of AGPAT4-IT1, CHKB-CPT1B, ENSG00000205041, ENSG00000258674, HSP90AB4P, LINC01347, MIR24-2, ADAT3, EGLN1P1, ENSG00000263278, ENSG00000268670, ENSG00000279233, LINC00176, MIR219A2, RPS20P22, and SLX1B-SULT1A4.

18. A method of differentially diagnosing a subject as having a specific subtype of ALS, or providing a prognosis to a subject diagnosed with ALS, the method comprising
a) detecting the level or activity of at least two biomarkers selected from the group consisting of: one or more transposable element, APOBR, APOC1, and APOC2 in a sample from the subject;
b) comparing the level or activity of the biomarker in the sample to the level or activity of the biomarker in a comparator control; and
c) diagnosing the subject as having ALS-Glia, or a poorer prognosis when the level of APOBR, APOC1, and APOC2 is increased in the sample as compared to the comparator control or diagnosing the subject as having ALS-Ox or ALS-TD, or a better prognosis when the activation of one or more transposable element is significantly increased as compared to the comparator control.

19. The method of claim 18, further comprising a step of administering a therapeutic agent for the treatment of the diagnosed ALS-subtype.

20. A method of differentially diagnosing a subject as having ALS or frontotemporal lobar degeneration (FTLD), the method comprising
a) detecting the level or activity of STH in a sample from the subject;
b) comparing the level or activity of STH in the sample to the level or activity of STH in a comparator control; and
c) diagnosing the subject as having ALS when the level of STH is decreased in the sample as compared to the comparator control or diagnosing the subject as having FTLD when the level of STH increased as compared to the comparator control.
